SEQUENCE LISTING (902 sequences)

SEQ ID NO: 1 - DNA, 73 bp, Artificial Sequence
  Description of Artificial Sequence Synthetic oligonucleotide
gggccagtggcgcaatggataacgcgtctgacttcagatcagaagattccaggttcgactcctggctggctcg

SEQ ID NO: 2 - DNA, 73 bp, Artificial Sequence
  Description of Artificial Sequence Synthetic oligonucleotide
gggccagtggcgcaatggataacgcgtctgacttcagatcagaagattgtaggttcgactcctacctggctcg

SEQ ID NO: 3 - DNA, 73 bp, Artificial Sequence
  Description of Artificial Sequence Synthetic oligonucleotide
gggccagtggcgcaatggataacgcgtctgacttcagatcagaagattctaggttcgactcctggctggctcg

SEQ ID NO: 4 - DNA, 73 bp, Artificial Sequence
  Description of Artificial Sequence Synthetic oligonucleotide
ggccgcgtggcctaatggataaggcgtctgatttcagatcagaagattgagggttcgagtcccttcgtggtcg

SEQ ID NO: 5 - DNA, 73 bp, Artificial Sequence
  Description of Artificial Sequence Synthetic oligonucleotide
ggccgcgtggcctaatggataaggcgtctgatttcagatcagaagattgggggttcgagtcccttcgtggtcg

SEQ ID NO: 6 - DNA, 73 bp, Artificial Sequence
  Description of Artificial Sequence Synthetic oligonucleotide
gacccagtggcctaatggataaggcatcagccttcagagctggggattgtgggttcgagtcccatctgggtcg

SEQ ID NO: 7 - DNA, 73 bp, Artificial Sequence
  Description of Artificial Sequence Synthetic oligonucleotide
gccccagtggcctaatggataaggcactggccttcaaagccagggattgtgggttcgagtcccacctggggta

SEQ ID NO: 8 - DNA, 73 bp, Artificial Sequence
  Description of Artificial Sequence Synthetic oligonucleotide
gccccagtggcctaatggataaggcactggccttcaaagccagggattgtgggttcgagtcccacctggggtg

SEQ ID NO: 9 - DNA, 73 bp, Artificial Sequence
  Description of Artificial Sequence Synthetic oligonucleotide
gccccggtggcctaatggataaggcattggccttcaaagccagggattgtgggttcgagtcccacccggggta

SEQ ID NO: 10 - DNA, 73 bp, Artificial Sequence
  Description of Artificial Sequence Synthetic oligonucleotide
gccccagtggcctaatggataaggcattggccttcaaagccagggattgtgggttcgagtcccatctggggtg

SEQ ID NO: 11 - DNA, 73 bp, Artificial Sequence
  Description of Artificial Sequence Synthetic oligonucleotide
ggccgcgtggcctaatggataaggcgtctgacttcagatcagaagattgcaggttcgagtcctgccgcggtcg

SEQ ID NO: 12 - DNA, 73 bp, Artificial Sequence
  Description of Artificial Sequence Synthetic oligonucleotide
gaccgcgtggcctaatggataaggcgtctgacttcagatcagaagattgagggttcgagtcccttcgtggtcg

SEQ ID NO: 13 - DNA, 73 bp, Artificial Sequence
  Description of Artificial Sequence Synthetic oligonucleotide
gaccacgtggcctaatggataaggcgtctgacttcagatcagaagattgagggttcgaatcccttcgtggttg

SEQ ID NO: 14 - DNA, 73 bp, Artificial Sequence
  Description of Artificial Sequence Synthetic oligonucleotide
gaccacgtggcctaacggataaggcgtctgacttcagatcagaagattgagggttcgaatcccttcgtggtta

SEQ ID NO: 15 - DNA, 87 bp, Artificial Sequence
  Description of Artificial Sequence Synthetic oligonucleotide
ggctctgtggcgcaatggatagcgcattggacttcaagtgacgagaaagcgattcaaaggttgtgggttcgaatcccaccagagtcg

SEQ ID NO: 16 - DNA, 73 bp, Artificial Sequence
  Description of Artificial Sequence Synthetic oligonucleotide
ggctctgtggcgcaatggatagcgcattggacttcaaattcaaaggttgtgggttcgaatcccaccagagtcg

SEQ ID NO: 17 - DNA, 85 bp, Artificial Sequence
  Description of Artificial Sequence Synthetic oligonucleotide
ggctccgtggcgcaatggatagcgcattggacttcaagaggctgaaggcattcaaaggttccgggttcgagtcccggcggagtcg

SEQ ID NO: 18 - DNA, 73 bp, Artificial Sequence
  Description of Artificial Sequence Synthetic oligonucleotide
ggctccgtggcgcaatggatagcgcattggacttcaaattcaaaggttccgggttcgagtcccggcggagtcg

SEQ ID NO: 19 - DNA, 86 bp, Artificial Sequence
  Description of Artificial Sequence Synthetic oligonucleotide
ggctctgtggcgcaatggatagcgcattggacttcaagcatgattgagagattcaaaggttgcgggttcgagtcccgccagagtcg

SEQ ID NO: 20 - DNA, 73 bp, Artificial Sequence
  Description of Artificial Sequence Synthetic oligonucleotide
ggctctgtggcgcaatggatagcgcattggacttcaaattcaaaggttgcgggttcgagtcccgccagagtcg

SEQ ID NO: 21 - DNA, 86 bp, Artificial Sequence
  Description of Artificial Sequence Synthetic oligonucleotide
ggctctgtggcgcaatggatagcgcattggacttcaagacaaatggaggcattcaaaggttgtgggttcgagtcccaccagagtcg

SEQ ID NO: 22 - DNA, 73 bp, Artificial Sequence
  Description of Artificial Sequence Synthetic oligonucleotide
ggctctgtggcgcaatggatagcgcattggacttcaaattcaaaggttgtgggttcgagtcccaccagagtcg

SEQ ID NO: 23 - DNA, 74 bp, Artificial Sequence
  Description of Artificial Sequence Synthetic oligonucleotide
gtctctgtggcgcaatggacgagcgcgctggacttcaaatccagaggttctgggttcgagtcccggcagagatg

SEQ ID NO: 24 - DNA, 86 bp, Artificial Sequence
  Description of Artificial Sequence Synthetic oligonucleotide
ggctctgtggagcaatggatagcacattggacttcaagcatgaccgagagattcaaaggttgcgggttcgagtcccaccagagttg

SEQ ID NO: 25 - DNA, 73 bp, Artificial Sequence
  Description of Artificial Sequence Synthetic oligonucleotide
ggctctgtggagcaatggatagcacattggacttcaaattcaaaggttgcgggttcgagtcccaccagagttg

SEQ ID NO: 26 - DNA, 200 bp, Artificial Sequence
  Description of Artificial Sequence Synthetic polynucleotide
ctacccagaggcaggcgggagactcccccgagcgtccaataagagcgccgccaatggagccgcccgcccgcgggggtgcagagggacttccgggtgaggtcctccgctacttccctccccacggaaaagatagaccagtctgacgcgagcctgaaggcggctacacgctttaagctaagtaaaggcaccttctcgctggc

SEQ ID NO: 27 - DNA, 200 bp, Artificial Sequence
  Description of Artificial Sequence Synthetic polynucleotide
acttgtatgttgtttttatctgtcagtttgttaatcccaagattccctttggaaataaagcgaaattgaccgtagtggttatgaccaacttctagtctaaacttaattcttggaactcaaggatctgagcaaacaactgtcagggtgacacattgcttaaacggtgacagcggtcgagagccttgtcccggatggagagt

SEQ ID NO: 28 - DNA, 51 bp, Artificial Sequence
  Description of Artificial Sequence Synthetic oligonucleotide
ctgagacctctaagagccttatcttgatttgaagggatgagggtggttgtg

SEQ ID NO: 29 - DNA, 51 bp, Artificial Sequence
  Description of Artificial Sequence Synthetic oligonucleotide
acaagccttttcagctttagagggtgagcaaaggatgtgggatctgagaac

SEQ ID NO: 30 - DNA, 51 bp, Artificial Sequence
  Description of Artificial Sequence Synthetic oligonucleotide
ctaatagaaagaaatgagaccgcctgatggaaaaatgtgaaagtaaacttt

SEQ ID NO: 31 - DNA, 1854 bp, Artificial Sequence
  Description of Artificial Sequence Synthetic polynucleotide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggcccaacggtaccggatccaccggccgccaccatgggaagcccaaagaagaagcgtaaggtaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagtgaaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagccccaagaaaaagcggaaggtgtaa

SEQ ID NO: 32 - DNA, 104 bp, Artificial Sequence
  Description of Artificial Sequence Synthetic polynucleotide
acttgtatgttgtttttatctgtcagtttgttaatcccaagattccctttggaaataaagcgaaattgaccgtagtggttatgaccaacttctagtctaaactt

SEQ ID NO: 33 - DNA, 269 bp, Artificial Sequence
  Description of Artificial Sequence Synthetic polynucleotide
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgggcggaggaaggcaccttctcgctggc

SEQ ID NO: 34 - DNA, 46 bp, Artificial Sequence
  Description of Artificial Sequence Synthetic oligonucleotide
acttgtatgttgtttttatctgtcagtttgttaatcccaagattcc

SEQ ID NO: 35 - DNA, 74 bp, Artificial Sequence
  Description of Artificial Sequence Synthetic oligonucleotide
gtctctgtggcgcaatggacgagcgcgctggacttcaaatccagaggttccgggttcgagtcccggcagagatg

SEQ ID NO: 36 - DNA, 72 bp, Artificial Sequence
  Description of Artificial Sequence Synthetic oligonucleotide
ggtcccatggtgtaatggttagcactctggactttaaatccagcgatccgagttcaaatctcggtgggacct

SEQ ID NO: 37 - DNA, 72 bp, Artificial Sequence
  Description of Artificial Sequence Synthetic oligonucleotide
ggttccatggtgtaatggttagcactctggactttaaatccagcgacccgagttcaaatctcggtgggacct

SEQ ID NO: 38 - DNA, 72 bp, Artificial Sequence
  Description of Artificial Sequence Synthetic oligonucleotide
ggttccatggtgtaatggttagcactctggactttaaatccagcgatccgagttcaaatctcggtggaacct

SEQ ID NO: 39 - DNA, 72 bp, Artificial Sequence
  Description of Artificial Sequence Synthetic oligonucleotide
ggttccatggtgtaatggttagcactctggactttaaatccagcgatccgagttcaaatctcggtgggacct

SEQ ID NO: 40 - DNA, 72 bp, Artificial Sequence
  Description of Artificial Sequence Synthetic oligonucleotide
ggttccatggtgtaatggtgagcactctggactttaaatccagcgatccgagttcaaatctcggtgggacct

SEQ ID NO: 41 - DNA, 72 bp, Artificial Sequence
  Description of Artificial Sequence Synthetic oligonucleotide
ggttccatggtgtaatggctagcactctggactttaaatccagcgatccgagttcaaatctcggtgggattt

SEQ ID NO: 42 - DNA, 72 bp, Artificial Sequence
  Description of Artificial Sequence Synthetic oligonucleotide
ggttccatggtgtaatggttagcactctggactttaaatccagccatacaagttcaaatctcagtggaacct

SEQ ID NO: 43 - DNA, 71 bp, Artificial Sequence
  Description of Artificial Sequence Synthetic oligonucleotide
ggttccttggtgtaagatgagcactctggattttaaatccagcgatcagagttcaaatctcggtgggacct

SEQ ID NO: 44 - DNA, 72 bp, Artificial Sequence
  Description of Artificial Sequence Synthetic oligonucleotide
ggtcccatggtgtaatggttagcactctggactttaaatccagcaatctgagttcaaatctcggtgggacct

SEQ ID NO: 45 - DNA, 72 bp, Artificial Sequence
  Description of Artificial Sequence Synthetic oligonucleotide
ggccccatggtgtaatggttagcactctggactttaaatccagcgatccgagttcaaatctcggtgggacct

SEQ ID NO: 46 - DNA, 72 bp, Artificial Sequence
  Description of Artificial Sequence Synthetic oligonucleotide
ggtctcatggtgtaatggttagcacactggactttaagtccagcaatccgagttcgagtcttggtgagacca

SEQ ID NO: 47 - DNA, 72 bp, Artificial Sequence
  Description of Artificial Sequence Synthetic oligonucleotide
ggacccatggtgtaatggttagcactctggactttaaatccagcaatccaagttcaaatctcggtgggacct

SEQ ID NO: 48 - DNA, 72 bp, Artificial Sequence
  Description of Artificial Sequence Synthetic oligonucleotide
gtttccatggtgtaatggttggcactctggactttaaatccagcaatccaagttcaagtctctgtgggacct

SEQ ID NO: 49 - DNA, 106 bp, Artificial Sequence
  Description of Artificial Sequence Synthetic polynucleotide
gtcaggatggccgagtggtctaaggcgccagactctagctatggcttcctcgctctgagggttctggtctcccctggaggcgtgggttcgaatcccacttctgaca

SEQ ID NO: 50 - DNA, 105 bp, Artificial Sequence
  Description of Artificial Sequence Synthetic polynucleotide
gtcaggatggccgagtggtctaaggcgccagactctagcttagcttccctgtctggggattctggtctccgtatggaggcgtgggttcgaatcccacttctgaca

SEQ ID NO: 51 - DNA, 107 bp, Artificial Sequence
  Description of Artificial Sequence Synthetic polynucleotide
gtcaggatggccgagtggtctaaggcgccagactctaggtgacaagccttacctacgggtgttctggtctccgaatggaggcgtgggttcgaatcccacttctgaca

SEQ ID NO: 52 - DNA, 106 bp, Artificial Sequence
  Description of Artificial Sequence Synthetic polynucleotide
gtcaggatggccgagtggtctaaggcgccagactctagcgttcgcttcctctactgagggttctggtctccgtgtggaggcgtgggttcgaatcccacttctgaca

SEQ ID NO: 53 - DNA, 106 bp, Artificial Sequence
  Description of Artificial Sequence Synthetic polynucleotide
gtcaggatggccgagtggtctaaggcgccagacttcagctatggcttcctcgctctgagggttctggtctcccctggaggcgtgggttcgaatcccacttctgaca

SEQ ID NO: 54 - DNA, 105 bp, Artificial Sequence
  Description of Artificial Sequence Synthetic polynucleotide
gtcaggatggccgagtggtctaaggcgccagacttcagcttagcttccctgtctggggattctggtctccgtatggaggcgtgggttcgaatcccacttctgaca

SEQ ID NO: 55 - DNA, 107 bp, Artificial Sequence
  Description of Artificial Sequence Synthetic polynucleotide
gtcaggatggccgagtggtctaaggcgccagacttcaggtgacaagccttacctacgggtgttctggtctccgaatggaggcgtgggttcgaatcccacttctgaca

SEQ ID NO: 56 - DNA, 106 bp, Artificial Sequence
  Description of Artificial Sequence Synthetic polynucleotide
gtcaggatggccgagtggtctaaggcgccagacttcagcgttcgcttcctctactgagggttctggtctccgtgtggaggcgtgggttcgaatcccacttctgaca

SEQ ID NO: 57 - DNA, 106 bp, Artificial Sequence
  Description of Artificial Sequence Synthetic polynucleotide
gtcaggatggccgagtggtctaaggcgccagactttagctatggcttcctcgctctgagggttctggtctcccctggaggcgtgggttcgaatcccacttctgaca

SEQ ID NO: 58 - DNA, 105 bp, Artificial Sequence
  Description of Artificial Sequence Synthetic polynucleotide
gtcaggatggccgagtggtctaaggcgccagactttagcttagcttccctgtctggggattctggtctccgtatggaggcgtgggttcgaatcccacttctgaca

SEQ ID NO: 59 - DNA, 107 bp, Artificial Sequence
  Description of Artificial Sequence Synthetic polynucleotide
gtcaggatggccgagtggtctaaggcgccagactttaggtgacaagccttacctacgggtgttctggtctccgaatggaggcgtgggttcgaatcccacttctgaca

SEQ ID NO: 60 - DNA, 106 bp, Artificial Sequence
  Description of Artificial Sequence Synthetic polynucleotide
gtcaggatggccgagtggtctaaggcgccagactttagcgttcgcttcctctactgagggttctggtctccgtgtggaggcgtgggttcgaatcccacttctgaca

SEQ ID NO: 61 - DNA, 89 bp, Artificial Sequence
  Description of Artificial Sequence Synthetic oligonucleotide
ccttcgatagctcagttggtagagcggaggactctagagttactagaatagtgatccttaggtcgctggttcgaatccggctcgaagga

SEQ ID NO: 62 - DNA, 89 bp, Artificial Sequence
  Description of Artificial Sequence Synthetic oligonucleotide
ccttcgatagctcagttggtagagcggaggactctagtcagtacaatatggtaatccttaggtcgctggttcgattccggctcgaagga

SEQ ID NO: 63 - DNA, 89 bp, Artificial Sequence
  Description of Artificial Sequence Synthetic oligonucleotide
ccttcgatagctcagctggtagagcggaggactctaggcttgtggctgtggacatccttaggtcgctggttcgattccggctcgaagga

SEQ ID NO: 64 - DNA, 93 bp, Artificial Sequence
  Description of Artificial Sequence Synthetic oligonucleotide
ccttcgatagctcagctggtagagcggaggactctagctaactccccgttagaagacatccttaggtcgctggttcgactccggctcgaagga

SEQ ID NO: 65 - DNA, 89 bp, Artificial Sequence
  Description of Artificial Sequence Synthetic oligonucleotide
ctttcgatagttcagttggtagagcggaggactctagagtattaacgttggtgatccttaggtcgctggttcgagtccggctcgaagga

SEQ ID NO: 66 - DNA, 89 bp, Artificial Sequence
  Description of Artificial Sequence Synthetic oligonucleotide
ccttcgatagctcagttggtagagcggaggactttagagttactagaatagtgatccttaggtcgctggttcgaatccggctcgaagga

SEQ ID NO: 67 - DNA, 89 bp, Artificial Sequence
  Description of Artificial Sequence Synthetic oligonucleotide
ccttcgatagctcagttggtagagcggaggactttagtcagtacaatatggtaatccttaggtcgctggttcgattccggctcgaagga

SEQ ID NO: 68 - DNA, 89 bp, Artificial Sequence
  Description of Artificial Sequence Synthetic oligonucleotide
ccttcgatagctcagctggtagagcggaggactttaggcttgtggctgtggacatccttaggtcgctggttcgattccggctcgaagga

SEQ ID NO: 69 - DNA, 93 bp, Artificial Sequence
  Description of Artificial Sequence Synthetic oligonucleotide
ccttcgatagctcagctggtagagcggaggactttagctaactccccgttagaagacatccttaggtcgctggttcgactccggctcgaagga

SEQ ID NO: 70 - DNA, 89 bp, Artificial Sequence
  Description of Artificial Sequence Synthetic oligonucleotide
ctttcgatagttcagttggtagagcggaggactttagagtattaacgttggtgatccttaggtcgctggttcgagtccggctcgaagga

SEQ ID NO: 71 - DNA, 72 bp, Artificial Sequence
  Description of Artificial Sequence Synthetic oligonucleotide
gggggtatagctcagtggtagagcatttgacttcagatcaagaggtccctggttcaaatccaggtgccccct

SEQ ID NO: 72 - DNA, 72 bp, Artificial Sequence
  Description of Artificial Sequence Synthetic oligonucleotide
gggggtatagctcaggggtagagcatttgacttcagatcaagaggtccctggttcaaatccagatgccccct

SEQ ID NO: 73 - DNA, 72 bp, Artificial Sequence
  Description of Artificial Sequence Synthetic oligonucleotide
gggggtatagctcaggggtagagtatttggcttcagatcaagaggtccctggttcaaatccaggtgccccct

SEQ ID NO: 74 - DNA, 72 bp, Artificial Sequence
  Description of Artificial Sequence Synthetic oligonucleotide
gggggtatagctcaggggtagagcatttgacttcagatcaagaggtccttggttcaaatccaggtgtcccct

SEQ ID NO: 75 - DNA, 72 bp, Artificial Sequence
  Description of Artificial Sequence Synthetic oligonucleotide
gggggtatagctcagaggtagagcatttgacttcagatcaagagatctctggttcaaatccaggtgccccct

SEQ ID NO: 76 - DNA, 72 bp, Artificial Sequence
  Description of Artificial Sequence Synthetic oligonucleotide
gggggtatagctcaggggtagagcatttgacttcagatcaagaggtccctagttcaaatccaggtgccccct

SEQ ID NO: 77 - DNA, 72 bp, Artificial Sequence
  Description of Artificial Sequence Synthetic oligonucleotide
ggtggtatagctcaggggtagagcatttgacttcagatcaagagatccctggttcgaatccaggtgccccct

SEQ ID NO: 78 - DNA, 72 bp, Artificial Sequence
  Description of Artificial Sequence Synthetic oligonucleotide
gggggtataactcaggggtagagcatttgacttcagatcaagaggtccctggttcaaatccaggtgccccct

SEQ ID NO: 79 - DNA, 72 bp, Artificial Sequence
  Description of Artificial Sequence Synthetic oligonucleotide
tggggtatagctcaggggtagagcatttgacttcagatcaagaggtccctggttcaaatccaggtgccccct

SEQ ID NO: 80 - DNA, 72 bp, Artificial Sequence
  Description of Artificial Sequence Synthetic oligonucleotide
gggggtatagctcagaggaagagcatttgacttcagatcaagaggtccctgattcaaatccaggtgccccct

SEQ ID NO: 81 - DNA, 72 bp, Artificial Sequence
  Description of Artificial Sequence Synthetic oligonucleotide
gggggtaaagctcaggggtagagcatttgacttcagattaagaggtccctggttcaaatccaggtaccccct

SEQ ID NO: 82 - DNA, 72 bp, Artificial Sequence
  Description of Artificial Sequence Synthetic oligonucleotide
gggggtatagctcagtggtagagcatttgacttcagatcaagaggtccctggttcaaatccgggtgccccct

SEQ ID NO: 83 - DNA, 72 bp, Artificial Sequence
  Description of Artificial Sequence Synthetic oligonucleotide
ggggttatagctcaggtgtagagcatttgacttcagatcaagaggtccctggttcaaatccaggtgccccct

SEQ ID NO: 84 - DNA, 72 bp, Artificial Sequence
  Description of Artificial Sequence Synthetic oligonucleotide
gggggtatagctcaggggtagagcatttgacttcagatcacgaggtccctggttcaaatcgaggtgccccct

SEQ ID NO: 85 - DNA, 72 bp, Artificial Sequence
  Description of Artificial Sequence Synthetic oligonucleotide
gggggtatagctcaggggtggagcatttgacttcagatcaaggggtccctgtttcaaatccaggtgccccct

SEQ ID NO: 86 - DNA, 72 bp, Artificial Sequence
  Description of Artificial Sequence Synthetic oligonucleotide
gggggtatagctcagtggtagagcatttgacttcagatcaagaggtccccggttcaaatccgggtgccccct

SEQ ID NO: 87 - DNA, 72 bp, Artificial Sequence
  Description of Artificial Sequence Synthetic oligonucleotide
gggggtatagctcaggggtagagcatttgacttcagatcaagaggtccctggttcaaatccaggtgccccct

SEQ ID NO: 88 - DNA, 72 bp, Artificial Sequence
  Description of Artificial Sequence Synthetic oligonucleotide
gggggtatagctcaggggtagagcatttgacttcagatcaagaggtccctggttcaaatccgggtgccccct

SEQ ID NO: 89 - DNA, 72 bp, Artificial Sequence
  Description of Artificial Sequence Synthetic oligonucleotide
gggggtatagctcaggggtagagcatttgacttcagatcaagaggtccctggttcaaatccaggtaccccct

SEQ ID NO: 90 - DNA, 72 bp, Artificial Sequence
  Description of Artificial Sequence Synthetic oligonucleotide
gggggtatagctcaggggtagagcatttgacttcagatcaagaggtccccggttcaaatccgggtgccccct

SEQ ID NO: 91 - DNA, 72 bp, Artificial Sequence
  Description of Artificial Sequence Synthetic oligonucleotide
gggggcatagctcaggggtagagcatttgacttcagatcaagaggtccccggttcaaatccgggtgctccct

SEQ ID NO: 92 - DNA, 72 bp, Artificial Sequence
  Description of Artificial Sequence Synthetic oligonucleotide
gggggtatagctcaggggtagagcatttgacttcagattaagaggtccctggttcaaatccaggtgccccct

SEQ ID NO: 93 - DNA, 72 bp, Artificial Sequence
  Description of Artificial Sequence Synthetic oligonucleotide
tccctggtggtctagtggttaggattcggcgctctaaccgccgcggcccgggttcgattcccggtcagggaa

SEQ ID NO: 94 - DNA, 72 bp, Artificial Sequence
  Description of Artificial Sequence Synthetic oligonucleotide
tccctggtggtctagtggttaggatttggcgctctaaccgccgcggcctgggttcgattcccggtcagggaa

SEQ ID NO: 95 - DNA, 72 bp, Artificial Sequence
  Description of Artificial Sequence Synthetic oligonucleotide
tccctggtggtctagtggttaggctttggtgctctaacctccatggcccaggtttgattcctggtcagggaa

SEQ ID NO: 96 - DNA, 72 bp, Artificial Sequence
  Description of Artificial Sequence Synthetic oligonucleotide
tccctggtggtctagtggttaggattcggcgctttaaccgccgcggcccgggttcgattcccggtcagggaa

SEQ ID NO: 97 - DNA, 72 bp, Artificial Sequence
  Description of Artificial Sequence Synthetic oligonucleotide
tccctggtggtctagtggttaggatttggcgctttaaccgccgcggcctgggttcgattcccggtcagggaa

SEQ ID NO: 98 - DNA, 72 bp, Artificial Sequence
  Description of Artificial Sequence Synthetic oligonucleotide
tccctggtggtctagtggttaggctttggtgctttaacctccatggcccaggtttgattcctggtcagggaa

SEQ ID NO: 99 - DNA, 72 bp, Artificial Sequence
  Description of Artificial Sequence Synthetic oligonucleotide
tcccacatggtctagcggttaggattcctggttctaacccaggcggcccgggttcgactcccggtgtgggaa

SEQ ID NO: 100 - DNA, 72 bp, Artificial Sequence
  Description of Artificial Sequence Synthetic oligonucleotide
tcccatatggtctagcggttaggattcctggttctaacccaggcggcccgggttcgactcccggtatgggaa

SEQ ID NO: 101 - DNA, 72 bp, Artificial Sequence
  Description of Artificial Sequence Synthetic oligonucleotide
tccctggtggtctagtggctaggattcggcgctctaaccgccgcggcccgggttcgattcccggtcagggaa

SEQ ID NO: 102 - DNA, 72 bp, Artificial Sequence
  Description of Artificial Sequence Synthetic oligonucleotide
tcccacatggtctagcggttaggattcctggttttaacccaggcggcccgggttcgactcccggtgtgggaa

SEQ ID NO: 103 - DNA, 72 bp, Artificial Sequence
  Description of Artificial Sequence Synthetic oligonucleotide
tcccatatggtctagcggttaggattcctggttttaacccaggcggcccgggttcgactcccggtatgggaa

SEQ ID NO: 104 - DNA, 72 bp, Artificial Sequence
  Description of Artificial Sequence Synthetic oligonucleotide
tccctggtggtctagtggctaggattcggcgctttaaccgccgcggcccgggttcgattcccggtcagggaa

SEQ ID NO: 105 - DNA, 74 bp, Artificial Sequence
  Description of Artificial Sequence Synthetic oligonucleotide
gtttccgtagtgtagtggttagcgcgttcgccttcaaaagcgaaaggtccccggttcgaaaccgggcggaaaca

SEQ ID NO: 106 - DNA, 71 bp, Artificial Sequence
  Description of Artificial Sequence Synthetic oligonucleotide
gcgccgctggtgtagtggtatcatgcaagatttcaattcttgcgacccgggttcgattcccgggcggcgca

SEQ ID NO: 107 - DNA, 71 bp, Artificial Sequence
  Description of Artificial Sequence Synthetic oligonucleotide
gcattggtagttcaatggtagaattctcgccttcaacgcgggtgacccgggttcgattcccggccaatgca

SEQ ID NO: 108 - DNA, 71 bp, Artificial Sequence
  Description of Artificial Sequence Synthetic oligonucleotide
gcattggtggttcaatggtagaattctcgccttcaacgcgggtgacccgggttcgattcccggccaatgca

SEQ ID NO: 109 - DNA, 71 bp, Artificial Sequence
  Description of Artificial Sequence Synthetic oligonucleotide
gcattggtggttcaatggtagaattctcgccttcaactcgggtgacccgggttcgattcccggccaatgca

SEQ ID NO: 110 - DNA, 71 bp, Artificial Sequence
  Description of Artificial Sequence Synthetic oligonucleotide
gcatgggtggttcagtggtagaattctcgccttcaacgcgggaggcccgggttcgattcccggcccatgca

SEQ ID NO: 111 - DNA, 71 bp, Artificial Sequence
  Description of Artificial Sequence Synthetic oligonucleotide
gcattggtggttcagtggtagaattctcgccttcaacgcgggaggcccgggttcgattcccggccaatgca

SEQ ID NO: 112 - DNA, 71 bp, Artificial Sequence
  Description of Artificial Sequence Synthetic oligonucleotide
gcattggtggttcagtggtagaattctcgccttcaacgcgggaggcccgggtttgattcccggccaatgca

SEQ ID NO: 113 - DNA, 71 bp, Artificial Sequence
  Description of Artificial Sequence Synthetic oligonucleotide
gcattggtggttcagtggtagaattctcgccttcaacgcgggaggcccgggttcggttcccggccaatgca

SEQ ID NO: 114 - DNA, 72 bp, Artificial Sequence
  Description of Artificial Sequence Synthetic oligonucleotide
gcgttggtggtatagtggtgagcatagctgccttcaaagcagttgacccgggttcgattcccggccaacgca

SEQ ID NO: 115 - DNA, 82 bp, Artificial Sequence
  Description of Artificial Sequence Synthetic oligonucleotide
ggtagcgtggccgagcggtctaaggcgctggattctagctccagtctcttcgggggcgtgggttcgaatcccaccgctgcca

SEQ ID NO: 116 - DNA, 82 bp, Artificial Sequence
  Description of Artificial Sequence Synthetic oligonucleotide
ggtagtgtggccgagcggtctaaggcgctggattctagctccagtctcttcgggggcgtgggttcgaatcccaccgctgcca

SEQ ID NO: 117 - DNA, 82 bp, Artificial Sequence
  Description of Artificial Sequence Synthetic oligonucleotide
ggtagtgtggccgagcggtctaaggcgctggattctagctccagtctcttcgggggcgtgggttcgaatcccaccactgcca

SEQ ID NO: 118 - DNA, 82 bp, Artificial Sequence
  Description of Artificial Sequence Synthetic oligonucleotide
ggtagcgtggccgagcggtctaaggcgctggatttcagctccagtctcttcgggggcgtgggttcgaatcccaccgctgcca

SEQ ID NO: 119 - DNA, 82 bp, Artificial Sequence
  Description of Artificial Sequence Synthetic oligonucleotide
ggtagtgtggccgagcggtctaaggcgctggatttcagctccagtctcttcgggggcgtgggttcgaatcccaccgctgcca

SEQ ID NO: 120 - DNA, 82 bp, Artificial Sequence
  Description of Artificial Sequence Synthetic oligonucleotide
ggtagtgtggccgagcggtctaaggcgctggatttcagctccagtctcttcgggggcgtgggttcgaatcccaccactgcca

SEQ ID NO: 121 - DNA, 82 bp, Artificial Sequence
  Description of Artificial Sequence Synthetic oligonucleotide
ggtagcgtggccgagcggtctaaggcgctggattttagctccagtctcttcgggggcgtgggttcgaatcccaccgctgcca

SEQ ID NO: 122 - DNA, 82 bp, Artificial Sequence
  Description of Artificial Sequence Synthetic oligonucleotide
ggtagtgtggccgagcggtctaaggcgctggattttagctccagtctcttcgggggcgtgggttcgaatcccaccgctgcca

SEQ ID NO: 123 - DNA, 82 bp, Artificial Sequence
  Description of Artificial Sequence Synthetic oligonucleotide
ggtagtgtggccgagcggtctaaggcgctggattttagctccagtctcttcgggggcgtgggttcgaatcccaccactgcca

SEQ ID NO: 124 - DNA, 83 bp, Artificial Sequence
  Description of Artificial Sequence Synthetic oligonucleotide
gtcaggatggccgagtggtctaaggcgccagactctagttctggtctcccctggaggcgtgggttcgaatcccacttctgaca

SEQ ID NO: 125 - DNA, 84 bp, Artificial Sequence
  Description of Artificial Sequence Synthetic oligonucleotide
gtcaggatggccgagtggtctaaggcgccagactctagttctggtctccgtatggaggcgtgggttcgaatcccacttctgaca

SEQ ID NO: 126 - DNA, 84 bp, Artificial Sequence
  Description of Artificial Sequence Synthetic oligonucleotide
gtcaggatggccgagtggtctaaggcgccagactctagttctggtctccgaatggaggcgtgggttcgaatcccacttctgaca

SEQ ID NO: 127 - DNA, 84 bp, Artificial Sequence
  Description of Artificial Sequence Synthetic oligonucleotide
gtcaggatggccgagtggtctaaggcgccagactctagttctggtctccgtgtggaggcgtgggttcgaatcccacttctgaca

SEQ ID NO: 128 - DNA, 83 bp, Artificial Sequence
  Description of Artificial Sequence Synthetic oligonucleotide
gtcaggatggccgagtggtctaaggcgccagacttcagttctggtctcccctggaggcgtgggttcgaatcccacttctgaca

SEQ ID NO: 129 - DNA, 84 bp, Artificial Sequence
  Description of Artificial Sequence Synthetic oligonucleotide
gtcaggatggccgagtggtctaaggcgccagacttcagttctggtctccgtatggaggcgtgggttcgaatcccacttctgaca

SEQ ID NO: 130 - DNA, 84 bp, Artificial Sequence
  Description of Artificial Sequence Synthetic oligonucleotide
gtcaggatggccgagtggtctaaggcgccagacttcagttctggtctccgaatggaggcgtgggttcgaatcccacttctgaca

SEQ ID NO: 131 - DNA, 84 bp, Artificial Sequence
  Description of Artificial Sequence Synthetic oligonucleotide
gtcaggatggccgagtggtctaaggcgccagacttcagttctggtctccgtgtggaggcgtgggttcgaatcccacttctgaca

SEQ ID NO: 132 - DNA, 83 bp, Artificial Sequence
  Description of Artificial Sequence Synthetic oligonucleotide
gtcaggatggccgagtggtctaaggcgccagactttagttctggtctcccctggaggcgtgggttcgaatcccacttctgaca

SEQ ID NO: 133 - DNA, 84 bp, Artificial Sequence
  Description of Artificial Sequence Synthetic oligonucleotide
gtcaggatggccgagtggtctaaggcgccagactttagttctggtctccgtatggaggcgtgggttcgaatcccacttctgaca

SEQ ID NO: 134 - DNA, 84 bp, Artificial Sequence
  Description of Artificial Sequence Synthetic oligonucleotide
gtcaggatggccgagtggtctaaggcgccagactttagttctggtctccgaatggaggcgtgggttcgaatcccacttctgaca

SEQ ID NO: 135 - DNA, 84 bp, Artificial Sequence
  Description of Artificial Sequence Synthetic oligonucleotide
gtcaggatggccgagtggtctaaggcgccagactttagttctggtctccgtgtggaggcgtgggttcgaatcccacttctgaca

SEQ ID NO: 136 - DNA, 83 bp, Artificial Sequence
  Description of Artificial Sequence Synthetic oligonucleotide
gtcaggatggccgagcggtctaaggcgctgcgttctagtcgcagtctcccctggaggcgtgggttcgaatcccactcctgaca

SEQ ID NO: 137 - DNA, 83 bp, Artificial Sequence
  Description of Artificial Sequence Synthetic oligonucleotide
gtcaggatggccgagcggtctaaggcgctgcgttctagtcgcagtctcccctggaggcgtgggttcgaatcccacttctgaca

SEQ ID NO: 138 - DNA, 83 bp, Artificial Sequence
  Description of Artificial Sequence Synthetic oligonucleotide
gtcaggatggccgagtggtctaaggagctgtgttctagtcgcagtctcccctggaggcgtgggttcgaatcccactcctgaca

SEQ ID NO: 139 - DNA, 83 bp, Artificial Sequence
  Description of Artificial Sequence Synthetic oligonucleotide
gtcaggatggccgagcagtctaaggcactgcgttctagtcgcagtctcccctggaggcgtggattcgaatcccactcctgaca

SEQ ID NO: 140 - DNA, 83 bp, Artificial Sequence
  Description of Artificial Sequence Synthetic oligonucleotide
gtcaggatggccgagcggtctaaggcgctgcgtttcagtcgcagtctcccctggaggcgtgggttcgaatcccactcctgaca

SEQ ID NO: 141 - DNA, 83 bp, Artificial Sequence
  Description of Artificial Sequence Synthetic oligonucleotide
gtcaggatggccgagcggtctaaggcgctgcgtttcagtcgcagtctcccctggaggcgtgggttcgaatcccacttctgaca

SEQ ID NO: 142 - DNA, 83 bp, Artificial Sequence
  Description of Artificial Sequence Synthetic oligonucleotide
gtcaggatggccgagtggtctaaggagctgtgtttcagtcgcagtctcccctggaggcgtgggttcgaatcccactcctgaca

SEQ ID NO: 143 - DNA, 83 bp, Artificial Sequence
  Description of Artificial Sequence Synthetic oligonucleotide
gtcaggatggccgagcagtctaaggcactgcgtttcagtcgcagtctcccctggaggcgtggattcgaatcccactcctgaca

SEQ ID NO: 144 - DNA, 83 bp, Artificial Sequence
  Description of Artificial Sequence Synthetic oligonucleotide
gtcaggatggccgagcggtctaaggcgctgcgttttagtcgcagtctcccctggaggcgtgggttcgaatcccactcctgaca

SEQ ID NO: 145 - DNA, 83 bp, Artificial Sequence
  Description of Artificial Sequence Synthetic oligonucleotide
gtcaggatggccgagcggtctaaggcgctgcgttttagtcgcagtctcccctggaggcgtgggttcgaatcccacttctgaca

SEQ ID NO: 146 - DNA, 83 bp, Artificial Sequence
  Description of Artificial Sequence Synthetic oligonucleotide
gtcaggatggccgagtggtctaaggagctgtgttttagtcgcagtctcccctggaggcgtgggttcgaatcccactcctgaca

SEQ ID NO: 147 - DNA, 83 bp, Artificial Sequence
  Description of Artificial Sequence Synthetic oligonucleotide
gtcaggatggccgagcagtctaaggcactgcgttttagtcgcagtctcccctggaggcgtggattcgaatcccactcctgaca

SEQ ID NO: 148 - DNA, 83 bp, Artificial Sequence
  Description of Artificial Sequence Synthetic oligonucleotide
accagaatggccgagtggttaaggcgttggactctagatccaatggatttatatccgcgtgggttcgaaccccacttctggta

SEQ ID NO: 149 - DNA, 83 bp, Artificial Sequence
  Description of Artificial Sequence Synthetic oligonucleotide
accaggatggccgagtggttaaggcgttggactctagatccaatggacatatgtctgcgtgggttcgaaccccactcctggta

SEQ ID NO: 150 - DNA, 83 bp, Artificial Sequence
  Description of Artificial Sequence Synthetic oligonucleotide
actgggatggctgagtggttaaggcgttggactctagatccaatgggcggttgcctgcgtgggttcgaaccccactcccagta

SEQ ID NO: 151 - DNA, 83 bp, Artificial Sequence
  Description of Artificial Sequence Synthetic oligonucleotide
gatgggatggctgagaggttaaggctttggactctagatccaatgggcagatgcctgcgtgggtttgaaccccactcccaata

SEQ ID NO: 152 - DNA, 83 bp, Artificial Sequence
  Description of Artificial Sequence Synthetic oligonucleotide
accagaatggccgagtggttaaggcgttggacttcagatccaatggatttatatccgcgtgggttcgaaccccacttctggta

SEQ ID NO: 153 - DNA, 83 bp, Artificial Sequence
  Description of Artificial Sequence Synthetic oligonucleotide
accaggatggccgagtggttaaggcgttggacttcagatccaatggacatatgtctgcgtgggttcgaaccccactcctggta

SEQ ID NO: 154 - DNA, 83 bp, Artificial Sequence
  Description of Artificial Sequence Synthetic oligonucleotide
actgggatggctgagtggttaaggcgttggacttcagatccaatgggcggttgcctgcgtgggttcgaaccccactcccagta

SEQ ID NO: 155 - DNA, 83 bp, Artificial Sequence
  Description of Artificial Sequence Synthetic oligonucleotide
gatgggatggctgagaggttaaggctttggacttcagatccaatgggcagatgcctgcgtgggtttgaaccccactcccaata

SEQ ID NO: 156 - DNA, 83 bp, Artificial Sequence
  Description of Artificial Sequence Synthetic oligonucleotide
accagaatggccgagtggttaaggcgttggactttagatccaatggatttatatccgcgtgggttcgaaccccacttctggta

SEQ ID NO: 157 - DNA, 83 bp, Artificial Sequence
  Description of Artificial Sequence Synthetic oligonucleotide
accaggatggccgagtggttaaggcgttggactttagatccaatggacatatgtctgcgtgggttcgaaccccactcctggta

SEQ ID NO: 158 - DNA, 83 bp, Artificial Sequence
  Description of Artificial Sequence Synthetic oligonucleotide
actgggatggctgagtggttaaggcgttggactttagatccaatgggcggttgcctgcgtgggttcgaaccccactcccagta

SEQ ID NO: 159 - DNA, 83 bp, Artificial Sequence
  Description of Artificial Sequence Synthetic oligonucleotide
gatgggatggctgagaggttaaggctttggactttagatccaatgggcagatgcctgcgtgggtttgaaccccactcccaata

SEQ ID NO: 160 - DNA, 82 bp, Artificial Sequence
  Description of Artificial Sequence Synthetic oligonucleotide
ggtagcgtggccgagcggtctaaggcgctggattctagctccagtctcttcggaggcgtgggttcgaatcccaccgctgcca

SEQ ID NO: 161 - DNA, 82 bp, Artificial Sequence
  Description of Artificial Sequence Synthetic oligonucleotide
ggtagtgtggccgagcggtctaaggcgctggattctagctccagtctcttcggaggcgtgggttcgaatcccaccactgcca

SEQ ID NO: 162 - DNA, 82 bp, Artificial Sequence
  Description of Artificial Sequence Synthetic oligonucleotide
ggtagcgtggccgagtggtctaaggcgctggattctagctccagtcatttcgatggcgtgggttcgaatcccaccgctgcca

SEQ ID NO: 163 - DNA, 82 bp, Artificial Sequence
  Description of Artificial Sequence Synthetic oligonucleotide
ggtagcgtggccgagcggtctaaggcgctggatttcagctccagtctcttcggaggcgtgggttcgaatcccaccgctgcca

SEQ ID NO: 164 - DNA, 82 bp, Artificial Sequence
  Description of Artificial Sequence Synthetic oligonucleotide
ggtagtgtggccgagcggtctaaggcgctggatttcagctccagtctcttcggaggcgtgggttcgaatcccaccactgcca

SEQ ID NO: 165 - DNA, 82 bp, Artificial Sequence
  Description of Artificial Sequence Synthetic oligonucleotide
ggtagcgtggccgagtggtctaaggcgctggatttcagctccagtcatttcgatggcgtgggttcgaatcccaccgctgcca

SEQ ID NO: 166 - DNA, 82 bp, Artificial Sequence
  Description of Artificial Sequence Synthetic oligonucleotide
ggtagcgtggccgagcggtctaaggcgctggattttagctccagtctcttcggaggcgtgggttcgaatcccaccgctgcca

SEQ ID NO: 167 - DNA, 82 bp, Artificial Sequence
  Description of Artificial Sequence Synthetic oligonucleotide
ggtagtgtggccgagcggtctaaggcgctggattttagctccagtctcttcggaggcgtgggttcgaatcccaccactgcca

SEQ ID NO: 168 - DNA, 82 bp, Artificial Sequence
  Description of Artificial Sequence Synthetic oligonucleotide
ggtagcgtggccgagtggtctaaggcgctggattttagctccagtcatttcgatggcgtgggttcgaatcccaccgctgcca

SEQ ID NO: 169 - DNA, 73 bp, Artificial Sequence
  Description of Artificial Sequence Synthetic oligonucleotide
gcccagctagctcagttggtagagcgtgggactctaaatcctagggtcgtgggttcgaaccccacgttgggcg

SEQ ID NO: 170 - DNA, 73 bp, Artificial Sequence
  Description of Artificial Sequence Synthetic oligonucleotide
gcccagctagctcagtctgtagagcatgagactctaagtctcagggtcatgggttggagccccatgttgtgca

SEQ ID NO: 171 - DNA, 77 bp, Artificial Sequence
  Description of Artificial Sequence Synthetic oligonucleotide
gcctagctagttcagtcggtagagcatgagactctaaatctcaggttcatgagtttgagccccatgttggtttggca

SEQ ID NO: 172 - DNA, 73 bp, Artificial Sequence
  Description of Artificial Sequence Synthetic oligonucleotide
ccccggctagctcagtcagtagagcttgagaatctaaatctcagggtcgtgggttggagccccacgttgggcg

SEQ ID NO: 173 - DNA, 200 bp, Artificial Sequence
  Description of Artificial Sequence Synthetic polynucleotide
gatcaccggaagaggtgacagacacctcggggcccatgaacgtttggaattcgtaaggacatgagaatctcggtggttccgtgtctgcccgccatcgcggccaccggccacgggcccaagccaagtgtagcgaagcttagaaaaggttgcccaacgtcatgtggcttgagaaggctgccgggcgccttaagccgccagca

SEQ ID NO: 174 - DNA, 200 bp, Artificial Sequence
  Description of Artificial Sequence Synthetic polynucleotide
cactgaacctttttttggccttagaatccctgttttggggcctgcaggaagtagcaaccaacccgagcctccgcagggaatgcactgacctgtagaatggacgttcagcttccctccctgtgtctcaacacgattacatttcaggaacagcctgggctgggaggcactgcgcacgcgcgccgagtcgggcggaaaaataa

SEQ ID NO: 175 - DNA, 1854 bp, Artificial Sequence
  Description of Artificial Sequence Synthetic polynucleotide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggcccaacggtaccggatccaccggccgccaccatgggaagcccaaagaagaagcgtaaggtaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgtaatgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagccccaagaaaaagcggaaggtgtaa

SEQ ID NO: 176 - DNA, 1854 bp, Artificial Sequence
  Description of Artificial Sequence Synthetic polynucleotide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggcccaacggtaccggatccaccggccgccaccatgggaagcccaaagaagaagcgtaaggtaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgtagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagccccaagaaaaagcggaaggtgtaa

SEQ ID NO: 177 - DNA, 1981 bp, Artificial Sequence
  Description of Artificial Sequence Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtaccggatccaccggccgccaccatgggaagcccaaagaagaagcgtaaggtaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagtgaaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagccccaagaaaaagcggaaggtgtaa

SEQ ID NO: 178 - DNA, 72 bp, Artificial Sequence
  Description of Artificial Sequence Synthetic oligonucleotide
ggtcccatggtgtaatggttagcactctggactctaaatccagcgatccgagttcaaatctcggtgggacct

SEQ ID NO: 179 - DNA, 72 bp, Artificial Sequence
  Description of Artificial Sequence Synthetic oligonucleotide
ggttccatggtgtaatggttagcactctggactctaaatccagcgacccgagttcaaatctcggtgggacct

SEQ ID NO: 180 - DNA, 72 bp, Artificial Sequence
  Description of Artificial Sequence Synthetic oligonucleotide
ggttccatggtgtaatggttagcactctggactctaaatccagcgatccgagttcaaatctcggtggaacct

SEQ ID NO: 181 - DNA, 72 bp, Artificial Sequence
  Description of Artificial Sequence Synthetic oligonucleotide
ggttccatggtgtaatggttagcactctggactctaaatccagcgatccgagttcaaatctcggtgggacct

SEQ ID NO: 182 - DNA, 72 bp, Artificial Sequence
  Description of Artificial Sequence Synthetic oligonucleotide
ggttccatggtgtaatggtgagcactctggactctaaatccagcgatccgagttcaaatctcggtgggacct

SEQ ID NO: 183 - DNA, 72 bp, Artificial Sequence
  Description of Artificial Sequence Synthetic oligonucleotide
ggttccatggtgtaatggctagcactctggactctaaatccagcgatccgagttcaaatctcggtgggattt

SEQ ID NO: 184 - DNA, 72 bp, Artificial Sequence
  Description of Artificial Sequence Synthetic oligonucleotide
ggttccatggtgtaatggttagcactctggactctaaatccagccatacaagttcaaatctcagtggaacct

SEQ ID NO: 185 - DNA, 71 bp, Artificial Sequence
  Description of Artificial Sequence Synthetic oligonucleotide
ggttccttggtgtaagatgagcactctggattctaaatccagcgatcagagttcaaatctcggtgggacct

SEQ ID NO: 186 - DNA, 72 bp, Artificial Sequence
  Description of Artificial Sequence Synthetic oligonucleotide
ggtcccatggtgtaatggttagcactctggactctaaatccagcaatctgagttcaaatctcggtgggacct

SEQ ID NO: 187 - DNA, 72 bp, Artificial Sequence
  Description of Artificial Sequence Synthetic oligonucleotide
ggccccatggtgtaatggttagcactctggactctaaatccagcgatccgagttcaaatctcggtgggacct

SEQ ID NO: 188 - DNA, 72 bp, Artificial Sequence
  Description of Artificial Sequence Synthetic oligonucleotide
ggtctcatggtgtaatggttagcacactggactctaagtccagcaatccgagttcgagtcttggtgagacca

SEQ ID NO: 189 - DNA, 72 bp, Artificial Sequence
  Description of Artificial Sequence Synthetic oligonucleotide
ggacccatggtgtaatggttagcactctggactctaaatccagcaatccaagttcaaatctcggtgggacct

SEQ ID NO: 190 - DNA, 72 bp, Artificial Sequence
  Description of Artificial Sequence Synthetic oligonucleotide
gtttccatggtgtaatggttggcactctggactctaaatccagcaatccaagttcaagtctctgtgggacct

SEQ ID NO: 191 - DNA, 51 bp, Artificial Sequence
  Description of Artificial Sequence Synthetic oligonucleotide
ctgcaaagttctttgaaagagcaacaaaatggcttcaactatctgagtgac

SEQ ID NO: 192 - DNA, 51 bp, Artificial Sequence
  Description of Artificial Sequence Synthetic oligonucleotide
ctgcaaagttctttgaaagagcaataaaatggcttcaactatctgagtgac

SEQ ID NO: 193 - DNA, 51 bp, Artificial Sequence
  Description of Artificial Sequence Synthetic oligonucleotide
ctgagacctctaagagccttatctcgatttgaagggatgagggtggttgtg

SEQ ID NO: 194 - DNA, 51 bp, Artificial Sequence
  Description of Artificial Sequence Synthetic oligonucleotide
acaagccttttcagctttagagggcgagcaaaggatgtgggatctgagaac

SEQ ID NO: 195 - DNA, 51 bp, Artificial Sequence
  Description of Artificial Sequence Synthetic oligonucleotide
ctaatagaaagaaatgagaccgcccggtggaaaaatgtgaaagtaaacttt

SEQ ID NO: 196 - DNA, 73 bp, Artificial Sequence
  Description of Artificial Sequence Synthetic oligonucleotide
gcccggctagctcagtcggtagagcatgggactctaaatcccagggtcgtgggttcgagccccacgttgggcg

SEQ ID NO: 197 - DNA, 73 bp, Artificial Sequence
  Description of Artificial Sequence Synthetic oligonucleotide
gcccggctagctcagtcggtagagcatgagactctaaatctcagggtcgtgggttcgagccccacgttgggcg

SEQ ID NO: 198 - DNA, 73 bp, Artificial Sequence
  Description of Artificial Sequence Synthetic oligonucleotide
gcccagctagctcagtctgtagagcatgagactctaaatctcagggtcgtgagttcgagccccacgttgggtg

SEQ ID NO: 199 - DNA, 73 bp, Artificial Sequence
  Description of Artificial Sequence Synthetic oligonucleotide
gcccagatagctcagtgggtagagcatgagactctaaatctcagggtcatgggttcatgccccatgttgggta

SEQ ID NO: 200 - DNA, 73 bp, Artificial Sequence
  Description of Artificial Sequence Synthetic oligonucleotide
gtcctgctggctcagtcggtacagcatgggactctaaatcccagggtcgtgggttcgagctccacgttgggta

SEQ ID NO: 201 - DNA, 73 bp, Artificial Sequence
  Description of Artificial Sequence Synthetic oligonucleotide
gcctggctagctcagtccatagagcatgggactctaaatcccagggtcatgggttcgagccccatattaggca

SEQ ID NO: 202 - DNA, 73 bp, Artificial Sequence
  Description of Artificial Sequence Synthetic oligonucleotide
gcccagctagcttagttggtagagcatgagactctaaatctcagagtcatgggttcaggcctcatgtttggca

SEQ ID NO: 203 - DNA, 74 bp, Artificial Sequence
  Description of Artificial Sequence Synthetic oligonucleotide
aacctggctaggtcagttggtagatcatgagactctaaatctcagggtcatgggttcaagccccatgttggttt

SEQ ID NO: 204 - DNA, 73 bp, Artificial Sequence
  Description of Artificial Sequence Synthetic oligonucleotide
gcccagctagctcagttggtagagcgtgggactttaaatcctagggtcgtgggttcgaaccccacgttgggcg

SEQ ID NO: 205 - DNA, 73 bp, Artificial Sequence
  Description of Artificial Sequence Synthetic oligonucleotide
gcccagctagctcagtctgtagagcatgagactttaagtctcagggtcatgggttggagccccatgttgtgca

SEQ ID NO: 206 - DNA, 77 bp, Artificial Sequence
  Description of Artificial Sequence Synthetic oligonucleotide
gcctagctagttcagtcggtagagcatgagactttaaatctcaggttcatgagtttgagccccatgttggtttggca

SEQ ID NO: 207 - DNA, 73 bp, Artificial Sequence
  Description of Artificial Sequence Synthetic oligonucleotide
ccccggctagctcagtcagtagagcttgagaatttaaatctcagggtcgtgggttggagccccacgttgggcg

SEQ ID NO: 208 - DNA, 73 bp, Artificial Sequence
  Description of Artificial Sequence Synthetic oligonucleotide
gcccggctagctcagtcggtagagcatgggactttaaatcccagggtcgtgggttcgagccccacgttgggcg

SEQ ID NO: 209 - DNA, 73 bp, Artificial Sequence
  Description of Artificial Sequence Synthetic oligonucleotide
gcccggctagctcagtcggtagagcatgagactttaaatctcagggtcgtgggttcgagccccacgttgggcg

SEQ ID NO: 210 - DNA, 73 bp, Artificial Sequence
  Description of Artificial Sequence Synthetic oligonucleotide
gcccagctagctcagtctgtagagcatgagactttaaatctcagggtcgtgagttcgagccccacgttgggtg

SEQ ID NO: 211 - DNA, 73 bp, Artificial Sequence
  Description of Artificial Sequence Synthetic oligonucleotide
gcccagatagctcagtgggtagagcatgagactttaaatctcagggtcatgggttcatgccccatgttgggta

SEQ ID NO: 212 - DNA, 73 bp, Artificial Sequence
  Description of Artificial Sequence Synthetic oligonucleotide
gtcctgctggctcagtcggtacagcatgggactttaaatcccagggtcgtgggttcgagctccacgttgggta

SEQ ID NO: 213 - DNA, 73 bp, Artificial Sequence
  Description of Artificial Sequence Synthetic oligonucleotide
gcctggctagctcagtccatagagcatgggactttaaatcccagggtcatgggttcgagccccatattaggca

SEQ ID NO: 214 - DNA, 73 bp, Artificial Sequence
  Description of Artificial Sequence Synthetic oligonucleotide
gcccagctagcttagttggtagagcatgagactttaaatctcagagtcatgggttcaggcctcatgtttggca

SEQ ID NO: 215 - DNA, 74 bp, Artificial Sequence
  Description of Artificial Sequence Synthetic oligonucleotide
aacctggctaggtcagttggtagatcatgagactttaaatctcagggtcatgggttcaagccccatgttggttt

SEQ ID NO: 216 - DNA, 73 bp, Artificial Sequence
  Description of Artificial Sequence Synthetic oligonucleotide
gcccggatagctcagtcggtagagcatcagactctaaatctgagggtccagggttcaagtccctgttcgggcg

SEQ ID NO: 217 - DNA, 73 bp, Artificial Sequence
  Description of Artificial Sequence Synthetic oligonucleotide
gcctggatagctcagtcggtagagcatcagactctaaatctgagggtccagggttcaagtccctgttcaggcg

SEQ ID NO: 218 - DNA, 73 bp, Artificial Sequence
  Description of Artificial Sequence Synthetic oligonucleotide
gcctggatagctcaattggtagagcatcagactctaaatctgagggttcagggttcaagtccctgttcaggcg

SEQ ID NO: 219 - DNA, 73 bp, Artificial Sequence
  Description of Artificial Sequence Synthetic oligonucleotide
gcccagccagctcagtaggtagagtatgagactctaaatctcagggtggtgggttcgagccccatgttggggg

SEQ ID NO: 220 - DNA, 79 bp, Artificial Sequence
  Description of Artificial Sequence Synthetic oligonucleotide
tgtggtgtagctcagtcggtagagcatcagactctaaatctgagggtccagggttcaggtccctgttcgggtgccaaaa

SEQ ID NO: 221 - DNA, 73 bp, Artificial Sequence
  Description of Artificial Sequence Synthetic oligonucleotide
gcccggatagctcagtcggtagagcatcagactttaaatctgagggtccagggttcaagtccctgttcgggcg

SEQ ID NO: 222 - DNA, 73 bp, Artificial Sequence
  Description of Artificial Sequence Synthetic oligonucleotide
gcctggatagctcagtcggtagagcatcagactttaaatctgagggtccagggttcaagtccctgttcaggcg

SEQ ID NO: 223 - DNA, 73 bp, Artificial Sequence
  Description of Artificial Sequence Synthetic oligonucleotide
gcctggatagctcaattggtagagcatcagactttaaatctgagggttcagggttcaagtccctgttcaggcg

SEQ ID NO: 224 - DNA, 73 bp, Artificial Sequence
  Description of Artificial Sequence Synthetic oligonucleotide
gcccagccagctcagtaggtagagtatgagactttaaatctcagggtggtgggttcgagccccatgttggggg

SEQ ID NO: 225 - DNA, 79 bp, Artificial Sequence
  Description of Artificial Sequence Synthetic oligonucleotide
tgtggtgtagctcagtcggtagagcatcagactttaaatctgagggtccagggttcaggtccctgttcgggtgccaaaa

SEQ ID NO: 226 - DNA, 82 bp, Artificial Sequence
  Description of Artificial Sequence Synthetic oligonucleotide
gtagtcgtggccgagtggttaaggcgatggactctaaatccattggggtttccccgcgcaggttcgaatcctgccgactacg

SEQ ID NO: 227 - DNA, 82 bp, Artificial Sequence
  Description of Artificial Sequence Synthetic oligonucleotide
gtagtcgtggccgagtggttaaggcgatggactctaaatccattggggtctccccgcgcaggttcgaatcctgccgactacg

SEQ ID NO: 228 - DNA, 82 bp, Artificial Sequence
  Description of Artificial Sequence Synthetic oligonucleotide
gtagtcgtggccaagtgagtaaggcaatggactctaaatccattggggtctcccagcacaggttcaaatcctgctgactatg

SEQ ID NO: 229 - DNA, 82 bp, Artificial Sequence
  Description of Artificial Sequence Synthetic oligonucleotide
gtagtcgtggccgagtggttaaggcgatggacttcaaatccattggggtttccccgcgcaggttcgaatcctgccgactacg

SEQ ID NO: 230 - DNA, 82 bp, Artificial Sequence
  Description of Artificial Sequence Synthetic oligonucleotide
gtagtcgtggccgagtggttaaggcgatggacttcaaatccattggggtctccccgcgcaggttcgaatcctgccgactacg

SEQ ID NO: 231 - DNA, 82 bp, Artificial Sequence
  Description of Artificial Sequence Synthetic oligonucleotide
gtagtcgtggccaagtgagtaaggcaatggacttcaaatccattggggtctcccagcacaggttcaaatcctgctgactatg

SEQ ID NO: 232 - DNA, 82 bp, Artificial Sequence
  Description of Artificial Sequence Synthetic oligonucleotide
gtagtcgtggccgagtggttaaggcgatggactttaaatccattggggtttccccgcgcaggttcgaatcctgccgactacg

SEQ ID NO: 233 - DNA, 82 bp, Artificial Sequence
  Description of Artificial Sequence Synthetic oligonucleotide
gtagtcgtggccgagtggttaaggcgatggactttaaatccattggggtctccccgcgcaggttcgaatcctgccgactacg

SEQ ID NO: 234 - DNA, 82 bp, Artificial Sequence
  Description of Artificial Sequence Synthetic oligonucleotide
gtagtcgtggccaagtgagtaaggcaatggactttaaatccattggggtctcccagcacaggttcaaatcctgctgactatg

SEQ ID NO: 235 - DNA, 82 bp, Artificial Sequence
  Description of Artificial Sequence Synthetic oligonucleotide
gctgtgatggccgagtggttaaggcgttggactctaaatccaatggggtctccccgcgcaggttcgaatcctgctcacagcg

SEQ ID NO: 236 - DNA, 82 bp, Artificial Sequence
  Description of Artificial Sequence Synthetic oligonucleotide
gtcacggtggccgagtggttaaggcgttggactctaaatccaatggggtttccccgcacaggttcgaatcctgttcgtgacg

SEQ ID NO: 237 - DNA, 82 bp, Artificial Sequence
  Description of Artificial Sequence Synthetic oligonucleotide
gctgtgatggccgagtggttaaggcgttggactctaaatccaatgggttcttcccgcgcaggttcaaatcctgctcacagcg

SEQ ID NO: 238 - DNA, 82 bp, Artificial Sequence
  Description of Artificial Sequence Synthetic oligonucleotide
gctgtgatggccgagtggttaaggcgttggacttcaaatccaatggggtctccccgcgcaggttcgaatcctgctcacagcg

SEQ ID NO: 239 - DNA, 82 bp, Artificial Sequence
  Description of Artificial Sequence Synthetic oligonucleotide
gtcacggtggccgagtggttaaggcgttggacttcaaatccaatggggtttccccgcacaggttcgaatcctgttcgtgacg

SEQ ID NO: 240 - DNA, 82 bp, Artificial Sequence
  Description of Artificial Sequence Synthetic oligonucleotide
gctgtgatggccgagtggttaaggcgttggacttcaaatccaatgggttcttcccgcgcaggttcaaatcctgctcacagcg

SEQ ID NO: 241 - DNA, 82 bp, Artificial Sequence
  Description of Artificial Sequence Synthetic oligonucleotide
gctgtgatggccgagtggttaaggcgttggactttaaatccaatggggtctccccgcgcaggttcgaatcctgctcacagcg

SEQ ID NO: 242 - DNA, 82 bp, Artificial Sequence
  Description of Artificial Sequence Synthetic oligonucleotide
gtcacggtggccgagtggttaaggcgttggactttaaatccaatggggtttccccgcacaggttcgaatcctgttcgtgacg

SEQ ID NO: 243 - DNA, 82 bp, Artificial Sequence
  Description of Artificial Sequence Synthetic oligonucleotide
gctgtgatggccgagtggttaaggcgttggactttaaatccaatgggttcttcccgcgcaggttcaaatcctgctcacagcg

SEQ ID NO: 244 - DNA, 82 bp, Artificial Sequence
  Description of Artificial Sequence Synthetic oligonucleotide
gacgaggtggccgagtggttaaggcgatggactctaaatccattgtgctctgcacgcgtgggttcgaatcccaccttcgtcg

SEQ ID NO: 245 - DNA, 82 bp, Artificial Sequence
  Description of Artificial Sequence Synthetic oligonucleotide
gacgaggtggccgagtggttaaggcgatggactctaaatccattgtgctctgcacgcatgggttcgaatcccatcctcgtcg

SEQ ID NO: 246 - DNA, 82 bp, Artificial Sequence
  Description of Artificial Sequence Synthetic oligonucleotide
gacgaggtggccgagtggttaaggcgatggactctaaatccattgtgctttgcacgcgtgggttcgaatcccatcctcgtcg

SEQ ID NO: 247 - DNA, 82 bp, Artificial Sequence
  Description of Artificial Sequence Synthetic oligonucleotide
gacgaggtggccgagtggttaaggcgatggactctaaatccattgtgctctgcacgcgtgggttcgaatcccatcctcgtcg

SEQ ID NO: 248 - DNA, 82 bp, Artificial Sequence
  Description of Artificial Sequence Synthetic oligonucleotide
gatgaggtggccgagtggttaaggcgatggactctaaatccattgtgctctgcacgcatgggttcgaatcccatcctcatcg

SEQ ID NO: 249 - DNA, 82 bp, Artificial Sequence
  Description of Artificial Sequence Synthetic oligonucleotide
gacgaggtggccgagtggttaaggcgatggacttcaaatccattgtgctctgcacgcgtgggttcgaatcccaccttcgtcg

SEQ ID NO: 250 - DNA, 82 bp, Artificial Sequence
  Description of Artificial Sequence Synthetic oligonucleotide
gacgaggtggccgagtggttaaggcgatggacttcaaatccattgtgctctgcacgcatgggttcgaatcccatcctcgtcg

SEQ ID NO: 251 - DNA, 82 bp, Artificial Sequence
  Description of Artificial Sequence Synthetic oligonucleotide
gacgaggtggccgagtggttaaggcgatggacttcaaatccattgtgctttgcacgcgtgggttcgaatcccatcctcgtcg

SEQ ID NO: 252 - DNA, 82 bp, Artificial Sequence
  Description of Artificial Sequence Synthetic oligonucleotide
gacgaggtggccgagtggttaaggcgatggacttcaaatccattgtgctctgcacgcgtgggttcgaatcccatcctcgtcg

SEQ ID NO: 253 - DNA, 82 bp, Artificial Sequence
  Description of Artificial Sequence Synthetic oligonucleotide
gatgaggtggccgagtggttaaggcgatggacttcaaatccattgtgctctgcacgcatgggttcgaatcccatcctcatcg

SEQ ID NO: 254 - DNA, 82 bp, Artificial Sequence
  Description of Artificial Sequence Synthetic oligonucleotide
gacgaggtggccgagtggttaaggcgatggactttaaatccattgtgctctgcacgcgtgggttcgaatcccaccttcgtcg

SEQ ID NO: 255 - DNA, 82 bp, Artificial Sequence
  Description of Artificial Sequence Synthetic oligonucleotide
gacgaggtggccgagtggttaaggcgatggactttaaatccattgtgctctgcacgcatgggttcgaatcccatcctcgtcg

SEQ ID NO: 256 - DNA, 82 bp, Artificial Sequence
  Description of Artificial Sequence Synthetic oligonucleotide
gacgaggtggccgagtggttaaggcgatggactttaaatccattgtgctttgcacgcgtgggttcgaatcccatcctcgtcg

SEQ ID NO: 257 - DNA, 82 bp, Artificial Sequence
  Description of Artificial Sequence Synthetic oligonucleotide
gacgaggtggccgagtggttaaggcgatggactttaaatccattgtgctctgcacgcgtgggttcgaatcccatcctcgtcg

SEQ ID NO: 258 - DNA, 82 bp, Artificial Sequence
  Description of Artificial Sequence Synthetic oligonucleotide
gatgaggtggccgagtggttaaggcgatggactttaaatccattgtgctctgcacgcatgggttcgaatcccatcctcatcg

SEQ ID NO: 259 - DNA, 73 bp, Artificial Sequence
  Description of Artificial Sequence Synthetic oligonucleotide
gctgaaatagctcagttgggagagcattagactctagatctaaaggtccctggtttgatcccgggtttcggca

SEQ ID NO: 260 - DNA, 73 bp, Artificial Sequence
  Description of Artificial Sequence Synthetic oligonucleotide
gctgaaatagctcagttgggagagcattagacttcagatctaaaggtccctggtttgatcccgggtttcggca

SEQ ID NO: 261 - DNA, 73 bp, Artificial Sequence
  Description of Artificial Sequence Synthetic oligonucleotide
gctgaaatagctcagttgggagagcattagactttagatctaaaggtccctggtttgatcccgggtttcggca

SEQ ID NO: 262 - DNA, 82 bp, Artificial Sequence
  Description of Artificial Sequence Synthetic oligonucleotide
gcagcgatggccgagtggttaaggcgttggactctaaatccaatggggtctccccgcgcaggttcgaaccctgctcgctgcg

SEQ ID NO: 263 - DNA, 82 bp, Artificial Sequence
  Description of Artificial Sequence Synthetic oligonucleotide
gcagcgatggccgagtggttaaggcgttggacttcaaatccaatggggtctccccgcgcaggttcgaaccctgctcgctgcg

SEQ ID NO: 264 - DNA, 82 bp, Artificial Sequence
  Description of Artificial Sequence Synthetic oligonucleotide
gcagcgatggccgagtggttaaggcgttggactttaaatccaatggggtctccccgcgcaggttcgaaccctgctcgctgcg

SEQ ID NO: 265 - DNA, 72 bp, Artificial Sequence
  Description of Artificial Sequence Synthetic oligonucleotide
gacctcgtggcgcaatggtagcgcgtctgactctagatcagaaggttgcgtgttcaaatcacgtcggggtca

SEQ ID NO: 266 - DNA, 72 bp, Artificial Sequence
  Description of Artificial Sequence Synthetic oligonucleotide
gacctcgtggcgcaacggtagcgcgtctgactctagatcagaaggctgcgtgttcgaatcacgtcggggtca

SEQ ID NO: 267 - DNA, 72 bp, Artificial Sequence
  Description of Artificial Sequence Synthetic oligonucleotide
ggcctcgtggcgcaacggtagcgcgtctgactctagatcagaaggttgcgtgttcaaatcacgtcggggtca

SEQ ID NO: 268 - DNA, 72 bp, Artificial Sequence
  Description of Artificial Sequence Synthetic oligonucleotide
gacctcgtggcgcaacggtagcgcgtctgactctagatcagaaggttgcgtgttcaaatcacgtcggggtca

SEQ ID NO: 269 - DNA, 72 bp, Artificial Sequence
  Description of Artificial Sequence Synthetic oligonucleotide
gacctcgtggcgcaatggtagcgcgtctgactctagatcagaaggttgcgtgttcaagtcacgtcggggtca

SEQ ID NO: 270 - DNA, 72 bp, Artificial Sequence
  Description of Artificial Sequence Synthetic oligonucleotide
gacctcgtggcacaatggtagcacgtctgactctagatcagaaggttgcgtgttcaaatcacgtcggggtca

SEQ ID NO: 271 - DNA, 72 bp, Artificial Sequence
  Description of Artificial Sequence Synthetic oligonucleotide
gacctcgtggcgcaatggtagcgcgtctgacttcagatcagaaggttgcgtgttcaaatcacgtcggggtca

SEQ ID NO: 272 - DNA, 72 bp, Artificial Sequence
  Description of Artificial Sequence Synthetic oligonucleotide
gacctcgtggcgcaacggtagcgcgtctgacttcagatcagaaggctgcgtgttcgaatcacgtcggggtca

SEQ ID NO: 273 - DNA, 72 bp, Artificial Sequence
  Description of Artificial Sequence Synthetic oligonucleotide
ggcctcgtggcgcaacggtagcgcgtctgacttcagatcagaaggttgcgtgttcaaatcacgtcggggtca

SEQ ID NO: 274 - DNA, 72 bp, Artificial Sequence
  Description of Artificial Sequence Synthetic oligonucleotide
gacctcgtggcgcaacggtagcgcgtctgacttcagatcagaaggttgcgtgttcaaatcacgtcggggtca

SEQ ID NO: 275 - DNA, 72 bp, Artificial Sequence
  Description of Artificial Sequence Synthetic oligonucleotide
gacctcgtggcgcaatggtagcgcgtctgacttcagatcagaaggttgcgtgttcaagtcacgtcggggtca

SEQ ID NO: 276 - DNA, 72 bp, Artificial Sequence
  Description of Artificial Sequence Synthetic oligonucleotide
gacctcgtggcacaatggtagcacgtctgacttcagatcagaaggttgcgtgttcaaatcacgtcggggtca

SEQ ID NO: 277 - DNA, 73 bp, Artificial Sequence
  Description of Artificial Sequence Synthetic oligonucleotide
ccttcgatagctcagttggtagagcggaggactctagatccttaggtcgctggttcgaatccggctcgaagga

SEQ ID NO: 278 - DNA, 73 bp, Artificial Sequence
  Description of Artificial Sequence Synthetic oligonucleotide
ccttcgatagctcagttggtagagcggaggactctagatccttaggtcgctggttcgattccggctcgaagga

SEQ ID NO: 279 - DNA, 73 bp, Artificial Sequence
  Description of Artificial Sequence Synthetic oligonucleotide
ccttcgatagctcagctggtagagcggaggactctagatccttaggtcgctggttcgattccggctcgaagga

SEQ ID NO: 280 - DNA, 73 bp, Artificial Sequence
  Description of Artificial Sequence Synthetic oligonucleotide
ccttcgatagctcagctggtagagcggaggactctagatccttaggtcgctggttcgactccggctcgaagga

SEQ ID NO: 281 - DNA, 73 bp, Artificial Sequence
  Description of Artificial Sequence Synthetic oligonucleotide
ctttcgatagttcagttggtagagcggaggactctagatccttaggtcgctggttcgagtccggctcgaagga

SEQ ID NO: 282 - DNA, 73 bp, Artificial Sequence
  Description of Artificial Sequence Synthetic oligonucleotide
ccttcgatagctcagttggtagagcggaggactttagatccttaggtcgctggttcgaatccggctcgaagga

SEQ ID NO: 283 - DNA, 73 bp, Artificial Sequence
  Description of Artificial Sequence Synthetic oligonucleotide
ccttcgatagctcagttggtagagcggaggactttagatccttaggtcgctggttcgattccggctcgaagga

SEQ ID NO: 284 - DNA, 73 bp, Artificial Sequence
  Description of Artificial Sequence Synthetic oligonucleotide
ccttcgatagctcagctggtagagcggaggactttagatccttaggtcgctggttcgattccggctcgaagga

SEQ ID NO: 285 - DNA, 73 bp, Artificial Sequence
  Description of Artificial Sequence Synthetic oligonucleotide
ccttcgatagctcagctggtagagcggaggactttagatccttaggtcgctggttcgactccggctcgaagga

SEQ ID NO: 286 - DNA, 73 bp, Artificial Sequence
  Description of Artificial Sequence Synthetic oligonucleotide
ctttcgatagttcagttggtagagcggaggactttagatccttaggtcgctggttcgagtccggctcgaagga

SEQ ID NO: 287 - DNA, 72 bp, Artificial Sequence
  Description of Artificial Sequence Synthetic oligonucleotide
ggcctcgtggcgcaacggtagcgcgtctgacttcagatcagaaggttgcgggttcaaatcccgtcggggtca

SEQ ID NO: 288 - DNA, 72 bp, Artificial Sequence
  Description of Artificial Sequence Synthetic oligonucleotide
ggcctcgtggcgcaacggtagcgcgtctgacttcagatcagaaggttacgggttcaaatcccgtcggggtca

SEQ ID NO: 289 - DNA, 72 bp, Artificial Sequence
  Description of Artificial Sequence Synthetic oligonucleotide
ggcctcgtggcgcaacggtagcgcgtctgacttcagatcagaaggttccgggttcaaatcccggcggggtca

SEQ ID NO: 290 - DNA, 74 bp, Artificial Sequence
  Description of Artificial Sequence Synthetic oligonucleotide
cgtcggctctgtggcgcaatggatagcgcattggacttcaaattcaaaggttgtgggttcgagtcccagagtcg

SEQ ID NO: 291 - DNA, 77 bp, Artificial Sequence
  Description of Artificial Sequence Synthetic oligonucleotide
cgtcgccccagtggcctaatggataaggcactggccttcaaagccagggattgtgggttcgagtcccacctggggtg

SEQ ID NO: 292 - DNA, 77 bp, Artificial Sequence
  Description of Artificial Sequence Synthetic oligonucleotide
cgtcggctccgtggcgcaatggatagcgcattggacttcaaattcaaaggttccgggttcgagtcccggcggagtcg

SEQ ID NO: 293 - DNA, 77 bp, Artificial Sequence
  Description of Artificial Sequence Synthetic oligonucleotide
cgtcgccccagtggcctaatggataaggcattggccttcaaagccagggattgtgggttcgagtcccatctggggtg

SEQ ID NO: 294 - DNA, 77 bp, Artificial Sequence
  Description of Artificial Sequence Synthetic oligonucleotide
cgtcggctctgtggcgcaatggatagcgcattggacttcaaattcaaaggttgtgggttcgaatcccaccagagtcg

SEQ ID NO: 295 - DNA, 95 bp, Artificial Sequence
  Description of Artificial Sequence Synthetic oligonucleotide
cgtcggctctgtggcgcaatggatagcgcattggacttcaagctgagcctagtgtggtcattcaaaggttgtgggttcgagtcccaccagagtcg

SEQ ID NO: 296 - DNA, 77 bp, Artificial Sequence
  Description of Artificial Sequence Synthetic oligonucleotide
cgtcgccccggtggcctaatggataaggcattggccttcaaagccagggattgtgggttcgagtcccacccggggta

SEQ ID NO: 297 - DNA, 89 bp, Artificial Sequence
  Description of Artificial Sequence Synthetic oligonucleotide
cgtcggctccgtggcgcaatggatagcgcattggacttcaagaggctgaaggcattcaaaggttccgggttcgagtcccggcggagtcg

SEQ ID NO: 298 - DNA, 92 bp, Artificial Sequence
  Description of Artificial Sequence Synthetic oligonucleotide
cgtcggctctgtggcgcaatggatagcgcattggacttcaagtgacgaatagagcaattcaaaggttgtgggttcgaatcccaccagagtcg

SEQ ID NO: 299 - DNA, 77 bp, Artificial Sequence
  Description of Artificial Sequence Synthetic oligonucleotide
cgtcggccgcgtggcctaatggataaggcgtctgacttcagatcagaagattgcaggttcgagtcctgccgcggtcg

SEQ ID NO: 300 - DNA, 77 bp, Artificial Sequence
  Description of Artificial Sequence Synthetic oligonucleotide
cgtcgaccgcgtggcctaatggataaggcgtctgacttcagatcagaagattgagggttcgagtcccttcgtggtcg

SEQ ID NO: 301 - DNA, 90 bp, Artificial Sequence
  Description of Artificial Sequence Synthetic oligonucleotide
cgtcggctctgtggcgcaatggatagcgcattggacttcaagatagttagagaaattcaaaggttgtgggttcgagtcccaccagagtcg

SEQ ID NO: 302 - DNA, 76 bp, Artificial Sequence
  Description of Artificial Sequence Synthetic oligonucleotide
cgtcggttccatggtgtaatggtgagcactctggactctaaatccagcgatccgagttcgagtctcggtggaacct

SEQ ID NO: 303 - DNA, 76 bp, Artificial Sequence
  Description of Artificial Sequence Synthetic oligonucleotide
cgtcggccccatggtgtaatggttagcactctggactctaaatccagcgatccgagttcaaatctcggtgggacct

SEQ ID NO: 304 - DNA, 76 bp, Artificial Sequence
  Description of Artificial Sequence Synthetic oligonucleotide
cgtcggtcccatggtgtaatggttagcactctggactctaaatccagcaatccgagttcgaatctcggtgggacct

SEQ ID NO: 305 - DNA, 76 bp, Artificial Sequence
  Description of Artificial Sequence Synthetic oligonucleotide
cgtcggtcccatggtgtaatggttagcactctggactctaaatccagcgatccgagttcaaatctcggtgggacct

SEQ ID NO: 306 - DNA, 76 bp, Artificial Sequence
  Description of Artificial Sequence Synthetic oligonucleotide
cgtcggccccatggtgtaatggtcagcactctggactctaaatccagcgatccgagttcaaatctcggtgggaccc

SEQ ID NO: 307 - DNA, 76 bp, Artificial Sequence
  Description of Artificial Sequence Synthetic oligonucleotide
cgtcggttccatggtgtaatggtaagcactctggactctaaatccagcgatccgagttcgagtctcggtggaacct

SEQ ID NO: 308 - DNA, 76 bp, Artificial Sequence
  Description of Artificial Sequence Synthetic oligonucleotide
cgtcggttccatggtgtaatggttagcactctggactctaaatccggtaatccgagttcaaatctcggtggaacct

SEQ ID NO: 309 - DNA, 76 bp, Artificial Sequence
  Description of Artificial Sequence Synthetic oligonucleotide
cgtcggttccatggtgtaatggttagcactctggactctaaatccagcgatccgagttcaagtctcggtggaacct

SEQ ID NO: 310 - DNA, 76 bp, Artificial Sequence
  Description of Artificial Sequence Synthetic oligonucleotide
cgtcggttccatggtgtaatggtaagcactctggactttaaatccagcgatccgagttcgagtctcggtggaacct

SEQ ID NO: 311 - DNA, 76 bp, Artificial Sequence
  Description of Artificial Sequence Synthetic oligonucleotide
cgtcggccccatggtgtaatggttagcactctggactttaaatccagcgatccgagttcaaatctcggtgggacct

SEQ ID NO: 312 - DNA, 76 bp, Artificial Sequence
  Description of Artificial Sequence Synthetic oligonucleotide
cgtcggttccatggtgtaatggtgagcactctggactttaaatccagcgatccgagttcgagtctcggtggaacct

SEQ ID NO: 313 - DNA, 76 bp, Artificial Sequence
  Description of Artificial Sequence Synthetic oligonucleotide
cgtcggttccatggtgtaatggttagcactctggactttaaatccagcgatccgagttcaaatctcggtggaacct

SEQ ID NO: 314 - DNA, 76 bp, Artificial Sequence
  Description of Artificial Sequence Synthetic oligonucleotide
cgtcggtcccatggtgtaatggttagcactctggactttaaatccagcgatccgagttcaaatctcggtgggacct

SEQ ID NO: 315 - DNA, 76 bp, Artificial Sequence
  Description of Artificial Sequence Synthetic oligonucleotide
cgtcggtcccatggtgtaatggttagcactctggactttaaatccagcaatccgagttcgaatctcggtgggacct

SEQ ID NO: 316 - DNA, 76 bp, Artificial Sequence
  Description of Artificial Sequence Synthetic oligonucleotide
cgtcggttccatggtgtaatggttagcactctggactttaaatccggtaatccgagttcaaatctcggtggaacct

SEQ ID NO: 317 - DNA, 76 bp, Artificial Sequence
  Description of Artificial Sequence Synthetic oligonucleotide
cgtcggccccatggtgtaatggtcagcactctggactttaaatccagcgatccgagttcaaatctcggtgggaccc

SEQ ID NO: 318 - DNA, 76 bp, Artificial Sequence
  Description of Artificial Sequence Synthetic oligonucleotide
cgtcggttccatggtgtaatggttagcactctggactttaaatccagcgatccgagttcaagtctcggtggaacct

SEQ ID NO: 319 - DNA, 76 bp, Artificial Sequence
  Description of Artificial Sequence Synthetic oligonucleotide
cgtcgacctcgtggcgcaatggtagcgcgtctgactctagatcagaaggttgcgtgttcaagtcacgtcggggtca

SEQ ID NO: 320 - DNA, 76 bp, Artificial Sequence
  Description of Artificial Sequence Synthetic oligonucleotide
cgtcgacctcgtggcgcaacggtagcgcgtctgactctagatcagaaggttgcgtgttcaaatcacgtcggggtca

SEQ ID NO: 321 - DNA, 76 bp, Artificial Sequence
  Description of Artificial Sequence Synthetic oligonucleotide
cgtcggcctcgtggcgcaacggtagcgcgtctgactctagatcagaaggttgcgtgttcaaatcacgtcggggtca

SEQ ID NO: 322 - DNA, 76 bp, Artificial Sequence
  Description of Artificial Sequence Synthetic oligonucleotide
cgtcgacctcgtggcgcaacggtagcgcgtctgactctagatcagaaggctgcgtgttcgaatcacgtcggggtca

SEQ ID NO: 323 - DNA, 76 bp, Artificial Sequence
  Description of Artificial Sequence Synthetic oligonucleotide
cgtcgacctcgtggcgcaacggcagcgcgtctgactctagatcagaaggttgcgtgttcaaatcacgtcggggtca

SEQ ID NO: 324 - DNA, 76 bp, Artificial Sequence
  Description of Artificial Sequence Synthetic oligonucleotide
cgtctcccacatggtctagcggttaggattcctggttctaacccaggcggcccgggttcgactcccggtgtgggaa

SEQ ID NO: 325 - DNA, 76 bp, Artificial Sequence
  Description of Artificial Sequence Synthetic oligonucleotide
cgtctcccatatggtctagcggttaggattcctggttctaacccaggtggcccgggttcgactcccggtatgggaa

SEQ ID NO: 326 - DNA, 76 bp, Artificial Sequence
  Description of Artificial Sequence Synthetic oligonucleotide
cgtctccctggtggtctagtggctaggattcggcgctctaaccgccgcggcccgggttcgattcccggtcagggaa

SEQ ID NO: 327 - DNA, 76 bp, Artificial Sequence
  Description of Artificial Sequence Synthetic oligonucleotide
cgtctccctggtggtctagtggttaggattcggcgctctaaccgccgcggcccgggttcgattcccggtcagggaa

SEQ ID NO: 328 - DNA, 73 bp, Artificial Sequence
  Description of Artificial Sequence Synthetic oligonucleotide
cgtctccctggtctagtggctaggattcggcgctctaaccgccgcggcccgggttcgattcccggccagggaa

SEQ ID NO: 329 - DNA, 76 bp, Artificial Sequence; identical to SEQ ID NO: 324
  Description of Artificial Sequence Synthetic oligonucleotide

SEQ ID NO: 330 - DNA, 76 bp, Artificial Sequence; identical to SEQ ID NO: 325
  Description of Artificial Sequence Synthetic oligonucleotide

SEQ ID NO: 331 - DNA, 76 bp, Artificial Sequence; identical to SEQ ID NO: 327
  Description of Artificial Sequence Synthetic oligonucleotide

SEQ ID NO: 332 - DNA, 76 bp, Artificial Sequence
  Description of Artificial Sequence Synthetic oligonucleotide
cgtctccctggtggtctagtggttaggattcggcgctctaaccgccgcggcccgggttcgattcccggtcaggaaa

SEQ ID NO: 333 - DNA, 76 bp, Artificial Sequence
  Description of Artificial Sequence Synthetic oligonucleotide
cgtctccctggtggtctagtggctaggattcggcgctctaaccgccgcggcccgggttcgattcccggccagggaa

SEQ ID NO: 334 - DNA, 72 bp, Artificial Sequence; identical to SEQ ID NO: 273
  Description of Artificial Sequence Synthetic oligonucleotide

SEQ ID NO: 335 - DNA, 72 bp, Artificial Sequence; identical to SEQ ID NO: 275
  Description of Artificial Sequence Synthetic oligonucleotide

SEQ ID NO: 336 - DNA, 72 bp, Artificial Sequence; identical to SEQ ID NO: 274
  Description of Artificial Sequence Synthetic oligonucleotide

SEQ ID NO: 337 - DNA, 72 bp, Artificial Sequence; identical to SEQ ID NO: 272
  Description of Artificial Sequence Synthetic oligonucleotide

SEQ ID NO: 338 - DNA, 72 bp, Artificial Sequence
  Description of Artificial Sequence Synthetic oligonucleotide
gacctcgtggcgcaacggcagcgcgtctgacttcagatcagaaggttgcgtgttcaaatcacgtcggggtca

SEQ ID NO: 339 - DNA, 72 bp, Artificial Sequence
  Description of Artificial Sequence Synthetic oligonucleotide
gcgttggtggtatagtggttagcatagctgccttcaaagcagttgacccgggttcgattcccggccaacgca

SEQ ID NO: 340 - DNA, 72 bp, Artificial Sequence; identical to SEQ ID NO: 114
  Description of Artificial Sequence Synthetic oligonucleotide

SEQ ID NO: 341 - DNA, 72 bp, Artificial Sequence
  Description of Artificial Sequence Synthetic oligonucleotide
gcgttggtggtatagtggtaagcatagctgccttcaaagcagttgacccgggttcgattcccggccaacgca

SEQ ID NO: 342 - DNA, 72 bp, Artificial Sequence; identical to SEQ ID NO: 273
  Description of Artificial Sequence Synthetic oligonucleotide

SEQ ID NO: 343 - DNA, 72 bp, Artificial Sequence; identical to SEQ ID NO: 275
  Description of Artificial Sequence Synthetic oligonucleotide

SEQ ID NO: 344 - DNA, 72 bp, Artificial Sequence; identical to SEQ ID NO: 274
  Description of Artificial Sequence Synthetic oligonucleotide

SEQ ID NO: 345 - DNA, 72 bp, Artificial Sequence; identical to SEQ ID NO: 272
  Description of Artificial Sequence Synthetic oligonucleotide

SEQ ID NO: 346 - DNA, 72 bp, Artificial Sequence; identical to SEQ ID NO: 338
  Description of Artificial Sequence Synthetic oligonucleotide

SEQ ID NO: 347 - DNA, 72 bp, Artificial Sequence
  Description of Artificial Sequence Synthetic oligonucleotide
ggcctcatggtgcaacagtagtgtgtctgacttcagatcagaaggttgtatgttcaaatcacgtaggggtca

SEQ ID NO: 348 - DNA, 72 bp, Artificial Sequence; identical to SEQ ID NO: 267
  Description of Artificial Sequence Synthetic oligonucleotide

SEQ ID NO: 349 - DNA, 72 bp, Artificial Sequence; identical to SEQ ID NO: 269
  Description of Artificial Sequence Synthetic oligonucleotide

SEQ ID NO: 350 - DNA, 72 bp, Artificial Sequence; identical to SEQ ID NO: 268
  Description of Artificial Sequence Synthetic oligonucleotide

SEQ ID NO: 351 - DNA, 72 bp, Artificial Sequence; identical to SEQ ID NO: 266
  Description of Artificial Sequence Synthetic oligonucleotide

SEQ ID NO: 352 - DNA, 72 bp, Artificial Sequence
  Description of Artificial Sequence Synthetic oligonucleotide
gacctcgtggcgcaacggcagcgcgtctgactctagatcagaaggttgcgtgttcaaatcacgtcggggtca

SEQ ID NO: 353 - DNA, 72 bp, Artificial Sequence
  Description of Artificial Sequence Synthetic oligonucleotide
ggcctcatggtgcaacagtagtgtgtctgactctagatcagaaggttgtatgttcaaatcacgtaggggtca

SEQ ID NO: 354 - DNA, 71 bp, Artificial Sequence
  Description of Artificial Sequence Synthetic oligonucleotide
gcattggtggttcagtggtagaattctcgccttcaacgcgggagacccgggttcaattcccggccaatgca

SEQ ID NO: 355 - DNA, 71 bp, Artificial Sequence; identical to SEQ ID NO: 106
  Description of Artificial Sequence Synthetic oligonucleotide

SEQ ID NO: 356 - DNA, 71 bp, Artificial Sequence
  Description of Artificial Sequence Synthetic oligonucleotide
gcattggtggttcaatggtagaattctcgccttcaacgcaggagacccaggttcgattcctggccaatgca

SEQ ID NO: 357 - DNA, 71 bp, Artificial Sequence
  Description of Artificial Sequence Synthetic oligonucleotide
gcgttggtggtttagtggtagaattctcgccttcaatgcgggagacccgggttcaattcccggccactgca

SEQ ID NO: 358 - DNA, 71 bp, Artificial Sequence
  Description of Artificial Sequence Synthetic oligonucleotide
gccttggtggtgcagtggtagaattctcgccttcaacgtgggagacccgggttcaattcccggccaatgca

SEQ ID NO: 359 - DNA, 61 bp, Artificial Sequence
  Description of Artificial Sequence Synthetic oligonucleotide
ggtggttcagtggtagaattctcgccttcaacgcgggagacccgggtttaattcccggtca

SEQ ID NO: 360 - DNA, 61 bp, Artificial Sequence
  Description of Artificial Sequence Synthetic oligonucleotide
gtggtctagtggttaggattcagcgcttcaaccgccgcagcccgggttcgattcccggtca

SEQ ID NO: 361 - DNA, 71 bp, Artificial Sequence
  Description of Artificial Sequence Synthetic oligonucleotide
gcgtcagtggtttagtggtggaattcctgccttcaatgcacgagatccgtgttcaactcctggttggtgca

SEQ ID NO: 362 - DNA, 72 bp, Artificial Sequence
  Description of Artificial Sequence Synthetic oligonucleotide
gcgtcagtggttttagtggtggaattcctgccttcaatgcacgagatccgtgttcaactcctggttggtgca

SEQ ID NO: 363 - DNA, 71 bp, Artificial Sequence
  Description of Artificial Sequence Synthetic oligonucleotide
gcgttggcagttcagtggtagaattctcgccttcaacccgggagacctggattccatttccggcaaatgca

SEQ ID NO: 364 - DNA, 71 bp, Artificial Sequence; identical to SEQ ID NO: 110
  Description of Artificial Sequence Synthetic oligonucleotide

SEQ ID NO: 365 - DNA, 71 bp, Artificial Sequence; identical to SEQ ID NO: 111
  Description of Artificial Sequence Synthetic oligonucleotide

SEQ ID NO: 366 - DNA, 71 bp, Artificial Sequence
  Description of Artificial Sequence Synthetic oligonucleotide
gcattggtggttcagtggtagaattctcgccttcaacgcgggaggcccgggtttgattcccggccagtgca

SEQ ID NO: 367 - DNA, 71 bp, Artificial Sequence
  Description of Artificial Sequence Synthetic oligonucleotide
gcataggtggttcagtggtagaattcttgccttcaacgcaggaggcccaggtttgattcctggcccatgca

SEQ ID NO: 368 - DNA, 71 bp, Artificial Sequence
  Description of Artificial Sequence Synthetic oligonucleotide
gcattggtggttcagtggtagaattctcgccttcaatgcgggcggccgggcttcgattcctggccaatgca

SEQ ID NO: 369 - DNA, 71 bp, Artificial Sequence
  Description of Artificial Sequence Synthetic oligonucleotide
gcatgggtgattcagtggtagaattttcaccttcaatgcaggaggtccaggttcatttcctggcctatgca

SEQ ID NO: 370 - DNA, 72 bp, Artificial Sequence; identical to SEQ ID NO: 339
  Description of Artificial Sequence Synthetic oligonucleotide

SEQ ID NO: 371 - DNA, 72 bp, Artificial Sequence; identical to SEQ ID NO: 114
  Description of Artificial Sequence Synthetic oligonucleotide

SEQ ID NO: 372 - DNA, 72 bp, Artificial Sequence; identical to SEQ ID NO: 341
  Description of Artificial Sequence Synthetic oligonucleotide

SEQ ID NO: 373 - DNA, 72 bp, Artificial Sequence
  Description of Artificial Sequence Synthetic oligonucleotide
gcgttggtggtatagtggtgagcatagttgccttcaaagcagttgacccgggctcgattcccgcccaacgca

SEQ ID NO: 374 - DNA, 72 bp, Artificial Sequence
  Description of Artificial Sequence Synthetic oligonucleotide
gcgttggtggtatagtggtgagcatagttgccttcaaagcagttgacccgggctcgattcccggccaacgca

SEQ ID NO: 375 - DNA, 73 bp, Artificial Sequence; identical to SEQ ID NO: 1
  Description of Artificial Sequence Synthetic oligonucleotide

SEQ ID NO: 376 - DNA, 73 bp, Artificial Sequence; identical to SEQ ID NO: 3
  Description of Artificial Sequence Synthetic oligonucleotide

SEQ ID NO: 377 - DNA, 73 bp, Artificial Sequence; identical to SEQ ID NO: 4
  Description of Artificial Sequence Synthetic oligonucleotide

SEQ ID NO: 378 - DNA, 73 bp, Artificial Sequence; identical to SEQ ID NO: 6
  Description of Artificial Sequence Synthetic oligonucleotide

SEQ ID NO: 379 - DNA, 73 bp, Artificial Sequence; identical to SEQ ID NO: 7
  Description of Artificial Sequence Synthetic oligonucleotide

SEQ ID NO: 380 - DNA, 73 bp, Artificial Sequence; identical to SEQ ID NO: 8
  Description of Artificial Sequence Synthetic oligonucleotide

SEQ ID NO: 381 - DNA, 73 bp, Artificial Sequence; identical to SEQ ID NO: 9
  Description of Artificial Sequence Synthetic oligonucleotide

SEQ ID NO: 382 - DNA, 73 bp, Artificial Sequence; identical to SEQ ID NO: 10
  Description of Artificial Sequence Synthetic oligonucleotide

SEQ ID NO: 383 - DNA, 73 bp, Artificial Sequence
  Description of Artificial Sequence Synthetic oligonucleotide
gccccagtggcctgatggataaggtactggccttcaaagccagggattgtgggttcgagttccacctggggta

SEQ ID NO: 384 - DNA, 73 bp, Artificial Sequence; identical to SEQ ID NO: 11
  Description of Artificial Sequence Synthetic oligonucleotide

SEQ ID NO: 385 - DNA, 73 bp, Artificial Sequence
  Description of Artificial Sequence Synthetic oligonucleotide
gaccacgtggcctaatggataaggcgtctgacttcagatcagaagattgagggttcgaatccctccgtggtta

SEQ ID NO: 386 - DNA, 73 bp, Artificial Sequence; identical to SEQ ID NO: 12
  Description of Artificial Sequence Synthetic oligonucleotide

SEQ ID NO: 387 - DNA, 73 bp, Artificial Sequence
  Description of Artificial Sequence Synthetic oligonucleotide
gaccacgtggcctaatggataaggcgtctgacttcagatcagaagattgagggttcgaatcccttcgtggtta

SEQ ID NO: 388 - DNA, 73 bp, Artificial Sequence; identical to SEQ ID NO: 13
  Description of Artificial Sequence Synthetic oligonucleotide

SEQ ID NO: 389 - DNA, 73 bp, Artificial Sequence
  Description of Artificial Sequence Synthetic oligonucleotide
ggccgtgtggcctaatggataaggcgtctgacttcagatcaaaagattgcaggtttgagttctgccacggtcg

SEQ ID NO: 390 - DNA, 85 bp, Artificial Sequence; identical to SEQ ID NO: 17
  Description of Artificial Sequence Synthetic oligonucleotide

SEQ ID NO: 391 - DNA, 73 bp, Artificial Sequence; identical to SEQ ID NO: 18
  Description of Artificial Sequence Synthetic oligonucleotide

SEQ ID NO: 392 - DNA, 88 bp, Artificial Sequence
  Description of Artificial Sequence Synthetic oligonucleotide
ggctctgtggcgcaatggatagcgcattggacttcaagtgacgaatagagcaattcaaaggttgtgggttcgaatcccaccagagtcg

SEQ ID NO: 393 - DNA, 73 bp, Artificial Sequence; identical to SEQ ID NO: 16
  Description of Artificial Sequence Synthetic oligonucleotide

SEQ ID NO: 394 - DNA, 91 bp, Artificial Sequence
  Description of Artificial Sequence Synthetic oligonucleotide
ggctctgtggcgcaatggatagcgcattggacttcaagctgagcctagtgtggtcattcaaaggttgtgggttcgagtcccaccagagtcg

SEQ ID NO: 395 - DNA, 73 bp, Artificial Sequence; identical to SEQ ID NO: 22
  Description of Artificial Sequence Synthetic oligonucleotide

SEQ ID NO: 396 - DNA, 86 bp, Artificial Sequence
  Description of Artificial Sequence Synthetic oligonucleotide
ggctctgtggcgcaatggatagcgcattggacttcaagatagttagagaaattcaaaggttgtgggttcgagtcccaccagagtcg

SEQ ID NO: 397 - DNA, 74 bp, Artificial Sequence; identical to SEQ ID NO: 35
  Description of Artificial Sequence Synthetic oligonucleotide

SEQ ID NO: 398 - DNA, 87 bp, Artificial Sequence
  Description of Artificial Sequence Synthetic oligonucleotide
ggctctgtggcgcaatggatagcgcattggacttcaagcctaaatcaagagattcaaaggttgcgggttcgagtccctccagagtcg

SEQ ID NO: 399 - DNA, 73 bp, Artificial Sequence
  Description of Artificial Sequence Synthetic oligonucleotide
ggctctgtggcgcaatggatagcgcattggacttcaaattcaaaggttgcgggttcgagtccctccagagtcg

SEQ ID NO: 400 - DNA, 69 bp, Artificial Sequence
  Description of Artificial Sequence Synthetic oligonucleotide
ggcagcatagcagagtggttcaggttacaggttcaagatgtaaactgagttcaaatcccagttctgcca

SEQ ID NO: 401 - DNA, 57 bp, Artificial Sequence
  Description of Artificial Sequence Synthetic oligonucleotide
tggtgtaataggtagcacagagaattctagattctcaggggtaggttcaattcctat

SEQ ID NO: 402 - DNA, 72 bp, Artificial Sequence
  Description of Artificial Sequence Synthetic oligonucleotide
taggacatggtgtgataggtagcatggagaattctagattctcaggggtaggttcaattcctacagttctag

SEQ ID NO: 403 - DNA, 72 bp, Artificial Sequence
  Description of Artificial Sequence Synthetic oligonucleotide
taggacgtggtgtgataggtagcatggggaattctagattctcaggggtgggttcaattcctatagttctag

SEQ ID NO: 404 - DNA, 72 bp, Artificial Sequence
  Description of Artificial Sequence Synthetic oligonucleotide
taggacgtggtgtagtaggtagcatggagaatgctaaattctcaggggtaggttcaattcctatagttctag

SEQ ID NO: 405 - DNA, 72 bp, Artificial Sequence
  Description of Artificial Sequence Synthetic oligonucleotide
taggacatggtgtaataggtagaatggagaattctaaattctcaggggtaggttcaattcctatagttctag

SEQ ID NO: 406 - DNA, 72 bp, Artificial Sequence
  Description of Artificial Sequence Synthetic oligonucleotide
taggatgtggtgtattaggtagcacagagaattctagattctcaggggtaggttcgattcctataattctac

SEQ ID NO: 407 - DNA, 72 bp, Artificial Sequence
  Description of Artificial Sequence Synthetic oligonucleotide
taggacttggtgtaatgggtagcacagagaattctagattctcaggggtgggttcaattcctttcgtcctag

SEQ ID NO: 408 - DNA, 75 bp, Artificial Sequence
  Description of Artificial Sequence Synthetic oligonucleotide
tctaggatgtggtgtgataggtagcatggagaattctagattctcaggggtaggttcaattcctatattctagaa

SEQ ID NO: 409 - DNA, 72 bp, Artificial Sequence
  Description of Artificial Sequence Synthetic oligonucleotide
taggacgtggtgtgataggtagcatggagaattctagattctcagggatgggttcaattcctatagtcctag

SEQ ID NO: 410 - DNA, 72 bp, Artificial Sequence
  Description of Artificial Sequence Synthetic oligonucleotide
taggacgtggtgtgataggtagcacggagaattctagattctcagggatgggttcaattcctgtagttctag

SEQ ID NO: 411 - DNA, 72 bp, Artificial Sequence; identical to SEQ ID NO: 180
  Description of Artificial Sequence Synthetic oligonucleotide

SEQ ID NO: 412 - DNA, 72 bp, Artificial Sequence
  Description of Artificial Sequence Synthetic oligonucleotide
ggttccatggtgtaatggtgaccactttggactctaaatacagtgatcagagttcaagtctcactggaacct

SEQ ID NO: 413 - DNA, 72 bp, Artificial Sequence
  Description of Artificial Sequence Synthetic oligonucleotide
ggttccatggtgtaatggtgagggctttggactctaactacagtgatcagagttcaagtctcagtgggacct

SEQ ID NO: 414 - DNA, 73 bp, Artificial Sequence
  Description of Artificial Sequence Synthetic oligonucleotide
ggttccatggtgtaatggtaagcaccctggactctaaatccagcaaccagagttccagtctcagcgtggacct

SEQ ID NO: 415 - DNA, 72 bp, Artificial Sequence
  Description of Artificial Sequence Synthetic oligonucleotide
ggtagtgtagtctactggttaaacgcttgggctctaacattaacgtcctgggttcaaatcccagctttgtca

SEQ ID NO: 416 - DNA, 72 bp, Artificial Sequence
  Description of Artificial Sequence Synthetic oligonucleotide
ggttccatggtgtaatggttagcactctggactctaaatccagcgatccgagttcaagtctcggtggaacct

SEQ ID NO: 417 - DNA, 72 bp, Artificial Sequence
  Description of Artificial Sequence Synthetic oligonucleotide
ggttccatggtgtaatggtgagcactctggactctaaatccagcgatccgagttcgagtctcggtggaacct

SEQ ID NO: 418 - DNA, 72 bp, Artificial Sequence
  Description of Artificial Sequence Synthetic oligonucleotide
ggttccatggtgtaatggtaagcactctggactctaaatccagcgatccgagttcgagtctcggtggaacct

SEQ ID NO: 419 - DNA, 72 bp, Artificial Sequence
  Description of Artificial Sequence Synthetic oligonucleotide
ggttccatggtgtaatggttagcactctggactctaaatccggtaatccgagttcaaatctcggtggaacct

SEQ ID NO: 420 - DNA, 72 bp, Artificial Sequence
  Description of Artificial Sequence Synthetic oligonucleotide
ggccccatggtgtaatggtcagcactctggactctaaatccagcgatccgagttcaaatctcggtgggaccc

SEQ ID NO: 421 - DNA, 72 bp, Artificial Sequence
  Description of Artificial Sequence Synthetic oligonucleotide
ggttccatggtgtaatggtaagcactctggactctaaatccagccatctgagttcgagtctctgtggaacct

SEQ ID NO: 422 - DNA, 72 bp, Artificial Sequence
  Description of Artificial Sequence Synthetic oligonucleotide
ggttccatggtgtaatggtgagcactttggactctaaatacagtgatcagagttcaagtctcactgggacct

SEQ ID NO: 423 - DNA, 72 bp, Artificial Sequence
  Description of Artificial Sequence Synthetic oligonucleotide
ggttccatgggttaatggtgagcaccctggactctaaatcaagcgatccgagttcaaatctcggtggtacct

SEQ ID NO: 424 - DNA, 68 bp, Artificial Sequence
  Description of Artificial Sequence Synthetic oligonucleotide
gtttccatggtgtaatggtgagcactctggactctaaatccagaaatacattcaaagaattaagaaca

SEQ ID NO: 425 - DNA, 72 bp, Artificial Sequence; identical to SEQ ID NO: 178
  Description of Artificial Sequence Synthetic oligonucleotide

SEQ ID NO: 426 - DNA, 72 bp, Artificial Sequence
  Description of Artificial Sequence Synthetic oligonucleotide
ggtcccatggtgtaatggttagcactctggactctaaatccagcaatccgagttcgaatctcggtgggacct

SEQ ID NO: 427 - DNA, 72 bp, Artificial Sequence; identical to SEQ ID NO: 187
  Description of Artificial Sequence Synthetic oligonucleotide

SEQ ID NO: 428 - DNA, 72 bp, Artificial Sequence
  Description of Artificial Sequence Synthetic oligonucleotide
ggtcccatggtgtaatggttagcactctgggctctaaatccagcaatccgagttcgaatcttggtgggacct

SEQ ID NO: 429 - DNA, 73 bp, Artificial Sequence
  Description of Artificial Sequence Synthetic oligonucleotide
ggctgtgtacctcagtgggcaagggtatggactctaaagccagactatttgggttcaaatcccagcttggcct

SEQ ID NO: 430 - DNA, 73 bp, Artificial Sequence
  Description of Artificial Sequence Synthetic oligonucleotide
gaccatgtggcctaagggaaaagacatctcactctaggtcagaagattgagggttcaagtcctttcatggtca

SEQ ID NO: 431 - DNA, 74 bp, Artificial Sequence
  Description of Artificial Sequence Synthetic oligonucleotide
ggtacagtgttaaaggggagaaaaattgctgactctaaatacagtagacctaggtttgaatcctggctttacca

SEQ ID NO: 432 - DNA, 57 bp, Artificial Sequence
  Description of Artificial Sequence Synthetic oligonucleotide
tggtgtaataggtagcacagagaattttagattctcaggggtaggttcaattcctat

SEQ ID NO: 433 - DNA, 72 bp, Artificial Sequence
  Description of Artificial Sequence Synthetic oligonucleotide
taggacatggtgtgataggtagcatggagaattttagattctcaggggtaggttcaattcctacagttctag

SEQ ID NO: 434 - DNA, 72 bp, Artificial Sequence
  Description of Artificial Sequence Synthetic oligonucleotide
taggacgtggtgtgataggtagcatggggaattttagattctcaggggtgggttcaattcctatagttctag

SEQ ID NO: 435 - DNA, 72 bp, Artificial Sequence
  Description of Artificial Sequence Synthetic oligonucleotide
taggacgtggtgtagtaggtagcatggagaatgttaaattctcaggggtaggttcaattcctatagttctag

SEQ ID NO: 436 - DNA, 72 bp, Artificial Sequence
  Description of Artificial Sequence Synthetic oligonucleotide
taggacatggtgtaataggtagaatggagaattttaaattctcaggggtaggttcaattcctatagttctag

SEQ ID NO: 437 - DNA, 72 bp, Artificial Sequence
  Description of Artificial Sequence Synthetic oligonucleotide
taggatgtggtgtattaggtagcacagagaattttagattctcaggggtaggttcgattcctataattctac

SEQ ID NO: 438 - DNA, 72 bp, Artificial Sequence
  Description of Artificial Sequence Synthetic oligonucleotide
taggacttggtgtaatgggtagcacagagaattttagattctcaggggtgggttcaattcctttcgtcctag

SEQ ID NO: 439 - DNA, 75 bp, Artificial Sequence
  Description of Artificial Sequence Synthetic oligonucleotide
tctaggatgtggtgtgataggtagcatggagaattttagattctcaggggtaggttcaattcctatattctagaa

SEQ ID NO: 440 - DNA, 72 bp, Artificial Sequence
  Description of Artificial Sequence Synthetic oligonucleotide
taggacgtggtgtgataggtagcatggagaattttagattctcagggatgggttcaattcctatagtcctag

SEQ ID NO: 441 - DNA, 72 bp, Artificial Sequence
  Description of Artificial Sequence Synthetic oligonucleotide
taggacgtggtgtgataggtagcacggagaattttagattctcagggatgggttcaattcctgtagttctag

SEQ ID NO: 442 - DNA, 72 bp, Artificial Sequence; identical to SEQ ID NO: 38
  Description of Artificial Sequence Synthetic oligonucleotide

SEQ ID NO: 443 - DNA, 72 bp, Artificial Sequence
  Description of Artificial Sequence Synthetic oligonucleotide
ggttccatggtgtaatggtgaccactttggactttaaatacagtgatcagagttcaagtctcactggaacct

SEQ ID NO: 444 - DNA, 72 bp, Artificial Sequence
  Description of Artificial Sequence Synthetic oligonucleotide
ggttccatggtgtaatggtgagggctttggactttaactacagtgatcagagttcaagtctcagtgggacct

SEQ ID NO: 445 - DNA, 73 bp, Artificial Sequence
  Description of Artificial Sequence Synthetic oligonucleotide
ggttccatggtgtaatggtaagcaccctggactttaaatccagcaaccagagttccagtctcagcgtggacct

SEQ ID NO: 446 - DNA, 72 bp, Artificial Sequence
  Description of Artificial Sequence Synthetic oligonucleotide
ggtagtgtagtctactggttaaacgcttgggctttaacattaacgtcctgggttcaaatcccagctttgtca

SEQ ID NO: 447 - DNA, 72 bp, Artificial Sequence
  Description of Artificial Sequence Synthetic oligonucleotide
ggttccatggtgtaatggttagcactctggactttaaatccagcgatccgagttcaagtctcggtggaacct

SEQ ID NO: 448 - DNA, 72 bp, Artificial Sequence
  Description of Artificial Sequence Synthetic oligonucleotide
ggttccatggtgtaatggtgagcactctggactttaaatccagcgatccgagttcgagtctcggtggaacct

SEQ ID NO: 449 - DNA, 72 bp, Artificial Sequence
  Description of Artificial Sequence Synthetic oligonucleotide
ggttccatggtgtaatggtaagcactctggactttaaatccagcgatccgagttcgagtctcggtggaacct

SEQ ID NO: 450 - DNA, 72 bp, Artificial Sequence
  Description of Artificial Sequence Synthetic oligonucleotide
ggttccatggtgtaatggttagcactctggactttaaatccggtaatccgagttcaaatctcggtggaacct

SEQ ID NO: 451 - DNA, 72 bp, Artificial Sequence
  Description of Artificial Sequence Synthetic oligonucleotide
ggccccatggtgtaatggtcagcactctggactttaaatccagcgatccgagttcaaatctcggtgggaccc

SEQ ID NO: 452 - DNA, 72 bp, Artificial Sequence
  Description of Artificial Sequence Synthetic oligonucleotide
ggttccatggtgtaatggtaagcactctggactttaaatccagccatctgagttcgagtctctgtggaacct

SEQ ID NO: 453 - DNA, 72 bp, Artificial Sequence
  Description of Artificial Sequence Synthetic oligonucleotide
ggttccatggtgtaatggtgagcactttggactttaaatacagtgatcagagttcaagtctcactgggacct

SEQ ID NO: 454 - DNA, 72 bp, Artificial Sequence
  Description of Artificial Sequence Synthetic oligonucleotide
ggttccatgggttaatggtgagcaccctggactttaaatcaagcgatccgagttcaaatctcggtggtacct

SEQ ID NO: 455 - DNA, 68 bp, Artificial Sequence
  Description of Artificial Sequence Synthetic oligonucleotide
gtttccatggtgtaatggtgagcactctggactttaaatccagaaatacattcaaagaattaagaaca

SEQ ID NO: 456 - DNA, 72 bp, Artificial Sequence; identical to SEQ ID NO: 36
  Description of Artificial Sequence Synthetic oligonucleotide

SEQ ID NO: 457 - DNA, 72 bp, Artificial Sequence
  Description of Artificial Sequence Synthetic oligonucleotide
ggtcccatggtgtaatggttagcactctggactttaaatccagcaatccgagttcgaatctcggtgggacct

SEQ ID NO: 458 - DNA, 72 bp, Artificial Sequence; identical to SEQ ID NO: 45
  Description of Artificial Sequence Synthetic oligonucleotide

SEQ ID NO: 459 - DNA, 72 bp, Artificial Sequence
  Description of Artificial Sequence Synthetic oligonucleotide
ggtcccatggtgtaatggttagcactctgggctttaaatccagcaatccgagttcgaatcttggtgggacct

SEQ ID NO: 460 - DNA, 73 bp, Artificial Sequence
  Description of Artificial Sequence Synthetic oligonucleotide
ggctgtgtacctcagtgggcaagggtatggactttaaagccagactatttgggttcaaatcccagcttggcct

SEQ ID NO: 461 - DNA, 73 bp, Artificial Sequence
  Description of Artificial Sequence Synthetic oligonucleotide
gaccatgtggcctaagggaaaagacatctcactttaggtcagaagattgagggttcaagtcctttcatggtca

SEQ ID NO: 462 - DNA, 74 bp, Artificial Sequence
  Description of Artificial Sequence Synthetic oligonucleotide
ggtacagtgttaaaggggagaaaaattgctgactttaaatacagtagacctaggtttgaatcctggctttacca

SEQ ID NO: 463 - DNA, 72 bp, Artificial Sequence; identical to SEQ ID NO: 96
  Description of Artificial Sequence Synthetic oligonucleotide

SEQ ID NO: 464 - DNA, 72 bp, Artificial Sequence
  Description of Artificial Sequence Synthetic oligonucleotide
tccctggtggtctagtggttaggattcggcgctttaaccgccgcggcccgggttcgattcccggtcaggaaa

SEQ ID NO: 465 - DNA, 72 bp, Artificial Sequence
  Description of Artificial Sequence Synthetic oligonucleotide
cccctggtggtctagtgcttaggattcggtgctttaaccgctgctgcctgcgttcgattcccggtcagggaa

SEQ ID NO: 466 - DNA, 71 bp, Artificial Sequence
  Description of Artificial Sequence Synthetic oligonucleotide
tccttgatgtctagtggttaggatttggtgctttaactgcagcagcctgggttcatttctcagtcagggaa

SEQ ID NO: 467 - DNA, 72 bp, Artificial Sequence
  Description of Artificial Sequence Synthetic oligonucleotide
tcccatatggtctagcggttaggattcctggttttaacccaggtggcccgggttcgactcccggtatgggaa

SEQ ID NO: 468 - DNA, 73 bp, Artificial Sequence
  Description of Artificial Sequence Synthetic oligonucleotide
tccgtggtggtctagtggctaggattcggcgctttaaccgcctgcagctcgagttcgattcctggtcagggaa

SEQ ID NO: 469 - DNA, 69 bp, Artificial Sequence
  Description of Artificial Sequence Synthetic oligonucleotide
ccctgtggtctagtggctaagactttgtgctttaattgctgcatcctaggttcaattcccagtcaggga

SEQ ID NO: 470 - DNA, 72 bp, Artificial Sequence; identical to SEQ ID NO: 102
  Description of Artificial Sequence Synthetic oligonucleotide

SEQ ID NO: 471 - DNA, 72 bp, Artificial Sequence
  Description of Artificial Sequence Synthetic oligonucleotide
tccctggtggtctagtggctaggattcggcgctttaaccgccgcggcccgggttcgattcccggccagggaa

SEQ ID NO: 472 - DNA, 72 bp, Artificial Sequence; identical to SEQ ID NO: 104
  Description of Artificial Sequence Synthetic oligonucleotide

SEQ ID NO: 473 - DNA, 72 bp, Artificial Sequence
  Description of Artificial Sequence Synthetic oligonucleotide
gcgttggtggtgtagtggtgagcacagctgcctttaaagcagttaacgcgggttcgattcccgggtaacgaa

SEQ ID NO: 474 - DNA, 72 bp, Artificial Sequence
  Description of Artificial Sequence Synthetic oligonucleotide
tccttggtggtctagtggctaggattcggtgctttaacctgtgcggcccgggttcaattcccgatgaaggaa

SEQ ID NO: 475 - DNA, 72 bp, Artificial Sequence
  Description of Artificial Sequence Synthetic oligonucleotide
tgtctggtggtcaagtggctaggatttggcgctttaactgccgcggcccgcgttcgattcccggtcagggaa

SEQ ID NO: 476 - DNA, 73 bp, Artificial Sequence
  Description of Artificial Sequence Synthetic oligonucleotide
tccctggtggtctagtggctaggattcggcgctttaaccgcctgcagctcgagttcgattcctggtcagggaa

SEQ ID NO: 477 - DNA, 71 bp, Artificial Sequence
  Description of Artificial Sequence Synthetic oligonucleotide
gcaatggtggttcagtggtagaattctcgcctttaacacaggagacccgggttcaattcctgacccatgta

SEQ ID NO: 478 - DNA, 72 bp, Artificial Sequence; identical to SEQ ID NO: 93
  Description of Artificial Sequence Synthetic oligonucleotide

SEQ ID NO: 479 - DNA, 72 bp, Artificial Sequence
  Description of Artificial Sequence Synthetic oligonucleotide
tccctggtggtctagtggttaggattcggcgctctaaccgccgcggcccgggttcgattcccggtcaggaaa

SEQ ID NO: 480 - DNA, 72 bp, Artificial Sequence
  Description of Artificial Sequence Synthetic oligonucleotide
cccctggtggtctagtgcttaggattcggtgctctaaccgctgctgcctgcgttcgattcccggtcagggaa

SEQ ID NO: 481 - DNA, 71 bp, Artificial Sequence
  Description of Artificial Sequence Synthetic oligonucleotide
tccttgatgtctagtggttaggatttggtgctctaactgcagcagcctgggttcatttctcagtcagggaa

SEQ ID NO: 482 - DNA, 72 bp, Artificial Sequence
  Description of Artificial Sequence Synthetic oligonucleotide
tcccatatggtctagcggttaggattcctggttctaacccaggtggcccgggttcgactcccggtatgggaa

SEQ ID NO: 483 - DNA, 73 bp, Artificial Sequence
  Description of Artificial Sequence Synthetic oligonucleotide
tccgtggtggtctagtggctaggattcggcgctctaaccgcctgcagctcgagttcgattcctggtcagggaa

SEQ ID NO: 484 - DNA, 69 bp, Artificial Sequence
  Description of Artificial Sequence Synthetic oligonucleotide
ccctgtggtctagtggctaagactttgtgctctaattgctgcatcctaggttcaattcccagtcaggga

SEQ ID NO: 485 - DNA, 72 bp, Artificial Sequence; identical to SEQ ID NO: 99
  Description of Artificial Sequence Synthetic oligonucleotide

SEQ ID NO: 486 - DNA, 72 bp, Artificial Sequence
  Description of Artificial Sequence Synthetic oligonucleotide
tccctggtggtctagtggctaggattcggcgctctaaccgccgcggcccgggttcgattcccggccagggaa

SEQ ID NO: 487 - DNA, 72 bp, Artificial Sequence; identical to SEQ ID NO: 101
  Description of Artificial Sequence Synthetic oligonucleotide

SEQ ID NO: 488 - DNA, 72 bp, Artificial Sequence
  Description of Artificial Sequence Synthetic oligonucleotide
gcgttggtggtgtagtggtgagcacagctgcctctaaagcagttaacgcgggttcgattcccgggtaacgaa

SEQ ID NO: 489 - DNA, 72 bp, Artificial Sequence
  Description of Artificial Sequence Synthetic oligonucleotide
tccttggtggtctagtggctaggattcggtgctctaacctgtgcggcccgggttcaattcccgatgaaggaa

SEQ ID NO: 490 - DNA, 72 bp, Artificial Sequence
  Description of Artificial Sequence Synthetic oligonucleotide
tgtctggtggtcaagtggctaggatttggcgctctaactgccgcggcccgcgttcgattcccggtcagggaa

SEQ ID NO: 491 - DNA, 73 bp, Artificial Sequence
  Description of Artificial Sequence Synthetic oligonucleotide
tccctggtggtctagtggctaggattcggcgctctaaccgcctgcagctcgagttcgattcctggtcagggaa

SEQ ID NO: 492 - DNA, 71 bp, Artificial Sequence
  Description of Artificial Sequence Synthetic oligonucleotide
gcaatggtggttcagtggtagaattctcgcctctaacacaggagacccgggttcaattcctgacccatgta

SEQ ID NO: 493 - DNA, 93 bp, Artificial Sequence
  Description of Artificial Sequence Synthetic oligonucleotide
ccttcaatagttcagctggtagagcagaggactttagctacttcctcagtaggagacgtccttaggttgctggttcgattccagcttgaagga

SEQ ID NO: 494 - DNA, 73 bp, Artificial Sequence
  Description of Artificial Sequence Synthetic oligonucleotide
ccttcaatagttcagctggtagagcagaggactttaggtccttaggttgctggttcgattccagcttgaagga

SEQ ID NO: 495 - DNA, 64 bp, Artificial Sequence
  Description of Artificial Sequence Synthetic oligonucleotide
ggtaaaatggctgagtaagctttagactttaaatctaaagagagattgagctctctttttacca

SEQ ID NO: 496 - DNA, 66 bp, Artificial Sequence
  Description of Artificial Sequence Synthetic oligonucleotide
ggtaaaatgactgagtaagcattagactttaaatctaaagacagaggtcaagacctctttttacca

SEQ ID NO: 497 - DNA, 66 bp, Artificial Sequence
  Description of Artificial Sequence Synthetic oligonucleotide
ggtaaaatggctgagtaagcattagactttaaatctaaagacagaggtcaaggcctctttttacca

SEQ ID NO: 498 - DNA, 64 bp, Artificial Sequence
  Description of Artificial Sequence Synthetic oligonucleotide
ggtaaaatggctgagtaagcattagactttaaatctaaagacagaggtcaaggcctttttacca

SEQ ID NO: 499 - DNA, 91 bp, Artificial Sequence
  Description of Artificial Sequence Synthetic oligonucleotide
ccttcgatagctcagttggtagagcggaggactttagttggctgtgtccttagacatccttaggtcgctggttcgaatccggctcgaagga

SEQ ID NO: 500 - DNA, 73 bp, Artificial Sequence; identical to SEQ ID NO: 282
  Description of Artificial Sequence Synthetic oligonucleotide

SEQ ID NO: 501 - DNA, 73 bp, Artificial Sequence
  Description of Artificial Sequence Synthetic oligonucleotide
gggggtatagctcagggctagagctttttgactttagagcaagaggtccctggttcaaatccaggttctccct

SEQ ID NO: 502 - DNA, 61 bp, Artificial Sequence
  Description of Artificial Sequence Synthetic oligonucleotide
tatagctcagtggtagagcatttaactttagatcaagaggtccctggatcaactctgggtg

SEQ ID NO: 503 - DNA, 73 bp, Artificial Sequence
  Description of Artificial Sequence Synthetic oligonucleotide
gtcagtgttgcacaacggttaagtgaagaggctttaaacccagactggatgggttcaattcccatctctgccg

SEQ ID NO: 504 - DNA, 89 bp, Artificial Sequence
  Description of Artificial Sequence Synthetic oligonucleotide
ccttcgatagctcagttggtagagcggaggactttagtggatagggcgtggcaatccttaggtcgctggttcgattccggctcgaagga

SEQ ID NO: 505 - DNA, 73 bp, Artificial Sequence; identical to SEQ ID NO: 283
  Description of Artificial Sequence Synthetic oligonucleotide

SEQ ID NO: 506 - DNA, 89 bp, Artificial Sequence
  Description of Artificial Sequence Synthetic oligonucleotide
ccttcgatagctcagttggtagagcggaggactttaggctcattaagcaaggtatccttaggtcgctggttcgaatccggctcggagga

SEQ ID NO: 507 - DNA, 73 bp, Artificial Sequence
  Description of Artificial Sequence Synthetic oligonucleotide
ccttcgatagctcagttggtagagcggaggactttagatccttaggtcgctggttcgaatccggctcggagga

SEQ ID NO: 508 - DNA, 94 bp, Artificial Sequence
  Description of Artificial Sequence Synthetic oligonucleotide
ccttcgatagctcagctggtagagcggaggactttagattgtatagacatttgcggacatccttaggtcgctggttcgattccagctcgaagga

SEQ ID NO: 509 - DNA, 73 bp, Artificial Sequence
  Description of Artificial Sequence Synthetic oligonucleotide
ccttcgatagctcagctggtagagcggaggactttagatccttaggtcgctggttcgattccagctcgaagga

SEQ ID NO: 510 - DNA, 93 bp, Artificial Sequence
  Description of Artificial Sequence Synthetic oligonucleotide
ccttcgatagctcagctggtagagcggaggactttagctacttcctcagcaggagacatccttaggtcgctggttcgattccggctcgaagga

SEQ ID NO: 511 - DNA, 73 bp, Artificial Sequence; identical to SEQ ID NO: 284
  Description of Artificial Sequence Synthetic oligonucleotide

SEQ ID NO: 512 - DNA, 89 bp, Artificial Sequence
  Description of Artificial Sequence Synthetic oligonucleotide
ccttcgatagctcagctggtagagcggaggactttaggcgcgcgcccgtggccatccttaggtcgctggttcgattccggctcgaagga

SEQ ID NO: 513 - DNA, 73 bp, Artificial Sequence; identical to SEQ ID NO: 284
  Description of Artificial Sequence Synthetic oligonucleotide

SEQ ID NO: 514 - DNA, 94 bp, Artificial Sequence
  Description of Artificial Sequence Synthetic oligonucleotide
ccttcgatagctcagctggtagagcggaggactttagcctgtagaaacatttgtggacatccttaggtcgctggttcgattccggctcgaagga

SEQ ID NO: 515 - DNA, 73 bp, Artificial Sequence; identical to SEQ ID NO: 284
  Description of Artificial Sequence Synthetic oligonucleotide

SEQ ID NO: 516 - DNA, 94 bp, Artificial Sequence
  Description of Artificial Sequence Synthetic oligonucleotide
ccttcgatagctcagctggtagagcggaggactttagattgtacagacatttgcggacatccttaggtcgctggttcgattccggctcgaagga

SEQ ID NO: 517 - DNA, 73 bp, Artificial Sequence; identical to SEQ ID NO: 284
  Description of Artificial Sequence Synthetic oligonucleotide

SEQ ID NO: 518 - DNA, 89 bp, Artificial Sequence
  Description of Artificial Sequence Synthetic oligonucleotide
ccttcgatagctcagctggtagagcggaggactttagtacttaatgtgtggtcatccttaggtcgctggttcgattccggctcgaagga

SEQ ID NO: 519 - DNA, 73 bp, Artificial Sequence; identical to SEQ ID NO: 284
  Description of Artificial Sequence Synthetic oligonucleotide

SEQ ID NO: 520 - DNA, 89 bp, Artificial Sequence
  Description of Artificial Sequence Synthetic oligonucleotide
ccttcgatagctcagctggtagagcggaggactttaggggtttgaatgtggtcatccttaggtcgctggttcgaatccggctcggagga

SEQ ID NO: 521 - DNA, 73 bp, Artificial Sequence
  Description of Artificial Sequence Synthetic oligonucleotide
ccttcgatagctcagctggtagagcggaggactttagatccttaggtcgctggttcgaatccggctcggagga

SEQ ID NO: 522 - DNA, 94 bp, Artificial Sequence
  Description of Artificial Sequence Synthetic oligonucleotide
ccttcgatagctcagctggtagagcggaggactttagactgcggaaacgtttgtggacatccttaggtcgctggttcaattccggctcgaagga

SEQ ID NO: 523 - DNA, 73 bp, Artificial Sequence
  Description of Artificial Sequence Synthetic oligonucleotide
ccttcgatagctcagctggtagagcggaggactttagatccttaggtcgctggttcaattccggctcgaagga

SEQ ID NO: 524 - DNA, 90 bp, Artificial Sequence
  Description of Artificial Sequence Synthetic oligonucleotide
ctttcgatagctcagttggtagagcggaggactttaggttcattaaactaaggcatccttaggtcgctggttcgaatccggctcgaagga

SEQ ID NO: 525 - DNA, 73 bp, Artificial Sequence
  Description of Artificial Sequence Synthetic oligonucleotide
ctttcgatagctcagttggtagagcggaggactttagatccttaggtcgctggttcgaatccggctcgaagga

SEQ ID NO: 526 - DNA, 88 bp, Artificial Sequence
  Description of Artificial Sequence Synthetic oligonucleotide
tcttcaatagctcagctggtagagcggaggactttaggtgcacgcccgtggccattcttaggtgctggtttgattccgacttggagag

SEQ ID NO: 527 - DNA, 72 bp, Artificial Sequence
  Description of Artificial Sequence Synthetic oligonucleotide
tcttcaatagctcagctggtagagcggaggactttagattcttaggtgctggtttgattccgacttggagag

SEQ ID NO: 528 - DNA, 66 bp, Artificial Sequence
  Description of Artificial Sequence Synthetic oligonucleotide
ggtaaaatggctgagtgaagcattggactttaaatctaaagacaggggttaagcctctttttacca

SEQ ID NO: 529 - DNA, 66 bp, Artificial Sequence
  Description of Artificial Sequence Synthetic oligonucleotide
ggtaaaatggctgagcaagcattggactttaaatctaaagacagatgttgagccatctttttagca

SEQ ID NO: 530 - DNA, 66 bp, Artificial Sequence
  Description of Artificial Sequence Synthetic oligonucleotide
ggtaaaatggctgagtgaagcattggactttaaatctaaagacaggggctaagcctctttttacca

SEQ ID NO: 531 - DNA, 66 bp, Artificial Sequence
  Description of Artificial Sequence Synthetic oligonucleotide
ggtaaaatggctgagcaagcattagactttaaatctaaagacagaggttaaggcctctttttacca

SEQ ID NO: 532 - DNA, 66 bp, Artificial Sequence
  Description of Artificial Sequence Synthetic oligonucleotide
ggtaaaatggctgagtaagcattagactttaaatctaaagacagaggtcaaggcctcttttttcct

SEQ ID NO: 533 - DNA, 67 bp, Artificial Sequence
  Description of Artificial Sequence Synthetic oligonucleotide
ggtaaaatggctgagcaagcattagactttaaatctgaaaacagaggtcaaaggtctctttttacca

SEQ ID NO: 534 - DNA, 66 bp, Artificial Sequence; identical to SEQ ID NO: 497
  Description of Artificial Sequence Synthetic oligonucleotide

SEQ ID NO: 535 - DNA, 66 bp, Artificial Sequence
  Description of Artificial Sequence Synthetic oligonucleotide
ggtaaaatgactgaataagccttagactttaaatctgaagacagaggtcaaggcctctttttacca

SEQ ID NO: 536 - DNA, 66 bp, Artificial Sequence
  Description of Artificial Sequence Synthetic oligonucleotide
ggtaaaatggctgagtaagcattggactttaaatctaaagacagaggtcaagacctctttttacca

SEQ ID NO: 537 - DNA, 66 bp, Artificial Sequence
  Description of Artificial Sequence Synthetic oligonucleotide
ggtaaaatggctgagtaaagcattagactttaaatctaaggacagaggctaaacctctttttacca

SEQ ID NO: 538 - DNA, 93 bp, Artificial Sequence
  Description of Artificial Sequence Synthetic oligonucleotide
ccttcaatagttcagctggtagagcagaggactctagctacttcctcagtaggagacgtccttaggttgctggttcgattccagcttgaagga

SEQ ID NO: 539 - DNA, 73 bp, Artificial Sequence
  Description of Artificial Sequence Synthetic oligonucleotide
ccttcaatagttcagctggtagagcagaggactctaggtccttaggttgctggttcgattccagcttgaagga

SEQ ID NO: 540 - DNA, 64 bp, Artificial Sequence
  Description of Artificial Sequence Synthetic oligonucleotide
ggtaaaatggctgagtaagctttagactctaaatctaaagagagattgagctctctttttacca

SEQ ID NO: 541 - DNA, 66 bp, Artificial Sequence
  Description of Artificial Sequence Synthetic oligonucleotide
ggtaaaatgactgagtaagcattagactctaaatctaaagacagaggtcaagacctctttttacca

SEQ ID NO: 542 - DNA, 66 bp, Artificial Sequence
  Description of Artificial Sequence Synthetic oligonucleotide
ggtaaaatggctgagtaagcattagactctaaatctaaagacagaggtcaaggcctctttttacca

SEQ ID NO: 543 - DNA, 64 bp, Artificial Sequence
  Description of Artificial Sequence Synthetic oligonucleotide
ggtaaaatggctgagtaagcattagactctaaatctaaagacagaggtcaaggcctttttacca

SEQ ID NO: 544 - DNA, 91 bp, Artificial Sequence
  Description of Artificial Sequence Synthetic oligonucleotide
ccttcgatagctcagttggtagagcggaggactctagttggctgtgtccttagacatccttaggtcgctggttcgaatccggctcgaagga

SEQ ID NO: 545 - DNA, 73 bp, Artificial Sequence; identical to SEQ ID NO: 277
  Description of Artificial Sequence Synthetic oligonucleotide

SEQ ID NO: 546 - DNA, 73 bp, Artificial Sequence
  Description of Artificial Sequence Synthetic oligonucleotide
gggggtatagctcagggctagagctttttgactctagagcaagaggtccctggttcaaatccaggttctccct

SEQ ID NO: 547 - DNA, 61 bp, Artificial Sequence
  Description of Artificial Sequence Synthetic oligonucleotide
tatagctcagtggtagagcatttaactctagatcaagaggtccctggatcaactctgggtg

SEQ ID NO: 548 - DNA, 73 bp, Artificial Sequence
  Description of Artificial Sequence Synthetic oligonucleotide
gtcagtgttgcacaacggttaagtgaagaggctctaaacccagactggatgggttcaattcccatctctgccg

SEQ ID NO: 549 - DNA, 89 bp, Artificial Sequence
  Description of Artificial Sequence Synthetic oligonucleotide
ccttcgatagctcagttggtagagcggaggactctagtggatagggcgtggcaatccttaggtcgctggttcgattccggctcgaagga

SEQ ID NO: 550 - DNA, 73 bp, Artificial Sequence; identical to SEQ ID NO: 278
  Description of Artificial Sequence Synthetic oligonucleotide

SEQ ID NO: 551 - DNA, 89 bp, Artificial Sequence
  Description of Artificial Sequence Synthetic oligonucleotide
ccttcgatagctcagttggtagagcggaggactctaggctcattaagcaaggtatccttaggtcgctggttcgaatccggctcggagga

SEQ ID NO: 552 - DNA, 73 bp, Artificial Sequence
  Description of Artificial Sequence Synthetic oligonucleotide
ccttcgatagctcagttggtagagcggaggactctagatccttaggtcgctggttcgaatccggctcggagga

SEQ ID NO: 553 - DNA, 94 bp, Artificial Sequence
  Description of Artificial Sequence Synthetic oligonucleotide
ccttcgatagctcagctggtagagcggaggactctagattgtatagacatttgcggacatccttaggtcgctggttcgattccagctcgaagga

SEQ ID NO: 554 - DNA, 73 bp, Artificial Sequence
  Description of Artificial Sequence Synthetic oligonucleotide
ccttcgatagctcagctggtagagcggaggactctagatccttaggtcgctggttcgattccagctcgaagga

SEQ ID NO: 555 - DNA, 93 bp, Artificial Sequence
  Description of Artificial Sequence Synthetic oligonucleotide
ccttcgatagctcagctggtagagcggaggactctagctacttcctcagcaggagacatccttaggtcgctggttcgattccggctcgaagga

SEQ ID NO: 556 - DNA, 73 bp, Artificial Sequence; identical to SEQ ID NO: 279
  Description of Artificial Sequence Synthetic oligonucleotide

SEQ ID NO: 557 - DNA, 89 bp, Artificial Sequence
  Description of Artificial Sequence Synthetic oligonucleotide
ccttcgatagctcagctggtagagcggaggactctaggcgcgcgcccgtggccatccttaggtcgctggttcgattccggctcgaagga

SEQ ID NO: 558 - DNA, 73 bp, Artificial Sequence; identical to SEQ ID NO: 279
  Description of Artificial Sequence Synthetic oligonucleotide

SEQ ID NO: 559 - DNA, 94 bp, Artificial Sequence
  Description of Artificial Sequence Synthetic oligonucleotide
ccttcgatagctcagctggtagagcggaggactctagcctgtagaaacatttgtggacatccttaggtcgctggttcgattccggctcgaagga

SEQ ID NO: 560 - DNA, 73 bp, Artificial Sequence; identical to SEQ ID NO: 279
  Description of Artificial Sequence Synthetic oligonucleotide

SEQ ID NO: 561 - DNA, 94 bp, Artificial Sequence
  Description of Artificial Sequence Synthetic oligonucleotide
ccttcgatagctcagctggtagagcggaggactctagattgtacagacatttgcggacatccttaggtcgctggttcgattccggctcgaagga

SEQ ID NO: 562 - DNA, 73 bp, Artificial Sequence; identical to SEQ ID NO: 279
  Description of Artificial Sequence Synthetic oligonucleotide

SEQ ID NO: 563 - DNA, 89 bp, Artificial Sequence
  Description of Artificial Sequence Synthetic oligonucleotide
ccttcgatagctcagctggtagagcggaggactctagtacttaatgtgtggtcatccttaggtcgctggttcgattccggctcgaagga

SEQ ID NO: 564 - DNA, 73 bp, Artificial Sequence; identical to SEQ ID NO: 279
  Description of Artificial Sequence Synthetic oligonucleotide

SEQ ID NO: 565 - DNA, 89 bp, Artificial Sequence
  Description of Artificial Sequence Synthetic oligonucleotide
ccttcgatagctcagctggtagagcggaggactctaggggtttgaatgtggtcatccttaggtcgctggttcgaatccggctcggagga

SEQ ID NO: 566 - DNA, 73 bp, Artificial Sequence
  Description of Artificial Sequence Synthetic oligonucleotide
ccttcgatagctcagctggtagagcggaggactctagatccttaggtcgctggttcgaatccggctcggagga

SEQ ID NO: 567 - DNA, 94 bp, Artificial Sequence
  Description of Artificial Sequence Synthetic oligonucleotide
ccttcgatagctcagctggtagagcggaggactctagactgcggaaacgtttgtggacatccttaggtcgctggttcaattccggctcgaagga

SEQ ID NO: 568 - DNA, 73 bp, Artificial Sequence
  Description of Artificial Sequence Synthetic oligonucleotide
ccttcgatagctcagctggtagagcggaggactctagatccttaggtcgctggttcaattccggctcgaagga

SEQ ID NO: 569 - DNA, 90 bp, Artificial Sequence
  Description of Artificial Sequence Synthetic oligonucleotide
ctttcgatagctcagttggtagagcggaggactctaggttcattaaactaaggcatccttaggtcgctggttcgaatccggctcgaagga

SEQ ID NO: 570 - DNA, 73 bp, Artificial Sequence
  Description of Artificial Sequence Synthetic oligonucleotide
ctttcgatagctcagttggtagagcggaggactctagatccttaggtcgctggttcgaatccggctcgaagga

SEQ ID NO: 571 - DNA, 88 bp, Artificial Sequence
  Description of Artificial Sequence Synthetic oligonucleotide
tcttcaatagctcagctggtagagcggaggactctaggtgcacgcccgtggccattcttaggtgctggtttgattccgacttggagag

SEQ ID NO: 572 - DNA, 72 bp, Artificial Sequence
  Description of Artificial Sequence Synthetic oligonucleotide
tcttcaatagctcagctggtagagcggaggactctagattcttaggtgctggtttgattccgacttggagag

SEQ ID NO: 573 - DNA, 66 bp, Artificial Sequence
  Description of Artificial Sequence Synthetic oligonucleotide
ggtaaaatggctgagtgaagcattggactctaaatctaaagacaggggttaagcctctttttacca

SEQ ID NO: 574 - DNA, 66 bp, Artificial Sequence
  Description of Artificial Sequence Synthetic oligonucleotide
ggtaaaatggctgagcaagcattggactctaaatctaaagacagatgttgagccatctttttagca

SEQ ID NO: 575 - DNA, 66 bp, Artificial Sequence
  Description of Artificial Sequence Synthetic oligonucleotide
ggtaaaatggctgagtgaagcattggactctaaatctaaagacaggggctaagcctctttttacca

SEQ ID NO: 576 - DNA, 66 bp, Artificial Sequence
  Description of Artificial Sequence Synthetic oligonucleotide
ggtaaaatggctgagcaagcattagactctaaatctaaagacagaggttaaggcctctttttacca

SEQ ID NO: 577 - DNA, 66 bp, Artificial Sequence
  Description of Artificial Sequence Synthetic oligonucleotide
ggtaaaatggctgagtaagcattagactctaaatctaaagacagaggtcaaggcctcttttttcct

SEQ ID NO: 578 - DNA, 67 bp, Artificial Sequence
  Description of Artificial Sequence Synthetic oligonucleotide
ggtaaaatggctgagcaagcattagactctaaatctgaaaacagaggtcaaaggtctctttttacca

SEQ ID NO: 579 - DNA, 66 bp, Artificial Sequence; identical to SEQ ID NO: 542
  Description of Artificial Sequence Synthetic oligonucleotide

SEQ ID NO: 580 - DNA, 66 bp, Artificial Sequence
  Description of Artificial Sequence Synthetic oligonucleotide
ggtaaaatgactgaataagccttagactctaaatctgaagacagaggtcaaggcctctttttacca

SEQ ID NO: 581 - DNA, 66 bp, Artificial Sequence
  Description of Artificial Sequence Synthetic oligonucleotide
ggtaaaatggctgagtaagcattggactctaaatctaaagacagaggtcaagacctctttttacca

SEQ ID NO: 582 - DNA, 66 bp, Artificial Sequence
  Description of Artificial Sequence Synthetic oligonucleotide
ggtaaaatggctgagtaaagcattagactctaaatctaaggacagaggctaaacctctttttacca

SEQ ID NO: 583 - DNA, 75 bp, Artificial Sequence
  Description of Artificial Sequence Synthetic oligonucleotide
gttaagatggcagagcctggtaattgcattaaacttaaaattttataatcagaggttcaactcctcttcttaaca

SEQ ID NO: 584 - DNA, 75 bp, Artificial Sequence
  Description of Artificial Sequence Synthetic oligonucleotide
gttaagatggcagagcccggcaattgcattagacttaaaactttataatcagaggttcaactcctctcattaaca

SEQ ID NO: 585 - DNA, 82 bp, Artificial Sequence
  Description of Artificial Sequence Synthetic oligonucleotide
ggtagcgtggccgagcggtctaaggcgctggattttagctccagtctcttcgggggcgtgggttcaaatcccaccgctgcca

SEQ ID NO: 586 - DNA, 82 bp, Artificial Sequence
  Description of Artificial Sequence Synthetic oligonucleotide
ggtagcgtggccgagtggtctaagacgctggattttagctccagtctcttcgggggcgtgggtttgaatcccaccgctgcca

SEQ ID NO: 587 - DNA, 73 bp, Artificial Sequence
  Description of Artificial Sequence Synthetic oligonucleotide
gggccagtggctcaatggataatgcgtctgactttaaatcagaagattccagccttgactcctggctggctca

SEQ ID NO: 588 - DNA, 82 bp, Artificial Sequence
  Description of Artificial Sequence Synthetic oligonucleotide
ggtagggtggccgagcggtctaaggcactgtattttaactccagtctcttcagaggcatgggtttgaatcccactgctgcca

SEQ ID NO: 589 - DNA, 65 bp, Artificial Sequence
  Description of Artificial Sequence Synthetic oligonucleotide
gccgagcggtctaaggctccggattttagcgccggtgtcttcggaggcatgggttcgaattccac

SEQ ID NO: 590 - DNA, 106 bp, Artificial Sequence
  Description of Artificial Sequence Synthetic polynucleotide
gtcaggatggccgagtggtctaaggcgccagactttagctaagcttcctccgcggtggggattctggtctccaatggaggcgtgggttcgaatcccacttctgaca

SEQ ID NO: 591 - DNA, 83 bp, Artificial Sequence
  Description of Artificial Sequence Synthetic oligonucleotide
gtcaggatggccgagtggtctaaggcgccagactttagttctggtctccaatggaggcgtgggttcgaatcccacttctgaca

SEQ ID NO: 592 - DNA, 105 bp, Artificial Sequence
  Description of Artificial Sequence Synthetic polynucleotide
gtcaggatggccgagtggtctaaggcgccagactttagcttggcttcctcgtgttgaggattctggtctccaatggaggcgtgggttcgaatcccacttctgaca

SEQ ID NO: 593 - DNA, 83 bp, Artificial Sequence; identical to SEQ ID NO: 591
  Description of Artificial Sequence Synthetic oligonucleotide

SEQ ID NO: 594 - DNA, 108 bp, Artificial Sequence
  Description of Artificial Sequence Synthetic polynucleotide
gtcaggatggccgagtggtctaaggcgccagactttagcttactgcttcctgtgttcgggtcttctggtctccgtatggaggcgtgggttcgaatcccacttctgaca

SEQ ID NO: 595 - DNA, 84 bp, Artificial Sequence; identical to SEQ ID NO: 133
  Description of Artificial Sequence Synthetic oligonucleotide

SEQ ID NO: 596 - DNA, 107 bp, Artificial Sequence
  Description of Artificial Sequence Synthetic polynucleotide
gtcaggatggccgagtggtctaaggcgccagactttagttgctacttcccaggtttggggcttctggtctccgcatggaggcgtgggttcgaatcccacttctgaca

SEQ ID NO: 597 - DNA, 84 bp, Artificial Sequence
  Description of Artificial Sequence Synthetic oligonucleotide
gtcaggatggccgagtggtctaaggcgccagactttagttctggtctccgcatggaggcgtgggttcgaatcccacttctgaca

SEQ ID NO: 598 - DNA, 106 bp, Artificial Sequence
  Description of Artificial Sequence Synthetic polynucleotide
gtcaggatggccgagtggtctaaggcgccagactttaggtaagcaccttgcctgcgggctttctggtctccggatggaggcgtgggttcgaatcccacttctgaca

SEQ ID NO: 599 - DNA, 85 bp, Artificial Sequence
  Description of Artificial Sequence Synthetic oligonucleotide
gtcaggatggccgagtggtctaaggcgccagactttagtttctggtctccggatggaggcgtgggttcgaatcccacttctgaca

SEQ ID NO: 600 - DNA, 74 bp, Artificial Sequence
  Description of Artificial Sequence Synthetic oligonucleotide
gcctccttagtgcagtaggtagcgcatcagtctttaaatctgaatggtcctgagttcaagcctcagagggggca

SEQ ID NO: 601 - DNA, 84 bp, Artificial Sequence
  Description of Artificial Sequence Synthetic oligonucleotide
gtcaggatggccgagcagtcttaaggcgctgcgttttaatcgcaccctccgctggaggcgtgggttcgaatcccacttttgaca

SEQ ID NO: 602 - DNA, 60 bp, Artificial Sequence
  Description of Artificial Sequence Synthetic oligonucleotide
ggttccatggtgtaatggtgagcactctggactttaaatccagaagtagtgctggaacaa

SEQ ID NO: 603 - DNA, 83 bp, Artificial Sequence
  Description of Artificial Sequence Synthetic oligonucleotide
gtcagggtggctgagcagtctgaggggctgcgttttagtcgcagtctgccctggaggcgtgggttcgaatcccactcctgaaa

SEQ ID NO: 604 - DNA, 83 bp, Artificial Sequence
  Description of Artificial Sequence Synthetic oligonucleotide
accaggatggccgagtggttaaggcgttggactttagatccaatggacatatgtccgcgtgggttcgaaccccactcctggta

SEQ ID NO: 605 - DNA, 83 bp, Artificial Sequence
  Description of Artificial Sequence Synthetic oligonucleotide
accgggatggccgagtggttaaggcgttggactttagatccaatgggctggtgcccgcgtgggttcgaaccccactctcggta

SEQ ID NO: 606 - DNA, 83 bp, Artificial Sequence
  Description of Artificial Sequence Synthetic oligonucleotide
accagaatggccgagtggttaaggcgttggactttagatccaatggattcatatccgcgtgggttcgaaccccacttctggta

SEQ ID NO: 607 - DNA, 83 bp, Artificial Sequence
  Description of Artificial Sequence Synthetic oligonucleotide
accgggatggctgagtggttaaggcgttggactttagatccaatggacaggtgtccgcgtgggttcgagccccactcccggta

SEQ ID NO: 608 - DNA, 83 bp, Artificial Sequence
  Description of Artificial Sequence Synthetic oligonucleotide
actcatttggctgagtggttaaggcattggactttagatccaatggagtagtggctgtgtgggtttaaaccccactactggta

SEQ ID NO: 609 - DNA, 69 bp, Artificial Sequence
  Description of Artificial Sequence Synthetic oligonucleotide
gagaaagtcatcgtagttacgaagttggctttaacccagttttgggaggttcaattccttcctttctct

SEQ ID NO: 610 - DNA, 84 bp, Artificial Sequence
  Description of Artificial Sequence Synthetic oligonucleotide
accaggatggccaagtagttaaaggcactggactttagagccaatggacatatgtctgtgtgggtttgaaccccactcctggtg

SEQ ID NO: 611 - DNA, 82 bp, Artificial Sequence; identical to SEQ ID NO: 166
  Description of Artificial Sequence Synthetic oligonucleotide

SEQ ID NO: 612 - DNA, 82 bp, Artificial Sequence; identical to SEQ ID NO: 123
  Description of Artificial Sequence Synthetic oligonucleotide

SEQ ID NO: 613 - DNA, 82 bp, Artificial Sequence; identical to SEQ ID NO: 168
  Description of Artificial Sequence Synthetic oligonucleotide

SEQ ID NO: 614 - DNA, 82 bp, Artificial Sequence
  Description of Artificial Sequence Synthetic oligonucleotide
ggtagtgtggttgaatggtctaaggcactgaattttagctccagtctctttggggacgtgggtttaaatcccactgctgcaa

SEQ ID NO: 615 - DNA, 75 bp, Artificial Sequence
  Description of Artificial Sequence Synthetic oligonucleotide
gttaagatggcagagcctggtaattgcactaaacttaaaattttataatcagaggttcaactcctcttcttaaca

SEQ ID NO: 616 - DNA, 75 bp, Artificial Sequence
  Description of Artificial Sequence Synthetic oligonucleotide
gttaagatggcagagcccggcaattgcactagacttaaaactttataatcagaggttcaactcctctcattaaca

SEQ ID NO: 617 - DNA, 82 bp, Artificial Sequence
  Description of Artificial Sequence Synthetic oligonucleotide
ggtagcgtggccgagcggtctaaggcgctggattctagctccagtctcttcgggggcgtgggttcaaatcccaccgctgcca

SEQ ID NO: 618 - DNA, 82 bp, Artificial Sequence
  Description of Artificial Sequence Synthetic oligonucleotide
ggtagcgtggccgagtggtctaagacgctggattctagctccagtctcttcgggggcgtgggtttgaatcccaccgctgcca

SEQ ID NO: 619 - DNA, 73 bp, Artificial Sequence
  Description of Artificial Sequence Synthetic oligonucleotide
gggccagtggctcaatggataatgcgtctgactctaaatcagaagattccagccttgactcctggctggctca

SEQ ID NO: 620 - DNA, 82 bp, Artificial Sequence
  Description of Artificial Sequence Synthetic oligonucleotide
ggtagggtggccgagcggtctaaggcactgtattctaactccagtctcttcagaggcatgggtttgaatcccactgctgcca

SEQ ID NO: 621 - DNA, 65 bp, Artificial Sequence
  Description of Artificial Sequence Synthetic oligonucleotide
gccgagcggtctaaggctccggattctagcgccggtgtcttcggaggcatgggttcgaattccac

SEQ ID NO: 622 - DNA, 106 bp, Artificial Sequence
  Description of Artificial Sequence Synthetic polynucleotide
gtcaggatggccgagtggtctaaggcgccagactctagctaagcttcctccgcggtggggattctggtctccaatggaggcgtgggttcgaatcccacttctgaca

SEQ ID NO: 623 - DNA, 83 bp, Artificial Sequence
  Description of Artificial Sequence Synthetic oligonucleotide
gtcaggatggccgagtggtctaaggcgccagactctagttctggtctccaatggaggcgtgggttcgaatcccacttctgaca

SEQ ID NO: 624 - DNA, 105 bp, Artificial Sequence
  Description of Artificial Sequence Synthetic polynucleotide
gtcaggatggccgagtggtctaaggcgccagactctagcttggcttcctcgtgttgaggattctggtctccaatggaggcgtgggttcgaatcccacttctgaca

SEQ ID NO: 625 - DNA, 83 bp, Artificial Sequence; identical to SEQ ID NO: 623
  Description of Artificial Sequence Synthetic oligonucleotide

SEQ ID NO: 626 - DNA, 108 bp, Artificial Sequence
  Description of Artificial Sequence Synthetic polynucleotide
gtcaggatggccgagtggtctaaggcgccagactctagcttactgcttcctgtgttcgggtcttctggtctccgtatggaggcgtgggttcgaatcccacttctgaca

SEQ ID NO: 627 - DNA, 84 bp, Artificial Sequence; identical to SEQ ID NO: 125
  Description of Artificial Sequence Synthetic oligonucleotide

SEQ ID NO: 628 - DNA, 107 bp, Artificial Sequence
  Description of Artificial Sequence Synthetic polynucleotide
gtcaggatggccgagtggtctaaggcgccagactctagttgctacttcccaggtttggggcttctggtctccgcatggaggcgtgggttcgaatcccacttctgaca

SEQ ID NO: 629 - DNA, 84 bp, Artificial Sequence
  Description of Artificial Sequence Synthetic oligonucleotide
gtcaggatggccgagtggtctaaggcgccagactctagttctggtctccgcatggaggcgtgggttcgaatcccacttctgaca

SEQ ID NO: 630 - DNA, 106 bp, Artificial Sequence
  Description of Artificial Sequence Synthetic polynucleotide
gtcaggatggccgagtggtctaaggcgccagactctaggtaagcaccttgcctgcgggctttctggtctccggatggaggcgtgggttcgaatcccacttctgaca

SEQ ID NO: 631 - DNA, 85 bp, Artificial Sequence
  Description of Artificial Sequence Synthetic oligonucleotide
gtcaggatggccgagtggtctaaggcgccagactctagtttctggtctccggatggaggcgtgggttcgaatcccacttctgaca

SEQ ID NO: 632 - DNA, 74 bp, Artificial Sequence
  Description of Artificial Sequence Synthetic oligonucleotide
gcctccttagtgcagtaggtagcgcatcagtctctaaatctgaatggtcctgagttcaagcctcagagggggca

SEQ ID NO: 633 - DNA, 84 bp, Artificial Sequence
  Description of Artificial Sequence Synthetic oligonucleotide
gtcaggatggccgagcagtcttaaggcgctgcgttctaatcgcaccctccgctggaggcgtgggttcgaatcccacttttgaca

SEQ ID NO: 634 - DNA, 60 bp, Artificial Sequence
  Description of Artificial Sequence Synthetic oligonucleotide
ggttccatggtgtaatggtgagcactctggactctaaatccagaagtagtgctggaacaa

SEQ ID NO: 635 - DNA, 83 bp, Artificial Sequence
  Description of Artificial Sequence Synthetic oligonucleotide
gtcagggtggctgagcagtctgaggggctgcgttctagtcgcagtctgccctggaggcgtgggttcgaatcccactcctgaaa

SEQ ID NO: 636 - DNA, 83 bp, Artificial Sequence
  Description of Artificial Sequence Synthetic oligonucleotide
accaggatggccgagtggttaaggcgttggactctagatccaatggacatatgtccgcgtgggttcgaaccccactcctggta

SEQ ID NO: 637 - DNA, 83 bp, Artificial Sequence
  Description of Artificial Sequence Synthetic oligonucleotide
accgggatggccgagtggttaaggcgttggactctagatccaatgggctggtgcccgcgtgggttcgaaccccactctcggta

SEQ ID NO: 638 - DNA, 83 bp, Artificial Sequence
  Description of Artificial Sequence Synthetic oligonucleotide
accagaatggccgagtggttaaggcgttggactctagatccaatggattcatatccgcgtgggttcgaaccccacttctggta

SEQ ID NO: 639 - DNA, 83 bp, Artificial Sequence
  Description of Artificial Sequence Synthetic oligonucleotide
accgggatggctgagtggttaaggcgttggactctagatccaatggacaggtgtccgcgtgggttcgagccccactcccggta

SEQ ID NO: 640 - DNA, 83 bp, Artificial Sequence
  Description of Artificial Sequence Synthetic oligonucleotide
actcatttggctgagtggttaaggcattggactctagatccaatggagtagtggctgtgtgggtttaaaccccactactggta

SEQ ID NO: 641 - DNA, 69 bp, Artificial Sequence
  Description of Artificial Sequence Synthetic oligonucleotide
gagaaagtcatcgtagttacgaagttggctctaacccagttttgggaggttcaattccttcctttctct

SEQ ID NO: 642 - DNA, 84 bp, Artificial Sequence
  Description of Artificial Sequence Synthetic oligonucleotide
accaggatggccaagtagttaaaggcactggactctagagccaatggacatatgtctgtgtgggtttgaaccccactcctggtg

SEQ ID NO: 643 - DNA, 82 bp, Artificial Sequence; identical to SEQ ID NO: 160
  Description of Artificial Sequence Synthetic oligonucleotide

SEQ ID NO: 644 - DNA, 82 bp, Artificial Sequence; identical to SEQ ID NO: 117
  Description of Artificial Sequence Synthetic oligonucleotide

SEQ ID NO: 645 - DNA, 82 bp, Artificial Sequence; identical to SEQ ID NO: 162
  Description of Artificial Sequence Synthetic oligonucleotide

SEQ ID NO: 646 - DNA, 82 bp, Artificial Sequence
  Description of Artificial Sequence Synthetic oligonucleotide
ggtagtgtggttgaatggtctaaggcactgaattctagctccagtctctttggggacgtgggtttaaatcccactgctgcaa

SEQ ID NO: 647 - DNA, 75 bp, Artificial Sequence
  Description of Artificial Sequence Synthetic oligonucleotide
gttaagatggcagagcctggtaattgcatcaaacttaaaattttataatcagaggttcaactcctcttcttaaca

SEQ ID NO: 648 - DNA, 75 bp, Artificial Sequence
  Description of Artificial Sequence Synthetic oligonucleotide
gttaagatggcagagcccggcaattgcatcagacttaaaactttataatcagaggttcaactcctctcattaaca

SEQ ID NO: 649 - DNA, 82 bp, Artificial Sequence
  Description of Artificial Sequence Synthetic oligonucleotide
ggtagcgtggccgagcggtctaaggcgctggatttcagctccagtctcttcgggggcgtgggttcaaatcccaccgctgcca

SEQ ID NO: 650 - DNA, 82 bp, Artificial Sequence
  Description of Artificial Sequence Synthetic oligonucleotide
ggtagcgtggccgagtggtctaagacgctggatttcagctccagtctcttcgggggcgtgggtttgaatcccaccgctgcca

SEQ ID NO: 651 - DNA, 73 bp, Artificial Sequence
  Description of Artificial Sequence Synthetic oligonucleotide
gggccagtggctcaatggataatgcgtctgacttcaaatcagaagattccagccttgactcctggctggctca

SEQ ID NO: 652 - DNA, 82 bp, Artificial Sequence
  Description of Artificial Sequence Synthetic oligonucleotide
ggtagggtggccgagcggtctaaggcactgtatttcaactccagtctcttcagaggcatgggtttgaatcccactgctgcca

SEQ ID NO: 653 - DNA, 65 bp, Artificial Sequence
  Description of Artificial Sequence Synthetic oligonucleotide
gccgagcggtctaaggctccggatttcagcgccggtgtcttcggaggcatgggttcgaattccac

SEQ ID NO: 654 - DNA, 106 bp, Artificial Sequence
  Description of Artificial Sequence Synthetic polynucleotide
gtcaggatggccgagtggtctaaggcgccagacttcagctaagcttcctccgcggtggggattctggtctccaatggaggcgtgggttcgaatcccacttctgaca

SEQ ID NO: 655 - DNA, 83 bp, Artificial Sequence
  Description of Artificial Sequence Synthetic oligonucleotide
gtcaggatggccgagtggtctaaggcgccagacttcagttctggtctccaatggaggcgtgggttcgaatcccacttctgaca

SEQ ID NO: 656 - DNA, 105 bp, Artificial Sequence
  Description of Artificial Sequence Synthetic polynucleotide
gtcaggatggccgagtggtctaaggcgccagacttcagcttggcttcctcgtgttgaggattctggtctccaatggaggcgtgggttcgaatcccacttctgaca

SEQ ID NO: 657 - DNA, 83 bp, Artificial Sequence; identical to SEQ ID NO: 655
  Description of Artificial Sequence Synthetic oligonucleotide

SEQ ID NO: 658 - DNA, 108 bp, Artificial Sequence
  Description of Artificial Sequence Synthetic polynucleotide
gtcaggatggccgagtggtctaaggcgccagacttcagcttactgcttcctgtgttcgggtcttctggtctccgtatggaggcgtgggttcgaatcccacttctgaca

SEQ ID NO: 659 - DNA, 84 bp, Artificial Sequence; identical to SEQ ID NO: 129
  Description of Artificial Sequence Synthetic oligonucleotide

SEQ ID NO: 660 - DNA, 107 bp, Artificial Sequence
  Description of Artificial Sequence Synthetic polynucleotide
gtcaggatggccgagtggtctaaggcgccagacttcagttgctacttcccaggtttggggcttctggtctccgcatggaggcgtgggttcgaatcccacttctgaca

SEQ ID NO: 661 - DNA, 84 bp, Artificial Sequence
  Description of Artificial Sequence Synthetic oligonucleotide
gtcaggatggccgagtggtctaaggcgccagacttcagttctggtctccgcatggaggcgtgggttcgaatcccacttctgaca

SEQ ID NO: 662 - DNA, 106 bp, Artificial Sequence
  Description of Artificial Sequence Synthetic polynucleotide
gtcaggatggccgagtggtctaaggcgccagacttcaggtaagcaccttgcctgcgggctttctggtctccggatggaggcgtgggttcgaatcccacttctgaca

SEQ ID NO: 663 - DNA, 85 bp, Artificial Sequence
  Description of Artificial Sequence Synthetic oligonucleotide
gtcaggatggccgagtggtctaaggcgccagacttcagtttctggtctccggatggaggcgtgggttcgaatcccacttctgaca

SEQ ID NO: 664 - DNA, 74 bp, Artificial Sequence
  Description of Artificial Sequence Synthetic oligonucleotide
gcctccttagtgcagtaggtagcgcatcagtcttcaaatctgaatggtcctgagttcaagcctcagagggggca

SEQ ID NO: 665 - DNA, 84 bp, Artificial Sequence
  Description of Artificial Sequence Synthetic oligonucleotide
gtcaggatggccgagcagtcttaaggcgctgcgtttcaatcgcaccctccgctggaggcgtgggttcgaatcccacttttgaca

SEQ ID NO: 666 - DNA, 60 bp, Artificial Sequence
  Description of Artificial Sequence Synthetic oligonucleotide
ggttccatggtgtaatggtgagcactctggacttcaaatccagaagtagtgctggaacaa

SEQ ID NO: 667 - DNA, 83 bp, Artificial Sequence
  Description of Artificial Sequence Synthetic oligonucleotide
gtcagggtggctgagcagtctgaggggctgcgtttcagtcgcagtctgccctggaggcgtgggttcgaatcccactcctgaaa

SEQ ID NO: 668 - DNA, 83 bp, Artificial Sequence
  Description of Artificial Sequence Synthetic oligonucleotide
accaggatggccgagtggttaaggcgttggacttcagatccaatggacatatgtccgcgtgggttcgaaccccactcctggta

SEQ ID NO: 669 - DNA, 83 bp, Artificial Sequence
  Description of Artificial Sequence Synthetic oligonucleotide
accgggatggccgagtggttaaggcgttggacttcagatccaatgggctggtgcccgcgtgggttcgaaccccactctcggta

SEQ ID NO: 670 - DNA, 83 bp, Artificial Sequence
  Description of Artificial Sequence Synthetic oligonucleotide
accagaatggccgagtggttaaggcgttggacttcagatccaatggattcatatccgcgtgggttcgaaccccacttctggta

SEQ ID NO: 671 - DNA, 83 bp, Artificial Sequence
  Description of Artificial Sequence Synthetic oligonucleotide
accgggatggctgagtggttaaggcgttggacttcagatccaatggacaggtgtccgcgtgggttcgagccccactcccggta

SEQ ID NO: 672 - DNA, 83 bp, Artificial Sequence
  Description of Artificial Sequence Synthetic oligonucleotide
actcatttggctgagtggttaaggcattggacttcagatccaatggagtagtggctgtgtgggtttaaaccccactactggta

SEQ ID NO: 673 - DNA, 69 bp, Artificial Sequence
  Description of Artificial Sequence Synthetic oligonucleotide
gagaaagtcatcgtagttacgaagttggcttcaacccagttttgggaggttcaattccttcctttctct

SEQ ID NO: 674 - DNA, 84 bp, Artificial Sequence
  Description of Artificial Sequence Synthetic oligonucleotide
accaggatggccaagtagttaaaggcactggacttcagagccaatggacatatgtctgtgtgggtttgaaccccactcctggtg

SEQ ID NO: 675 - DNA, 82 bp, Artificial Sequence; identical to SEQ ID NO: 163
  Description of Artificial Sequence Synthetic oligonucleotide

SEQ ID NO: 676 - DNA, 82 bp, Artificial Sequence; identical to SEQ ID NO: 120
  Description of Artificial Sequence Synthetic oligonucleotide

SEQ ID NO: 677 - DNA, 82 bp, Artificial Sequence; identical to SEQ ID NO: 165
  Description of Artificial Sequence Synthetic oligonucleotide

SEQ ID NO: 678 - DNA, 82 bp, Artificial Sequence
  Description of Artificial Sequence Synthetic oligonucleotide
ggtagtgtggttgaatggtctaaggcactgaatttcagctccagtctctttggggacgtgggtttaaatcccactgctgcaa

SEQ ID NO: 679 - DNA, 70 bp, Artificial Sequence
  Description of Artificial Sequence Synthetic oligonucleotide
gagaaggtcacagaggttatgggattggctttaaaccagtctgtggggggttcgattccctcctttttca

SEQ ID NO: 680 - DNA, 70 bp, Artificial Sequence
  Description of Artificial Sequence Synthetic oligonucleotide
gagaaggtcatagaggttatgggattggctttaaaccagtctctggggggttcgattccctcctttttca

SEQ ID NO: 681 - DNA, 69 bp, Artificial Sequence
  Description of Artificial Sequence Synthetic oligonucleotide
gaaaaagtcataggggttatgaggctggctttaaaccagccttaggaggttcaattccttccttttttg

SEQ ID NO: 682 - DNA, 74 bp, Artificial Sequence
  Description of Artificial Sequence Synthetic oligonucleotide
ggccggttagctcagttggttagagcgtgctgctttaaatgccagggtcgaggtttcgatccccgtacgggcct

SEQ ID NO: 683 - DNA, 82 bp, Artificial Sequence; identical to SEQ ID NO: 232
  Description of Artificial Sequence Synthetic oligonucleotide

SEQ ID NO: 684 - DNA, 82 bp, Artificial Sequence
  Description of Artificial Sequence Synthetic oligonucleotide
gtagtcgtggccgagtggttaaggcgatggactttaaatccattggggtttccccacgcaggttcgaatcctgccgactacg

SEQ ID NO: 685 - DNA, 82 bp, Artificial Sequence
  Description of Artificial Sequence Synthetic oligonucleotide
gtagtcgtggccgagtggttaaggtgatggactttaaacccattggggtctccccgcgcaggttcgaatcctgccgactacg

SEQ ID NO: 686 - DNA, 72 bp, Artificial Sequence
  Description of Artificial Sequence Synthetic oligonucleotide
gggtgtatggctcaggggtagagaatttgactttagatcaagaggtccctggttcaaatccaggtgccccct

SEQ ID NO: 687 - DNA, 78 bp, Artificial Sequence
  Description of Artificial Sequence Synthetic oligonucleotide
agttgtagctgagtggttaaggcaacgagctttaaattcgttggtttctctctgtgcaggtttgaatcctgctaatta

SEQ ID NO: 688 - DNA, 70 bp, Artificial Sequence
  Description of Artificial Sequence Synthetic oligonucleotide
caagaaattcatagaggttatgggattggctttaaaccagtttcaggaggttcgattccttcctttttgg

SEQ ID NO: 689 - DNA, 82 bp, Artificial Sequence; identical to SEQ ID NO: 241
  Description of Artificial Sequence Synthetic oligonucleotide

SEQ ID NO: 690 - DNA, 82 bp, Artificial Sequence
  Description of Artificial Sequence Synthetic oligonucleotide
gctgtgatggccgagtggttaaggcgttggactttaaatccaatggggtctccccgcgcaggttcaaatcctgctcacagcg

SEQ ID NO: 691 - DNA, 82 bp, Artificial Sequence
  Description of Artificial Sequence Synthetic oligonucleotide
gctgtgatggccgagtggttaaggtgttggactttaaatccaatgggggttccccgcgcaggttcaaatcctgctcacagcg

SEQ ID NO: 692 - DNA, 82 bp, Artificial Sequence; identical to SEQ ID NO: 242
  Description of Artificial Sequence Synthetic oligonucleotide

SEQ ID NO: 693 - DNA, 82 bp, Artificial Sequence
  Description of Artificial Sequence Synthetic oligonucleotide
gacgaggtggccgagtggttaaggcgatggactttaaatccattgtgctctgcacgcgtgggttcgaatcccaccctcgtcg

SEQ ID NO: 694 - DNA, 82 bp, Artificial Sequence; identical to SEQ ID NO: 254
  Description of Artificial Sequence Synthetic oligonucleotide

SEQ ID NO: 695 - DNA, 74 bp, Artificial Sequence
  Description of Artificial Sequence Synthetic oligonucleotide
ggccggttagctcagttggttagagcgtgctttaactaatgccagggtcgaggtttcgatccccgtacgggcct

SEQ ID NO: 696 - DNA, 82 bp, Artificial Sequence
  Description of Artificial Sequence Synthetic oligonucleotide
gacgaggtggccgagtggttaaggcgatggactttaaatccattgtgctctgcacacgtgggttcgaatcccatcctcgtcg

SEQ ID NO: 697 - DNA, 78 bp, Artificial Sequence
  Description of Artificial Sequence Synthetic oligonucleotide
gaggcctggccgagtggttaaggcgatggactttaaatccattgtgctctgcacgcgtgggttcgaatcccatcctcg

SEQ ID NO: 698 - DNA, 82 bp, Artificial Sequence; identical to SEQ ID NO: 264
  Description of Artificial Sequence Synthetic oligonucleotide

SEQ ID NO: 699 - DNA, 82 bp, Artificial Sequence; identical to SEQ ID NO: 232
  Description of Artificial Sequence Synthetic oligonucleotide

SEQ ID NO: 700 - DNA, 82 bp, Artificial Sequence; identical to SEQ ID NO: 233
  Description of Artificial Sequence Synthetic oligonucleotide

SEQ ID NO: 701 - DNA, 82 bp, Artificial Sequence
  Description of Artificial Sequence Synthetic oligonucleotide
gtagtcgtggccgagtggttaaggcgatggactttaaatccattggggtttccccgcgcaggttcgaatcctgtcggctacg

SEQ ID NO: 702 - DNA, 70 bp, Artificial Sequence
  Description of Artificial Sequence Synthetic oligonucleotide
gagaaggtcacagaggttatgggattggctctaaaccagtctgtggggggttcgattccctcctttttca

SEQ ID NO: 703 - DNA, 70 bp, Artificial Sequence
  Description of Artificial Sequence Synthetic oligonucleotide
gagaaggtcatagaggttatgggattggctctaaaccagtctctggggggttcgattccctcctttttca

SEQ ID NO: 704 - DNA, 69 bp, Artificial Sequence
  Description of Artificial Sequence Synthetic oligonucleotide
gaaaaagtcataggggttatgaggctggctctaaaccagccttaggaggttcaattccttccttttttg

SEQ ID NO: 705 - DNA, 74 bp, Artificial Sequence
  Description of Artificial Sequence Synthetic oligonucleotide
ggccggttagctcagttggttagagcgtgctgctctaaatgccagggtcgaggtttcgatccccgtacgggcct

SEQ ID NO: 706 - DNA, 82 bp, Artificial Sequence; identical to SEQ ID NO: 226
  Description of Artificial Sequence Synthetic oligonucleotide

SEQ ID NO: 707 - DNA, 82 bp, Artificial Sequence
  Description of Artificial Sequence Synthetic oligonucleotide
gtagtcgtggccgagtggttaaggcgatggactctaaatccattggggtttccccacgcaggttcgaatcctgccgactacg

SEQ ID NO: 708 - DNA, 82 bp, Artificial Sequence
  Description of Artificial Sequence Synthetic oligonucleotide
gtagtcgtggccgagtggttaaggtgatggactctaaacccattggggtctccccgcgcaggttcgaatcctgccgactacg

SEQ ID NO: 709 - DNA, 72 bp, Artificial Sequence
  Description of Artificial Sequence Synthetic oligonucleotide
gggtgtatggctcaggggtagagaatttgactctagatcaagaggtccctggttcaaatccaggtgccccct

SEQ ID NO: 710 - DNA, 78 bp, Artificial Sequence
  Description of Artificial Sequence Synthetic oligonucleotide
agttgtagctgagtggttaaggcaacgagctctaaattcgttggtttctctctgtgcaggtttgaatcctgctaatta

SEQ ID NO: 711 - DNA, 70 bp, Artificial Sequence
  Description of Artificial Sequence Synthetic oligonucleotide
caagaaattcatagaggttatgggattggctctaaaccagtttcaggaggttcgattccttcctttttgg

SEQ ID NO: 712 - DNA, 82 bp, Artificial Sequence; identical to SEQ ID NO: 235
  Description of Artificial Sequence Synthetic oligonucleotide

SEQ ID NO: 713 - DNA, 82 bp, Artificial Sequence
  Description of Artificial Sequence Synthetic oligonucleotide
gctgtgatggccgagtggttaaggcgttggactctaaatccaatggggtctccccgcgcaggttcaaatcctgctcacagcg

SEQ ID NO: 714 - DNA, 82 bp, Artificial Sequence
  Description of Artificial Sequence Synthetic oligonucleotide
gctgtgatggccgagtggttaaggtgttggactctaaatccaatgggggttccccgcgcaggttcaaatcctgctcacagcg

SEQ ID NO: 715 - DNA, 82 bp, Artificial Sequence; identical to SEQ ID NO: 236
  Description of Artificial Sequence Synthetic oligonucleotide

SEQ ID NO: 716 - DNA, 82 bp, Artificial Sequence
  Description of Artificial Sequence Synthetic oligonucleotide
gacgaggtggccgagtggttaaggcgatggactctaaatccattgtgctctgcacgcgtgggttcgaatcccaccctcgtcg

SEQ ID NO: 717 - DNA, 82 bp, Artificial Sequence; identical to SEQ ID NO: 244
  Description of Artificial Sequence Synthetic oligonucleotide

SEQ ID NO: 718 - DNA, 74 bp, Artificial Sequence
  Description of Artificial Sequence Synthetic oligonucleotide
ggccggttagctcagttggttagagcgtgctctaactaatgccagggtcgaggtttcgatccccgtacgggcct

SEQ ID NO: 719 - DNA, 82 bp, Artificial Sequence
  Description of Artificial Sequence Synthetic oligonucleotide
gacgaggtggccgagtggttaaggcgatggactctaaatccattgtgctctgcacacgtgggttcgaatcccatcctcgtcg

SEQ ID NO: 720 - DNA, 78 bp, Artificial Sequence
  Description of Artificial Sequence Synthetic oligonucleotide
gaggcctggccgagtggttaaggcgatggactctaaatccattgtgctctgcacgcgtgggttcgaatcccatcctcg

SEQ ID NO: 721 - DNA, 82 bp, Artificial Sequence; identical to SEQ ID NO: 262
  Description of Artificial Sequence Synthetic oligonucleotide

SEQ ID NO: 722 - DNA, 82 bp, Artificial Sequence; identical to SEQ ID NO: 226
  Description of Artificial Sequence Synthetic oligonucleotide

SEQ ID NO: 723 - DNA, 82 bp, Artificial Sequence; identical to SEQ ID NO: 227
  Description of Artificial Sequence Synthetic oligonucleotide

SEQ ID NO: 724 - DNA, 82 bp, Artificial Sequence
  Description of Artificial Sequence Synthetic oligonucleotide
gtagtcgtggccgagtggttaaggcgatggactctaaatccattggggtttccccgcgcaggttcgaatcctgtcggctacg

SEQ ID NO: 725 - DNA, 70 bp, Artificial Sequence
  Description of Artificial Sequence Synthetic oligonucleotide
gagaaggtcacagaggttatgggattggcttcaaaccagtctgtggggggttcgattccctcctttttca

SEQ ID NO: 726 - DNA, 70 bp, Artificial Sequence
  Description of Artificial Sequence Synthetic oligonucleotide
gagaaggtcatagaggttatgggattggcttcaaaccagtctctggggggttcgattccctcctttttca

SEQ ID NO: 727 - DNA, 69 bp, Artificial Sequence
  Description of Artificial Sequence Synthetic oligonucleotide
gaaaaagtcataggggttatgaggctggcttcaaaccagccttaggaggttcaattccttccttttttg

SEQ ID NO: 728 - DNA, 74 bp, Artificial Sequence
  Description of Artificial Sequence Synthetic oligonucleotide
ggccggttagctcagttggttagagcgtgctgcttcaaatgccagggtcgaggtttcgatccccgtacgggcct

SEQ ID NO: 729 - DNA, 82 bp, Artificial Sequence; identical to SEQ ID NO: 229
  Description of Artificial Sequence Synthetic oligonucleotide

SEQ ID NO: 730 - DNA, 82 bp, Artificial Sequence
  Description of Artificial Sequence Synthetic oligonucleotide
gtagtcgtggccgagtggttaaggcgatggacttcaaatccattggggtttccccacgcaggttcgaatcctgccgactacg

SEQ ID NO: 731 - DNA, 82 bp, Artificial Sequence
  Description of Artificial Sequence Synthetic oligonucleotide
gtagtcgtggccgagtggttaaggtgatggacttcaaacccattggggtctccccgcgcaggttcgaatcctgccgactacg

SEQ ID NO: 732 - DNA, 72 bp, Artificial Sequence
  Description of Artificial Sequence Synthetic oligonucleotide
gggtgtatggctcaggggtagagaatttgacttcagatcaagaggtccctggttcaaatccaggtgccccct

SEQ ID NO: 733 - DNA, 78 bp, Artificial Sequence
  Description of Artificial Sequence Synthetic oligonucleotide
agttgtagctgagtggttaaggcaacgagcttcaaattcgttggtttctctctgtgcaggtttgaatcctgctaatta

SEQ ID NO: 734 - DNA, 70 bp, Artificial Sequence
  Description of Artificial Sequence Synthetic oligonucleotide
caagaaattcatagaggttatgggattggcttcaaaccagtttcaggaggttcgattccttcctttttgg

SEQ ID NO: 735 - DNA, 82 bp, Artificial Sequence; identical to SEQ ID NO: 238
  Description of Artificial Sequence Synthetic oligonucleotide

SEQ ID NO: 736 - DNA, 82 bp, Artificial Sequence
  Description of Artificial Sequence Synthetic oligonucleotide
gctgtgatggccgagtggttaaggcgttggacttcaaatccaatggggtctccccgcgcaggttcaaatcctgctcacagcg

SEQ ID NO: 737 - DNA, 82 bp, Artificial Sequence
  Description of Artificial Sequence Synthetic oligonucleotide
gctgtgatggccgagtggttaaggtgttggacttcaaatccaatgggggttccccgcgcaggttcaaatcctgctcacagcg

SEQ ID NO: 738 - DNA, 82 bp, Artificial Sequence; identical to SEQ ID NO: 239
  Description of Artificial Sequence Synthetic oligonucleotide

SEQ ID NO: 739 - DNA, 82 bp, Artificial Sequence
  Description of Artificial Sequence Synthetic oligonucleotide
gacgaggtggccgagtggttaaggcgatggacttcaaatccattgtgctctgcacgcgtgggttcgaatcccaccctcgtcg

SEQ ID NO: 740 - DNA, 82 bp, Artificial Sequence; identical to SEQ ID NO: 249
  Description of Artificial Sequence Synthetic oligonucleotide

SEQ ID NO: 741 - DNA, 74 bp, Artificial Sequence
  Description of Artificial Sequence Synthetic oligonucleotide
ggccggttagctcagttggttagagcgtgcttcaactaatgccagggtcgaggtttcgatccccgtacgggcct

SEQ ID NO: 742 - DNA, 82 bp, Artificial Sequence
  Description of Artificial Sequence Synthetic oligonucleotide
gacgaggtggccgagtggttaaggcgatggacttcaaatccattgtgctctgcacacgtgggttcgaatcccatcctcgtcg

SEQ ID NO: 743 - DNA, 78 bp, Artificial Sequence
  Description of Artificial Sequence Synthetic oligonucleotide
gaggcctggccgagtggttaaggcgatggacttcaaatccattgtgctctgcacgcgtgggttcgaatcccatcctcg

SEQ ID NO: 744 - DNA, 82 bp, Artificial Sequence; identical to SEQ ID NO: 263
  Description of Artificial Sequence Synthetic oligonucleotide

SEQ ID NO: 745 - DNA, 82 bp, Artificial Sequence; identical to SEQ ID NO: 229
  Description of Artificial Sequence Synthetic oligonucleotide

SEQ ID NO: 746 - DNA, 82 bp, Artificial Sequence; identical to SEQ ID NO: 230
  Description of Artificial Sequence Synthetic oligonucleotide

SEQ ID NO: 747 - DNA, 82 bp, Artificial Sequence
  Description of Artificial Sequence Synthetic oligonucleotide
gtagtcgtggccgagtggttaaggcgatggacttcaaatccattggggtttccccgcgcaggttcgaatcctgtcggctacg

SEQ ID NO: 748 - DNA, 73 bp, Artificial Sequence
  Description of Artificial Sequence Synthetic oligonucleotide
gcccagctagctcagtcggtagagcataagactttaaatctcagggttgtggattcgtgccccatgctgggtg

SEQ ID NO: 749 - DNA, 71 bp, Artificial Sequence
  Description of Artificial Sequence Synthetic oligonucleotide
ctgcagctagctcagtcggtagagcatgagactttaaatctcagggtcatgggttcgtgccccatgttggg

SEQ ID NO: 750 - DNA, 70 bp, Artificial Sequence
  Description of Artificial Sequence Synthetic oligonucleotide
ccagcatgtctcagtcggtatagtgtgagactttaaatctcagggtcgtgggttcaagccccacattggg

SEQ ID NO: 751 - DNA, 73 bp, Artificial Sequence
  Description of Artificial Sequence Synthetic oligonucleotide
gtctagctagatcagttggtagagcataagactttaaatctcagggtcatgggtttgagccctacgttgggcg

SEQ ID NO: 752 - DNA, 73 bp, Artificial Sequence
  Description of Artificial Sequence Synthetic oligonucleotide
gcccagctagctcagccggtagagcacaagactttaaatctcagggtcgtgggtttgagccctgtgttgagca

SEQ ID NO: 753 - DNA, 69 bp, Artificial Sequence
  Description of Artificial Sequence Synthetic oligonucleotide
ccgaatagcttagttgatgaagcgtgagactttaaatctcagggtagtgggttcaagccccacattgga

SEQ ID NO: 754 - DNA, 74 bp, Artificial Sequence
  Description of Artificial Sequence Synthetic oligonucleotide
gcctggctacctcagttggtagagcatgggactttaaatcccagagtcagtgggttcaagcctcacattgagtg

SEQ ID NO: 755 - DNA, 73 bp, Artificial Sequence
  Description of Artificial Sequence Synthetic oligonucleotide
gcccggctagctcagtcggtagagcatgagaccttaaatctcagggtcgtgggttcgagccccacgttgggcg

SEQ ID NO: 756 - DNA, 73 bp, Artificial Sequence
  Description of Artificial Sequence Synthetic oligonucleotide
gcccggctagctcagtcggtagagcatgggactttaaatctcagggtcgtgggttcgagccccacgttgggcg

SEQ ID NO: 757 - DNA, 73 bp, Artificial Sequence
  Description of Artificial Sequence Synthetic oligonucleotide
gcccggctagctcagtcgatagagcatgagactttaaatctcagggtcgtgggttcgagccgcacgttgggcg

SEQ ID NO: 758 - DNA, 73 bp, Artificial Sequence
  Description of Artificial Sequence Synthetic oligonucleotide
gcccagctagctcagtcggtagagcatgagactttaaatctcagggtcatgggtttgagccccacgtttggtg

SEQ ID NO: 759 - DNA, 73 bp, Artificial Sequence
  Description of Artificial Sequence Synthetic oligonucleotide
gcctggctagctcagtcggcaaagcatgagactttaaatctcagggtcgtgggctcgagctccatgttgggcg

SEQ ID NO: 760 - DNA, 73 bp, Artificial Sequence
  Description of Artificial Sequence Synthetic oligonucleotide
gcccgactacctcagtcggtggagcatgggactttacatcccagggttgtgggttcgagccccacattgggca

SEQ ID NO: 761 - DNA, 73 bp, Artificial Sequence
  Description of Artificial Sequence Synthetic oligonucleotide
ccccggctggctcagtcagtagatcatgagactttaaatctcagggtcgtgggttcacgccccacactgggcg

SEQ ID NO: 762 - DNA, 65 bp, Artificial Sequence
  Description of Artificial Sequence Synthetic oligonucleotide
gcgctagtcagtagagcatgagactttaaatctcagggtcgtgggttcgagccccacatcgggcg

SEQ ID NO: 763 - DNA, 73 bp, Artificial Sequence
  Description of Artificial Sequence Synthetic oligonucleotide
gcctggatagctcagttggtagagcatcagactttaaatctgagggtccagggttcaagtccctgttcaggca

SEQ ID NO: 764 - DNA, 73 bp, Artificial Sequence
  Description of Artificial Sequence Synthetic oligonucleotide
gccaggatagttcaggtggtagagcatcagactttaaacctgagggttcagggttcaagtctctgtttgggcg

SEQ ID NO: 765 - DNA, 73 bp, Artificial Sequence
  Description of Artificial Sequence Synthetic oligonucleotide
acccagatagctcagtcagtagagcatcagactttaaatctgagggtccaaggttcatgtccctttttgggtg

SEQ ID NO: 766 - DNA, 73 bp, Artificial Sequence
  Description of Artificial Sequence Synthetic oligonucleotide
acctgggtagcttagttggtagagcattggactttaaatttgagggcccaggtttcaagtccctgtttgggtg

SEQ ID NO: 767 - DNA, 74 bp, Artificial Sequence
  Description of Artificial Sequence Synthetic oligonucleotide
gcctgggtagctcagtcggtagagctatcagactttaagcctgaggattcagggttcaatcccttgctggggcg

SEQ ID NO: 768 - DNA, 62 bp, Artificial Sequence
  Description of Artificial Sequence Synthetic oligonucleotide
gatagctcagttgatagagcatcagactttaaatctgagggtccagggttcatgtccctgtt

SEQ ID NO: 769 - DNA, 73 bp, Artificial Sequence
  Description of Artificial Sequence Synthetic oligonucleotide
gttggggtaactcagttggtagagtagcagactttacatctgagggtccagggtttaagtccatgtccaggca

SEQ ID NO: 770 - DNA, 73 bp, Artificial Sequence
  Description of Artificial Sequence Synthetic oligonucleotide
gcctggatagctcagttggtagagcatcagactttaaatctgagggtccagggttcaagtccctgttcaggcg

SEQ ID NO: 771 - DNA, 73 bp, Artificial Sequence; identical to SEQ ID NO: 222
  Description of Artificial Sequence Synthetic oligonucleotide

SEQ ID NO: 772 - DNA, 73 bp, Artificial Sequence
  Description of Artificial Sequence Synthetic oligonucleotide
gcccggatagctcagtcggtagagcatcagactttaaatctgagggtccggggttcaagtccctgttcgggcg

SEQ ID NO: 773 - DNA, 73 bp, Artificial Sequence
  Description of Artificial Sequence Synthetic oligonucleotide
gcctgggtagctcagtcggtagagcatcagactttaaatctgagggtccagggttcaagtccctgtccaggcg

SEQ ID NO: 774 - DNA, 73 bp, Artificial Sequence
  Description of Artificial Sequence Synthetic oligonucleotide
gcctggatagctcagttggtagaacatcagactttaaatctgacggtgcagggttcaagtccctgttcaggcg

SEQ ID NO: 775 - DNA, 73 bp, Artificial Sequence
  Description of Artificial Sequence Synthetic oligonucleotide
gcccggagagctcagtgggtagagcatcagactttaaatctgagggtccagggttcaagtcctcgttcgggca

SEQ ID NO: 776 - DNA, 73 bp, Artificial Sequence
  Description of Artificial Sequence Synthetic oligonucleotide
acctgggtagctcagtaggtagaacatcagactttaaatctgagggtctagggttcaagtccctgtccaggcg

SEQ ID NO: 777 - DNA, 76 bp, Artificial Sequence
  Description of Artificial Sequence Synthetic oligonucleotide
gcctggatagctccttcggtagagcatcatcagactttaaatgtgagggtccagggttcaagttcctgtttgggcg

SEQ ID NO: 778 - DNA, 73 bp, Artificial Sequence
  Description of Artificial Sequence Synthetic oligonucleotide
gcccagctagctcagtcggtagagcataagactctaaatctcagggttgtggattcgtgccccatgctgggtg

SEQ ID NO: 779 - DNA, 71 bp, Artificial Sequence
  Description of Artificial Sequence Synthetic oligonucleotide
ctgcagctagctcagtcggtagagcatgagactctaaatctcagggtcatgggttcgtgccccatgttggg

SEQ ID NO: 780 - DNA, 70 bp, Artificial Sequence
  Description of Artificial Sequence Synthetic oligonucleotide
ccagcatgtctcagtcggtatagtgtgagactctaaatctcagggtcgtgggttcaagccccacattggg

SEQ ID NO: 781 - DNA, 73 bp, Artificial Sequence
  Description of Artificial Sequence Synthetic oligonucleotide
gtctagctagatcagttggtagagcataagactctaaatctcagggtcatgggtttgagccctacgttgggcg

SEQ ID NO: 782 - DNA, 73 bp, Artificial Sequence
  Description of Artificial Sequence Synthetic oligonucleotide
gcccagctagctcagccggtagagcacaagactctaaatctcagggtcgtgggtttgagccctgtgttgagca

SEQ ID NO: 783 - DNA, 69 bp, Artificial Sequence
  Description of Artificial Sequence Synthetic oligonucleotide
ccgaatagcttagttgatgaagcgtgagactctaaatctcagggtagtgggttcaagccccacattgga

SEQ ID NO: 784 - DNA, 74 bp, Artificial Sequence
  Description of Artificial Sequence Synthetic oligonucleotide
gcctggctacctcagttggtagagcatgggactctaaatcccagagtcagtgggttcaagcctcacattgagtg

SEQ ID NO: 785 - DNA, 73 bp, Artificial Sequence
  Description of Artificial Sequence Synthetic oligonucleotide
gcccggctagctcagtcggtagagcatgagaccctaaatctcagggtcgtgggttcgagccccacgttgggcg

SEQ ID NO: 786 - DNA, 73 bp, Artificial Sequence
  Description of Artificial Sequence Synthetic oligonucleotide
gcccggctagctcagtcggtagagcatgggactctaaatctcagggtcgtgggttcgagccccacgttgggcg

SEQ ID NO: 787 - DNA, 73 bp, Artificial Sequence
  Description of Artificial Sequence Synthetic oligonucleotide
gcccggctagctcagtcgatagagcatgagactctaaatctcagggtcgtgggttcgagccgcacgttgggcg

SEQ ID NO: 788 - DNA, 73 bp, Artificial Sequence
  Description of Artificial Sequence Synthetic oligonucleotide
gcccagctagctcagtcggtagagcatgagactctaaatctcagggtcatgggtttgagccccacgtttggtg

SEQ ID NO: 789 - DNA, 73 bp, Artificial Sequence
  Description of Artificial Sequence Synthetic oligonucleotide
gcctggctagctcagtcggcaaagcatgagactctaaatctcagggtcgtgggctcgagctccatgttgggcg

SEQ ID NO: 790 - DNA, 73 bp, Artificial Sequence
  Description of Artificial Sequence Synthetic oligonucleotide
gcccgactacctcagtcggtggagcatgggactctacatcccagggttgtgggttcgagccccacattgggca

SEQ ID NO: 791 - DNA, 73 bp, Artificial Sequence
  Description of Artificial Sequence Synthetic oligonucleotide
ccccggctggctcagtcagtagatcatgagactctaaatctcagggtcgtgggttcacgccccacactgggcg

SEQ ID NO: 792 - DNA, 65 bp, Artificial Sequence
  Description of Artificial Sequence Synthetic oligonucleotide
gcgctagtcagtagagcatgagactctaaatctcagggtcgtgggttcgagccccacatcgggcg

SEQ ID NO: 793 - DNA, 73 bp, Artificial Sequence
  Description of Artificial Sequence Synthetic oligonucleotide
gcctggatagctcagttggtagagcatcagactctaaatctgagggtccagggttcaagtccctgttcaggca

SEQ ID NO: 794 - DNA, 73 bp, Artificial Sequence
  Description of Artificial Sequence Synthetic oligonucleotide
gccaggatagttcaggtggtagagcatcagactctaaacctgagggttcagggttcaagtctctgtttgggcg

SEQ ID NO: 795 - DNA, 73 bp, Artificial Sequence
  Description of Artificial Sequence Synthetic oligonucleotide
acccagatagctcagtcagtagagcatcagactctaaatctgagggtccaaggttcatgtccctttttgggtg

SEQ ID NO: 796 - DNA, 73 bp, Artificial Sequence
  Description of Artificial Sequence Synthetic oligonucleotide
acctgggtagcttagttggtagagcattggactctaaatttgagggcccaggtttcaagtccctgtttgggtg

SEQ ID NO: 797 - DNA, 74 bp, Artificial Sequence
  Description of Artificial Sequence Synthetic oligonucleotide
gcctgggtagctcagtcggtagagctatcagactctaagcctgaggattcagggttcaatcccttgctggggcg

SEQ ID NO: 798 - DNA, 62 bp, Artificial Sequence
  Description of Artificial Sequence Synthetic oligonucleotide
gatagctcagttgatagagcatcagactctaaatctgagggtccagggttcatgtccctgtt

SEQ ID NO: 799 - DNA, 73 bp, Artificial Sequence
  Description of Artificial Sequence Synthetic oligonucleotide
gttggggtaactcagttggtagagtagcagactctacatctgagggtccagggtttaagtccatgtccaggca

SEQ ID NO: 800 - DNA, 73 bp, Artificial Sequence
  Description of Artificial Sequence Synthetic oligonucleotide
gcctggatagctcagttggtagagcatcagactctaaatctgagggtccagggttcaagtccctgttcaggcg

SEQ ID NO: 801 - DNA, 73 bp, Artificial Sequence; identical to SEQ ID NO: 217
  Description of Artificial Sequence Synthetic oligonucleotide

SEQ ID NO: 802 - DNA, 73 bp, Artificial Sequence
  Description of Artificial Sequence Synthetic oligonucleotide
gcccggatagctcagtcggtagagcatcagactctaaatctgagggtccggggttcaagtccctgttcgggcg

SEQ ID NO: 803 - DNA, 73 bp, Artificial Sequence
  Description of Artificial Sequence Synthetic oligonucleotide
gcctgggtagctcagtcggtagagcatcagactctaaatctgagggtccagggttcaagtccctgtccaggcg

SEQ ID NO: 804 - DNA, 73 bp, Artificial Sequence
  Description of Artificial Sequence Synthetic oligonucleotide
gcctggatagctcagttggtagaacatcagactctaaatctgacggtgcagggttcaagtccctgttcaggcg

SEQ ID NO: 805 - DNA, 73 bp, Artificial Sequence
  Description of Artificial Sequence Synthetic oligonucleotide
gcccggagagctcagtgggtagagcatcagactctaaatctgagggtccagggttcaagtcctcgttcgggca

SEQ ID NO: 806 - DNA, 73 bp, Artificial Sequence
  Description of Artificial Sequence Synthetic oligonucleotide
acctgggtagctcagtaggtagaacatcagactctaaatctgagggtctagggttcaagtccctgtccaggcg

SEQ ID NO: 807 - DNA, 76 bp, Artificial Sequence
  Description of Artificial Sequence Synthetic oligonucleotide
gcctggatagctccttcggtagagcatcatcagactctaaatgtgagggtccagggttcaagttcctgtttgggcg

SEQ ID NO: 808 - DNA, 75 bp, Artificial Sequence
  Description of Artificial Sequence Synthetic oligonucleotide
ggcagaatggtgcagcggttcagcacccaggctcttcagccagctgttgcctgggctcaaatcccagctctgcca

SEQ ID NO: 809 - DNA, 176 bp, Artificial Sequence
  Description of Artificial Sequence Synthetic polynucleotide
ggctgtatagctcagtggtagagcatttgacttcagaatcctatactcaggggaaggagaactgggggtttctcagtgggtcaaaggacttgtagtggtaaatcaaaagcaactctataagctatgtaacaaactttaaagtcatatgtagctgggttcaaatcctgtttctgcca

SEQ ID NO: 810 - DNA, 79 bp, Artificial Sequence
  Description of Artificial Sequence Synthetic oligonucleotide
ggctgtatagctcagtggtagagcatttgacttcagctttaaagtcatatgtagctgggttcaaatcctgtttctgcca

SEQ ID NO: 811 - DNA, 72 bp, Artificial Sequence
  Description of Artificial Sequence Synthetic oligonucleotide
gggggcatagctcagtggtagagcatttgacttcagatcaagaggtccctggttcaaatccaggtgccccct

SEQ ID NO: 812 - DNA, 72 bp, Artificial Sequence
  Description of Artificial Sequence Synthetic oligonucleotide
gggggtatagctcaggggtagagcatttgacttcagatcaagaggtccctggttcaaatccaggtgcccccc

SEQ ID NO: 813 - DNA, 72 bp, Artificial Sequence
  Description of Artificial Sequence Synthetic oligonucleotide
gggggtatagcttagcggtagagcatttgacttcagatcaagaggtccccggttcaaatccgggtgccccct

SEQ ID NO: 814 - DNA, 72 bp, Artificial Sequence
  Description of Artificial Sequence Synthetic oligonucleotide
gggggtatagcttaggggtagagcatttgacttcagatcaaaaggtccctggttcaaatccaggtgcccctt

SEQ ID NO: 815 - DNA, 72 bp, Artificial Sequence
  Description of Artificial Sequence Synthetic oligonucleotide
gggggtatagctcaggggtagagcatttgacttcagatcaagaggtccccagttcaaatctgggtgccccct

SEQ ID NO: 816 - DNA, 72 bp, Artificial Sequence
  Description of Artificial Sequence Synthetic oligonucleotide
gggggtatagctcaggggtagagcatttgacttcagatcaagaagtccccggttcaaatccgggtgccccct

SEQ ID NO: 817 - DNA, 72 bp, Artificial Sequence
  Description of Artificial Sequence Synthetic oligonucleotide
gggggtatagctcaggggtagagcatttgacttcagatcaagaggtctctggttcaaatccaggtgccccct

SEQ ID NO: 818 - DNA, 72 bp, Artificial Sequence
  Description of Artificial Sequence Synthetic oligonucleotide
gggggtatagctcaggggtagagcacttgacttcagatcaagaagtccttggttcaaatccaggtgccccct

SEQ ID NO: 819 - DNA, 72 bp, Artificial Sequence
  Description of Artificial Sequence Synthetic oligonucleotide
ggggatatagctcaggggtagagcatttgacttcagatcaagaggtccccggttcaaatccgggtgcccccc

SEQ ID NO: 820 - DNA, 72 bp, Artificial Sequence
  Description of Artificial Sequence Synthetic oligonucleotide
gggggtatagttcaggggtagagcatttgacttcagatcaagaggtccctggttcaaatccaggtgccccct

SEQ ID NO: 821 - DNA, 72 bp, Artificial Sequence
  Description of Artificial Sequence Synthetic oligonucleotide
gggggtatagctcaggggtagagcatttgacttcaaatcaagaggtccctgattcaaatccaggtgccccct

SEQ ID NO: 822 - DNA, 72 bp, Artificial Sequence
  Description of Artificial Sequence Synthetic oligonucleotide
gggcgtatagctcaggggtagagcatttgacttcagatcaagaggtccccagttcaaatctgggtgccccct

SEQ ID NO: 823 - DNA, 72 bp, Artificial Sequence
  Description of Artificial Sequence Synthetic oligonucleotide
gggggtatagctcacaggtagagcatttgacttcagatcaagaggtccccggttcaaatctgggtgccccct

SEQ ID NO: 824 - DNA, 70 bp, Artificial Sequence
  Description of Artificial Sequence Synthetic oligonucleotide
gggcgtatagctcaggggtagagcatttgacttcagatcaagaggtccccagttcaaatctgggtgccca

SEQ ID NO: 825 - DNA, 72 bp, Artificial Sequence
  Description of Artificial Sequence Synthetic oligonucleotide
gggggtatagctcacaggtagagcatttgacttcagatcaagaggtccccggttcaaatccggttactccct

SEQ ID NO: 826 - DNA, 72 bp, Artificial Sequence
  Description of Artificial Sequence Synthetic oligonucleotide
gggggtagggctcagggatagagcatttgacttcagatcaagaggtccccggttcgaatctaggtgccccct

SEQ ID NO: 827 - DNA, 66 bp, Artificial Sequence
  Description of Artificial Sequence Synthetic oligonucleotide
ggtatatctcagggggcagagcatttgacttcagatcaagaggtccccggttgaaatccgggtgct

SEQ ID NO: 828 - DNA, 72 bp, Artificial Sequence
  Description of Artificial Sequence Synthetic oligonucleotide
gggggtatagctcaggggtagagcacttgacttcagatcaagaggtccctggttcaaatccaggtgccccct

SEQ ID NO: 829 - DNA, 72 bp, Artificial Sequence; identical to SEQ ID NO: 82
  Description of Artificial Sequence Synthetic oligonucleotide

SEQ ID NO: 830 - DNA, 73 bp, Artificial Sequence
  Description of Artificial Sequence Synthetic oligonucleotide
gggggtatagctcagtgggtagagcatttgacttcagatcaagaggtccccggttcaaatccgggtgccccct

SEQ ID NO: 831 - DNA, 72 bp, Artificial Sequence
  Description of Artificial Sequence Synthetic oligonucleotide
gggggtgtagctcagtggtagagcatttgacttcagatcaagaggtccctggttcaaatccaggtgccccct

SEQ ID NO: 832 - DNA, 72 bp, Artificial Sequence; identical to SEQ ID NO: 90
  Description of Artificial Sequence Synthetic oligonucleotide

SEQ ID NO: 833 - DNA, 72 bp, Artificial Sequence; identical to SEQ ID NO: 87
  Description of Artificial Sequence Synthetic oligonucleotide

SEQ ID NO: 834 - DNA, 72 bp, Artificial Sequence; identical to SEQ ID NO: 271
  Description of Artificial Sequence Synthetic oligonucleotide

SEQ ID NO: 835 - DNA, 72 bp, Artificial Sequence; identical to SEQ ID NO: 276
  Description of Artificial Sequence Synthetic oligonucleotide

SEQ ID NO: 836 - DNA, 106 bp, Artificial Sequence
  Description of Artificial Sequence Synthetic polynucleotide
gaagcggtggctcaatggtagagctttcgacttcaattaaatcttggaaattccacggaataagattgcaatcgaagggttgcaggttcaattcctgtccgtttca

SEQ ID NO: 837 - DNA, 72 bp, Artificial Sequence
  Description of Artificial Sequence Synthetic oligonucleotide
gaagcggtggctcaatggtagagctttcgacttcaaatcgaagggttgcaggttcaattcctgtccgtttca

SEQ ID NO: 838 - DNA, 72 bp, Artificial Sequence
  Description of Artificial Sequence Synthetic oligonucleotide
ggcctcatggtgcaacagtagtgtgtctgacttcagatcagaaggttgtatgttcaaatcacataggggtca

SEQ ID NO: 839 - DNA, 72 bp, Artificial Sequence
  Description of Artificial Sequence Synthetic oligonucleotide
gacctcgtggtgaaatggtagcatgtttgacttcaaatcaggaggttgtgtgttcaagtcacatcagggtca

SEQ ID NO: 840 - DNA, 72 bp, Artificial Sequence
  Description of Artificial Sequence Synthetic oligonucleotide
gaccttgtggcgcaatggtagcatgtttgacttcaaatcaggaggttgtgtgttcaagtcacatcagggtca

SEQ ID NO: 841 - DNA, 72 bp, Artificial Sequence
  Description of Artificial Sequence Synthetic oligonucleotide
gacctcgtggcgcaacggtagcgcgtctgacttcagatcagaaggctgcgtgttcgaatcacgccggggtca

SEQ ID NO: 842 - DNA, 72 bp, Artificial Sequence
  Description of Artificial Sequence Synthetic oligonucleotide
gaccttgtggctcaatggtagcgcatctgacttcagatcaggaggttgcacgttcaaatcatgccggggtca

SEQ ID NO: 843 - DNA, 72 bp, Artificial Sequence
  Description of Artificial Sequence Synthetic oligonucleotide
gaccttgtggcgcaacggtagcgcgtctgacttcagatcagaaggttgcgtgttcaaatcacgtcggggtca

SEQ ID NO: 844 - DNA, 72 bp, Artificial Sequence
  Description of Artificial Sequence Synthetic oligonucleotide
gacctcgtggcgcaacggtagcgcgtctgacttcagatcagaaggttgcgtattcaaatcacgtcggggtca

SEQ ID NO: 845 - DNA, 72 bp, Artificial Sequence
  Description of Artificial Sequence Synthetic oligonucleotide
gacctcgtggcgcaacggcagcgcgtctgacttcacattagaaggttgcgtgttcaaatcacgtcggggtca

SEQ ID NO: 846 - DNA, 72 bp, Artificial Sequence
  Description of Artificial Sequence Synthetic oligonucleotide
gacctcatggcgcaacggtagcgcgtctgacttcagatcagaaggttgcgtgttcaaatcacatcggggtca

SEQ ID NO: 847 - DNA, 72 bp, Artificial Sequence
  Description of Artificial Sequence Synthetic oligonucleotide
gacctcgtggtgcaacggtagcgcgtatgatttcagatcagaaggttgcgtgttcaaatcacgtcggggtca

SEQ ID NO: 848 - DNA, 72 bp, Artificial Sequence
  Description of Artificial Sequence Synthetic oligonucleotide
gacctcgtagcgcaacggtagcgcgtctgacttcagatcagaaggttgcgtgttcaaatcacgtcggggtca

SEQ ID NO: 849 - DNA, 76 bp, Artificial Sequence
  Description of Artificial Sequence Synthetic oligonucleotide
aggggtatagctcaattggcagagcgtcggtcttcaaaaccgaaggttgtaggttcgattcctactgcccctgcca

SEQ ID NO: 850 - DNA, 72 bp, Artificial Sequence
  Description of Artificial Sequence Synthetic oligonucleotide
gacctcatggcgcaacggtagcgcgtctgacttcagatcagaaggttgcgtgttcaaatcacgtcggggtca

SEQ ID NO: 851 - DNA, 72 bp, Artificial Sequence
  Description of Artificial Sequence Synthetic oligonucleotide
gacctcgtggcgcaacggtagcgcgtctaacttcagatcagaaggttgcgtgttcaaatcacgtcggggtca

SEQ ID NO: 852 - DNA, 73 bp, Artificial Sequence
  Description of Artificial Sequence Synthetic oligonucleotide
acgggagtagctcagttggtagagcaccggtcttcaaaaccgggtgtcgggagttcgagcctctcctcccgtg

SEQ ID NO: 853 - DNA, 72 bp, Artificial Sequence
  Description of Artificial Sequence Synthetic oligonucleotide
gacctcgtggcgcaacggtagcgcgtctgacttcagatcagaaggttgcatgttcaaatcacgtcggggtca

SEQ ID NO: 854 - DNA, 72 bp, Artificial Sequence
  Description of Artificial Sequence Synthetic oligonucleotide
gactccgtggcgcaacggtagcgcgtccgacttcagatcggaaggttgcgtgttcaaatcacgtcggggtca

SEQ ID NO: 855 - DNA, 72 bp, Artificial Sequence
  Description of Artificial Sequence Synthetic oligonucleotide
gactccgtggcgcaacggtagcgcgtctgacttcagatcagaaggttgcgtgttcaaatcacgtcggggtca

SEQ ID NO: 856 - DNA, 72 bp, Artificial Sequence
  Description of Artificial Sequence Synthetic oligonucleotide
ggcctcgtggcgcaacggtagcgcgtctgactccagatcagaaggttgcgtgttcaaatcacgtcggggtca

SEQ ID NO: 857 - DNA, 72 bp, Artificial Sequence
  Description of Artificial Sequence Synthetic oligonucleotide
ggcctcgtggcgcaacggtagcacgtctgactccagatcagaaggttgcgtgttcaaatcacgtcggggtca

SEQ ID NO: 858 - DNA, 73 bp, Artificial Sequence
  Description of Artificial Sequence Synthetic oligonucleotide
cggcctcgtggcgcaacggtagcacgtctgacttcagatcagaaggttgcgtgttcaaatcacgtcggggtca

SEQ ID NO: 859 - DNA, 72 bp, Artificial Sequence
  Description of Artificial Sequence Synthetic oligonucleotide
ggcctcgtcgcgcaacggtagcgcgtctgactccagatcagaaggttgcgtgttcaaatcacgtcggggtca

SEQ ID NO: 860 - DNA, 72 bp, Artificial Sequence
  Description of Artificial Sequence Synthetic oligonucleotide
ggcctcgtcgcgcaacggtagcgcgtctgacttcagatcagaaggttgcgtgttcaaatcacgtcggggtca

SEQ ID NO: 861 - DNA, 72 bp, Artificial Sequence
  Description of Artificial Sequence Synthetic oligonucleotide
ggcctcgtcgcgcaacggtagcacgtctgactccagatcagaaggttgcgtgttcaaatcacgtcggggtca

SEQ ID NO: 862 - DNA, 72 bp, Artificial Sequence
  Description of Artificial Sequence Synthetic oligonucleotide
ggcctcgtcgcgcaacggtagcacgtctgacttcagatcagaaggttgcgtgttcaaatcacgtcggggtca

SEQ ID NO: 863 - protein, 2009 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 864 - protein, 1998 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 865 - protein, 1981 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 866 - protein, 1997 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 867 - protein, 1980 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 868 - protein, 1195 aa, Homo sapiens
MFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 869 - DNA, 100 bp, Artificial Sequence
  Description of Artificial Sequence Synthetic polynucleotide
ccaaaacatcttttactgtagtatctacttaccatactacccaagaatggcacactgctcacatcttcaaaagcttaaaccaagagcactacacaggtgc

SEQ ID NO: 870 - DNA, 100 bp, Artificial Sequence
  Description of Artificial Sequence Synthetic polynucleotide
tgtgtgtcggggccggtaccctgcttccggttcccgcacgcattcccggattgcagtgcggaccccttctgtaagcgcgcgataaagcgcggttttggaa

SEQ ID NO: 871 - DNA, 100 bp, Artificial Sequence
  Description of Artificial Sequence Synthetic polynucleotide
tcatgtcatataagtagaaccatacaatatatatataaaatccaggttaatagccaatcttacaacatttctcatattttttgcagttgctaagccatgg

SEQ ID NO: 872 - DNA, 100 bp, Artificial Sequence
  Description of Artificial Sequence Synthetic polynucleotide
acattacaatacatatcaacatatcaccataattaaattgcaagtcttcgtcaaaagcaagccttaaaggagtatcccaaaaacacattttccccagaag

SEQ ID NO: 873 - DNA, 100 bp, Artificial Sequence
  Description of Artificial Sequence Synthetic polynucleotide
agacctttagagcgtggttaaacccatatgttgggatttatgctgcttttatggtagcaataccctatattaagatttgaagtagacccggaaagttagt

SEQ ID NO: 874 - DNA, 100 bp, Artificial Sequence
  Description of Artificial Sequence Synthetic polynucleotide
gttcatgaaagaataaataaatgtttaaaaaaaaaaaaaactgaggtaaatttctatattctttcataaaagcagtttaaagacgaacgtttttcgaggt

SEQ ID NO: 875 - DNA, 100 bp, Artificial Sequence
  Description of Artificial Sequence Synthetic polynucleotide
gctgggtctcggtgacactgacgacgggaggcgcggtcggaagagcgcggggccgtcgcctctggcttaacatagcagatgcgctgagactccaacaggt

SEQ ID NO: 876 - DNA, 100 bp, Artificial Sequence
  Description of Artificial Sequence Synthetic polynucleotide
cagtggcggcgaaaactctctgcgttctggagggagggtgcgggcaggaggaggtagaggatgccttgtaagcggagcaaaaacaaggttcaacgtctgc

SEQ ID NO: 877 - DNA, 100 bp, Artificial Sequence
  Description of Artificial Sequence Synthetic polynucleotide
caaatcacttgcctctcggcgcgagaccgcgatgcgcgggggcgggagcgtgatgatggcatcgcgtaaggagagggtgtgagaagccggatcctgtggt

SEQ ID NO: 878 - DNA, 100 bp, Artificial Sequence
  Description of Artificial Sequence Synthetic polynucleotide
ccctgtgtccgaagaggtctgcgttgcgacttacgtggtagtgcttggaaggtgcggagtagatgagagataagtgaatgtggacaaacctgtcacgtag

SEQ ID NO: 879 - DNA, 100 bp, Artificial Sequence
  Description of Artificial Sequence Synthetic polynucleotide
gagcggagctcagagggtgcgcgctccgccctttcgcgggcctggcatgagcgcagtggttgttacactaaagtgtctccgcctgtcgaatattctcgtg

SEQ ID NO: 880 - DNA, 100 bp, Artificial Sequence
  Description of Artificial Sequence Synthetic polynucleotide
gtgtcactggtttcaaatcaacctcaatttttttggagacgtgagtgctgagcattttttcttcagtgaagtgacttggcagccaaaatcgccaacgccc

SEQ ID NO: 881 - DNA, 100 bp, Artificial Sequence
  Description of Artificial Sequence Synthetic polynucleotide
tcctggcatgtcccgcccaagtcccttagccccgctccccaaccctgccccattcccactctagtacccgtaagctacaagacgccgccgttcgtcgggt

SEQ ID NO: 882 - DNA, 100 bp, Artificial Sequence
  Description of Artificial Sequence Synthetic polynucleotide
tgctcagtcgtcctgccgggcgggccctgaggttgcaagggacggaggaagtttcgtgcgtgcgcccttcctatagcgcccagtagaactgacagtacct

SEQ ID NO: 883 - DNA, 100 bp, Artificial Sequence
  Description of Artificial Sequence Synthetic polynucleotide
tcctcggattacgcatgctcagtgcaatcttcggttgcctggactagcgctccggtttttctgtgctgaacctcaggggacgccgacacacgtacacgtc

SEQ ID NO: 884 - DNA, 100 bp, Artificial Sequence
  Description of Artificial Sequence Synthetic polynucleotide
gataatttcctgaaagaaaagatcaattcgatgttaccaaatctgggatatccagaaaaattttcttcttctcctaggagaaaaactatcaaatgtcagg

SEQ ID NO: 885 - DNA, 100 bp, Artificial Sequence
  Description of Artificial Sequence Synthetic polynucleotide
tctctcacggcaaactgttgcagactgtagagacgctatgccaagaatcttttacttaaaagcaggaatagattcaataggcaacttcactgcacatgta

SEQ ID NO: 886 - DNA, 100 bp, Artificial Sequence
  Description of Artificial Sequence Synthetic polynucleotide
caacctccccttctcaaggagcaggtggattggtcccgagctagctggtgggcggaggtgacgtttttataagttgctcaagagacggtaacaaccgacg

SEQ ID NO: 887 - DNA, 100 bp, Artificial Sequence
  Description of Artificial Sequence Synthetic polynucleotide
gtggaacttccactgaattactcttttcgcatgtaagatcactgaaccgtgataatcattgatcctatttgtagaactgtatgaaacagttccctaagga

SEQ ID NO: 888 - DNA, 100 bp, Artificial Sequence
  Description of Artificial Sequence Synthetic polynucleotide
tcgctcaacaggcggccagggtgcgagcagtgaagctgcggcacgccggagcgtttaatggccatcaaattggcctctctaggaggtagctgcagccgga

SEQ ID NO: 889 - DNA, 51 bp, Artificial Sequence
  Description of Artificial Sequence Synthetic oligonucleotide
atagaaagaaatgagactgctcgatagaaaaatgtgaaagtaaactttgat

SEQ ID NO: 890 - DNA, 51 bp, Artificial Sequence
  Description of Artificial Sequence Synthetic oligonucleotide
atagaaagaaatgagactgctcgacagaaaaatgtgaaagtaaactttgat

SEQ ID NO: 891 - DNA, 51 bp, Artificial Sequence
  Description of Artificial Sequence Synthetic oligonucleotide
tataatgcaatgaaaaaattaggatagaaaaaaccgcaaaagcctatacct

SEQ ID NO: 892 - DNA, 51 bp, Artificial Sequence
  Description of Artificial Sequence Synthetic oligonucleotide
tataatgcaatgaaaaaattaggacagaaaaaaccgcaaaagcctatacct

SEQ ID NO: 893 - DNA, 51 bp, Artificial Sequence
  Description of Artificial Sequence Synthetic oligonucleotide
gagaagtttgatcccgatgcaacttagttcatggaatttgaaaaattatct

SEQ ID NO: 894 - DNA, 51 bp, Artificial Sequence
  Description of Artificial Sequence Synthetic oligonucleotide
gagaagtttgatcccgatgcaactcagttcatggaatttgaaaaattatct

SEQ ID NO: 895 - DNA, 20 bp, Artificial Sequence
  Description of Artificial Sequence Synthetic oligonucleotide
aaaggcaccttctcgctggc

SEQ ID NO: 896 - DNA, 17 bp, Artificial Sequence
  Description of Artificial Sequence Synthetic oligonucleotide
acttgtatgttgttttt

SEQ ID NO: 897 - DNA, 10 bp, Artificial Sequence
  Description of Artificial Sequence Synthetic oligonucleotide
tctcgctggc

SEQ ID NO: 898 - DNA, 6063 bp, Artificial Sequence
  Description of Artificial Sequence Synthetic polynucleotide
atggagcaaacagtgcttgtaccaccaggacctgacagcttcaacttcttcaccagagaatcccttgcagctattgaaaggcgcattgcagaagagaaggctaagaatcccaagccagacaaaaaagatgatgatgaaaatggcccaaagccaaacagtgacttggaagctgggaagaaccttccatttatctatggagacattcctccagagatggtgtcggagcctctggaggacctggacccctactatatcaataagaagacttttatagtattgaataaagggaaggccatcttccggttcagtgccacctccgccctgtacattttaacacccttcaatcctcttaggaaaatagctattaagattttggtacactcattattcagcatgttaatcatgtgcactattttgacaaactgtgtatttatgacaatgagtaaccctcccgactggacaaagaatgtggagtacaccttcacaggaatatatacttttgaatcactaataaaaattattgcaaggggcttctgtttagaagattttactttccttcgcgacccatggaactggctggacttcactgtcattacattcgcatatgtgacggagtttgtggacctgggcaatgtctcagcattgagaacattcagagttcttcgagcattgaaaactatttcagtcattccaggcctgaagaccatcgtgggggccctgatccagtcggtgaagaagctgtctgacgtcatgatactcactgtgttctgtctcagtgtgttcgcactcatcgggttgcagctcttcatgggcaacctgaggaataaatgtgtacagtggcctcccaccaacgcttcccttgaggaacatagcatagagaagaatataactatggattacaatggcacacttgtaaatgaaaccgtgttcgagtttgactggaaatcatacattcaagactcaagatatcattatttcctggagggtgttttagatgcactgctgtgtggaaatagctctgatgcaggccaatgtccagaaggatatatgtgtgtaaaagctggtagaaaccctaattatggttacacaagctttgataccttcagttgggcatttttgtccctgtttcgactgatgactcaggacttctgggaaaatctataccaactgacattgcgtgctgctggcaaaacctacatgatattttttgtgctggtcattttcttgggctcattctacctgataaacttgatcctggctgtggtggccatggcctatgaggagcagaatcaggccacactggaggaggctgaacagaaagaggcagaatttcagcagatgttggagcaacttaagaagcagcaagaggctgcacagcaggcagcggctacaacagcctcagaacattccagggagcccagtgcagcaggcaggctctcagatagctcttcagaagcctctaagttgagttcgaagagtgctaaagaaagacgaaatcggaggaaaaaaaggaaacagaaagagcagtctggaggagaagagaaagatgatgatgaattccacaagtctgagtctgaagacagcatcaggaggaaggggtttcgcttctccatagaagggaatagactgacatatgaaaagaggtactcttccccgcatcagtctctgttaagcattcgtggttccctgttctccccaagacgcaatagcagaacaagtcttttcagctttagagggcgagccaaggatgtggggtctgagaatgactttgctgatgatgaacacagcacctttgaggataatgagagccgtagagactcactgttcgttccccgaagacacggagagcgacgcaacagtaacctgagccagaccagcaggtcctcccgaatgctggcggtgtttccagccaatgggaagatgcacagcacggtggattgcaatggtgtggtttccttggttggtggaccctcagttcccacatcgccagttggacagcttctgccagagggaacaaccactgaaactgagatgagaaagaggaggtcgagctctttccatgtttccatggactttctagaagatccttcccagaggcaaagggcaatgagcatagccagcatcttaacaaatacagtagaagaactagaagaatccaggcagaaatgtccaccctgttggtataaattttccaacatattcttaatttgggactgttctccatattggctgaaagttaaacatattgtcaacctggtggtgatggacccatttgttgatctggccattaccatctgcattgtgttaaatacgctcttcatggctatggagcactaccccatgactgaacatttcaaccatgttcttacagtgggaaacttggtcttcactgggattttcacagcagaaatgttcctgaaaatcatcgcaatggatccttactattacttccaagaaggctggaatatctttgatggtttcattgtgacactcagcctggtagaacttggccttgccaatgtggaaggattgtcagttctccgttcatttcgactgctccgagtgttcaagttggcaaagtcttggcccacactgaatatgctcattaagatcattggtaactcggtgggagcactgggcaacctgactctggtgttggccatcattgtctttatttttgccgtggttggcatgcagctgtttggaaaaagttacaaagattgtgtctgcaaaattgccactgactgcaaactcccacgttggcacatgaacgacttcttccactcgttcctgatcgtgttccgcgtgctgtgtggggagtggatagagaccatgtgggactgcatggaggtggcaggacaagctatgtgccttactgtcttcatgatggtcatggtgattgggaaccttgtggtcttgaacctctttctggccttgcttctgagctcatttagtgcagacaaccttgcagccactgatgatgacaatgagatgaacaacctgcagattgctgtggacaggatgcacaaaggaatagcttatgtaaaaagaaaaatatatgaattcattcaacaatcctttgttaagaaacagaagattctagatgaaattaagccacttgatgatctaaacaacagaaaagacaattgtatctctaaccacacaacagaaattgggaaagatctggactgtctgaaagatgtgaatggaaccacaagtggcatagggacgggcagcagtgtggagaagtacatcattgatgagagtgattatatgtcattcataaacaaccccagcctcactgtgactgtgcccattgctgtgggagagtctgactttgagaacttaaacacagaagactttagcagtgaatcagatctagaagaaagcaaagagaaactcaacgaaagcagtagctcctcagagggaagcacagtagacattggggcgcctgcagaggaacagcctgtcattgaaccagaagaaacccttgagcccgaagcttgcttcactgaaggctgtgtccagagattcaagtgctgtcaaatcagcgtggaagaaggaagagggaaacagtggtggaacctacggaggacgtgcttccgaatagttgaacacaactggtttgagaccttcattgtgttcatgattctcctgagtagtggtgccctggcctttgaggatatatatattgatcagcgaaagacgatcaaaaccatgctggagtatgctgacaaagtcttcacttacattttcatcctggagatgctcctcaaatgggtggcctatggctatcaaacatacttcaccaatgcctggtgttggctagacttcttaattgttgatgtttcattggtcagtttaacagcaaatgccttgggttactctgaactcggggccatcaaatccctaaggacactaagagctctgagacccctaagagccttatcacgatttgaagggatgagggtggttgtgaatgccctgttaggagcaattccatccatcatgaatgtgcttctggtttgccttatattctggctaattttcagcatcatgggcgtaaatttgtttgctggcaaattctaccactgtgttaacaccacaactggtgacatatttgagatcagcgaagtcaataatcattctgattgcctaaaactaatagaaagaaatgagaccgcccggtggaaaaatgtgaaagtaaactttgataatgtaggatttgggtatctttctttgcttcaagttgccacatttaagggctggatggatatcatgtatgctgcagttgattccagaaatgttgaactacagcctaagtatgaggaaagcctgtacatgtatttgtacttcgtcatcttcatcatcttcgggtccttctttaccctgaacctgtttattggtgtcattatcgacaatttcaaccagcaaaagaagaagtttggaggtcaagacatctttatgacagaagaacagaagaaatactataatgcaatgaagaaattaggatcaaaaaagccacaaaagcctatccctcgacctggaaacaaatttcaaggaatggtttttgactttgtaaccagacaagtgtttgatatcagcatcatgatcctcatctgtctgaacatggtgaccatgatggtggaaacggatgaccagagcgattatgtgacaagcattttgtcacgcatcaacctggtgttcatcgtcctgttcaccggcgagtgtgtgctcaagctcatctcgctccgccattattatttcaccattggatggaacattttcgattttgtggtggtcatcctctccattgtagggatgtttcttgcggagctaatagaaaagtattttgtgtctcctaccctgttccgagtcatccgcctggccaggattggacgaatcctacgcctgatcaaaggtgccaaggggatccgcacgctgctctttgctctgatgatgtcccttcctgcgctgtttaacatcggcctcctgctttttctcgtcatgttcatctacgccatctttgggatgtccaactttgcctatgttaagagggaagttgggattgatgacatgttcaactttgagaccttcggcaacagcatgatctgcctgttccaaatcaccacctctgcgggctgggatggactgctggcccccatcctcaacagcaaaccccctgactgtgaccctaataaagttaaccctggaagctcggtgaagggagactgtgggaacccatctgtggggattttcttttttgtcagctacatcatcatatccttcctggttgtggtgaacatgtacattgctgtcatcctggagaacttcagcgttgccacagaagaaagtgcagagcctctgagtgaggacgactttgagatgttctacgaggtctgggagaagttcgaccctgacgccacccagttcatggaatttgaaaaattatctcagtttgcagctgctctagaaccccctctcaatttgccacaaccaaacaaacttcagctcattgccatggacctgcccatggtgagtggagaccgcatccactgcctggacatcttatttgcttttacaaagcgggtgttgggtgagagtggagagatggatgctcttcgaatccagatggaagagcggttcatggcttccaacccctccaaggtctcttatcagcccatcactactacattaaaacgcaaacaagaggaggtgtcagctgttatcattcagcgagcttataggcgccaccttttgaagcgaacagtaaaacaagcttcattcacatacaataagaacaaactcaaaggtggggctaatcttcttgtaaaagaagacatgctcattgacagaataaacgaaaactctattacggagaaaactgacctgacaatgtccacagcagcttgtccgccctcctacgatcgggtgacaaagccaatcgtggagaaacacgagcaggaagggaaggatgaaaaagccaaagggaaagactacaaagaccatgacggtgattataaagatcatgacatcgattacaaggatgacgatgacaagtaa

SEQ ID NO: 899 - DNA, 6063 bp, Artificial Sequence
  Description of Artificial Sequence Synthetic polynucleotide
atggagcaaacagtgcttgtaccaccaggacctgacagcttcaacttcttcaccagagaatcccttgcagctattgaaaggcgcattgcagaagagaaggctaagaatcccaagccagacaaaaaagatgatgatgaaaatggcccaaagccaaacagtgacttggaagctgggaagaaccttccatttatctatggagacattcctccagagatggtgtcggagcctctggaggacctggacccctactatatcaataagaagacttttatagtattgaataaagggaaggccatcttccggttcagtgccacctccgccctgtacattttaacacccttcaatcctcttaggaaaatagctattaagattttggtacactcattattcagcatgttaatcatgtgcactattttgacaaactgtgtatttatgacaatgagtaaccctcccgactggacaaagaatgtggagtacaccttcacaggaatatatacttttgaatcactaataaaaattattgcaaggggcttctgtttagaagattttactttccttcgcgacccatggaactggctggacttcactgtcattacattcgcatatgtgacggagtttgtggacctgggcaatgtctcagcattgagaacattcagagttcttcgagcattgaaaactatttcagtcattccaggcctgaagaccatcgtgggggccctgatccagtcggtgaagaagctgtctgacgtcatgatactcactgtgttctgtctcagtgtgttcgcactcatcgggttgcagctcttcatgggcaacctgaggaataaatgtgtacagtggcctcccaccaacgcttcccttgaggaacatagcatagagaagaatataactatggattacaatggcacacttgtaaatgaaaccgtgttcgagtttgactggaaatcatacattcaagactcaagatatcattatttcctggagggtgttttagatgcactgctgtgtggaaatagctctgatgcaggccaatgtccagaaggatatatgtgtgtaaaagctggtagaaaccctaattatggttacacaagctttgataccttcagttgggcatttttgtccctgtttcgactgatgactcaggacttctgggaaaatctataccaactgacattgcgtgctgctggcaaaacctacatgatattttttgtgctggtcattttcttgggctcattctacctgataaacttgatcctggctgtggtggccatggcctatgaggagcagaatcaggccacactggaggaggctgaacagaaagaggcagaatttcagcagatgttggagcaacttaagaagcagcaagaggctgcacagcaggcagcggctacaacagcctcagaacattccagggagcccagtgcagcaggcaggctctcagatagctcttcagaagcctctaagttgagttcgaagagtgctaaagaaagacgaaatcggaggaaaaaaaggaaacagaaagagcagtctggaggagaagagaaagatgatgatgaattccacaagtctgagtctgaagacagcatcaggaggaaggggtttcgcttctccatagaagggaatagactgacatatgaaaagaggtactcttccccgcatcagtctctgttaagcattcgtggttccctgttctccccaagacgcaatagcagaacaagtcttttcagctttagagggcgagccaaggatgtggggtctgagaatgactttgctgatgatgaacacagcacctttgaggataatgagagccgtagagactcactgttcgttccccgaagacacggagagcgacgcaacagtaacctgagccagaccagcaggtcctcccgaatgctggcggtgtttccagccaatgggaagatgcacagcacggtggattgcaatggtgtggtttccttggttggtggaccctcagttcccacatcgccagttggacagcttctgccagagggaacaaccactgaaactgagatgagaaagaggaggtcgagctctttccatgtttccatggactttctagaagatccttcccagaggcaaagggcaatgagcatagccagcatcttaacaaatacagtagaagaactagaagaatccaggcagaaatgtccaccctgttggtataaattttccaacatattcttaatttgggactgttctccatattggctgaaagttaaacatattgtcaacctggtggtgatggacccatttgttgatctggccattaccatctgcattgtgttaaatacgctcttcatggctatggagcactaccccatgactgaacatttcaaccatgttcttacagtgggaaacttggtcttcactgggattttcacagcagaaatgttcctgaaaatcatcgcaatggatccttactattacttccaagaaggctggaatatctttgatggtttcattgtgacactcagcctggtagaacttggccttgccaatgtggaaggattgtcagttctccgttcatttcgactgctccgagtgttcaagttggcaaagtcttggcccacactgaatatgctcattaagatcattggtaactcggtgggagcactgggcaacctgactctggtgttggccatcattgtctttatttttgccgtggttggcatgcagctgtttggaaaaagttacaaagattgtgtctgcaaaattgccactgactgcaaactcccacgttggcacatgaacgacttcttccactcgttcctgatcgtgttccgcgtgctgtgtggggagtggatagagaccatgtgggactgcatggaggtggcaggacaagctatgtgccttactgtcttcatgatggtcatggtgattgggaaccttgtggtcttgaacctctttctggccttgcttctgagctcatttagtgcagacaaccttgcagccactgatgatgacaatgagatgaacaacctgcagattgctgtggacaggatgcacaaaggaatagcttatgtaaaaagaaaaatatatgaattcattcaacaatcctttgttaagaaacagaagattctagatgaaattaagccacttgatgatctaaacaacagaaaagacaattgtatctctaaccacacaacagaaattgggaaagatctggactgtctgaaagatgtgaatggaaccacaagtggcatagggacgggcagcagtgtggagaagtacatcattgatgagagtgattatatgtcattcataaacaaccccagcctcactgtgactgtgcccattgctgtgggagagtctgactttgagaacttaaacacagaagactttagcagtgaatcagatctagaagaaagcaaagagaaactcaacgaaagcagtagctcctcagagggaagcacagtagacattggggcgcctgcagaggaacagcctgtcattgaaccagaagaaacccttgagcccgaagcttgcttcactgaaggctgtgtccagagattcaagtgctgtcaaatcagcgtggaagaaggaagagggaaacagtggtggaacctacggaggacgtgcttccgaatagttgaacacaactggtttgagaccttcattgtgttcatgattctcctgagtagtggtgccctggcctttgaggatatatatattgatcagcgaaagacgatcaaaaccatgctggagtatgctgacaaagtcttcacttacattttcatcctggagatgctcctcaaatgggtggcctatggctatcaaacatacttcaccaatgcctggtgttggctagacttcttaattgttgatgtttcattggtcagtttaacagcaaatgccttgggttactctgaactcggggccatcaaatccctaaggacactaagagctctgagacccctaagagccttatcacgatttgaagggatgagggtggttgtgaatgccctgttaggagcaattccatccatcatgaatgtgcttctggtttgccttatattctggctaattttcagcatcatgggcgtaaatttgtttgctggcaaattctaccactgtgttaacaccacaactggtgacatatttgagatcagcgaagtcaataatcattctgattgcctaaaactaatagaaagaaatgagaccgcctgatggaaaaatgtgaaagtaaactttgataatgtaggatttgggtatctttctttgcttcaagttgccacatttaagggctggatggatatcatgtatgctgcagttgattccagaaatgttgaactacagcctaagtatgaggaaagcctgtacatgtatttgtacttcgtcatcttcatcatcttcgggtccttctttaccctgaacctgtttattggtgtcattatcgacaatttcaaccagcaaaagaagaagtttggaggtcaagacatctttatgacagaagaacagaagaaatactataatgcaatgaagaaattaggatcaaaaaagccacaaaagcctatccctcgacctggaaacaaatttcaaggaatggtttttgactttgtaaccagacaagtgtttgatatcagcatcatgatcctcatctgtctgaacatggtgaccatgatggtggaaacggatgaccagagcgattatgtgacaagcattttgtcacgcatcaacctggtgttcatcgtcctgttcaccggcgagtgtgtgctcaagctcatctcgctccgccattattatttcaccattggatggaacattttcgattttgtggtggtcatcctctccattgtagggatgtttcttgcggagctaatagaaaagtattttgtgtctcctaccctgttccgagtcatccgcctggccaggattggacgaatcctacgcctgatcaaaggtgccaaggggatccgcacgctgctctttgctctgatgatgtcccttcctgcgctgtttaacatcggcctcctgctttttctcgtcatgttcatctacgccatctttgggatgtccaactttgcctatgttaagagggaagttgggattgatgacatgttcaactttgagaccttcggcaacagcatgatctgcctgttccaaatcaccacctctgcgggctgggatggactgctggcccccatcctcaacagcaaaccccctgactgtgaccctaataaagttaaccctggaagctcggtgaagggagactgtgggaacccatctgtggggattttcttttttgtcagctacatcatcatatccttcctggttgtggtgaacatgtacattgctgtcatcctggagaacttcagcgttgccacagaagaaagtgcagagcctctgagtgaggacgactttgagatgttctacgaggtctgggagaagttcgaccctgacgccacccagttcatggaatttgaaaaattatctcagtttgcagctgctctagaaccccctctcaatttgccacaaccaaacaaacttcagctcattgccatggacctgcccatggtgagtggagaccgcatccactgcctggacatcttatttgcttttacaaagcgggtgttgggtgagagtggagagatggatgctcttcgaatccagatggaagagcggttcatggcttccaacccctccaaggtctcttatcagcccatcactactacattaaaacgcaaacaagaggaggtgtcagctgttatcattcagcgagcttataggcgccaccttttgaagcgaacagtaaaacaagcttcattcacatacaataagaacaaactcaaaggtggggctaatcttcttgtaaaagaagacatgctcattgacagaataaacgaaaactctattacggagaaaactgacctgacaatgtccacagcagcttgtccgccctcctacgatcgggtgacaaagccaatcgtggagaaacacgagcaggaagggaaggatgaaaaagccaaagggaaagactacaaagaccatgacggtgattataaagatcatgacatcgattacaaggatgacgatgacaagtaa

SEQ ID NO: 900 - DNA, 55 bp, Artificial Sequence
  Description of Artificial Sequence Synthetic oligonucleotide
agcgctccggtttttctgtgctgaacctcaggggacgccgacacacgtacacgtc

SEQ ID NO: 901 - protein, 22 aa, Artificial Sequence
  Description of Artificial Sequence Synthetic peptide
DYKDHDGDYKDHDIDYKDDDDK

SEQ ID NO: 902 - protein, 7 aa, Tobacco etch virus
  MOD_RES (7)..(7): Gly or Ser
ENLYFQX

(19) United States
(12) Patent Application Publication
(10) Pub. No.: US 2024/0384300 A1
(43) Pub. Date: Nov. 21, 2024

(54) METHODS AND COMPOSITIONS FOR TREATING A PREMATURE TERMINATION CODON-MEDIATED DISORDER

(71) Applicant: Tevard Biosciences, Inc., Cambridge, MA (US)

(72) Inventors: Peter M. Eimon, Boston, MA (US); Sean McFarland, Newton, MA (US)

(21) Appl. No.: 18/558,926

(22) PCT Filed: May 5, 2022
(86) PCT No.: PCT/US2022/027765

§ 371 Date: Nov. 3, 2023

Related U.S. Application Data
(60) Provisional application No. 63/184,514, filed on May 5, 2021.

(51) Int. Cl. C12N 15/864; A61K 48/00; C12N 15/113

(52) U.S. Cl. CPC C12N 15/8645; A61K 48/005; C12N 15/113; C12N 2750/14143

(57) ABSTRACT

The invention relates generally to expression vectors and pharmaceutical compositions comprising a first, second, and/or third modified tRNA and the use of expression vectors and pharmaceutical compositions to express in a mammalian cell a functional gene product encoded by a gene containing a premature termination codon and/or to treat a disorder mediated by a premature termination codon, e.g., Dravet syndrome.

### CROSS-REFERENCE TO RELATED APPLICATIONS

This application claims the benefit of and priority to U.S. Provisional Patent Application No. 63/184,514, filed May 5, 2021, which is incorporated herein by reference in its entirety for all purposes.

### SEQUENCE LISTING

The instant application contains a Sequence Listing which has been submitted electronically in ASCII format and is hereby incorporated by reference in its entirety. Said ASCII copy, created on Apr. 26, 2022, is named TVD-004WO_SL.txt and is 402,848 bytes in size.

### FIELD OF THE INVENTION

The invention relates generally to methods and compositions for expressing a gene product encoded by a gene containing a premature termination codon and/or treating a disorder mediated by a premature termination codon.

### BACKGROUND

Protein synthesis is directed by a genetic code that includes 61 three-base-pair codons encoding amino acids that are incorporated into the protein being synthesized and 3 three-base-pair codons (referred to as stop or termination codons) that terminate the synthesis of a protein. When a nucleic acid sequence encoding a protein is mutated to contain a premature termination codon rather than a codon for the next amino acid, the resulting protein is prematurely terminated, which is often nonfunctional or less functional than the untruncated or full length protein. Such mutations, termed nonsense mutations, are often associated with, or are a causative agent in numerous different genetic diseases.

A number of disorders are associated with, or are caused by, nonsense mutations. These include epilepsies, for example, Dravet Syndrome, Genetic Epilepsy with Febrile Seizures (GEFS), Benign Familial Infantile Epilepsy (BFIE), Early Infantile Epileptic Encephalopathy (EIEE), Lennox-Gastaut Syndrome, Rett Syndrome, PPM-X Syndrome, Ohtahara Syndrome, Episodic Ataxia, Hemiplegic Migraine, Iditiopathic Generalized Epilepsy, FOXG1 Syndrome, Familial Focal Epilepsy with Variable Foci (FFEVF), Childhood-Onset Epileptic Encephalopathy, SYNGAP1-Related Intellectual Disability, Pyridoxine-Dependent Epilepsy, Familial Infantile Myoclonic Epilepsy (FIME), Myoclonic Astatic Epilepsy, X-Linked Intellectual Disability, Partial Epilepsy and Episodic Ataxia, Febrile Seizures, Autosomal Dominant Partial Epilepsy with Auditory Features (ADPEAF), PNPO-Deficiency, Progressive Myoclonus Epilepsy, Action Myoclonus-Renal Failure (AMRF), CDKL5 deficiency disorder, and Benign Familial Infantile Seizures (BFIS).

By way of example, Dravet Syndrome is a rare and catastrophic form of intractable epilepsy that begins in infancy. Initially, patients experience prolonged seizures. In their second year, additional types of seizure begin to occur, which typically coincide with a developmental decline, possibly due to repeated cerebral hypoxia. This leads to poor development of language and motor skills. Mutations in SCN1A (encoding the voltage-gated sodium channel a subunit Nav1.1), SCN1B (encoding the voltage-gated sodium channel β1 subunit), SCN2A (encoding Nav1.2), SCN3A (encoding Nav1.3), SCN9A (encoding Nav1.7), GABRG2 (encoding the γ-aminobutyric acid receptor γ2 subunit), GABRD (encoding the γ-aminobutyric acid receptor Δ subunit) and/or PCDH19 (encoding Protocadherin-19) genes have been linked to Dravet Syndrome.

Dravet syndrome may be caused by a nonsense mutation in, for example, the SCN1A gene, resulting in a premature termination codon and a lack of or reduced amount of untruncated or functional protein. The SCN1A gene normally codes for the neuronal voltage-gated sodium channel a subunit, Na(V)1.1. In mouse models, loss-of-function mutations in SCN1A have been observed to result in a decrease in sodium currents and impaired excitability of GABAergic interneurons of the hippocampus.

Despite the efforts made to date, there is a need in the art for improved compositions and methods for treating disorders mediated by premature termination codons, such as Dravet syndrome.

### SUMMARY OF THE INVENTION

The invention is based, in part, upon the discovery that is possible to express multiple (e.g., two or three) suppressor tRNAs using a single expression vector. Each suppressor tRNA permits an amino acid to be incorporated into a gene product encoded by a gene in a mammalian cell at a position that would otherwise result in a truncated gene product caused by a premature termination codon (PTC) in the gene. Expression of multiple suppressor tRNAs from a single expression vector allows for the single expression vector to treat a disease mediated by multiple, different PTCs in the same subject and/or treat a disease mediated by multiple, different PTCs in multiple, different subjects. The invention is further based, in part, upon the discovery of optimal combinations of suppressor tRNAs that allow for treatment of the greatest possible patient populations.

Accordingly, in one aspect, the invention provides an expression vector comprising: (a) a first nucleotide sequence encoding a first suppressor tRNA that comprises an anticodon that hybridizes to a first premature stop codon (e.g., TGA), and is capable of being aminoacylated with a first amino acid; (b) a second nucleotide sequence encoding a second suppressor tRNA that comprises an anticodon that hybridizes to a second premature stop codon (e.g., TAG), and is capable of being aminoacylated with a second amino acid; and optionally, (c) a third nucleotide sequence encoding a third suppressor tRNA that comprises an anticodon that hybridizes to a third premature stop codon (e.g., TAA), and is capable of being aminoacylated with a third amino acid.

In certain embodiments, the first amino acid is selected from arginine, tryptophan, cysteine, serine, glycine, and leucine (e.g., the first amino acid is arginine). In certain embodiments, the second amino acid is selected from glutamine, glutamic acid, tyrosine, tryptophan, lysine, serine, and leucine (e.g., the second amino acid is glutamine). In certain embodiments, the third amino acid is selected from glutamine, glutamic acid, tyrosine, lysine, serine, and leucine. In certain embodiments, the second and third amino acid are the same, for example, the second and third amino acid are selected from glutamine, glutamic acid, tyrosine, lysine, serine, and leucine.

In certain embodiments: (i) the first amino acid is arginine, the second amino acid is glutamine, and the third amino acid is lysine; (ii) the first amino acid is arginine, the second amino acid is glutamine, and the third amino acid is glutamic acid; (iii) the first amino acid is arginine, the second amino acid is glutamine, and the third amino acid is tyrosine; (iv) the first amino acid is arginine, the second amino acid is glutamine, and the third amino acid is leucine; (v) the first amino acid is arginine, the second amino acid is tryptophan, and the third amino acid is glutamic acid; or (vi) the first amino acid is arginine, the second amino acid is tyrosine, and the third amino acid is glutamic acid. In certain embodiments: (i) the first amino acid is arginine, the second amino acid is glutamine, and the third amino acid is glutamine; (ii) the first amino acid is arginine, the second amino acid is glutamine, and the third amino acid is glutamic acid; (iii) the first amino acid is arginine, the second amino acid is glutamine, and the third amino acid is lysine; (iv) the first amino acid is arginine, the second amino acid is tryptophan, and the third amino acid is glutamine; or (v) the first amino acid is arginine, the second amino acid is glutamic acid, and the third amino acid is glutamine. In certain embodiments: (i) the first amino acid is arginine, the second amino acid is glutamine, and the third amino acid is glutamine; (ii) the first amino acid is tryptophan, the second amino acid is glutamic acid, and the third amino acid is glutamic acid; (iii) the first amino acid is cysteine, the second amino acid is tyrosine, and the third amino acid is tyrosine; (iv) the first amino acid is serine, the second amino acid is lysine, and the third amino acid is lysine; (v) the first amino acid is glycine, the second amino acid is serine, and the third amino acid is serine; or (vi) the first amino acid is leucine, the second amino acid is leucine, and the third amino acid is leucine.

In certain embodiments, the first, second, and/or third suppressor tRNA comprises a nucleotide sequence set forth in TABLE 2 or TABLE 3. For example, (i) when the first amino acid is arginine, the first suppressor tRNA may comprise a nucleotide sequence selected from SEQ ID NOs: 6-9, 11, 16-22, and 35, (ii) when the second amino acid is glutamine, the second suppressor tRNA may comprise a nucleotide sequence selected from SEQ ID NOs: 178-182, 186, and 187, and/or (iii) when the third amino acid is glutamine, the third suppressor tRNA may comprise a nucleotide sequence selected from SEQ ID NOs: 36-40, 44, and 45.

In certain embodiments, the expression vector comprises 1, 2, 3, 4, or more than 4 copy numbers of the nucleotide sequence encoding the first, second, and/or third suppressor tRNA.

In certain embodiments, the expression vector comprises a nucleotide sequence corresponding to a genomic DNA sequence flanking a wild-type tRNA gene. For example, in certain embodiments, the expression vector comprises a nucleotide sequence set forth in TABLE 4. In certain embodiments, the nucleotide sequence set forth in TABLE 4 is selected from SEQ ID NOs: 869-888. In certain embodiments, the nucleotide sequence set forth in TABLE 4 is operably linked to the nucleotide sequence encoding the first, second, and/or third suppressor tRNA. In certain embodiments, in the expression vector, the nucleotide sequence set forth in TABLE 4 is 5′ to the nucleotide sequence encoding the first, second, and/or third suppressor tRNA. In certain embodiments, in the expression vector, the nucleotide sequence set forth in TABLE 4 is immediately 5′ to (i.e., adjacent) the nucleotide sequence encoding the first, second, and/or third suppressor tRNA.

In certain embodiments, the expression vector is a viral vector, e.g., a DNA virus vector, e.g., an adeno-associated virus (AAV) vector.

In another aspect, the invention provides a pharmaceutical composition comprising any of the foregoing expression vectors and a pharmaceutically acceptable excipient.

In another aspect, the invention provides a pharmaceutical composition comprising: (a) a first suppressor tRNA that comprises an anticodon that hybridizes to a first premature stop codon (e.g., TGA), and is capable of being aminoacylated with a first amino acid; (b) a second suppressor tRNA that comprises an anticodon that hybridizes to a second premature stop codon (e.g., TAG), and is capable of being aminoacylated with a second amino acid; and optionally, (c) a third suppressor tRNA that comprises an anticodon that hybridizes to a third premature stop codon (e.g., TAA), and is capable of being aminoacylated with a third amino acid.

In certain embodiments, the first, second, and/or third suppressor tRNA comprises a nucleotide sequence set forth in TABLE 2 or TABLE 3. For example, (i) when the first amino acid is arginine, the first suppressor tRNA may comprise a nucleotide sequence selected from SEQ ID NOs: 6-9, 11, 16-22, and 35, (ii) when the second amino acid is glutamine, the second suppressor tRNA may comprise a nucleotide sequence selected from SEQ ID NOs: 178-182, 186, and 187, and/or (iii) when the third amino acid is glutamine, the third suppressor tRNA may comprise a nucleotide sequence selected from SEQ ID NOs: 36-40, 44, and 45.

In certain embodiments, the first, second, and/or third suppressor tRNA comprises one or more naturally occurring nucleotide modifications, e.g., selected from 5-methyl uridine, 5-carbamoylmethyluridine, 5-carbamoyl-methyl-2-O-methyluridine, 5-methoxy-carbonylmethyluridine, 5-methoxycarbonylmethyl-2-thiouridine, pseudouridine, dihydrouridine, 1-methyladenosine, and inosine. In certain embodiments, the tRNA is not conjugated to, or associated with, another moiety, e.g., a carrier particle, e.g., an aminolipid particle. In certain embodiments, the composition does not comprise a nanoparticle and/or an aminolipid delivery compound.

In another aspect, the invention provides a method of expressing in a mammalian cell a functional gene product encoded by a gene containing a premature termination codon, the method comprising contacting the cell with an effective amount of any of the foregoing expression vectors or pharmaceutical compositions, thereby permitting an amino acid to be incorporated into the gene product at a position that would otherwise result in a truncated gene product caused by the premature termination codon.

In another aspect, the invention provides a method of expressing in a mammalian cell a functional gene product encoded by a gene containing a first, second, and/or third premature termination codon, the method comprising contacting the cell with effective amount of: (a) a first expression vector comprising a nucleotide sequence encoding a first suppressor tRNA that comprises an anticodon that hybridizes to a first premature stop codon (e.g., TGA), and is capable of being aminoacylated with a first amino acid; (b) a second expression vector comprising a nucleotide sequence encoding a second suppressor tRNA that comprises an anticodon that hybridizes to a second premature stop codon (e.g., TAG), and is capable of being aminoacylated with a second amino acid; and optionally, (c) a third expression vector comprising a nucleotide sequence encoding a third suppressor tRNA that comprises an anticodon that hybridizes to a third premature stop codon (e.g., TAA), and is capable of being aminoacylated with a third amino acid, thereby permitting an amino acid to be incorporated into the gene product at a position that would otherwise result in a truncated gene product caused by the premature termination codon.

In another aspect, the invention provides a method of expressing in a mammalian cell a functional gene product encoded by a gene containing a first, second, and/or third premature termination codon, the method comprising contacting the cell with effective amount of: (a) a first suppressor tRNA that comprises an anticodon that hybridizes to a first premature stop codon (e.g., TGA), and is capable of being aminoacylated with a first amino acid; (b) a second suppressor tRNA that comprises an anticodon that hybridizes to a second premature stop codon (e.g., TAG), and is capable of being aminoacylated with a second amino acid; and optionally, (c) a third suppressor tRNA that comprises an anticodon that hybridizes to a third premature stop codon (e.g., TAA), and is capable of being aminoacylated with a third amino acid, thereby permitting an amino acid to be incorporated into the gene product at a position that would otherwise result in a truncated gene product caused by the premature termination codon.

In certain embodiments of any of the foregoing methods, the first, second, and/or third suppressor tRNA comprises a nucleotide sequence set forth in TABLE 2 or TABLE 3. For example, (i) when the first amino acid is arginine, the first suppressor tRNA may comprise a nucleotide sequence selected from SEQ ID NOs: 6-9, 11, 16-22, and 35, (ii) when the second amino acid is glutamine, the second suppressor tRNA may comprise a nucleotide sequence selected from SEQ ID NOs: 178-182, 186, and 187, and/or (iii) when the third amino acid is glutamine, the third suppressor tRNA may comprise a nucleotide sequence selected from SEQ ID NOs: 36-40, 44, and 45.

In certain embodiments of any of the foregoing methods, the gene is a gene set forth in TABLE 5 or TABLE 6. In certain embodiments, the gene is an SCN1A or dystrophin gene.

In certain embodiments of any of the foregoing methods, the cell is a human cell. In certain embodiments, the cell is a central nervous system cell, e.g., a neuron. In certain embodiments, the tRNA becomes aminoacylated in the cell.

In another aspect, the invention provides a method of treating a premature termination codon-mediated disorder in a subject (or a population of subjects) in need thereof, wherein the subject(s) have a gene with a first, second, and/or third premature termination codon, the method comprising administering to the subject(s) an effective amount of any of the foregoing expression vectors or any of the foregoing pharmaceutical compositions, thereby to treat the disorder in the subject.

In another aspect, the invention provides a method of treating a premature termination codon-mediated disorder in a subject (or a population of subjects) in need thereof wherein the subject(s) have a gene with a first, second, and/or third premature termination codon, the method comprising administering to the subject(s) an effective amount of: (a) a first expression vector comprising a nucleotide sequence encoding a first suppressor tRNA that comprises an anticodon that hybridizes to a first premature stop codon (e.g., TGA), and is capable of being aminoacylated with a first amino acid; (b) a second expression vector comprising a nucleotide sequence encoding a second suppressor tRNA that comprises an anticodon that hybridizes to a second premature stop codon (e.g., TAG), and is capable of being aminoacylated with a second amino acid; and optionally, (c) a third expression vector comprising a nucleotide sequence encoding a third suppressor tRNA that comprises an anticodon that hybridizes to a third premature stop codon (e.g., TAA), and is capable of being aminoacylated with a third amino acid; thereby to treat the disorder in the subject(s).

In another aspect, the invention provides a method of treating a premature termination codon-mediated disorder in a subject (or a population of subjects) in need thereof wherein the subject(s) have a gene with a first, second, and/or third premature termination codon, the method comprising administering to the subject(s) an effective amount of: (a) a first suppressor tRNA that comprises an anticodon that hybridizes to a first premature stop codon (e.g., TGA), and is capable of being aminoacylated with a first amino acid; (b) a second suppressor tRNA that comprises an anticodon that hybridizes to a second premature stop codon (e.g., TAG), and is capable of being aminoacylated with a second amino acid; and optionally, (c) a third suppressor tRNA that comprises an anticodon that hybridizes to a third premature stop codon (e.g., TAA), and is capable of being aminoacylated with a third amino acid; thereby to treat the disorder in the subject(s).

In certain embodiments of any of the foregoing methods, the first, second, and/or third suppressor tRNA comprises a nucleotide sequence set forth in TABLE 2 or TABLE 3. For example, (i) when the first amino acid is arginine, the first suppressor tRNA may comprise a nucleotide sequence selected from SEQ ID NOs: 6-9, 11, 16-22, and 35, (ii) when the second amino acid is glutamine, the second suppressor tRNA may comprise a nucleotide sequence selected from SEQ ID NOs: 178-182, 186, and 187, and/or (iii) when the third amino acid is glutamine, the third suppressor tRNA may comprise a nucleotide sequence selected from SEQ ID NOs: 36-40, 44, and 45.

In certain embodiments of any of the foregoing methods, the disorder is a disorder set forth in TABLE 5 or TABLE 6. In certain embodiments, the disorder is Dravet Syndrome or Duchenne Muscular Dystrophy.

In another aspect, the invention provides a method of treating Dravet Syndrome in a subject (or a population of subjects) in need thereof wherein the subject(s) have an SCN1A gene with a first, second, and/or third premature termination codon, the method comprising administering to the subject an effective amount of an expression vector comprising: (a) a first nucleotide sequence encoding a first suppressor tRNA that comprises an anticodon that hybridizes to the first premature stop codon (e.g., TGA), and is capable of being aminoacylated with a first amino acid; (b) a second nucleotide sequence encoding a second suppressor tRNA that comprises an anticodon that hybridizes to the second premature stop codon (e.g., TAG), and is capable of being aminoacylated with a second amino acid; and optionally, (c) a third nucleotide sequence encoding a third suppressor tRNA that comprises an anticodon that hybridizes to the third premature stop codon (e.g., TAA), and is capable of being aminoacylated with a third amino acid; thereby to treat Dravet Syndrome in the subject(s). In certain embodiments, (i) the first amino acid is arginine, the second amino acid is glutamine, and the third amino acid is lysine; (ii) the first amino acid is arginine, the second amino acid is glutamine, and the third amino acid is glutamic acid; (iii) the first amino acid is arginine, the second amino acid is glutamine, and the third amino acid is tyrosine; (iv) the first amino acid is arginine, the second amino acid is glutamine, and the third amino acid is leucine; (v) the first amino acid is arginine, the second amino acid is tryptophan, and the third amino acid is glutamic acid; or (vi) the first amino acid is arginine, the second amino acid is tyrosine, and the third amino acid is glutamic acid.

In another aspect, the invention provides a method of treating Duchenne Muscular Dystrophy in a subject (or a population of subjects) in need thereof wherein the subject(s) have a dystrophin gene with a first, second, and/or third premature termination codon, the method comprising administering to the subject(s) an effective amount of an expression vector comprising: (a) a first nucleotide sequence encoding a first suppressor tRNA that comprises an anticodon that hybridizes to the first premature stop codon (e.g., TGA), and is capable of being aminoacylated with a first amino acid; (b) a second nucleotide sequence encoding a second suppressor tRNA that comprises an anticodon that hybridizes to the second premature stop codon (e.g., TAG), and is capable of being aminoacylated with a second amino acid; and optionally, (c) a third nucleotide sequence encoding a third suppressor tRNA that comprises an anticodon that hybridizes to the third premature stop codon (e.g., TAA), and is capable of being aminoacylated with a third amino acid; thereby to treat Duchenne Muscular Dystrophy in the subject(s). In certain embodiments: (i) the first amino acid is arginine, the second amino acid is glutamine, and the third amino acid is glutamine; (ii) the first amino acid is arginine, the second amino acid is glutamine, and the third amino acid is glutamic acid; (iii) the first amino acid is arginine, the second amino acid is glutamine, and the third amino acid is lysine; (iv) the first amino acid is arginine, the second amino acid is tryptophan, and the third amino acid is glutamine; or (v) the first amino acid is arginine, the second amino acid is glutamic acid, and the third amino acid is glutamine.

These and other aspects and features of the invention are described in the following detailed description and claims.

### DESCRIPTION OF THE DRAWINGS

The invention can be more completely understood with reference to the following drawings.

FIG. 1 is a schematic representation of a transcript (e.g., an SCN1A transcript) containing a premature termination codon (PTC) which leads to a truncated protein product (e.g., a protein product in a subject with Dravet syndrome). Native termination codons are indicated as shaded circles, and premature termination codons are indicated as unshaded circles. Expression of a suppressor tRNA (e.g., an anticodon modified arginine tRNA) charged with its cognate amino acid (A.A.) allows read-through of the PTC and facilitates expression of the full-length protein.

FIG. 2A is a consensus tRNA secondary structure. The numbering of the residues is based on the tRNA numbering system described in Steinberg et al. (1993) NUCLEIC ACIDS RES. 21:3011-15. FIG. 2B is a table showing the modification profile for tRNA sequences from the cytosol of certain eukaryotic organisms. The ratios in the table indicate the frequency of occurrence of listed nucleotide at the numbered position shown in FIG. 2A. The abbreviations for the modified residues are defined in Motorin et al. (2005) “Transfer RNA Modification,” ENCYCLOPEDIA OF LIFE SCIENCES, John Wily & Sons, Inc.

FIG. 3 is a bar graph showing the global frequencies of nonsense mutations. Data is from ˜16,000 entries for pathogenic nonsense mutations in ClinVar.

FIG. 4 is a bar graph showing the frequencies of nonsense mutations in SCN1A. Data is from ClinVar and the Guangzhou SCN1A mutation database.

FIG. 5 is a bar graph showing the frequencies of nonsense mutations in Duchenne/Becker muscular dystrophy. Data is from the Leiden database.

FIG. 6 is a schematic representation of an exemplary expression vector encoding three suppressor tRNAs that facilitate read-through of three different premature termination codons (PTC).

FIG. 7 depicts an exemplary EGFP reporter with a PTC (TGA) in place of an Arginine codon (CGA) and a suppressor tRNA. Native termination codons are indicated as shaded circles, and premature termination codons are indicated as unshaded circles. In the depicted example, a standard Arginine tRNA (with an anticodon that binds CGA) will result in no read-through of the PTC in EGFP, and a non-functional truncated EGFP protein. An Arg>TGA suppressor tRNA (an Arginine tRNA with a modified anticodon that binds TGA/UGA) allows for read-through of the PTC in EGFP resulting in full-length, functional EGFP protein.

FIG. 8 depicts fluorescent images of EGFP reporter expression in HEK293 cells transiently co-transfected with (i) a plasmid encoding the Tristop suppressor and (ii) a plasmid encoding either an EGFP reporter with a PTC (TGA) in place of an Arginine codon (CGA, “R96*TGA”), an EGFP reporter with a PTC (TAA) in place of an Glutamine codon (CAG, “Q69*TAA”), or an EGFP reporter with a PTC (TAG) in place of an Glutamine codon (CAG, “Q69*TAG”). The readthrough activity of the Tristop suppressor was compared to the activity of separate expression vectors encoding only an Arginine to TGA (R>TGA) suppressor (“R→TGA Suppressor (115)”), only a Glutamine to TAA (Q>TAA) suppressor (“Q→TAA Suppressor (157)”), and only a Glutamine to TAG (Q>TAG) suppressor (“Q→TAG Suppressor (196)”).

FIG. 9 depicts EGFP expression in HEK293 cells co-transfected as described for FIG. 8. EGFP expression was analyzed by flow cytometry and readthrough activity is presented as the percentage of viable cells that express EGFP above background. Controls (without any suppressor tRNA) are depicted on the right, where “R96*TGA” indicates the EGFP reporter with a PTC (TGA) in place of an Arginine codon, “Q69*TAA” indicates the EGFP reporter with a PTC (TAA) in place of an Glutamine codon, “Q69*TAG” indicates the EGFP reporter with a PTC (TAG) in place of an Glutamine codon, and “EGFP” indicates the wild-type EGFP reporter.

FIG. 10 is a bar graph depicting cell viability in cells transfected with the indicated suppressor tRNA. “Mock” indicates mock-transfected cells, and “Control” indicates cells transfected with an expression vector that does not contain a suppressor tRNA.

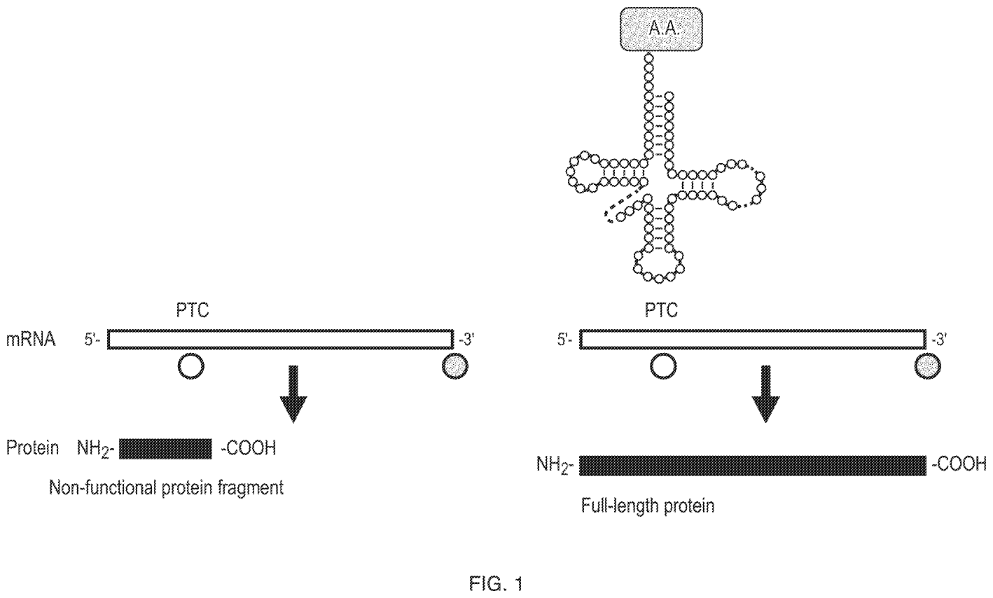

### DETAILED DESCRIPTION

The invention is based, in part, upon the discovery that is possible to express multiple (e.g., two or three) suppressor tRNAs using a single expression vector. Each suppressor tRNA permits an amino acid to be incorporated into a gene product encoded by a gene in a mammalian cell at a position that would otherwise result in a truncated gene product caused by a premature termination codon (PTC) in the gene. Expression of multiple suppressor tRNAs from a single expression vector allows for the single expression vector to treat a disease mediated by multiple, different PTCs in the same subject and/or treat a disease mediated by multiple, different PTCs in multiple, different subjects. The invention is further based, in part, upon the discovery of optimal combinations of suppressor tRNAs that allow for treatment of the greatest possible patient populations.

Accordingly, in one aspect, the invention provides an expression vector comprising: (a) a first nucleotide sequence encoding a first suppressor tRNA that comprises an anticodon that hybridizes to a first premature stop codon (e.g., TGA), and is capable of being aminoacylated with a first amino acid; (b) a second nucleotide sequence encoding a second suppressor tRNA that comprises an anticodon that hybridizes to a second premature stop codon (e.g., TAG), and is capable of being aminoacylated with a second amino acid; and optionally, (c) a third nucleotide sequence encoding a third suppressor tRNA that comprises an anticodon that hybridizes to a third premature stop codon (e.g., TAA), and is capable of being aminoacylated with a third amino acid.

In certain embodiments, the first amino acid is selected from arginine, tryptophan, cysteine, serine, glycine, and leucine (e.g., the first amino acid is arginine). In certain embodiments, the second amino acid is selected from glutamine, glutamic acid, tyrosine, tryptophan, lysine, serine, and leucine (e.g., the second amino acid is glutamine). In certain embodiments, the third amino acid is selected from glutamine, glutamic acid, tyrosine, lysine, serine, and leucine. In certain embodiments, the second and third amino acid are the same, for example, the second and third amino acid are selected from glutamine, glutamic acid, tyrosine, lysine, serine, and leucine.

In certain embodiments: (i) the first amino acid is arginine, the second amino acid is glutamine, and the third amino acid is lysine; (ii) the first amino acid is arginine, the second amino acid is glutamine, and the third amino acid is glutamic acid; (iii) the first amino acid is arginine, the second amino acid is glutamine, and the third amino acid is tyrosine; (iv) the first amino acid is arginine, the second amino acid is glutamine, and the third amino acid is leucine; (v) the first amino acid is arginine, the second amino acid is tryptophan, and the third amino acid is glutamic acid; or (vi) the first amino acid is arginine, the second amino acid is tyrosine, and the third amino acid is glutamic acid. In certain embodiments: (i) the first amino acid is arginine, the second amino acid is glutamine, and the third amino acid is glutamine; (ii) the first amino acid is arginine, the second amino acid is glutamine, and the third amino acid is glutamic acid; (iii) the first amino acid is arginine, the second amino acid is glutamine, and the third amino acid is lysine; (iv) the first amino acid is arginine, the second amino acid is tryptophan, and the third amino acid is glutamine; or (v) the first amino acid is arginine, the second amino acid is glutamic acid, and the third amino acid is glutamine. In certain embodiments: (i) the first amino acid is arginine, the second amino acid is glutamine, and the third amino acid is glutamine; (ii) the first amino acid is tryptophan, the second amino acid is glutamic acid, and the third amino acid is glutamic acid; (iii) the first amino acid is cysteine, the second amino acid is tyrosine, and the third amino acid is tyrosine; (iv) the first amino acid is serine, the second amino acid is lysine, and the third amino acid is lysine; (v) the first amino acid is glycine, the second amino acid is serine, and the third amino acid is serine; or (vi) the first amino acid is leucine, the second amino acid is leucine, and the third amino acid is leucine.

In certain embodiments, the expression vector comprises, in order (e.g., in a 5′ to 3′ orientation): (i) the first nucleotide sequence, the second nucleotide sequence, and the third nucleotide sequence; (ii) the first nucleotide sequence, the third nucleotide sequence, and the second nucleotide sequence; (iii) the second nucleotide sequence, the first nucleotide sequence, and the third nucleotide sequence; (iv) the second nucleotide sequence, the third nucleotide sequence, and the first nucleotide sequence; (v) the third nucleotide sequence, the first nucleotide sequence, and the second nucleotide sequence; or (vi) the third nucleotide sequence, the second nucleotide sequence, and the first nucleotide sequence.

In another aspect, the invention provides a pharmaceutical composition comprising any of the foregoing expression vectors and a pharmaceutically acceptable excipient.

In another aspect, the invention provides a pharmaceutical composition comprising: (a) a first suppressor tRNA that comprises an anticodon that hybridizes to a first premature stop codon (e.g., TGA), and is capable of being aminoacylated with a first amino acid; (b) a second suppressor tRNA that comprises an anticodon that hybridizes to a second premature stop codon (e.g., TAG), and is capable of being aminoacylated with a second amino acid; and optionally, (c) a third suppressor tRNA that comprises an anticodon that hybridizes to a third premature stop codon (e.g., TAA), and is capable of being aminoacylated with a third amino acid.

In another aspect, the invention provides a method of expressing in a mammalian cell a functional gene product encoded by a gene containing a premature termination codon, the method comprising contacting the cell with an effective amount of any of the foregoing expression vectors or pharmaceutical compositions, thereby permitting an amino acid to be incorporated into the gene product at a position that would otherwise result in a truncated gene product caused by the premature termination codon.

In another aspect, the invention provides a method of expressing in a mammalian cell a functional gene product encoded by a gene containing a first, second, and/or third premature termination codon, the method comprising contacting the cell with effective amount of: (a) a first expression vector comprising a nucleotide sequence encoding a first suppressor tRNA that comprises an anticodon that hybridizes to a first premature stop codon (e.g., TGA), and is capable of being aminoacylated with a first amino acid; (b) a second expression vector comprising a nucleotide sequence encoding a second suppressor tRNA that comprises an anticodon that hybridizes to a second premature stop codon (e.g., TAG), and is capable of being aminoacylated with a second amino acid; and optionally, (c) a third expression vector comprising a nucleotide sequence encoding a third suppressor tRNA that comprises an anticodon that hybridizes to a third premature stop codon (e.g., TAA), and is capable of being aminoacylated with a third amino acid; thereby permitting an amino acid to be incorporated into the gene product at a position that would otherwise result in a truncated gene product caused by the premature termination codon.

In another aspect, the invention provides a method of expressing in a mammalian cell a functional gene product encoded by a gene containing a first, second, and/or third premature termination codon, the method comprising contacting the cell with effective amount of: (a) a first suppressor tRNA that comprises an anticodon that hybridizes to a first premature stop codon (e.g., TGA), and is capable of being aminoacylated with a first amino acid; (b) a second suppressor tRNA that comprises an anticodon that hybridizes to a second premature stop codon (e.g., TAG), and is capable of being aminoacylated with a second amino acid; and optionally, (c) a third suppressor tRNA that comprises an anticodon that hybridizes to a third premature stop codon (e.g., TAA), and is capable of being aminoacylated with a third amino acid; thereby permitting an amino acid to be incorporated into the gene product at a position that would otherwise result in a truncated gene product caused by the premature termination codon.

In certain embodiments of any of the foregoing methods, the cell contains less truncated gene product than a cell without the tRNA. For example, in certain embodiments, the cell contains less than about 5%, about 10%, about 20%, about 30%, about 40%, about 50%, about 60%, about 70%, about 80%, or about 90% of the truncated gene product relative to a cell without the tRNA. In certain embodiments, the cell contains from about 5% to about 80%, about 5% to about 60%, about 5% to about 40%, about 5% to about 20%, about 5% to about 10%, about 10% to about 80%, about 10% to about 60%, about 10% to about 40%, about 10% to about 20%, about 20% to about 80%, about 20% to about 60%, about 20% to about 40%, about 40% to about 80%, about 40% to about 60%, or about 60% to about 80% of the truncated gene product relative to a cell without the tRNA. In certain embodiments, there is no detectable truncated gene product in the cell. Truncated gene product amount or expression may be measured by any method known in the art, for example, Western blot or ELISA.

In certain embodiments, the cell contains a greater amount of functional gene product than a cell without the tRNA. For example, in certain embodiments, the method increases the amount of functional gene product in a cell, tissue, or subject by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 250%, about 300%, about 350%, about 400%, about 450%, or about 500% relative to a cell, tissue, or subject without the tRNA. In certain embodiments, the method increases the amount of functional gene product in a cell, tissue, or subject, by from about 20% to about 200%, about 20% to about 180%, about 20% to about 160%, about 20% to about 140%, about 20% to about 120%, about 20% to about 100%, about 20% to about 80%, about 20% to about 60%, about 20% to about 40%, about 40% to about 200%, about 40% to about 180%, about 40% to about 160%, about 40% to about 140%, about 40% to about 120%, about 40% to about 100%, about 40% to about 80%, about 40% to about 60%, about 60% to about 200%, about 60% to about 180%, about 60% to about 160%, about 60% to about 140%, about 60% to about 120%, about 60% to about 100%, about 60% to about 80%, about 80% to about 200%, about 80% to about 180%, about 80% to about 160%, about 80% to about 140%, about 80% to about 120%, about 80% to about 100%, about 100% to about 200%, about 100% to about 180%, about 100% to about 160%, about 100% to about 140%, about 100% to about 120%, about 120% to about 200%, about 120% to about 180%, about 120% to about 160%, about 120% to about 140%, about 140% to about 200%, about 140% to about 180%, about 140% to about 160%, about 160% to about 200%, about 160% to about 180%, or about 180% to about 200% relative to a cell, tissue, or subject without the tRNA. Functional gene product amount or expression may be measured by any method known in the art, for example, Western blot or ELISA.

In certain embodiments, the tRNA permits an amino acid to be incorporated into the gene product at a position corresponding to a premature termination codon (i.e., the tRNA permits read-through of the premature termination codon), but the tRNA does not permit a substantial amount of amino acid to be incorporated into a gene product at a position corresponding to a native stop codon (i.e., the tRNA does not permit read-through of a native stop codon). For example, in certain embodiments, a disclosed tRNA does not increase read-through of a native stop codon (or all native stop codons) in a cell, tissue, or subject, or increases read-through by less than about 1%, about 2%, about 3%, about 4%, about 5%, about 10%, about 20%, about 30%, about 40%, or about 50%, relative to a cell, tissue, or subject that has not been contacted with the tRNA. Read-through of a native stop codon may be measured by any method known in the art, for example, ribosome profiling.

In another aspect, the invention provides a method of treating a premature termination codon-mediated disorder in a subject (or a population of subjects) in need thereof, wherein the subject(s) have a gene with a first, second, and/or third premature termination codon, the method comprising administering to the subject an effective amount of any of the foregoing expression vectors or any of the foregoing pharmaceutical compositions, thereby to treat the disorder in the subject(s).

In another aspect, the invention provides a method of treating a premature termination codon-mediated disorder in a subject (or a population of subjects) in need thereof wherein the subject(s) have a gene with a first, second, and/or third premature termination codon, the method comprising administering to the subject(s) an effective amount of: (a) a first expression vector comprising a nucleotide sequence encoding a first suppressor tRNA that comprises an anticodon that hybridizes to a first premature stop codon (e.g., TGA), and is capable of being aminoacylated with a first amino acid; (b) a second expression vector comprising a nucleotide sequence encoding a second suppressor tRNA that comprises an anticodon that hybridizes to a second premature stop codon (e.g., TAG), and is capable of being aminoacylated with a second amino acid; and optionally, (c) a third expression vector comprising a nucleotide sequence encoding a third suppressor tRNA that comprises an anticodon that hybridizes to a third premature stop codon (e.g., TAA), and is capable of being aminoacylated with a third amino acid; thereby to treat the disorder in the subject(s).

In another aspect, the invention provides a method of treating a premature termination codon-mediated disorder in a subject (or a population of subjects) in need thereof wherein the subject(s) have a gene with a first, second, and/or third premature termination codon, the method comprising administering to the subject(s) an effective amount of: (a) a first suppressor tRNA that comprises an anticodon that hybridizes to a first premature stop codon (e.g., TGA), and is capable of being aminoacylated with a first amino acid; (b) a second suppressor tRNA that comprises an anticodon that hybridizes to a second premature stop codon (e.g., TAG), and is capable of being aminoacylated with a second amino acid; and optionally, (c) a third suppressor tRNA that comprises an anticodon that hybridizes to a third premature stop codon (e.g., TAA), and is capable of being aminoacylated with a third amino acid; thereby to treat the disorder in the subject(s).

I. tRNAs and Suppressor tRNAs

During protein synthesis, a transfer RNA (tRNA) delivers an amino acid to a ribosome for incorporation into a growing protein (polypeptide) chain. tRNAs typically are about 70 to 100 nucleotides in length, and active tRNAs contain a 3′ CCA sequence that may be transcribed into the tRNA during its synthesis or may be added later during post-transcriptional processing. During aminoacylation, the amino acid that is attached to a given tRNA molecule is covalently attached to the 2′ or 3′ hydroxyl group of the 3′-terminal ribose to form an aminoacyl-tRNA (aa-tRNA). It is understood that an amino acid can spontaneously migrate from the 2′-hydroxyl group to the 3′-hydroxyl group and vice versa, but it is incorporated into a growing protein chain at the ribosome from the 3′-OH position. A loop at the other end of the folded aa-tRNA molecule contains a sequence of three bases known as the anticodon. When this anticodon sequence hybridizes or base-pairs with a complementary three-base codon sequence in a ribosome-bound messenger RNA (mRNA), the aa-tRNA binds to the ribosome and its amino acid is incorporated into the polypeptide chain being synthesized by the ribosome. Because all tRNAs that base-pair with a specific codon are aminoacylated with a single specific amino acid, the translation of the genetic code is effected by tRNAs. Each of the 61 non-termination codons in an mRNA directs the binding of its cognate aa-tRNA and the addition of a single specific amino acid to the growing polypeptide chain being synthesized by the ribosome.

tRNAs are generally highly conserved and are often functional across species. Accordingly, a tRNA derived from a bacterial tRNA, a non-mammalian eukaryotic tRNA, or a mammalian (e.g., human) tRNA may be useful in the practice of the invention. Nucleotide sequences encoding naturally occurring human tRNAs are known and generally available to those of skill in the art through sources such as Genbank. See also Sprinzl et al. (2005) NUCLEIC ACIDS RES. 33: D139-40; Buckland et al. (1996) GENOMICS 35 (1): 164-71; Schimmel et al. (Eds.) (1979) “Transfer-RNA: Structure, Properties, and Recognition,” Cold Spring Harbor Laboratory; Agris (1983) “The Modified Nucleosides of Transfer RNA, II,” Alan R. Liss Inc. tRNAs are generally highly conserved and are often functional across species.

Suppressor tRNAs are modified tRNAs that insert a suitable amino acid at a mutant site, e.g., a PTC, in protein encoding gene. The use of the word in suppressor is based on the fact, that under certain circumstance, the modified tRNA “suppresses” the phenotypic effect of the coding mutation. Suppressor tRNAs typically contain a mutation (modification) in either the anticodon, changing codon specificity, or at some position that alters the aminoacylation identity of the tRNA.

In certain embodiments, a tRNA (e.g., a suppressor tRNA) contains a modified anticodon region, such that the modified anticodon hybridizes with a different codon than the corresponding naturally occurring anticodon. In certain embodiments, the modified anticodon hybridizes with a termination codon, e.g., a PTC, and as a result, the tRNA incorporates an amino acid into a gene product rather than terminating protein synthesis. In certain embodiments, the modified anticodon hybridizes with a premature termination codon and, and as a result, the tRNA incorporates an amino acid into a gene product at a position that would otherwise result in a truncated gene product caused by the premature termination codon.

In certain embodiments, a tRNA comprises an anticodon that hybridizes to a codon selected from UAG (i.e., an “amber” termination codon), UGA (i.e., an “opal” termination codon), and UAA (i.e., an “ochre” termination codon). In certain embodiments, the anticodon hybridizes to a codon selected from UGA to UAA. In certain embodiments, the anticodon hybridizes to UGA. In certain embodiments, a tRNA comprises an anticodon that hybridizes to a non-standard termination codon, e.g., a 4-nucleotide codon (See, for example, Moore et al. (2000) J. MOL. BIOL. 298:195, and Hohsaka et al. (1999) J. AM. CHEM. SOC. 121:12194).

In certain embodiments, the tRNA is aminoacylated or is capable of being aminoacylated with any natural amino acid. For example, a tRNA may be capable of being aminoacylated with alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In certain embodiments the tRNA is capable of being aminoacylated with serine, leucine, glutamine, or arginine. In certain embodiments the tRNA is capable of being aminoacylated with glutamine or arginine. In certain embodiments the tRNA is capable of being aminoacylated with arginine.

In certain embodiments, the tRNA (i) comprises an anticodon that hybridizes to a codon as indicated in TABLE 1, and (ii) is aminoacylated or is capable of being aminoacylated with an amino acid as indicated in TABLE 1.

TABLE 1

| | | |
|---|---|---|
| codon: UAG | codon: UGA | codon: UAA |
| amino acid: alanine | amino acid: alanine | amino acid: alanine |
| codon: UAG | codon: UGA | codon: UAA |
| amino acid: arginine | amino acid: arginine | amino acid: arginine |
| codon: UAG | codon: UGA | codon: UAA |
| amino acid: asparagine | amino acid: asparagine | amino acid: asparagine |
| codon: UAG | codon: UGA | codon: UAA |
| amino acid: aspartic acid | amino acid: aspartic acid | amino acid: aspartic acid |
| codon: UAG | codon: UGA | codon: UAA |
| amino acid: cysteine | amino acid: cysteine | amino acid: cysteine |
| codon: UAG | codon: UGA | codon: UAA |
| amino acid: glutamine | amino acid: glutamine | amino acid: glutamine |
| codon: UAG | codon: UGA | codon: UAA |
| amino acid: glutamic acid | amino acid: glutamic acid | amino acid: glutamic acid |
| codon: UAG | codon: UGA | codon: UAA |
| amino acid: glycine | amino acid: glycine | amino acid: glycine |
| codon: UAG | codon: UGA | codon: UAA |
| amino acid: histidine | amino acid: histidine | amino acid: histidine |
| codon: UAG | codon: UGA | codon: UAA |
| amino acid: isoleucine | amino acid: isoleucine | amino acid: isoleucine |
| codon: UAG | codon: UGA | codon: UAA |
| amino acid: leucine | amino acid: leucine | amino acid: leucine |
| codon: UAG | codon: UGA | codon: UAA |
| amino acid: lysine | amino acid: lysine | amino acid: lysine |
| codon: UAG | codon: UGA | codon: UAA |
| amino acid: methionine | amino acid: methionine | amino acid: methionine |
| codon: UAG | codon: UGA | codon: UAA |
| amino acid: phenylalanine | amino acid: phenylalanine | amino acid: phenylalanine |
| codon: UAG | codon: UGA | codon: UAA |
| amino acid: proline | amino acid: proline | amino acid: proline |
| codon: UAG | codon: UGA | codon: UAA |
| amino acid: serine | amino acid: serine | amino acid: serine |
| codon: UAG | codon: UGA | codon: UAA |
| amino acid: threonine | amino acid: threonine | amino acid: threonine |
| codon: UAG | codon: UGA | codon: UAA |
| amino acid: tryptophan | amino acid: tryptophan | amino acid: tryptophan |
| codon: UAG | codon: UGA | codon: UAA |
| amino acid: tyrosine | amino acid: tyrosine | amino acid: tyrosine |
| codon: UAG | codon: UGA | codon: UAA |
| amino acid: valine | amino acid: valine | amino acid: valine |

In certain embodiments, the tRNA comprises, consists essentially of, or consists of a nucleotide sequence shown in TABLE 2. In certain embodiments, the tRNA comprises, consists essentially of, or consists of a nucleotide sequence having 80%, 85%, 86%, 87%, 88%, 89%, 90%, 91%, 92%, 93%, 94%, 95%, 96%, 97%, 98%, or 99% sequence identity to a nucleotide sequence shown in TABLE 2. In certain embodiments, the tRNA comprises, consists essentially of, or consists of a nucleotide sequence selected from SEQ ID NOs: 19-21, 37, 39, 40, 44, 179, 181, 182, and 186, or a nucleotide sequence having 80%, 85%, 86%, 87%, 88%, 89%, 90%, 91%, 92%, 93%, 94%, 95%, 96%, 97%, 98%, or 99% sequence identity to a nucleotide sequence selected from SEQ ID NOs: 19-21, 37, 39, 40, 44, 179, 181, 182, and 186. It is understood that, throughout the description (e.g., TABLES 2 and 3, and the Sequence Listing), in each instance where a tRNA comprises, consists essentially of, or consists of a nucleotide sequence including one or more thymines (T), a tRNA is also contemplated that comprises, consists essentially of, or consists of the same nucleotide sequence including a uracil (U) in place of one or more of the thymines (T), or a uracil (U) in place of all the thymines (T). Similarly, in each instance where a tRNA comprises, consists essentially of, or consists of a nucleotide sequence including one or more uracils (U), a tRNA is also contemplated that comprises, consists essentially of, or consists of a nucleotide sequence including a thymine (T) in place of the one or more of the uracils (U), or a thymine (T) in place of all the uracils (U).

TABLE 2

| SEQ ID NO | Suppressor tRNA Sequence (anticodon shown in lowercase) |
|---|---|
| 2 | GGGCCAGTGGCGCAATGGATAACGCGTCTGACTtcaGATCAGAAGATTGTAG |
| | GTTCGACTCCTACCTGGCTCG |
| 5 | GGCCGCGTGGCCTAATGGATAAGGCGTCTGATTtcaGATCAGAAGATTGGGG |
| | GTTCGAGTCCCTTCGTGGTCG |
| 14 | GACCACGTGGCCTAACGGATAAGGCGTCTGACTtcaGATCAGAAGATTGAGG |
| | GTTCGAATCCCTTCGTGGTTA |
| 15 | GGCTCTGTGGCGCAATGGATAGCGCATTGGACTtcaAGTGACGAGAAAGCGA |
| | TTCAAAGGTTGTGGGTTCGAATCCCACCAGAGTCG |
| 19 | GGCTCTGTGGCGCAATGGATAGCGCATTGGACTtcaAGCATGATTGAGAGAT |
| | TCAAAGGTTGCGGGTTCGAGTCCCGCCAGAGTCG |
| 20 | GGCTCTGTGGCGCAATGGATAGCGCATTGGACTtcaAATTCAAAGGTTGCGG |
| | GTTCGAGTCCCGCCAGAGTCG |
| 21 | GGCTCTGTGGCGCAATGGATAGCGCATTGGACTtcaAGACAAATGGAGGCAT |
| | TCAAAGGTTGTGGGTTCGAGTCCCACCAGAGTCG |
| 23 | GTCTCTGTGGCGCAATGGACGAGCGCGCTGGACTtcaAATCCAGAGGTTCTG |
| | GGTTCGAGTCCCGGCAGAGATG |
| 24 | GGCTCTGTGGAGCAATGGATAGCACATTGGACTtcaAGCATGACCGAGAGAT |
| | TCAAAGGTTGCGGGTTCGAGTCCCACCAGAGTTG |
| 25 | GGCTCTGTGGAGCAATGGATAGCACATTGGACTtcaAATTCAAAGGTTGCGG |
| | GTTCGAGTCCCACCAGAGTTG |
| 37 | GGTTCCATGGTGTAATGGTTAGCACTCTGGACTttaAATCCAGCGACCCGAG |
| | TTCAAATCTCGGTGGGACCT |
| 39 | GGTTCCATGGTGTAATGGTTAGCACTCTGGACTttaAATCCAGCGATCCGAG |
| | TTCAAATCTCGGTGGGACCT |
| 40 | GGTTCCATGGTGTAATGGTGAGCACTCTGGACTttaAATCCAGCGATCCGAG |
| | TTCAAATCTCGGTGGGACCT |
| 41 | GGTTCCATGGTGTAATGGCTAGCACTCTGGACTttaAATCCAGCGATCCGAG |
| | TTCAAATCTCGGTGGGATTT |
| 42 | GGTTCCATGGTGTAATGGTTAGCACTCTGGACTttaAATCCAGCCATACAAG |
| | TTCAAATCTCAGTGGAACCT |
| 43 | GGTTCCTTGGTGTAAGATGAGCACTCTGGATTttaAATCCAGCGATCAGAGT |
| | TCAAATCTCGGTGGGACCT |
| 44 | GGTCCCATGGTGTAATGGTTAGCACTCTGGACTttaAATCCAGCAATCTGAG |
| | TTCAAATCTCGGTGGGACCT |
| 46 | GGTCTCATGGTGTAATGGTTAGCACACTGGACTttaAGTCCAGCAATCCGAG |
| | TTCGAGTCTTGGTGAGACCA |
| 47 | GGACCCATGGTGTAATGGTTAGCACTCTGGACTttaAATCCAGCAATCCAAG |
| | TTCAAATCTCGGTGGGACCT |
| 48 | GTTTCCATGGTGTAATGGTTGGCACTCTGGACTttaAATCCAGCAATCCAAG |
| | TTCAAGTCTCTGTGGGACCT |
| 49 | GTCAGGATGGCCGAGTGGTCTAAGGCGCCAGACTctaGCTATGGCTTCCTCG |
| | CTCTGAGGGTTCTGGTCTCCCCTGGAGGCGTGGGTTCGAATCCCACTTCTGA |
| | CA |
| 50 | GTCAGGATGGCCGAGTGGTCTAAGGCGCCAGACTctaGCTTAGCTTCCCTGT |
| | CTGGGGATTCTGGTCTCCGTATGGAGGCGTGGGTTCGAATCCCACTTCTGAC |
| | A |
| 51 | GTCAGGATGGCCGAGTGGTCTAAGGCGCCAGACTctaGGTGACAAGCCTTAC |
| | CTACGGGTGTTCTGGTCTCCGAATGGAGGCGTGGGTTCGAATCCCACTTCTG |
| | ACA |
| 52 | GTCAGGATGGCCGAGTGGTCTAAGGCGCCAGACTctaGCGTTCGCTTCCTCT |
| | ACTGAGGGTTCTGGTCTCCGTGTGGAGGCGTGGGTTCGAATCCCACTTCTGA |
| | CA |
| 53 | GTCAGGATGGCCGAGTGGTCTAAGGCGCCAGACTtcaGCTATGGCTTCCTCG |
| | CTCTGAGGGTTCTGGTCTCCCCTGGAGGCGTGGGTTCGAATCCCACTTCTGA |
| | CA |
| 54 | GTCAGGATGGCCGAGTGGTCTAAGGCGCCAGACTtcaGCTTAGCTTCCCTGT |
| | CTGGGGATTCTGGTCTCCGTATGGAGGCGTGGGTTCGAATCCCACTTCTGAC |
| | A |
| 55 | GTCAGGATGGCCGAGTGGTCTAAGGCGCCAGACTtcaGGTGACAAGCCTTAC |
| | CTACGGGTGTTCTGGTCTCCGAATGGAGGCGTGGGTTCGAATCCCACTTCTG |
| | ACA |
| 56 | GTCAGGATGGCCGAGTGGTCTAAGGCGCCAGACTtcaGCGTTCGCTTCCTCT |
| | ACTGAGGGTTCTGGTCTCCGTGTGGAGGCGTGGGTTCGAATCCCACTTCTGA |
| | CA |
| 57 | GTCAGGATGGCCGAGTGGTCTAAGGCGCCAGACTttaGCTATGGCTTCCTCG |
| | CTCTGAGGGTTCTGGTCTCCCCTGGAGGCGTGGGTTCGAATCCCACTTCTGA |
| | CA |
| 58 | GTCAGGATGGCCGAGTGGTCTAAGGCGCCAGACTttaGCTTAGCTTCCCTGT |
| | CTGGGGATTCTGGTCTCCGTATGGAGGCGTGGGTTCGAATCCCACTTCTGAC |
| | A |
| 59 | GTCAGGATGGCCGAGTGGTCTAAGGCGCCAGACTttaGGTGACAAGCCTTAC |
| | CTACGGGTGTTCTGGTCTCCGAATGGAGGCGTGGGTTCGAATCCCACTTCTG |
| | ACA |
| 60 | GTCAGGATGGCCGAGTGGTCTAAGGCGCCAGACTttaGCGTTCGCTTCCTCT |
| | ACTGAGGGTTCTGGTCTCCGTGTGGAGGCGTGGGTTCGAATCCCACTTCTGA |
| | CA |
| 61 | CCTTCGATAGCTCAGTTGGTAGAGCGGAGGACTctaGAGTTACTAGAATAGT |
| | GATCCTTAGGTCGCTGGTTCGAATCCGGCTCGAAGGA |
| 62 | CCTTCGATAGCTCAGTTGGTAGAGCGGAGGACTctaGTCAGTACAATATGGT |
| | AATCCTTAGGTCGCTGGTTCGATTCCGGCTCGAAGGA |
| 63 | CCTTCGATAGCTCAGCTGGTAGAGCGGAGGACTctaGGCTTGTGGCTGTGGA |
| | CATCCTTAGGTCGCTGGTTCGATTCCGGCTCGAAGGA |
| 64 | CCTTCGATAGCTCAGCTGGTAGAGCGGAGGACTctaGCTAACTCCCCGTTAG |
| | AAGACATCCTTAGGTCGCTGGTTCGACTCCGGCTCGAAGGA |
| 65 | CTTTCGATAGTTCAGTTGGTAGAGCGGAGGACTctaGAGTATTAACGTTGGT |
| | GATCCTTAGGTCGCTGGTTCGAGTCCGGCTCGAAGGA |
| 66 | CCTTCGATAGCTCAGTTGGTAGAGCGGAGGACTttaGAGTTACTAGAATAGT |
| | GATCCTTAGGTCGCTGGTTCGAATCCGGCTCGAAGGA |
| 67 | CCTTCGATAGCTCAGTTGGTAGAGCGGAGGACTttaGTCAGTACAATATGGT |
| | AATCCTTAGGTCGCTGGTTCGATTCCGGCTCGAAGGA |
| 68 | CCTTCGATAGCTCAGCTGGTAGAGCGGAGGACTttaGGCTTGTGGCTGTGGA |
| | CATCCTTAGGTCGCTGGTTCGATTCCGGCTCGAAGGA |
| 69 | CCTTCGATAGCTCAGCTGGTAGAGCGGAGGACTttaGCTAACTCCCCGTTAG |
| | AAGACATCCTTAGGTCGCTGGTTCGACTCCGGCTCGAAGGA |
| 70 | CTTTCGATAGTTCAGTTGGTAGAGCGGAGGACTttaGAGTATTAACGTTGGT |
| | GATCCTTAGGTCGCTGGTTCGAGTCCGGCTCGAAGGA |
| 71 | GGGGGTATAGCTCAGTGGTAGAGCATTTGACTtcaGATCAAGAGGTCCCTGG |
| | TTCAAATCCAGGTGCCCCCT |
| 72 | GGGGGTATAGCTCAGGGGTAGAGCATTTGACTtcaGATCAAGAGGTCCCTGG |
| | TTCAAATCCAGATGCCCCCT |
| 73 | GGGGGTATAGCTCAGGGGTAGAGTATTTGGCTtcaGATCAAGAGGTCCCTGG |
| | TTCAAATCCAGGTGCCCCCT |
| 74 | GGGGGTATAGCTCAGGGGTAGAGCATTTGACTtcaGATCAAGAGGTCCTTGG |
| | TTCAAATCCAGGTGTCCCCT |
| 75 | GGGGGTATAGCTCAGAGGTAGAGCATTTGACTtcaGATCAAGAGATCTCTGG |
| | TTCAAATCCAGGTGCCCCCT |
| 76 | GGGGGTATAGCTCAGGGGTAGAGCATTTGACTtcaGATCAAGAGGTCCCTAG |
| | TTCAAATCCAGGTGCCCCCT |
| 77 | GGTGGTATAGCTCAGGGGTAGAGCATTTGACTtcaGATCAAGAGATCCCTGG |
| | TTCGAATCCAGGTGCCCCCT |
| 78 | GGGGGTATAACTCAGGGGTAGAGCATTTGACTtcaGATCAAGAGGTCCCTGG |
| | TTCAAATCCAGGTGCCCCCT |
| 79 | TGGGGTATAGCTCAGGGGTAGAGCATTTGACTtcaGATCAAGAGGTCCCTGG |
| | TTCAAATCCAGGTGCCCCCT |
| 80 | GGGGGTATAGCTCAGAGGAAGAGCATTTGACTtcaGATCAAGAGGTCCCTGA |
| | TTCAAATCCAGGTGCCCCCT |
| 81 | GGGGGTAAAGCTCAGGGGTAGAGCATTTGACTtcaGATTAAGAGGTCCCTGG |
| | TTCAAATCCAGGTACCCCCT |
| 83 | GGGGTTATAGCTCAGGTGTAGAGCATTTGACTtcaGATCAAGAGGTCCCTGG |
| | TTCAAATCCAGGTGCCCCCT |
| 84 | GGGGGTATAGCTCAGGGGTAGAGCATTTGACTtcaGATCACGAGGTCCCTGG |
| | TTCAAATCGAGGTGCCCCCT |
| 85 | GGGGGTATAGCTCAGGGGTGGAGCATTTGACTtcaGATCAAGGGGTCCCTGT |
| | TTCAAATCCAGGTGCCCCCT |
| 86 | GGGGGTATAGCTCAGTGGTAGAGCATTTGACTtcaGATCAAGAGGTCCCCGG |
| | TTCAAATCCGGGTGCCCCCT |
| 88 | GGGGGTATAGCTCAGGGGTAGAGCATTTGACTtcaGATCAAGAGGTCCCTGG |
| | TTCAAATCCGGGTGCCCCCT |
| 89 | GGGGGTATAGCTCAGGGGTAGAGCATTTGACTtcaGATCAAGAGGTCCCTGG |
| | TTCAAATCCAGGTACCCCCT |
| 91 | GGGGGCATAGCTCAGGGGTAGAGCATTTGACTtcaGATCAAGAGGTCCCCGG |
| | TTCAAATCCGGGTGCTCCCT |
| 92 | GGGGGTATAGCTCAGGGGTAGAGCATTTGACTtcaGATTAAGAGGTCCCTGG |
| | TTCAAATCCAGGTGCCCCCT |
| 94 | TCCCTGGTGGTCTAGTGGTTAGGATTTGGCGCTctaACCGCCGCGGCCTGGG |
| | TTCGATTCCCGGTCAGGGAA |
| 95 | TCCCTGGTGGTCTAGTGGTTAGGCTTTGGTGCTctaACCTCCATGGCCCAGG |
| | TTTGATTCCTGGTCAGGGAA |
| 97 | TCCCTGGTGGTCTAGTGGTTAGGATTTGGCGCTttaACCGCCGCGGCCTGGG |
| | TTCGATTCCCGGTCAGGGAA |
| 98 | TCCCTGGTGGTCTAGTGGTTAGGCTTTGGTGCTttaACCTCCATGGCCCAGG |
| | TTTGATTCCTGGTCAGGGAA |
| 100 | TCCCATATGGTCTAGCGGTTAGGATTCCTGGTTctaACCCAGGCGGCCCGGG |
| | TTCGACTCCCGGTATGGGAA |
| 103 | TCCCATATGGTCTAGCGGTTAGGATTCCTGGTTttaACCCAGGCGGCCCGGG |
| | TTCGACTCCCGGTATGGGAA |
| 105 | GTTTCCGTAGTGTAGTGGTTAGCGCGTTCGCCTtcaAAAGCGAAAGGTCCCC |
| | GGTTCGAAACCGGGCGGAAACA |
| 107 | GCATTGGTAGTTCAATGGTAGAATTCTCGCCTtcaACGCGGGTGACCCGGGT |
| | TCGATTCCCGGCCAATGCA |
| 108 | GCATTGGTGGTTCAATGGTAGAATTCTCGCCTtcaACGCGGGTGACCCGGGT |
| | TCGATTCCCGGCCAATGCA |
| 109 | GCATTGGTGGTTCAATGGTAGAATTCTCGCCTtcaACTCGGGTGACCCGGGT |
| | TCGATTCCCGGCCAATGCA |
| 112 | GCATTGGTGGTTCAGTGGTAGAATTCTCGCCTtcaACGCGGGAGGCCCGGGT |
| | TTGATTCCCGGCCAATGCA |
| 113 | GCATTGGTGGTTCAGTGGTAGAATTCTCGCCTtcaACGCGGGAGGCCCGGGT |
| | TCGGTTCCCGGCCAATGCA |
| 115 | GGTAGCGTGGCCGAGCGGTCTAAGGCGCTGGATTctaGCTCCAGTCTCTTCG |
| | GGGGCGTGGGTTCGAATCCCACCGCTGCCA |
| 116 | GGTAGTGTGGCCGAGCGGTCTAAGGCGCTGGATTctaGCTCCAGTCTCTTCG |
| | GGGGCGTGGGTTCGAATCCCACCGCTGCCA |
| 118 | GGTAGCGTGGCCGAGCGGTCTAAGGCGCTGGATTtcaGCTCCAGTCTCTTCG |
| | GGGGCGTGGGTTCGAATCCCACCGCTGCCA |
| 119 | GGTAGTGTGGCCGAGCGGTCTAAGGCGCTGGATTtcaGCTCCAGTCTCTTCG |
| | GGGGCGTGGGTTCGAATCCCACCGCTGCCA |
| 121 | GGTAGCGTGGCCGAGCGGTCTAAGGCGCTGGATTttaGCTCCAGTCTCTTCG |
| | GGGGCGTGGGTTCGAATCCCACCGCTGCCA |
| 122 | GGTAGTGTGGCCGAGCGGTCTAAGGCGCTGGATTttaGCTCCAGTCTCTTCG |
| | GGGGCGTGGGTTCGAATCCCACCGCTGCCA |
| 124 | GTCAGGATGGCCGAGTGGTCTAAGGCGCCAGACTctaGTTCTGGTCTCCCCT |
| | GGAGGCGTGGGTTCGAATCCCACTTCTGACA |
| 126 | GTCAGGATGGCCGAGTGGTCTAAGGCGCCAGACTctaGTTCTGGTCTCCGAA |
| | TGGAGGCGTGGGTTCGAATCCCACTTCTGACA |
| 127 | GTCAGGATGGCCGAGTGGTCTAAGGCGCCAGACTctaGTTCTGGTCTCCGTG |
| | TGGAGGCGTGGGTTCGAATCCCACTTCTGACA |
| 128 | GTCAGGATGGCCGAGTGGTCTAAGGCGCCAGACTtcaGTTCTGGTCTCCCCT |
| | GGAGGCGTGGGTTCGAATCCCACTTCTGACA |
| 130 | GTCAGGATGGCCGAGTGGTCTAAGGCGCCAGACTtcaGTTCTGGTCTCCGAA |
| | TGGAGGCGTGGGTTCGAATCCCACTTCTGACA |
| 131 | GTCAGGATGGCCGAGTGGTCTAAGGCGCCAGACTtcaGTTCTGGTCTCCGTG |
| | TGGAGGCGTGGGTTCGAATCCCACTTCTGACA |
| 132 | GTCAGGATGGCCGAGTGGTCTAAGGCGCCAGACTttaGTTCTGGTCTCCCCT |
| | GGAGGCGTGGGTTCGAATCCCACTTCTGACA |
| 134 | GTCAGGATGGCCGAGTGGTCTAAGGCGCCAGACTttaGTTCTGGTCTCCGAA |
| | TGGAGGCGTGGGTTCGAATCCCACTTCTGACA |
| 135 | GTCAGGATGGCCGAGTGGTCTAAGGCGCCAGACTttaGTTCTGGTCTCCGTG |
| | TGGAGGCGTGGGTTCGAATCCCACTTCTGACA |
| 136 | GTCAGGATGGCCGAGCGGTCTAAGGCGCTGCGTTctaGTCGCAGTCTCCCCT |
| | GGAGGCGTGGGTTCGAATCCCACTCCTGACA |
| 137 | GTCAGGATGGCCGAGCGGTCTAAGGCGCTGCGTTctaGTCGCAGTCTCCCCT |
| | GGAGGCGTGGGTTCGAATCCCACTTCTGACA |
| 138 | GTCAGGATGGCCGAGTGGTCTAAGGAGCTGTGTTctaGTCGCAGTCTCCCCT |
| | GGAGGCGTGGGTTCGAATCCCACTCCTGACA |
| 139 | GTCAGGATGGCCGAGCAGTCTAAGGCACTGCGTTctaGTCGCAGTCTCCCCT |
| | GGAGGCGTGGATTCGAATCCCACTCCTGACA |
| 140 | GTCAGGATGGCCGAGCGGTCTAAGGCGCTGCGTTtcaGTCGCAGTCTCCCCT |
| | GGAGGCGTGGGTTCGAATCCCACTCCTGACA |
| 141 | GTCAGGATGGCCGAGCGGTCTAAGGCGCTGCGTTtcaGTCGCAGTCTCCCCT |
| | GGAGGCGTGGGTTCGAATCCCACTTCTGACA |
| 142 | GTCAGGATGGCCGAGTGGTCTAAGGAGCTGTGTTtcaGTCGCAGTCTCCCCT |
| | GGAGGCGTGGGTTCGAATCCCACTCCTGACA |
| 143 | GTCAGGATGGCCGAGCAGTCTAAGGCACTGCGTTtcaGTCGCAGTCTCCCCT |
| | GGAGGCGTGGATTCGAATCCCACTCCTGACA |
| 144 | GTCAGGATGGCCGAGCGGTCTAAGGCGCTGCGTTttaGTCGCAGTCTCCCCT |
| | GGAGGCGTGGGTTCGAATCCCACTCCTGACA |
| 145 | GTCAGGATGGCCGAGCGGTCTAAGGCGCTGCGTTttaGTCGCAGTCTCCCCT |
| | GGAGGCGTGGGTTCGAATCCCACTTCTGACA |
| 146 | GTCAGGATGGCCGAGTGGTCTAAGGAGCTGTGTTttaGTCGCAGTCTCCCCT |
| | GGAGGCGTGGGTTCGAATCCCACTCCTGACA |
| 147 | GTCAGGATGGCCGAGCAGTCTAAGGCACTGCGTTttaGTCGCAGTCTCCCCT |
| | GGAGGCGTGGATTCGAATCCCACTCCTGACA |
| 148 | ACCAGAATGGCCGAGTGGTTAAGGCGTTGGACTctaGATCCAATGGATTTAT |
| | ATCCGCGTGGGTTCGAACCCCACTTCTGGTA |
| 149 | ACCAGGATGGCCGAGTGGTTAAGGCGTTGGACTctaGATCCAATGGACATAT |
| | GTCTGCGTGGGTTCGAACCCCACTCCTGGTA |
| 150 | ACTGGGATGGCTGAGTGGTTAAGGCGTTGGACTctaGATCCAATGGGCGGTT |
| | GCCTGCGTGGGTTCGAACCCCACTCCCAGTA |
| 151 | GATGGGATGGCTGAGAGGTTAAGGCTTTGGACTctaGATCCAATGGGCAGAT |
| | GCCTGCGTGGGTTTGAACCCCACTCCCAATA |
| 152 | ACCAGAATGGCCGAGTGGTTAAGGCGTTGGACTtcaGATCCAATGGATTTAT |
| | ATCCGCGTGGGTTCGAACCCCACTTCTGGTA |
| 153 | ACCAGGATGGCCGAGTGGTTAAGGCGTTGGACTtcaGATCCAATGGACATAT |
| | GTCTGCGTGGGTTCGAACCCCACTCCTGGTA |
| 154 | ACTGGGATGGCTGAGTGGTTAAGGCGTTGGACTtcaGATCCAATGGGCGGTT |
| | GCCTGCGTGGGTTCGAACCCCACTCCCAGTA |
| 155 | GATGGGATGGCTGAGAGGTTAAGGCTTTGGACTtcaGATCCAATGGGCAGAT |
| | GCCTGCGTGGGTTTGAACCCCACTCCCAATA |
| 156 | ACCAGAATGGCCGAGTGGTTAAGGCGTTGGACTttaGATCCAATGGATTTAT |
| | ATCCGCGTGGGTTCGAACCCCACTTCTGGTA |
| 157 | ACCAGGATGGCCGAGTGGTTAAGGCGTTGGACTttaGATCCAATGGACATAT |
| | GTCTGCGTGGGTTCGAACCCCACTCCTGGTA |
| 158 | ACTGGGATGGCTGAGTGGTTAAGGCGTTGGACTttaGATCCAATGGGCGGTT |
| | GCCTGCGTGGGTTCGAACCCCACTCCCAGTA |
| 159 | GATGGGATGGCTGAGAGGTTAAGGCTTTGGACTttaGATCCAATGGGCAGAT |
| | GCCTGCGTGGGTTTGAACCCCACTCCCAATA |
| 161 | GGTAGTGTGGCCGAGCGGTCTAAGGCGCTGGATTctaGCTCCAGTCTCTTCG |
| | GAGGCGTGGGTTCGAATCCCACCACTGCCA |
| 164 | GGTAGTGTGGCCGAGCGGTCTAAGGCGCTGGATTtcaGCTCCAGTCTCTTCG |
| | GAGGCGTGGGTTCGAATCCCACCACTGCCA |
| 167 | GGTAGTGTGGCCGAGCGGTCTAAGGCGCTGGATTttaGCTCCAGTCTCTTCG |
| | GAGGCGTGGGTTCGAATCCCACCACTGCCA |
| 169 | GCCCAGCTAGCTCAGTTGGTAGAGCGTGGGACTctaAATCCTAGGGTCGTGG |
| | GTTCGAACCCCACGTTGGGCG |
| 170 | GCCCAGCTAGCTCAGTCTGTAGAGCATGAGACTctaAGTCTCAGGGTCATGG |
| | GTTGGAGCCCCATGTTGTGCA |
| 171 | GCCTAGCTAGTTCAGTCGGTAGAGCATGAGACTctaAATCTCAGGTTCATGA |
| | GTTTGAGCCCCATGTTGGTTTGGCA |
| 172 | CCCCGGCTAGCTCAGTCAGTAGAGCTTGAGAATctaAATCTCAGGGTCGTGG |
| | GTTGGAGCCCCACGTTGGGCG |
| 179 | GGTTCCATGGTGTAATGGTTAGCACTCTGGACTctaAATCCAGCGACCCGAG |
| | TTCAAATCTCGGTGGGACCT |
| 181 | GGTTCCATGGTGTAATGGTTAGCACTCTGGACTctaAATCCAGCGATCCGAG |
| | TTCAAATCTCGGTGGGACCT |
| 182 | GGTTCCATGGTGTAATGGTGAGCACTCTGGACTctaAATCCAGCGATCCGAG |
| | TTCAAATCTCGGTGGGACCT |
| 183 | GGTTCCATGGTGTAATGGCTAGCACTCTGGACTctaAATCCAGCGATCCGAG |
| | TTCAAATCTCGGTGGGATTT |
| 184 | GGTTCCATGGTGTAATGGTTAGCACTCTGGACTctaAATCCAGCCATACAAG |
| | TTCAAATCTCAGTGGAACCT |
| 185 | GGTTCCTTGGTGTAAGATGAGCACTCTGGATTctaAATCCAGCGATCAGAGT |
| | TCAAATCTCGGTGGGACCT |
| 186 | GGTCCCATGGTGTAATGGTTAGCACTCTGGACTctaAATCCAGCAATCTGAG |
| | TTCAAATCTCGGTGGGACCT |
| 188 | GGTCTCATGGTGTAATGGTTAGCACACTGGACTctaAGTCCAGCAATCCGAG |
| | TTCGAGTCTTGGTGAGACCA |
| 189 | GGACCCATGGTGTAATGGTTAGCACTCTGGACTctaAATCCAGCAATCCAAG |
| | TTCAAATCTCGGTGGGACCT |
| 190 | GTTTCCATGGTGTAATGGTTGGCACTCTGGACTctaAATCCAGCAATCCAAG |
| | TTCAAGTCTCTGTGGGACCT |
| 196 | GCCCGGCTAGCTCAGTCGGTAGAGCATGGGACTctaAATCCCAGGGTCGTGG |
| | GTTCGAGCCCCACGTTGGGCG |
| 197 | GCCCGGCTAGCTCAGTCGGTAGAGCATGAGACTctaAATCTCAGGGTCGTGG |
| | GTTCGAGCCCCACGTTGGGCG |
| 198 | GCCCAGCTAGCTCAGTCTGTAGAGCATGAGACTctaAATCTCAGGGTCGTGA |
| | GTTCGAGCCCCACGTTGGGTG |
| 199 | GCCCAGATAGCTCAGTGGGTAGAGCATGAGACTctaAATCTCAGGGTCATGG |
| | GTTCATGCCCCATGTTGGGTA |
| 200 | GTCCTGCTGGCTCAGTCGGTACAGCATGGGACTctaAATCCCAGGGTCGTGG |
| | GTTCGAGCTCCACGTTGGGTA |
| 201 | GCCTGGCTAGCTCAGTCCATAGAGCATGGGACTctaAATCCCAGGGTCATGG |
| | GTTCGAGCCCCATATTAGGCA |
| 202 | GCCCAGCTAGCTTAGTTGGTAGAGCATGAGACTctaAATCTCAGAGTCATGG |
| | GTTCAGGCCTCATGTTTGGCA |
| 203 | AACCTGGCTAGGTCAGTTGGTAGATCATGAGACTctaAATCTCAGGGTCATG |
| | GGTTCAAGCCCCATGTTGGTTT |
| 204 | GCCCAGCTAGCTCAGTTGGTAGAGCGTGGGACTttaAATCCTAGGGTCGTGG |
| | GTTCGAACCCCACGTTGGGCG |
| 205 | GCCCAGCTAGCTCAGTCTGTAGAGCATGAGACTttaAGTCTCAGGGTCATGG |
| | GTTGGAGCCCCATGTTGTGCA |
| 206 | GCCTAGCTAGTTCAGTCGGTAGAGCATGAGACTttaAATCTCAGGTTCATGA |
| | GTTTGAGCCCCATGTTGGTTTGGCA |
| 207 | CCCCGGCTAGCTCAGTCAGTAGAGCTTGAGAATttaAATCTCAGGGTCGTGG |
| | GTTGGAGCCCCACGTTGGGCG |
| 208 | GCCCGGCTAGCTCAGTCGGTAGAGCATGGGACTttaAATCCCAGGGTCGTGG |
| | GTTCGAGCCCCACGTTGGGCG |
| 209 | GCCCGGCTAGCTCAGTCGGTAGAGCATGAGACTttaAATCTCAGGGTCGTGG |
| | GTTCGAGCCCCACGTTGGGCG |
| 210 | GCCCAGCTAGCTCAGTCTGTAGAGCATGAGACTttaAATCTCAGGGTCGTGA |
| | GTTCGAGCCCCACGTTGGGTG |
| 211 | GCCCAGATAGCTCAGTGGGTAGAGCATGAGACTttaAATCTCAGGGTCATGG |
| | GTTCATGCCCCATGTTGGGTA |
| 212 | GTCCTGCTGGCTCAGTCGGTACAGCATGGGACTttaAATCCCAGGGTCGTGG |
| | GTTCGAGCTCCACGTTGGGTA |
| 213 | GCCTGGCTAGCTCAGTCCATAGAGCATGGGACTttaAATCCCAGGGTCATGG |
| | GTTCGAGCCCCATATTAGGCA |
| 214 | GCCCAGCTAGCTTAGTTGGTAGAGCATGAGACTttaAATCTCAGAGTCATGG |
| | GTTCAGGCCTCATGTTTGGCA |
| 215 | AACCTGGCTAGGTCAGTTGGTAGATCATGAGACTttaAATCTCAGGGTCATG |
| | GGTTCAAGCCCCATGTTGGTTT |
| 216 | GCCCGGATAGCTCAGTCGGTAGAGCATCAGACTctaAATCTGAGGGTCCAGG |
| | GTTCAAGTCCCTGTTCGGGCG |
| 218 | GCCTGGATAGCTCAATTGGTAGAGCATCAGACTctaAATCTGAGGGTTCAGG |
| | GTTCAAGTCCCTGTTCAGGCG |
| 219 | GCCCAGCCAGCTCAGTAGGTAGAGTATGAGACTctaAATCTCAGGGTGGTGG |
| | GTTCGAGCCCCATGTTGGGGG |
| 220 | TGTGGTGTAGCTCAGTCGGTAGAGCATCAGACTctaAATCTGAGGGTCCAGG |
| | GTTCAGGTCCCTGTTCGGGTGCCAAAA |
| 221 | GCCCGGATAGCTCAGTCGGTAGAGCATCAGACTttaAATCTGAGGGTCCAGG |
| | GTTCAAGTCCCTGTTCGGGCG |
| 223 | GCCTGGATAGCTCAATTGGTAGAGCATCAGACTttaAATCTGAGGGTTCAGG |
| | GTTCAAGTCCCTGTTCAGGCG |
| 224 | GCCCAGCCAGCTCAGTAGGTAGAGTATGAGACTttaAATCTCAGGGTGGTGG |
| | GTTCGAGCCCCATGTTGGGGG |
| 225 | TGTGGTGTAGCTCAGTCGGTAGAGCATCAGACTttaAATCTGAGGGTCCAGG |
| | GTTCAGGTCCCTGTTCGGGTGCCAAAA |
| 228 | GTAGTCGTGGCCAAGTGAGTAAGGCAATGGACTctaAATCCATTGGGGTCTC |
| | CCAGCACAGGTTCAAATCCTGCTGACTATG |
| 231 | GTAGTCGTGGCCAAGTGAGTAAGGCAATGGACTtcaAATCCATTGGGGTCTC |
| | CCAGCACAGGTTCAAATCCTGCTGACTATG |
| 234 | GTAGTCGTGGCCAAGTGAGTAAGGCAATGGACTttaAATCCATTGGGGTCTC |
| | CCAGCACAGGTTCAAATCCTGCTGACTATG |
| 237 | GCTGTGATGGCCGAGTGGTTAAGGCGTTGGACTctaAATCCAATGGGTTCTT |
| | CCCGCGCAGGTTCAAATCCTGCTCACAGCG |
| 240 | GCTGTGATGGCCGAGTGGTTAAGGCGTTGGACTtcaAATCCAATGGGTTCTT |
| | CCCGCGCAGGTTCAAATCCTGCTCACAGCG |
| 243 | GCTGTGATGGCCGAGTGGTTAAGGCGTTGGACTttaAATCCAATGGGTTCTT |
| | CCCGCGCAGGTTCAAATCCTGCTCACAGCG |
| 245 | GACGAGGTGGCCGAGTGGTTAAGGCGATGGACTctaAATCCATTGTGCTCTG |
| | CACGCATGGGTTCGAATCCCATCCTCGTCG |
| 246 | GACGAGGTGGCCGAGTGGTTAAGGCGATGGACTctaAATCCATTGTGCTTTG |
| | CACGCGTGGGTTCGAATCCCATCCTCGTCG |
| 247 | GACGAGGTGGCCGAGTGGTTAAGGCGATGGACTctaAATCCATTGTGCTCTG |
| | CACGCGTGGGTTCGAATCCCATCCTCGTCG |
| 248 | GATGAGGTGGCCGAGTGGTTAAGGCGATGGACTctaAATCCATTGTGCTCTG |
| | CACGCATGGGTTCGAATCCCATCCTCATCG |
| 250 | GACGAGGTGGCCGAGTGGTTAAGGCGATGGACTtcaAATCCATTGTGCTCTG |
| | CACGCATGGGTTCGAATCCCATCCTCGTCG |
| 251 | GACGAGGTGGCCGAGTGGTTAAGGCGATGGACTtcaAATCCATTGTGCTTTG |
| | CACGCGTGGGTTCGAATCCCATCCTCGTCG |
| 252 | GACGAGGTGGCCGAGTGGTTAAGGCGATGGACTtcaAATCCATTGTGCTCTG |
| | CACGCGTGGGTTCGAATCCCATCCTCGTCG |
| 253 | GATGAGGTGGCCGAGTGGTTAAGGCGATGGACTtcaAATCCATTGTGCTCTG |
| | CACGCATGGGTTCGAATCCCATCCTCATCG |
| 255 | GACGAGGTGGCCGAGTGGTTAAGGCGATGGACTttaAATCCATTGTGCTCTG |
| | CACGCATGGGTTCGAATCCCATCCTCGTCG |
| 256 | GACGAGGTGGCCGAGTGGTTAAGGCGATGGACTttaAATCCATTGTGCTTTG |
| | CACGCGTGGGTTCGAATCCCATCCTCGTCG |
| 257 | GACGAGGTGGCCGAGTGGTTAAGGCGATGGACTttaAATCCATTGTGCTCTG |
| | CACGCGTGGGTTCGAATCCCATCCTCGTCG |
| 258 | GATGAGGTGGCCGAGTGGTTAAGGCGATGGACTttaAATCCATTGTGCTCTG |
| | CACGCATGGGTTCGAATCCCATCCTCATCG |
| 259 | GCTGAAATAGCTCAGTTGGGAGAGCATTAGACTctaGATCTAAAGGTCCCTG |
| | GTTTGATCCCGGGTTTCGGCA |
| 260 | GCTGAAATAGCTCAGTTGGGAGAGCATTAGACTtcaGATCTAAAGGTCCCTG |
| | GTTTGATCCCGGGTTTCGGCA |
| 261 | GCTGAAATAGCTCAGTTGGGAGAGCATTAGACTttaGATCTAAAGGTCCCTG |
| | GTTTGATCCCGGGTTTCGGCA |
| 265 | GACCTCGTGGCGCAATGGTAGCGCGTCTGACTctaGATCAGAAGGTTGCGTG |
| | TTCAAATCACGTCGGGGTCA |
| 270 | GACCTCGTGGCACAATGGTAGCACGTCTGACTctaGATCAGAAGGTTGCGTG |
| | TTCAAATCACGTCGGGGTCA |
| 280 | CCTTCGATAGCTCAGCTGGTAGAGCGGAGGACTctaGATCCTTAGGTCGCTG |
| | GTTCGACTCCGGCTCGAAGGA |
| 281 | CTTTCGATAGTTCAGTTGGTAGAGCGGAGGACTctaGATCCTTAGGTCGCTG |
| | GTTCGAGTCCGGCTCGAAGGA |
| 285 | CCTTCGATAGCTCAGCTGGTAGAGCGGAGGACTttaGATCCTTAGGTCGCTG |
| | GTTCGACTCCGGCTCGAAGGA |
| 286 | CTTTCGATAGTTCAGTTGGTAGAGCGGAGGACTttaGATCCTTAGGTCGCTG |
| | GTTCGAGTCCGGCTCGAAGGA |

In certain embodiments, the tRNA comprises, consists essentially of, or consists of a nucleotide sequence shown in TABLE 3. In certain embodiments, the tRNA comprises, consists essentially of, or consists of a nucleotide sequence having 80%, 85%, 86%, 87%, 88%, 89%, 90%, 91%, 92%, 93%, 94%, 95%, 96%, 97%, 98%, or 99% sequence identity to a nucleotide sequence shown in TABLE 3. In certain embodiments, the tRNA comprises, consists essentially of, or consists of a nucleotide sequence selected from SEQ ID NOs: 6-9, 11, 16-18, 22, 35, 36, 38, 45, 178, 180, and 187, or a nucleotide sequence having 80%, 85%, 86%, 87%, 88%, 89%, 90%, 91%, 92%, 93%, 94%, 95%, 96%, 97%, 98%, or 99% sequence identity to a nucleotide sequence selected from SEQ ID NOs: 6-9, 11, 16-18, 22, 35, 36, 38, 45, 178, 180, and 187.

TABLE 3

| SEQ ID NO | Suppressor tRNA Sequence |
|---|---|
| 1 | GGGCCAGTGGCGCAATGGATAACGCGTCTGACTtcaGATCAGAAGATTCCAG |
| | GTTCGACTCCTGGCTGGCTCG |
| 3 | GGGCCAGTGGCGCAATGGATAACGCGTCTGACTtcaGATCAGAAGATTCTAG |
| | GTTCGACTCCTGGCTGGCTCG |
| 4 | GGCCGCGTGGCCTAATGGATAAGGCGTCTGATTtcaGATCAGAAGATTGAGG |
| | GTTCGAGTCCCTTCGTGGTCG |
| 6 | GACCCAGTGGCCTAATGGATAAGGCATCAGCCTtcaGAGCTGGGGATTGTGG |
| | GTTCGAGTCCCATCTGGGTCG |
| 7 | GCCCCAGTGGCCTAATGGATAAGGCACTGGCCTtcaAAGCCAGGGATTGTGG |
| | GTTCGAGTCCCACCTGGGGTA |
| 8 | GCCCCAGTGGCCTAATGGATAAGGCACTGGCCTtcaAAGCCAGGGATTGTGG |
| | GTTCGAGTCCCACCTGGGGTG |
| 9 | GCCCCGGTGGCCTAATGGATAAGGCATTGGCCTtcaAAGCCAGGGATTGTGG |
| | GTTCGAGTCCCACCCGGGGTA |
| 10 | GCCCCAGTGGCCTAATGGATAAGGCATTGGCCTtcaAAGCCAGGGATTGTGG |
| | GTTCGAGTCCCATCTGGGGTG |
| 11 | GGCCGCGTGGCCTAATGGATAAGGCGTCTGACTtcaGATCAGAAGATTGCAG |
| | GTTCGAGTCCTGCCGCGGTCG |
| 12 | GACCGCGTGGCCTAATGGATAAGGCGTCTGACTtcaGATCAGAAGATTGAGG |
| | GTTCGAGTCCCTTCGTGGTCG |
| 13 | GACCACGTGGCCTAATGGATAAGGCGTCTGACTtcaGATCAGAAGATTGAGG |
| | GTTCGAATCCCTTCGTGGTTG |
| 16 | GGCTCTGTGGCGCAATGGATAGCGCATTGGACTtcaAATTCAAAGGTTGTGG |
| | GTTCGAATCCCACCAGAGTCG |
| 17 | GGCTCCGTGGCGCAATGGATAGCGCATTGGACTtcaAGAGGCTGAAGGCATT |
| | CAAAGGTTCCGGGTTCGAGTCCCGGCGGAGTCG |
| 18 | GGCTCCGTGGCGCAATGGATAGCGCATTGGACTtcaAATTCAAAGGTTCCGG |
| | GTTCGAGTCCCGGCGGAGTCG |
| 22 | GGCTCTGTGGCGCAATGGATAGCGCATTGGACTtcaAATTCAAAGGTTGTGG |
| | GTTCGAGTCCCACCAGAGTCG |
| 35 | GTCTCTGTGGCGCAATGGACGAGCGCGCTGGACTtcaAATCCAGAGGTTCCG |
| | GGTTCGAGTCCCGGCAGAGATG |
| 36 | GGTCCCATGGTGTAATGGTTAGCACTCTGGACTttaAATCCAGCGATCCGAG |
| | TTCAAATCTCGGTGGGACCT |
| 38 | GGTTCCATGGTGTAATGGTTAGCACTCTGGACTttaAATCCAGCGATCCGAG |
| | TTCAAATCTCGGTGGAACCT |
| 45 | GGCCCCATGGTGTAATGGTTAGCACTCTGGACTttaAATCCAGCGATCCGAG |
| | TTCAAATCTCGGTGGGACCT |
| 178 | GGTCCCATGGTGTAATGGTTAGCACTCTGGACTctaAATCCAGCGATCCGAG |
| | TTCAAATCTCGGTGGGACCT |
| 180 | GGTTCCATGGTGTAATGGTTAGCACTCTGGACTctaAATCCAGCGATCCGAG |
| | TTCAAATCTCGGTGGAACCT |
| 187 | GGCCCCATGGTGTAATGGTTAGCACTCTGGACTctaAATCCAGCGATCCGAG |
| | TTCAAATCTCGGTGGGACCT |
| 287 | GGCCTCGTGGCGCAACGGTAGCGCGTCTGACTTCAGATCAGAAGGTTGCGGG |
| | TTCAAATCCCGTCGGGGTCA |
| 288 | GGCCTCGTGGCGCAACGGTAGCGCGTCTGACTTCAGATCAGAAGGTTACGGG |
| | TTCAAATCCCGTCGGGGTCA |
| 289 | GGCCTCGTGGCGCAACGGTAGCGCGTCTGACTTCAGATCAGAAGGTTCCGGG |
| | TTCAAATCCCGGCGGGGTCA |
| 290 | CGTCGGCTCTGTGGCGCAATGGATAGCGCATTGGACTTCAAATTCAAAGGTT |
| | GTGGGTTCGAGTCCCAGAGTCG |
| 291 | CGTCGCCCCAGTGGCCTAATGGATAAGGCACTGGCCTTCAAAGCCAGGGATT |
| | GTGGGTTCGAGTCCCACCTGGGGTG |
| 292 | CGTCGGCTCCGTGGCGCAATGGATAGCGCATTGGACTTCAAATTCAAAGGTT |
| | CCGGGTTCGAGTCCCGGCGGAGTCG |
| 293 | CGTCGCCCCAGTGGCCTAATGGATAAGGCATTGGCCTTCAAAGCCAGGGATT |
| | GTGGGTTCGAGTCCCATCTGGGGTG |
| 294 | CGTCGGCTCTGTGGCGCAATGGATAGCGCATTGGACTTCAAATTCAAAGGTT |
| | GTGGGTTCGAATCCCACCAGAGTCG |
| 295 | CGTCGGCTCTGTGGCGCAATGGATAGCGCATTGGACTTCAAGCTGAGCCTAG |
| | TGTGGTCATTCAAAGGTTGTGGGTTCGAGTCCCACCAGAGTCG |
| 296 | CGTCGCCCCGGTGGCCTAATGGATAAGGCATTGGCCTTCAAAGCCAGGGATT |
| | GTGGGTTCGAGTCCCACCCGGGGTA |
| 297 | CGTCGGCTCCGTGGCGCAATGGATAGCGCATTGGACTTCAAGAGGCTGAAGG |
| | CATTCAAAGGTTCCGGGTTCGAGTCCCGGCGGAGTCG |
| 298 | CGTCGGCTCTGTGGCGCAATGGATAGCGCATTGGACTTCAAGTGACGAATAG |
| | AGCAATTCAAAGGTTGTGGGTTCGAATCCCACCAGAGTCG |
| 299 | CGTCGGCCGCGTGGCCTAATGGATAAGGCGTCTGACTTCAGATCAGAAGATT |
| | GCAGGTTCGAGTCCTGCCGCGGTCG |
| 300 | CGTCGACCGCGTGGCCTAATGGATAAGGCGTCTGACTTCAGATCAGAAGATT |
| | GAGGGTTCGAGTCCCTTCGTGGTCG |
| 301 | CGTCGGCTCTGTGGCGCAATGGATAGCGCATTGGACTTCAAGATAGTTAGAG |
| | AAATTCAAAGGTTGTGGGTTCGAGTCCCACCAGAGTCG |
| 302 | CGTCGGTTCCATGGTGTAATGGTGAGCACTCTGGACTCTAAATCCAGCGATC |
| | CGAGTTCGAGTCTCGGTGGAACCT |
| 303 | CGTCGGCCCCATGGTGTAATGGTTAGCACTCTGGACTCTAAATCCAGCGATC |
| | CGAGTTCAAATCTCGGTGGGACCT |
| 304 | CGTCGGTCCCATGGTGTAATGGTTAGCACTCTGGACTCTAAATCCAGCAATC |
| | CGAGTTCGAATCTCGGTGGGACCT |
| 305 | CGTCGGTCCCATGGTGTAATGGTTAGCACTCTGGACTCTAAATCCAGCGATC |
| | CGAGTTCAAATCTCGGTGGGACCT |
| 306 | CGTCGGCCCCATGGTGTAATGGTCAGCACTCTGGACTCTAAATCCAGCGATC |
| | CGAGTTCAAATCTCGGTGGGACCC |
| 307 | CGTCGGTTCCATGGTGTAATGGTAAGCACTCTGGACTCTAAATCCAGCGATC |
| | CGAGTTCGAGTCTCGGTGGAACCT |
| 308 | CGTCGGTTCCATGGTGTAATGGTTAGCACTCTGGACTCTAAATCCGGTAATC |
| | CGAGTTCAAATCTCGGTGGAACCT |
| 309 | CGTCGGTTCCATGGTGTAATGGTTAGCACTCTGGACTCTAAATCCAGCGATC |
| | CGAGTTCAAGTCTCGGTGGAACCT |
| 310 | CGTCGGTTCCATGGTGTAATGGTAAGCACTCTGGACTTTAAATCCAGCGATC |
| | CGAGTTCGAGTCTCGGTGGAACCT |
| 311 | CGTCGGCCCCATGGTGTAATGGTTAGCACTCTGGACTTTAAATCCAGCGATC |
| | CGAGTTCAAATCTCGGTGGGACCT |
| 312 | CGTCGGTTCCATGGTGTAATGGTGAGCACTCTGGACTTTAAATCCAGCGATC |
| | CGAGTTCGAGTCTCGGTGGAACCT |
| 313 | CGTCGGTTCCATGGTGTAATGGTTAGCACTCTGGACTTTAAATCCAGCGATC |
| | CGAGTTCAAATCTCGGTGGAACCT |
| 314 | CGTCGGTCCCATGGTGTAATGGTTAGCACTCTGGACTTTAAATCCAGCGATC |
| | CGAGTTCAAATCTCGGTGGGACCT |
| 315 | CGTCGGTCCCATGGTGTAATGGTTAGCACTCTGGACTTTAAATCCAGCAATC |
| | CGAGTTCGAATCTCGGTGGGACCT |
| 316 | CGTCGGTTCCATGGTGTAATGGTTAGCACTCTGGACTTTAAATCCGGTAATC |
| | CGAGTTCAAATCTCGGTGGAACCT |
| 317 | CGTCGGCCCCATGGTGTAATGGTCAGCACTCTGGACTTTAAATCCAGCGATC |
| | CGAGTTCAAATCTCGGTGGGACCC |
| 318 | CGTCGGTTCCATGGTGTAATGGTTAGCACTCTGGACTTTAAATCCAGCGATC |
| | CGAGTTCAAGTCTCGGTGGAACCT |
| 319 | CGTCGACCTCGTGGCGCAATGGTAGCGCGTCTGACTCTAGATCAGAAGGTTG |
| | CGTGTTCAAGTCACGTCGGGGTCA |
| 320 | CGTCGACCTCGTGGCGCAACGGTAGCGCGTCTGACTCTAGATCAGAAGGTTG |
| | CGTGTTCAAATCACGTCGGGGTCA |
| 321 | CGTCGGCCTCGTGGCGCAACGGTAGCGCGTCTGACTCTAGATCAGAAGGTTG |
| | CGTGTTCAAATCACGTCGGGGTCA |
| 322 | CGTCGACCTCGTGGCGCAACGGTAGCGCGTCTGACTCTAGATCAGAAGGCTG |
| | CGTGTTCGAATCACGTCGGGGTCA |
| 323 | CGTCGACCTCGTGGCGCAACGGCAGCGCGTCTGACTCTAGATCAGAAGGTTG |
| | CGTGTTCAAATCACGTCGGGGTCA |
| 324 | CGTCTCCCACATGGTCTAGCGGTTAGGATTCCTGGTTCTAACCCAGGCGGCC |
| | CGGGTTCGACTCCCGGTGTGGGAA |
| 325 | CGTCTCCCATATGGTCTAGCGGTTAGGATTCCTGGTTCTAACCCAGGTGGCC |
| | CGGGTTCGACTCCCGGTATGGGAA |
| 326 | CGTCTCCCTGGTGGTCTAGTGGCTAGGATTCGGCGCTCTAACCGCCGCGGCC |
| | CGGGTTCGATTCCCGGTCAGGGAA |
| 327 | CGTCTCCCTGGTGGTCTAGTGGTTAGGATTCGGCGCTCTAACCGCCGCGGCC |
| | CGGGTTCGATTCCCGGTCAGGGAA |
| 328 | CGTCTCCCTGGTCTAGTGGCTAGGATTCGGCGCTCTAACCGCCGCGGCCCGG |
| | GTTCGATTCCCGGCCAGGGAA |
| 329 | CGTCTCCCACATGGTCTAGCGGTTAGGATTCCTGGTTCTAACCCAGGCGGCC |
| | CGGGTTCGACTCCCGGTGTGGGAA |
| 330 | CGTCTCCCATATGGTCTAGCGGTTAGGATTCCTGGTTCTAACCCAGGTGGCC |
| | CGGGTTCGACTCCCGGTATGGGAA |
| 331 | CGTCTCCCTGGTGGTCTAGTGGTTAGGATTCGGCGCTCTAACCGCCGCGGCC |
| | CGGGTTCGATTCCCGGTCAGGGAA |
| 332 | CGTCTCCCTGGTGGTCTAGTGGTTAGGATTCGGCGCTCTAACCGCCGCGGCC |
| | CGGGTTCGATTCCCGGTCAGGAAA |
| 333 | CGTCTCCCTGGTGGTCTAGTGGCTAGGATTCGGCGCTCTAACCGCCGCGGCC |
| | CGGGTTCGATTCCCGGCCAGGGAA |
| 334 | GGCCTCGTGGCGCAACGGTAGCGCGTCTGACTTCAGATCAGAAGGTTGCGTG |
| | TTCAAATCACGTCGGGGTCA |
| 335 | GACCTCGTGGCGCAATGGTAGCGCGTCTGACTTCAGATCAGAAGGTTGCGTG |
| | TTCAAGTCACGTCGGGGTCA |
| 336 | GACCTCGTGGCGCAACGGTAGCGCGTCTGACTTCAGATCAGAAGGTTGCGTG |
| | TTCAAATCACGTCGGGGTCA |
| 337 | GACCTCGTGGCGCAACGGTAGCGCGTCTGACTTCAGATCAGAAGGCTGCGTG |
| | TTCGAATCACGTCGGGGTCA |
| 338 | GACCTCGTGGCGCAACGGCAGCGCGTCTGACTTCAGATCAGAAGGTTGCGTG |
| | TTCAAATCACGTCGGGGTCA |
| 339 | GCGTTGGTGGTATAGTGGTTAGCATAGCTGCCTTCAAAGCAGTTGACCCGGG |
| | TTCGATTCCCGGCCAACGCA |
| 340 | GCGTTGGTGGTATAGTGGTGAGCATAGCTGCCTTCAAAGCAGTTGACCCGGG |
| | TTCGATTCCCGGCCAACGCA |
| 341 | GCGTTGGTGGTATAGTGGTAAGCATAGCTGCCTTCAAAGCAGTTGACCCGGG |
| | TTCGATTCCCGGCCAACGCA |
| 342 | GGCCTCGTGGCGCAACGGTAGCGCGTCTGACTTCAGATCAGAAGGTTGCGTG |
| | TTCAAATCACGTCGGGGTCA |
| 343 | GACCTCGTGGCGCAATGGTAGCGCGTCTGACTTCAGATCAGAAGGTTGCGTG |
| | TTCAAGTCACGTCGGGGTCA |
| 344 | GACCTCGTGGCGCAACGGTAGCGCGTCTGACTTCAGATCAGAAGGTTGCGTG |
| | TTCAAATCACGTCGGGGTCA |
| 345 | GACCTCGTGGCGCAACGGTAGCGCGTCTGACTTCAGATCAGAAGGCTGCGTG |
| | TTCGAATCACGTCGGGGTCA |
| 346 | GACCTCGTGGCGCAACGGCAGCGCGTCTGACTTCAGATCAGAAGGTTGCGTG |
| | TTCAAATCACGTCGGGGTCA |
| 347 | GGCCTCATGGTGCAACAGTAGTGTGTCTGACTTCAGATCAGAAGGTTGTATG |
| | TTCAAATCACGTAGGGGTCA |
| 348 | GGCCTCGTGGCGCAACGGTAGCGCGTCTGACTCTAGATCAGAAGGTTGCGTG |
| | TTCAAATCACGTCGGGGTCA |
| 349 | GACCTCGTGGCGCAATGGTAGCGCGTCTGACTCTAGATCAGAAGGTTGCGTG |
| | TTCAAGTCACGTCGGGGTCA |
| 350 | GACCTCGTGGCGCAACGGTAGCGCGTCTGACTCTAGATCAGAAGGTTGCGTG |
| | TTCAAATCACGTCGGGGTCA |
| 351 | GACCTCGTGGCGCAACGGTAGCGCGTCTGACTCTAGATCAGAAGGCTGCGTG |
| | TTCGAATCACGTCGGGGTCA |
| 352 | GACCTCGTGGCGCAACGGCAGCGCGTCTGACTCTAGATCAGAAGGTTGCGTG |
| | TTCAAATCACGTCGGGGTCA |
| 353 | GGCCTCATGGTGCAACAGTAGTGTGTCTGACTCTAGATCAGAAGGTTGTATG |
| | TTCAAATCACGTAGGGGTCA |
| 354 | GCATTGGTGGTTCAGTGGTAGAATTCTCGCCTTCAACGCGGGAGACCCGGGT |
| | TCAATTCCCGGCCAATGCA |
| 355 | GCGCCGCTGGTGTAGTGGTATCATGCAAGATTTCAATTCTTGCGACCCGGGT |
| | TCGATTCCCGGGCGGCGCA |
| 356 | GCATTGGTGGTTCAATGGTAGAATTCTCGCCTTCAACGCAGGAGACCCAGGT |
| | TCGATTCCTGGCCAATGCA |
| 357 | GCGTTGGTGGTTTAGTGGTAGAATTCTCGCCTTCAATGCGGGAGACCCGGGT |
| | TCAATTCCCGGCCACTGCA |
| 358 | GCCTTGGTGGTGCAGTGGTAGAATTCTCGCCTTCAACGTGGGAGACCCGGGT |
| | TCAATTCCCGGCCAATGCA |
| 359 | GGTGGTTCAGTGGTAGAATTCTCGCCTTCAACGCGGGAGACCCGGGTTTAAT |
| | TCCCGGTCA |
| 360 | GTGGTCTAGTGGTTAGGATTCAGCGCTTCAACCGCCGCAGCCCGGGTTCGAT |
| | TCCCGGTCA |
| 361 | GCGTCAGTGGTTTAGTGGTGGAATTCCTGCCTTCAATGCACGAGATCCGTGT |
| | TCAACTCCTGGTTGGTGCA |
| 362 | GCGTCAGTGGTTTTAGTGGTGGAATTCCTGCCTTCAATGCACGAGATCCGTG |
| | TTCAACTCCTGGTTGGTGCA |
| 363 | GCGTTGGCAGTTCAGTGGTAGAATTCTCGCCTTCAACCCGGGAGACCTGGAT |
| | TCCATTTCCGGCAAATGCA |
| 364 | GCATGGGTGGTTCAGTGGTAGAATTCTCGCCTTCAACGCGGGAGGCCCGGGT |
| | TCGATTCCCGGCCCATGCA |
| 365 | GCATTGGTGGTTCAGTGGTAGAATTCTCGCCTTCAACGCGGGAGGCCCGGGT |
| | TCGATTCCCGGCCAATGCA |
| 366 | GCATTGGTGGTTCAGTGGTAGAATTCTCGCCTTCAACGCGGGAGGCCCGGGT |
| | TTGATTCCCGGCCAGTGCA |
| 367 | GCATAGGTGGTTCAGTGGTAGAATTCTTGCCTTCAACGCAGGAGGCCCAGGT |
| | TTGATTCCTGGCCCATGCA |
| 368 | GCATTGGTGGTTCAGTGGTAGAATTCTCGCCTTCAATGCGGGCGGCCGGGCT |
| | TCGATTCCTGGCCAATGCA |
| 369 | GCATGGGTGATTCAGTGGTAGAATTTTCACCTTCAATGCAGGAGGTCCAGGT |
| | TCATTTCCTGGCCTATGCA |
| 370 | GCGTTGGTGGTATAGTGGTTAGCATAGCTGCCTTCAAAGCAGTTGACCCGGG |
| | TTCGATTCCCGGCCAACGCA |
| 371 | GCGTTGGTGGTATAGTGGTGAGCATAGCTGCCTTCAAAGCAGTTGACCCGGG |
| | TTCGATTCCCGGCCAACGCA |
| 372 | GCGTTGGTGGTATAGTGGTAAGCATAGCTGCCTTCAAAGCAGTTGACCCGGG |
| | TTCGATTCCCGGCCAACGCA |
| 373 | GCGTTGGTGGTATAGTGGTGAGCATAGTTGCCTTCAAAGCAGTTGACCCGGG |
| | CTCGATTCCCGCCCAACGCA |
| 374 | GCGTTGGTGGTATAGTGGTGAGCATAGTTGCCTTCAAAGCAGTTGACCCGGG |
| | CTCGATTCCCGGCCAACGCA |
| 375 | GGGCCAGTGGCGCAATGGATAACGCGTCTGACTTCAGATCAGAAGATTCCAG |
| | GTTCGACTCCTGGCTGGCTCG |
| 376 | GGGCCAGTGGCGCAATGGATAACGCGTCTGACTTCAGATCAGAAGATTCTAG |
| | GTTCGACTCCTGGCTGGCTCG |
| 377 | GGCCGCGTGGCCTAATGGATAAGGCGTCTGATTTCAGATCAGAAGATTGAGG |
| | GTTCGAGTCCCTTCGTGGTCG |
| 378 | GACCCAGTGGCCTAATGGATAAGGCATCAGCCTTCAGAGCTGGGGATTGTGG |
| | GTTCGAGTCCCATCTGGGTCG |
| 379 | GCCCCAGTGGCCTAATGGATAAGGCACTGGCCTTCAAAGCCAGGGATTGTGG |
| | GTTCGAGTCCCACCTGGGGTA |
| 380 | GCCCCAGTGGCCTAATGGATAAGGCACTGGCCTTCAAAGCCAGGGATTGTGG |
| | GTTCGAGTCCCACCTGGGGTG |
| 381 | GCCCCGGTGGCCTAATGGATAAGGCATTGGCCTTCAAAGCCAGGGATTGTGG |
| | GTTCGAGTCCCACCCGGGGTA |
| 382 | GCCCCAGTGGCCTAATGGATAAGGCATTGGCCTTCAAAGCCAGGGATTGTGG |
| | GTTCGAGTCCCATCTGGGGTG |
| 383 | GCCCCAGTGGCCTGATGGATAAGGTACTGGCCTTCAAAGCCAGGGATTGTGG |
| | GTTCGAGTTCCACCTGGGGTA |
| 384 | GGCCGCGTGGCCTAATGGATAAGGCGTCTGACTTCAGATCAGAAGATTGCAG |
| | GTTCGAGTCCTGCCGCGGTCG |
| 385 | GACCACGTGGCCTAATGGATAAGGCGTCTGACTTCAGATCAGAAGATTGAGG |
| | GTTCGAATCCCTCCGTGGTTA |
| 386 | GACCGCGTGGCCTAATGGATAAGGCGTCTGACTTCAGATCAGAAGATTGAGG |
| | GTTCGAGTCCCTTCGTGGTCG |
| 387 | GACCACGTGGCCTAATGGATAAGGCGTCTGACTTCAGATCAGAAGATTGAGG |
| | GTTCGAATCCCTTCGTGGTTA |
| 388 | GACCACGTGGCCTAATGGATAAGGCGTCTGACTTCAGATCAGAAGATTGAGG |
| | GTTCGAATCCCTTCGTGGTTG |
| 389 | GGCCGTGTGGCCTAATGGATAAGGCGTCTGACTTCAGATCAAAAGATTGCAG |
| | GTTTGAGTTCTGCCACGGTCG |
| 390 | GGCTCCGTGGCGCAATGGATAGCGCATTGGACTTCAAGAGGCTGAAGGCATT |
| | CAAAGGTTCCGGGTTCGAGTCCCGGCGGAGTCG |
| 391 | GGCTCCGTGGCGCAATGGATAGCGCATTGGACTTCAAATTCAAAGGTTCCGG |
| | GTTCGAGTCCCGGCGGAGTCG |
| 392 | GGCTCTGTGGCGCAATGGATAGCGCATTGGACTTCAAGTGACGAATAGAGCA |
| | ATTCAAAGGTTGTGGGTTCGAATCCCACCAGAGTCG |
| 393 | GGCTCTGTGGCGCAATGGATAGCGCATTGGACTTCAAATTCAAAGGTTGTGG |
| | GTTCGAATCCCACCAGAGTCG |
| 394 | GGCTCTGTGGCGCAATGGATAGCGCATTGGACTTCAAGCTGAGCCTAGTGTG |
| | GTCATTCAAAGGTTGTGGGTTCGAGTCCCACCAGAGTCG |
| 395 | GGCTCTGTGGCGCAATGGATAGCGCATTGGACTTCAAATTCAAAGGTTGTGG |
| | GTTCGAGTCCCACCAGAGTCG |
| 396 | GGCTCTGTGGCGCAATGGATAGCGCATTGGACTTCAAGATAGTTAGAGAAAT |
| | TCAAAGGTTGTGGGTTCGAGTCCCACCAGAGTCG |
| 397 | GTCTCTGTGGCGCAATGGACGAGCGCGCTGGACTTCAAATCCAGAGGTTCCG |
| | GGTTCGAGTCCCGGCAGAGATG |
| 398 | GGCTCTGTGGCGCAATGGATAGCGCATTGGACTTCAAGCCTAAATCAAGAGA |
| | TTCAAAGGTTGCGGGTTCGAGTCCCTCCAGAGTCG |
| 399 | GGCTCTGTGGCGCAATGGATAGCGCATTGGACTTCAAATTCAAAGGTTGCGG |
| | GTTCGAGTCCCTCCAGAGTCG |
| 400 | GGCAGCATAGCAGAGTGGTTCAGGTTACAGGTTCAAGATGTAAACTGAGTTC |
| | AAATCCCAGTTCTGCCA |
| 401 | TGGTGTAATAGGTAGCACAGAGAATTCTAGATTCTCAGGGGTAGGTTCAATT |
| | CCTAT |
| 402 | TAGGACATGGTGTGATAGGTAGCATGGAGAATTCTAGATTCTCAGGGGTAGG |
| | TTCAATTCCTACAGTTCTAG |
| 403 | TAGGACGTGGTGTGATAGGTAGCATGGGGAATTCTAGATTCTCAGGGGTGGG |
| | TTCAATTCCTATAGTTCTAG |
| 404 | TAGGACGTGGTGTAGTAGGTAGCATGGAGAATGCTAAATTCTCAGGGGTAGG |
| | TTCAATTCCTATAGTTCTAG |
| 405 | TAGGACATGGTGTAATAGGTAGAATGGAGAATTCTAAATTCTCAGGGGTAGG |
| | TTCAATTCCTATAGTTCTAG |
| 406 | TAGGATGTGGTGTATTAGGTAGCACAGAGAATTCTAGATTCTCAGGGGTAGG |
| | TTCGATTCCTATAATTCTAC |
| 407 | TAGGACTTGGTGTAATGGGTAGCACAGAGAATTCTAGATTCTCAGGGGTGGG |
| | TTCAATTCCTTTCGTCCTAG |
| 408 | TCTAGGATGTGGTGTGATAGGTAGCATGGAGAATTCTAGATTCTCAGGGGTA |
| | GGTTCAATTCCTATATTCTAGAA |
| 409 | TAGGACGTGGTGTGATAGGTAGCATGGAGAATTCTAGATTCTCAGGGATGGG |
| | TTCAATTCCTATAGTCCTAG |
| 410 | TAGGACGTGGTGTGATAGGTAGCACGGAGAATTCTAGATTCTCAGGGATGGG |
| | TTCAATTCCTGTAGTTCTAG |
| 411 | GGTTCCATGGTGTAATGGTTAGCACTCTGGACTCTAAATCCAGCGATCCGAG |
| | TTCAAATCTCGGTGGAACCT |
| 412 | GGTTCCATGGTGTAATGGTGACCACTTTGGACTCTAAATACAGTGATCAGAG |
| | TTCAAGTCTCACTGGAACCT |
| 413 | GGTTCCATGGTGTAATGGTGAGGGCTTTGGACTCTAACTACAGTGATCAGAG |
| | TTCAAGTCTCAGTGGGACCT |
| 414 | GGTTCCATGGTGTAATGGTAAGCACCCTGGACTCTAAATCCAGCAACCAGAG |
| | TTCCAGTCTCAGCGTGGACCT |
| 415 | GGTAGTGTAGTCTACTGGTTAAACGCTTGGGCTCTAACATTAACGTCCTGGG |
| | TTCAAATCCCAGCTTTGTCA |
| 416 | GGTTCCATGGTGTAATGGTTAGCACTCTGGACTCTAAATCCAGCGATCCGAG |
| | TTCAAGTCTCGGTGGAACCT |
| 417 | GGTTCCATGGTGTAATGGTGAGCACTCTGGACTCTAAATCCAGCGATCCGAG |
| | TTCGAGTCTCGGTGGAACCT |
| 418 | GGTTCCATGGTGTAATGGTAAGCACTCTGGACTCTAAATCCAGCGATCCGAG |
| | TTCGAGTCTCGGTGGAACCT |
| 419 | GGTTCCATGGTGTAATGGTTAGCACTCTGGACTCTAAATCCGGTAATCCGAG |
| | TTCAAATCTCGGTGGAACCT |
| 420 | GGCCCCATGGTGTAATGGTCAGCACTCTGGACTCTAAATCCAGCGATCCGAG |
| | TTCAAATCTCGGTGGGACCC |
| 421 | GGTTCCATGGTGTAATGGTAAGCACTCTGGACTCTAAATCCAGCCATCTGAG |
| | TTCGAGTCTCTGTGGAACCT |
| 422 | GGTTCCATGGTGTAATGGTGAGCACTTTGGACTCTAAATACAGTGATCAGAG |
| | TTCAAGTCTCACTGGGACCT |
| 423 | GGTTCCATGGGTTAATGGTGAGCACCCTGGACTCTAAATCAAGCGATCCGAG |
| | TTCAAATCTCGGTGGTACCT |
| 424 | GTTTCCATGGTGTAATGGTGAGCACTCTGGACTCTAAATCCAGAAATACATT |
| | CAAAGAATTAAGAACA |
| 425 | GGTCCCATGGTGTAATGGTTAGCACTCTGGACTCTAAATCCAGCGATCCGAG |
| | TTCAAATCTCGGTGGGACCT |
| 426 | GGTCCCATGGTGTAATGGTTAGCACTCTGGACTCTAAATCCAGCAATCCGAG |
| | TTCGAATCTCGGTGGGACCT |
| 427 | GGCCCCATGGTGTAATGGTTAGCACTCTGGACTCTAAATCCAGCGATCCGAG |
| | TTCAAATCTCGGTGGGACCT |
| 428 | GGTCCCATGGTGTAATGGTTAGCACTCTGGGCTCTAAATCCAGCAATCCGAG |
| | TTCGAATCTTGGTGGGACCT |
| 429 | GGCTGTGTACCTCAGTGGGCAAGGGTATGGACTCTAAAGCCAGACTATTTGG |
| | GTTCAAATCCCAGCTTGGCCT |
| 430 | GACCATGTGGCCTAAGGGAAAAGACATCTCACTCTAGGTCAGAAGATTGAGG |
| | GTTCAAGTCCTTTCATGGTCA |
| 431 | GGTACAGTGTTAAAGGGGAGAAAAATTGCTGACTCTAAATACAGTAGACCTA |
| | GGTTTGAATCCTGGCTTTACCA |
| 432 | TGGTGTAATAGGTAGCACAGAGAATTTTAGATTCTCAGGGGTAGGTTCAATT |
| | CCTAT |
| 433 | TAGGACATGGTGTGATAGGTAGCATGGAGAATTTTAGATTCTCAGGGGTAGG |
| | TTCAATTCCTACAGTTCTAG |
| 434 | TAGGACGTGGTGTGATAGGTAGCATGGGGAATTTTAGATTCTCAGGGGTGGG |
| | TTCAATTCCTATAGTTCTAG |
| 435 | TAGGACGTGGTGTAGTAGGTAGCATGGAGAATGTTAAATTCTCAGGGGTAGG |
| | TTCAATTCCTATAGTTCTAG |
| 436 | TAGGACATGGTGTAATAGGTAGAATGGAGAATTTTAAATTCTCAGGGGTAGG |
| | TTCAATTCCTATAGTTCTAG |
| 437 | TAGGATGTGGTGTATTAGGTAGCACAGAGAATTTTAGATTCTCAGGGGTAGG |
| | TTCGATTCCTATAATTCTAC |
| 438 | TAGGACTTGGTGTAATGGGTAGCACAGAGAATTTTAGATTCTCAGGGGTGGG |
| | TTCAATTCCTTTCGTCCTAG |
| 439 | TCTAGGATGTGGTGTGATAGGTAGCATGGAGAATTTTAGATTCTCAGGGGTA |
| | GGTTCAATTCCTATATTCTAGAA |
| 440 | TAGGACGTGGTGTGATAGGTAGCATGGAGAATTTTAGATTCTCAGGGATGGG |
| | TTCAATTCCTATAGTCCTAG |
| 441 | TAGGACGTGGTGTGATAGGTAGCACGGAGAATTTTAGATTCTCAGGGATGGG |
| | TTCAATTCCTGTAGTTCTAG |
| 442 | GGTTCCATGGTGTAATGGTTAGCACTCTGGACTTTAAATCCAGCGATCCGAG |
| | TTCAAATCTCGGTGGAACCT |
| 443 | GGTTCCATGGTGTAATGGTGACCACTTTGGACTTTAAATACAGTGATCAGAG |
| | TTCAAGTCTCACTGGAACCT |
| 444 | GGTTCCATGGTGTAATGGTGAGGGCTTTGGACTTTAACTACAGTGATCAGAG |
| | TTCAAGTCTCAGTGGGACCT |
| 445 | GGTTCCATGGTGTAATGGTAAGCACCCTGGACTTTAAATCCAGCAACCAGAG |
| | TTCCAGTCTCAGCGTGGACCT |
| 446 | GGTAGTGTAGTCTACTGGTTAAACGCTTGGGCTTTAACATTAACGTCCTGGG |
| | TTCAAATCCCAGCTTTGTCA |
| 447 | GGTTCCATGGTGTAATGGTTAGCACTCTGGACTTTAAATCCAGCGATCCGAG |
| | TTCAAGTCTCGGTGGAACCT |
| 448 | GGTTCCATGGTGTAATGGTGAGCACTCTGGACTTTAAATCCAGCGATCCGAG |
| | TTCGAGTCTCGGTGGAACCT |
| 449 | GGTTCCATGGTGTAATGGTAAGCACTCTGGACTTTAAATCCAGCGATCCGAG |
| | TTCGAGTCTCGGTGGAACCT |
| 450 | GGTTCCATGGTGTAATGGTTAGCACTCTGGACTTTAAATCCGGTAATCCGAG |
| | TTCAAATCTCGGTGGAACCT |
| 451 | GGCCCCATGGTGTAATGGTCAGCACTCTGGACTTTAAATCCAGCGATCCGAG |
| | TTCAAATCTCGGTGGGACCC |
| 452 | GGTTCCATGGTGTAATGGTAAGCACTCTGGACTTTAAATCCAGCCATCTGAG |
| | TTCGAGTCTCTGTGGAACCT |
| 453 | GGTTCCATGGTGTAATGGTGAGCACTTTGGACTTTAAATACAGTGATCAGAG |
| | TTCAAGTCTCACTGGGACCT |
| 454 | GGTTCCATGGGTTAATGGTGAGCACCCTGGACTTTAAATCAAGCGATCCGAG |
| | TTCAAATCTCGGTGGTACCT |
| 455 | GTTTCCATGGTGTAATGGTGAGCACTCTGGACTTTAAATCCAGAAATACATT |
| | CAAAGAATTAAGAACA |
| 456 | GGTCCCATGGTGTAATGGTTAGCACTCTGGACTTTAAATCCAGCGATCCGAG |
| | TTCAAATCTCGGTGGGACCT |
| 457 | GGTCCCATGGTGTAATGGTTAGCACTCTGGACTTTAAATCCAGCAATCCGAG |
| | TTCGAATCTCGGTGGGACCT |
| 458 | GGCCCCATGGTGTAATGGTTAGCACTCTGGACTTTAAATCCAGCGATCCGAG |
| | TTCAAATCTCGGTGGGACCT |
| 459 | GGTCCCATGGTGTAATGGTTAGCACTCTGGGCTTTAAATCCAGCAATCCGAG |
| | TTCGAATCTTGGTGGGACCT |
| 460 | GGCTGTGTACCTCAGTGGGCAAGGGTATGGACTTTAAAGCCAGACTATTTGG |
| | GTTCAAATCCCAGCTTGGCCT |
| 461 | GACCATGTGGCCTAAGGGAAAAGACATCTCACTTTAGGTCAGAAGATTGAGG |
| | GTTCAAGTCCTTTCATGGTCA |
| 462 | GGTACAGTGTTAAAGGGGAGAAAAATTGCTGACTTTAAATACAGTAGACCTA |
| | GGTTTGAATCCTGGCTTTACCA |
| 463 | TCCCTGGTGGTCTAGTGGTTAGGATTCGGCGCTTTAACCGCCGCGGCCCGGG |
| | TTCGATTCCCGGTCAGGGAA |
| 464 | TCCCTGGTGGTCTAGTGGTTAGGATTCGGCGCTTTAACCGCCGCGGCCCGGG |
| | TTCGATTCCCGGTCAGGAAA |
| 465 | CCCCTGGTGGTCTAGTGCTTAGGATTCGGTGCTTTAACCGCTGCTGCCTGCG |
| | TTCGATTCCCGGTCAGGGAA |
| 466 | TCCTTGATGTCTAGTGGTTAGGATTTGGTGCTTTAACTGCAGCAGCCTGGGT |
| | TCATTTCTCAGTCAGGGAA |
| 467 | TCCCATATGGTCTAGCGGTTAGGATTCCTGGTTTTAACCCAGGTGGCCCGGG |
| | TTCGACTCCCGGTATGGGAA |
| 468 | TCCGTGGTGGTCTAGTGGCTAGGATTCGGCGCTTTAACCGCCTGCAGCTCGA |
| | GTTCGATTCCTGGTCAGGGAA |
| 469 | CCCTGTGGTCTAGTGGCTAAGACTTTGTGCTTTAATTGCTGCATCCTAGGTT |
| | CAATTCCCAGTCAGGGA |
| 470 | TCCCACATGGTCTAGCGGTTAGGATTCCTGGTTTTAACCCAGGCGGCCCGGG |
| | TTCGACTCCCGGTGTGGGAA |
| 471 | TCCCTGGTGGTCTAGTGGCTAGGATTCGGCGCTTTAACCGCCGCGGCCCGGG |
| | TTCGATTCCCGGCCAGGGAA |
| 472 | TCCCTGGTGGTCTAGTGGCTAGGATTCGGCGCTTTAACCGCCGCGGCCCGGG |
| | TTCGATTCCCGGTCAGGGAA |
| 473 | GCGTTGGTGGTGTAGTGGTGAGCACAGCTGCCTTTAAAGCAGTTAACGCGGG |
| | TTCGATTCCCGGGTAACGAA |
| 474 | TCCTTGGTGGTCTAGTGGCTAGGATTCGGTGCTTTAACCTGTGCGGCCCGGG |
| | TTCAATTCCCGATGAAGGAA |
| 475 | TGTCTGGTGGTCAAGTGGCTAGGATTTGGCGCTTTAACTGCCGCGGCCCGCG |
| | TTCGATTCCCGGTCAGGGAA |
| 476 | TCCCTGGTGGTCTAGTGGCTAGGATTCGGCGCTTTAACCGCCTGCAGCTCGA |
| | GTTCGATTCCTGGTCAGGGAA |
| 477 | GCAATGGTGGTTCAGTGGTAGAATTCTCGCCTTTAACACAGGAGACCCGGGT |
| | TCAATTCCTGACCCATGTA |
| 478 | TCCCTGGTGGTCTAGTGGTTAGGATTCGGCGCTCTAACCGCCGCGGCCCGGG |
| | TTCGATTCCCGGTCAGGGAA |
| 479 | TCCCTGGTGGTCTAGTGGTTAGGATTCGGCGCTCTAACCGCCGCGGCCCGGG |
| | TTCGATTCCCGGTCAGGAAA |
| 480 | CCCCTGGTGGTCTAGTGCTTAGGATTCGGTGCTCTAACCGCTGCTGCCTGCG |
| | TTCGATTCCCGGTCAGGGAA |
| 481 | TCCTTGATGTCTAGTGGTTAGGATTTGGTGCTCTAACTGCAGCAGCCTGGGT |
| | TCATTTCTCAGTCAGGGAA |
| 482 | TCCCATATGGTCTAGCGGTTAGGATTCCTGGTTCTAACCCAGGTGGCCCGGG |
| | TTCGACTCCCGGTATGGGAA |
| 483 | TCCGTGGTGGTCTAGTGGCTAGGATTCGGCGCTCTAACCGCCTGCAGCTCGA |
| | GTTCGATTCCTGGTCAGGGAA |
| 484 | CCCTGTGGTCTAGTGGCTAAGACTTTGTGCTCTAATTGCTGCATCCTAGGTT |
| | CAATTCCCAGTCAGGGA |
| 485 | TCCCACATGGTCTAGCGGTTAGGATTCCTGGTTCTAACCCAGGCGGCCCGGG |
| | TTCGACTCCCGGTGTGGGAA |
| 486 | TCCCTGGTGGTCTAGTGGCTAGGATTCGGCGCTCTAACCGCCGCGGCCCGGG |
| | TTCGATTCCCGGCCAGGGAA |
| 487 | TCCCTGGTGGTCTAGTGGCTAGGATTCGGCGCTCTAACCGCCGCGGCCCGGG |
| | TTCGATTCCCGGTCAGGGAA |
| 488 | GCGTTGGTGGTGTAGTGGTGAGCACAGCTGCCTCTAAAGCAGTTAACGCGGG |
| | TTCGATTCCCGGGTAACGAA |
| 489 | TCCTTGGTGGTCTAGTGGCTAGGATTCGGTGCTCTAACCTGTGCGGCCCGGG |
| | TTCAATTCCCGATGAAGGAA |
| 490 | TGTCTGGTGGTCAAGTGGCTAGGATTTGGCGCTCTAACTGCCGCGGCCCGCG |
| | TTCGATTCCCGGTCAGGGAA |
| 491 | TCCCTGGTGGTCTAGTGGCTAGGATTCGGCGCTCTAACCGCCTGCAGCTCGA |
| | GTTCGATTCCTGGTCAGGGAA |
| 492 | GCAATGGTGGTTCAGTGGTAGAATTCTCGCCTCTAACACAGGAGACCCGGGT |
| | TCAATTCCTGACCCATGTA |
| 493 | CCTTCAATAGTTCAGCTGGTAGAGCAGAGGACTTTAGCTACTTCCTCAGTAG |
| | GAGACGTCCTTAGGTTGCTGGTTCGATTCCAGCTTGAAGGA |
| 494 | CCTTCAATAGTTCAGCTGGTAGAGCAGAGGACTTTAGGTCCTTAGGTTGCTG |
| | GTTCGATTCCAGCTTGAAGGA |
| 495 | GGTAAAATGGCTGAGTAAGCTTTAGACTTTAAATCTAAAGAGAGATTGAGCT |
| | CTCTTTTTACCA |
| 496 | GGTAAAATGACTGAGTAAGCATTAGACTTTAAATCTAAAGACAGAGGTCAAG |
| | ACCTCTTTTTACCA |
| 497 | GGTAAAATGGCTGAGTAAGCATTAGACTTTAAATCTAAAGACAGAGGTCAAG |
| | GCCTCTTTTTACCA |
| 498 | GGTAAAATGGCTGAGTAAGCATTAGACTTTAAATCTAAAGACAGAGGTCAAG |
| | GCCTTTTTACCA |
| 499 | CCTTCGATAGCTCAGTTGGTAGAGCGGAGGACTTTAGTTGGCTGTGTCCTTA |
| | GACATCCTTAGGTCGCTGGTTCGAATCCGGCTCGAAGGA |
| 500 | CCTTCGATAGCTCAGTTGGTAGAGCGGAGGACTTTAGATCCTTAGGTCGCTG |
| | GTTCGAATCCGGCTCGAAGGA |
| 501 | GGGGGTATAGCTCAGGGCTAGAGCTTTTTGACTTTAGAGCAAGAGGTCCCTG |
| | GTTCAAATCCAGGTTCTCCCT |
| 502 | TATAGCTCAGTGGTAGAGCATTTAACTTTAGATCAAGAGGTCCCTGGATCAA |
| | CTCTGGGTG |
| 503 | GTCAGTGTTGCACAACGGTTAAGTGAAGAGGCTTTAAACCCAGACTGGATGG |
| | GTTCAATTCCCATCTCTGCCG |
| 504 | CCTTCGATAGCTCAGTTGGTAGAGCGGAGGACTTTAGTGGATAGGGCGTGGC |
| | AATCCTTAGGTCGCTGGTTCGATTCCGGCTCGAAGGA |
| 505 | CCTTCGATAGCTCAGTTGGTAGAGCGGAGGACTTTAGATCCTTAGGTCGCTG |
| | GTTCGATTCCGGCTCGAAGGA |
| 506 | CCTTCGATAGCTCAGTTGGTAGAGCGGAGGACTTTAGGCTCATTAAGCAAGG |
| | TATCCTTAGGTCGCTGGTTCGAATCCGGCTCGGAGGA |
| 507 | CCTTCGATAGCTCAGTTGGTAGAGCGGAGGACTTTAGATCCTTAGGTCGCTG |
| | GTTCGAATCCGGCTCGGAGGA |
| 508 | CCTTCGATAGCTCAGCTGGTAGAGCGGAGGACTTTAGATTGTATAGACATTT |
| | GCGGACATCCTTAGGTCGCTGGTTCGATTCCAGCTCGAAGGA |
| 509 | CCTTCGATAGCTCAGCTGGTAGAGCGGAGGACTTTAGATCCTTAGGTCGCTG |
| | GTTCGATTCCAGCTCGAAGGA |
| 510 | CCTTCGATAGCTCAGCTGGTAGAGCGGAGGACTTTAGCTACTTCCTCAGCAG |
| | GAGACATCCTTAGGTCGCTGGTTCGATTCCGGCTCGAAGGA |
| 511 | CCTTCGATAGCTCAGCTGGTAGAGCGGAGGACTTTAGATCCTTAGGTCGCTG |
| | GTTCGATTCCGGCTCGAAGGA |
| 512 | CCTTCGATAGCTCAGCTGGTAGAGCGGAGGACTTTAGGCGCGCGCCCGTGGC |
| | CATCCTTAGGTCGCTGGTTCGATTCCGGCTCGAAGGA |
| 513 | CCTTCGATAGCTCAGCTGGTAGAGCGGAGGACTTTAGATCCTTAGGTCGCTG |
| | GTTCGATTCCGGCTCGAAGGA |
| 514 | CCTTCGATAGCTCAGCTGGTAGAGCGGAGGACTTTAGCCTGTAGAAACATTT |
| | GTGGACATCCTTAGGTCGCTGGTTCGATTCCGGCTCGAAGGA |
| 515 | CCTTCGATAGCTCAGCTGGTAGAGCGGAGGACTTTAGATCCTTAGGTCGCTG |
| | GTTCGATTCCGGCTCGAAGGA |
| 516 | CCTTCGATAGCTCAGCTGGTAGAGCGGAGGACTTTAGATTGTACAGACATTT |
| | GCGGACATCCTTAGGTCGCTGGTTCGATTCCGGCTCGAAGGA |
| 517 | CCTTCGATAGCTCAGCTGGTAGAGCGGAGGACTTTAGATCCTTAGGTCGCTG |
| | GTTCGATTCCGGCTCGAAGGA |
| 518 | CCTTCGATAGCTCAGCTGGTAGAGCGGAGGACTTTAGTACTTAATGTGTGGT |
| | CATCCTTAGGTCGCTGGTTCGATTCCGGCTCGAAGGA |
| 519 | CCTTCGATAGCTCAGCTGGTAGAGCGGAGGACTTTAGATCCTTAGGTCGCTG |
| | GTTCGATTCCGGCTCGAAGGA |
| 520 | CCTTCGATAGCTCAGCTGGTAGAGCGGAGGACTTTAGGGGTTTGAATGTGGT |
| | CATCCTTAGGTCGCTGGTTCGAATCCGGCTCGGAGGA |
| 521 | CCTTCGATAGCTCAGCTGGTAGAGCGGAGGACTTTAGATCCTTAGGTCGCTG |
| | GTTCGAATCCGGCTCGGAGGA |
| 522 | CCTTCGATAGCTCAGCTGGTAGAGCGGAGGACTTTAGACTGCGGAAACGTTT |
| | GTGGACATCCTTAGGTCGCTGGTTCAATTCCGGCTCGAAGGA |
| 523 | CCTTCGATAGCTCAGCTGGTAGAGCGGAGGACTTTAGATCCTTAGGTCGCTG |
| | GTTCAATTCCGGCTCGAAGGA |
| 524 | CTTTCGATAGCTCAGTTGGTAGAGCGGAGGACTTTAGGTTCATTAAACTAAG |
| | GCATCCTTAGGTCGCTGGTTCGAATCCGGCTCGAAGGA |
| 525 | CTTTCGATAGCTCAGTTGGTAGAGCGGAGGACTTTAGATCCTTAGGTCGCTG |
| | GTTCGAATCCGGCTCGAAGGA |
| 526 | TCTTCAATAGCTCAGCTGGTAGAGCGGAGGACTTTAGGTGCACGCCCGTGGC |
| | CATTCTTAGGTGCTGGTTTGATTCCGACTTGGAGAG |
| 527 | TCTTCAATAGCTCAGCTGGTAGAGCGGAGGACTTTAGATTCTTAGGTGCTGG |
| | TTTGATTCCGACTTGGAGAG |
| 528 | GGTAAAATGGCTGAGTGAAGCATTGGACTTTAAATCTAAAGACAGGGGTTAA |
| | GCCTCTTTTTACCA |
| 529 | GGTAAAATGGCTGAGCAAGCATTGGACTTTAAATCTAAAGACAGATGTTGAG |
| | CCATCTTTTTAGCA |
| 530 | GGTAAAATGGCTGAGTGAAGCATTGGACTTTAAATCTAAAGACAGGGGCTAA |
| | GCCTCTTTTTACCA |
| 531 | GGTAAAATGGCTGAGCAAGCATTAGACTTTAAATCTAAAGACAGAGGTTAAG |
| | GCCTCTTTTTACCA |
| 532 | GGTAAAATGGCTGAGTAAGCATTAGACTTTAAATCTAAAGACAGAGGTCAAG |
| | GCCTCTTTTTTCCT |
| 533 | GGTAAAATGGCTGAGCAAGCATTAGACTTTAAATCTGAAAACAGAGGTCAAA |
| | GGTCTCTTTTTACCA |
| 534 | GGTAAAATGGCTGAGTAAGCATTAGACTTTAAATCTAAAGACAGAGGTCAAG |
| | GCCTCTTTTTACCA |
| 535 | GGTAAAATGACTGAATAAGCCTTAGACTTTAAATCTGAAGACAGAGGTCAAG |
| | GCCTCTTTTTACCA |
| 536 | GGTAAAATGGCTGAGTAAGCATTGGACTTTAAATCTAAAGACAGAGGTCAAG |
| | ACCTCTTTTTACCA |
| 537 | GGTAAAATGGCTGAGTAAAGCATTAGACTTTAAATCTAAGGACAGAGGCTAA |
| | ACCTCTTTTTACCA |
| 538 | CCTTCAATAGTTCAGCTGGTAGAGCAGAGGACTCTAGCTACTTCCTCAGTAG |
| | GAGACGTCCTTAGGTTGCTGGTTCGATTCCAGCTTGAAGGA |
| 539 | CCTTCAATAGTTCAGCTGGTAGAGCAGAGGACTCTAGGTCCTTAGGTTGCTG |
| | GTTCGATTCCAGCTTGAAGGA |
| 540 | GGTAAAATGGCTGAGTAAGCTTTAGACTCTAAATCTAAAGAGAGATTGAGCT |
| | CTCTTTTTACCA |
| 541 | GGTAAAATGACTGAGTAAGCATTAGACTCTAAATCTAAAGACAGAGGTCAAG |
| | ACCTCTTTTTACCA |
| 542 | GGTAAAATGGCTGAGTAAGCATTAGACTCTAAATCTAAAGACAGAGGTCAAG |
| | GCCTCTTTTTACCA |
| 543 | GGTAAAATGGCTGAGTAAGCATTAGACTCTAAATCTAAAGACAGAGGTCAAG |
| | GCCTTTTTACCA |
| 544 | CCTTCGATAGCTCAGTTGGTAGAGCGGAGGACTCTAGTTGGCTGTGTCCTTA |
| | GACATCCTTAGGTCGCTGGTTCGAATCCGGCTCGAAGGA |
| 545 | CCTTCGATAGCTCAGTTGGTAGAGCGGAGGACTCTAGATCCTTAGGTCGCTG |
| | GTTCGAATCCGGCTCGAAGGA |
| 546 | GGGGGTATAGCTCAGGGCTAGAGCTTTTTGACTCTAGAGCAAGAGGTCCCTG |
| | GTTCAAATCCAGGTTCTCCCT |
| 547 | TATAGCTCAGTGGTAGAGCATTTAACTCTAGATCAAGAGGTCCCTGGATCAA |
| | CTCTGGGTG |
| 548 | GTCAGTGTTGCACAACGGTTAAGTGAAGAGGCTCTAAACCCAGACTGGATGG |
| | GTTCAATTCCCATCTCTGCCG |
| 549 | CCTTCGATAGCTCAGTTGGTAGAGCGGAGGACTCTAGTGGATAGGGCGTGGC |
| | AATCCTTAGGTCGCTGGTTCGATTCCGGCTCGAAGGA |
| 550 | CCTTCGATAGCTCAGTTGGTAGAGCGGAGGACTCTAGATCCTTAGGTCGCTG |
| | GTTCGATTCCGGCTCGAAGGA |
| 551 | CCTTCGATAGCTCAGTTGGTAGAGCGGAGGACTCTAGGCTCATTAAGCAAGG |
| | TATCCTTAGGTCGCTGGTTCGAATCCGGCTCGGAGGA |
| 552 | CCTTCGATAGCTCAGTTGGTAGAGCGGAGGACTCTAGATCCTTAGGTCGCTG |
| | GTTCGAATCCGGCTCGGAGGA |
| 553 | CCTTCGATAGCTCAGCTGGTAGAGCGGAGGACTCTAGATTGTATAGACATTT |
| | GCGGACATCCTTAGGTCGCTGGTTCGATTCCAGCTCGAAGGA |
| 554 | CCTTCGATAGCTCAGCTGGTAGAGCGGAGGACTCTAGATCCTTAGGTCGCTG |
| | GTTCGATTCCAGCTCGAAGGA |
| 555 | CCTTCGATAGCTCAGCTGGTAGAGCGGAGGACTCTAGCTACTTCCTCAGCAG |
| | GAGACATCCTTAGGTCGCTGGTTCGATTCCGGCTCGAAGGA |
| 556 | CCTTCGATAGCTCAGCTGGTAGAGCGGAGGACTCTAGATCCTTAGGTCGCTG |
| | GTTCGATTCCGGCTCGAAGGA |
| 557 | CCTTCGATAGCTCAGCTGGTAGAGCGGAGGACTCTAGGCGCGCGCCCGTGGC |
| | CATCCTTAGGTCGCTGGTTCGATTCCGGCTCGAAGGA |
| 558 | CCTTCGATAGCTCAGCTGGTAGAGCGGAGGACTCTAGATCCTTAGGTCGCTG |
| | GTTCGATTCCGGCTCGAAGGA |
| 559 | CCTTCGATAGCTCAGCTGGTAGAGCGGAGGACTCTAGCCTGTAGAAACATTT |
| | GTGGACATCCTTAGGTCGCTGGTTCGATTCCGGCTCGAAGGA |
| 560 | CCTTCGATAGCTCAGCTGGTAGAGCGGAGGACTCTAGATCCTTAGGTCGCTG |
| | GTTCGATTCCGGCTCGAAGGA |
| 561 | CCTTCGATAGCTCAGCTGGTAGAGCGGAGGACTCTAGATTGTACAGACATTT |
| | GCGGACATCCTTAGGTCGCTGGTTCGATTCCGGCTCGAAGGA |
| 562 | CCTTCGATAGCTCAGCTGGTAGAGCGGAGGACTCTAGATCCTTAGGTCGCTG |
| | GTTCGATTCCGGCTCGAAGGA |
| 563 | CCTTCGATAGCTCAGCTGGTAGAGCGGAGGACTCTAGTACTTAATGTGTGGT |
| | CATCCTTAGGTCGCTGGTTCGATTCCGGCTCGAAGGA |
| 564 | CCTTCGATAGCTCAGCTGGTAGAGCGGAGGACTCTAGATCCTTAGGTCGCTG |
| | GTTCGATTCCGGCTCGAAGGA |
| 565 | CCTTCGATAGCTCAGCTGGTAGAGCGGAGGACTCTAGGGGTTTGAATGTGGT |
| | CATCCTTAGGTCGCTGGTTCGAATCCGGCTCGGAGGA |
| 566 | CCTTCGATAGCTCAGCTGGTAGAGCGGAGGACTCTAGATCCTTAGGTCGCTG |
| | GTTCGAATCCGGCTCGGAGGA |
| 567 | CCTTCGATAGCTCAGCTGGTAGAGCGGAGGACTCTAGACTGCGGAAACGTTT |
| | GTGGACATCCTTAGGTCGCTGGTTCAATTCCGGCTCGAAGGA |
| 568 | CCTTCGATAGCTCAGCTGGTAGAGCGGAGGACTCTAGATCCTTAGGTCGCTG |
| | GTTCAATTCCGGCTCGAAGGA |
| 569 | CTTTCGATAGCTCAGTTGGTAGAGCGGAGGACTCTAGGTTCATTAAACTAAG |
| | GCATCCTTAGGTCGCTGGTTCGAATCCGGCTCGAAGGA |
| 570 | CTTTCGATAGCTCAGTTGGTAGAGCGGAGGACTCTAGATCCTTAGGTCGCTG |
| | GTTCGAATCCGGCTCGAAGGA |
| 571 | TCTTCAATAGCTCAGCTGGTAGAGCGGAGGACTCTAGGTGCACGCCCGTGGC |
| | CATTCTTAGGTGCTGGTTTGATTCCGACTTGGAGAG |
| 572 | TCTTCAATAGCTCAGCTGGTAGAGCGGAGGACTCTAGATTCTTAGGTGCTGG |
| | TTTGATTCCGACTTGGAGAG |
| 573 | GGTAAAATGGCTGAGTGAAGCATTGGACTCTAAATCTAAAGACAGGGGTTAA |
| | GCCTCTTTTTACCA |
| 574 | GGTAAAATGGCTGAGCAAGCATTGGACTCTAAATCTAAAGACAGATGTTGAG |
| | CCATCTTTTTAGCA |
| 575 | GGTAAAATGGCTGAGTGAAGCATTGGACTCTAAATCTAAAGACAGGGGCTAA |
| | GCCTCTTTTTACCA |
| 576 | GGTAAAATGGCTGAGCAAGCATTAGACTCTAAATCTAAAGACAGAGGTTAAG |
| | GCCTCTTTTTACCA |
| 577 | GGTAAAATGGCTGAGTAAGCATTAGACTCTAAATCTAAAGACAGAGGTCAAG |
| | GCCTCTTTTTTCCT |
| 578 | GGTAAAATGGCTGAGCAAGCATTAGACTCTAAATCTGAAAACAGAGGTCAAA |
| | GGTCTCTTTTTACCA |
| 579 | GGTAAAATGGCTGAGTAAGCATTAGACTCTAAATCTAAAGACAGAGGTCAAG |
| | GCCTCTTTTTACCA |
| 580 | GGTAAAATGACTGAATAAGCCTTAGACTCTAAATCTGAAGACAGAGGTCAAG |
| | GCCTCTTTTTACCA |
| 581 | GGTAAAATGGCTGAGTAAGCATTGGACTCTAAATCTAAAGACAGAGGTCAAG |
| | ACCTCTTTTTACCA |
| 582 | GGTAAAATGGCTGAGTAAAGCATTAGACTCTAAATCTAAGGACAGAGGCTAA |
| | ACCTCTTTTTACCA |
| 583 | GTTAAGATGGCAGAGCCTGGTAATTGCATTAAACTTAAAATTTTATAATCAG |
| | AGGTTCAACTCCTCTTCTTAACA |
| 584 | GTTAAGATGGCAGAGCCCGGCAATTGCATTAGACTTAAAACTTTATAATCAG |
| | AGGTTCAACTCCTCTCATTAACA |
| 585 | GGTAGCGTGGCCGAGCGGTCTAAGGCGCTGGATTTTAGCTCCAGTCTCTTCG |
| | GGGGCGTGGGTTCAAATCCCACCGCTGCCA |
| 586 | GGTAGCGTGGCCGAGTGGTCTAAGACGCTGGATTTTAGCTCCAGTCTCTTCG |
| | GGGGCGTGGGTTTGAATCCCACCGCTGCCA |
| 587 | GGGCCAGTGGCTCAATGGATAATGCGTCTGACTTTAAATCAGAAGATTCCAG |
| | CCTTGACTCCTGGCTGGCTCA |
| 588 | GGTAGGGTGGCCGAGCGGTCTAAGGCACTGTATTTTAACTCCAGTCTCTTCA |
| | GAGGCATGGGTTTGAATCCCACTGCTGCCA |
| 589 | GCCGAGCGGTCTAAGGCTCCGGATTTTAGCGCCGGTGTCTTCGGAGGCATGG |
| | GTTCGAATTCCAC |
| 590 | GTCAGGATGGCCGAGTGGTCTAAGGCGCCAGACTTTAGCTAAGCTTCCTCCG |
| | CGGTGGGGATTCTGGTCTCCAATGGAGGCGTGGGTTCGAATCCCACTTCTGA |
| | CA |
| 591 | GTCAGGATGGCCGAGTGGTCTAAGGCGCCAGACTTTAGTTCTGGTCTCCAAT |
| | GGAGGCGTGGGTTCGAATCCCACTTCTGACA |
| 592 | GTCAGGATGGCCGAGTGGTCTAAGGCGCCAGACTTTAGCTTGGCTTCCTCGT |
| | GTTGAGGATTCTGGTCTCCAATGGAGGCGTGGGTTCGAATCCCACTTCTGAC |
| | A |
| 593 | GTCAGGATGGCCGAGTGGTCTAAGGCGCCAGACTTTAGTTCTGGTCTCCAAT |
| | GGAGGCGTGGGTTCGAATCCCACTTCTGACA |
| 594 | GTCAGGATGGCCGAGTGGTCTAAGGCGCCAGACTTTAGCTTACTGCTTCCTG |
| | TGTTCGGGTCTTCTGGTCTCCGTATGGAGGCGTGGGTTCGAATCCCACTTCT |
| | GACA |
| 595 | GTCAGGATGGCCGAGTGGTCTAAGGCGCCAGACTTTAGTTCTGGTCTCCGTA |
| | TGGAGGCGTGGGTTCGAATCCCACTTCTGACA |
| 596 | GTCAGGATGGCCGAGTGGTCTAAGGCGCCAGACTTTAGTTGCTACTTCCCAG |
| | GTTTGGGGCTTCTGGTCTCCGCATGGAGGCGTGGGTTCGAATCCCACTTCTG |
| | ACA |
| 597 | GTCAGGATGGCCGAGTGGTCTAAGGCGCCAGACTTTAGTTCTGGTCTCCGCA |
| | TGGAGGCGTGGGTTCGAATCCCACTTCTGACA |
| 598 | GTCAGGATGGCCGAGTGGTCTAAGGCGCCAGACTTTAGGTAAGCACCTTGCC |
| | TGCGGGCTTTCTGGTCTCCGGATGGAGGCGTGGGTTCGAATCCCACTTCTGA |
| | CA |
| 599 | GTCAGGATGGCCGAGTGGTCTAAGGCGCCAGACTTTAGTTTCTGGTCTCCGG |
| | ATGGAGGCGTGGGTTCGAATCCCACTTCTGACA |
| 600 | GCCTCCTTAGTGCAGTAGGTAGCGCATCAGTCTTTAAATCTGAATGGTCCTG |
| | AGTTCAAGCCTCAGAGGGGGCA |
| 601 | GTCAGGATGGCCGAGCAGTCTTAAGGCGCTGCGTTTTAATCGCACCCTCCGC |
| | TGGAGGCGTGGGTTCGAATCCCACTTTTGACA |
| 602 | GGTTCCATGGTGTAATGGTGAGCACTCTGGACTTTAAATCCAGAAGTAGTGC |
| | TGGAACAA |
| 603 | GTCAGGGTGGCTGAGCAGTCTGAGGGGCTGCGTTTTAGTCGCAGTCTGCCCT |
| | GGAGGCGTGGGTTCGAATCCCACTCCTGAAA |
| 604 | ACCAGGATGGCCGAGTGGTTAAGGCGTTGGACTTTAGATCCAATGGACATAT |
| | GTCCGCGTGGGTTCGAACCCCACTCCTGGTA |
| 605 | ACCGGGATGGCCGAGTGGTTAAGGCGTTGGACTTTAGATCCAATGGGCTGGT |
| | GCCCGCGTGGGTTCGAACCCCACTCTCGGTA |
| 606 | ACCAGAATGGCCGAGTGGTTAAGGCGTTGGACTTTAGATCCAATGGATTCAT |
| | ATCCGCGTGGGTTCGAACCCCACTTCTGGTA |
| 607 | ACCGGGATGGCTGAGTGGTTAAGGCGTTGGACTTTAGATCCAATGGACAGGT |
| | GTCCGCGTGGGTTCGAGCCCCACTCCCGGTA |
| 608 | ACTCATTTGGCTGAGTGGTTAAGGCATTGGACTTTAGATCCAATGGAGTAGT |
| | GGCTGTGTGGGTTTAAACCCCACTACTGGTA |
| 609 | GAGAAAGTCATCGTAGTTACGAAGTTGGCTTTAACCCAGTTTTGGGAGGTTC |
| | AATTCCTTCCTTTCTCT |
| 610 | ACCAGGATGGCCAAGTAGTTAAAGGCACTGGACTTTAGAGCCAATGGACATA |
| | TGTCTGTGTGGGTTTGAACCCCACTCCTGGTG |
| 611 | GGTAGCGTGGCCGAGCGGTCTAAGGCGCTGGATTTTAGCTCCAGTCTCTTCG |
| | GAGGCGTGGGTTCGAATCCCACCGCTGCCA |
| 612 | GGTAGTGTGGCCGAGCGGTCTAAGGCGCTGGATTTTAGCTCCAGTCTCTTCG |
| | GGGGCGTGGGTTCGAATCCCACCACTGCCA |
| 613 | GGTAGCGTGGCCGAGTGGTCTAAGGCGCTGGATTTTAGCTCCAGTCATTTCG |
| | ATGGCGTGGGTTCGAATCCCACCGCTGCCA |
| 614 | GGTAGTGTGGTTGAATGGTCTAAGGCACTGAATTTTAGCTCCAGTCTCTTTG |
| | GGGACGTGGGTTTAAATCCCACTGCTGCAA |
| 615 | GTTAAGATGGCAGAGCCTGGTAATTGCACTAAACTTAAAATTTTATAATCAG |
| | AGGTTCAACTCCTCTTCTTAACA |
| 616 | GTTAAGATGGCAGAGCCCGGCAATTGCACTAGACTTAAAACTTTATAATCAG |
| | AGGTTCAACTCCTCTCATTAACA |
| 617 | GGTAGCGTGGCCGAGCGGTCTAAGGCGCTGGATTCTAGCTCCAGTCTCTTCG |
| | GGGGCGTGGGTTCAAATCCCACCGCTGCCA |
| 618 | GGTAGCGTGGCCGAGTGGTCTAAGACGCTGGATTCTAGCTCCAGTCTCTTCG |
| | GGGGCGTGGGTTTGAATCCCACCGCTGCCA |
| 619 | GGGCCAGTGGCTCAATGGATAATGCGTCTGACTCTAAATCAGAAGATTCCAG |
| | CCTTGACTCCTGGCTGGCTCA |
| 620 | GGTAGGGTGGCCGAGCGGTCTAAGGCACTGTATTCTAACTCCAGTCTCTTCA |
| | GAGGCATGGGTTTGAATCCCACTGCTGCCA |
| 621 | GCCGAGCGGTCTAAGGCTCCGGATTCTAGCGCCGGTGTCTTCGGAGGCATGG |
| | GTTCGAATTCCAC |
| 622 | GTCAGGATGGCCGAGTGGTCTAAGGCGCCAGACTCTAGCTAAGCTTCCTCCG |
| | CGGTGGGGATTCTGGTCTCCAATGGAGGCGTGGGTTCGAATCCCACTTCTGA |
| | CA |
| 623 | GTCAGGATGGCCGAGTGGTCTAAGGCGCCAGACTCTAGTTCTGGTCTCCAAT |
| | GGAGGCGTGGGTTCGAATCCCACTTCTGACA |
| 624 | GTCAGGATGGCCGAGTGGTCTAAGGCGCCAGACTCTAGCTTGGCTTCCTCGT |
| | GTTGAGGATTCTGGTCTCCAATGGAGGCGTGGGTTCGAATCCCACTTCTGAC |
| | A |
| 625 | GTCAGGATGGCCGAGTGGTCTAAGGCGCCAGACTCTAGTTCTGGTCTCCAAT |
| | GGAGGCGTGGGTTCGAATCCCACTTCTGACA |
| 626 | GTCAGGATGGCCGAGTGGTCTAAGGCGCCAGACTCTAGCTTACTGCTTCCTG |
| | TGTTCGGGTCTTCTGGTCTCCGTATGGAGGCGTGGGTTCGAATCCCACTTCT |
| | GACA |
| 627 | GTCAGGATGGCCGAGTGGTCTAAGGCGCCAGACTCTAGTTCTGGTCTCCGTA |
| | TGGAGGCGTGGGTTCGAATCCCACTTCTGACA |
| 628 | GTCAGGATGGCCGAGTGGTCTAAGGCGCCAGACTCTAGTTGCTACTTCCCAG |
| | GTTTGGGGCTTCTGGTCTCCGCATGGAGGCGTGGGTTCGAATCCCACTTCTG |
| | ACA |
| 629 | GTCAGGATGGCCGAGTGGTCTAAGGCGCCAGACTCTAGTTCTGGTCTCCGCA |
| | TGGAGGCGTGGGTTCGAATCCCACTTCTGACA |
| 630 | GTCAGGATGGCCGAGTGGTCTAAGGCGCCAGACTCTAGGTAAGCACCTTGCC |
| | TGCGGGCTTTCTGGTCTCCGGATGGAGGCGTGGGTTCGAATCCCACTTCTGA |
| | CA |
| 631 | GTCAGGATGGCCGAGTGGTCTAAGGCGCCAGACTCTAGTTTCTGGTCTCCGG |
| | ATGGAGGCGTGGGTTCGAATCCCACTTCTGACA |
| 632 | GCCTCCTTAGTGCAGTAGGTAGCGCATCAGTCTCTAAATCTGAATGGTCCTG |
| | AGTTCAAGCCTCAGAGGGGGCA |
| 633 | GTCAGGATGGCCGAGCAGTCTTAAGGCGCTGCGTTCTAATCGCACCCTCCGC |
| | TGGAGGCGTGGGTTCGAATCCCACTTTTGACA |
| 634 | GGTTCCATGGTGTAATGGTGAGCACTCTGGACTCTAAATCCAGAAGTAGTGC |
| | TGGAACAA |
| 635 | GTCAGGGTGGCTGAGCAGTCTGAGGGGCTGCGTTCTAGTCGCAGTCTGCCCT |
| | GGAGGCGTGGGTTCGAATCCCACTCCTGAAA |
| 636 | ACCAGGATGGCCGAGTGGTTAAGGCGTTGGACTCTAGATCCAATGGACATAT |
| | GTCCGCGTGGGTTCGAACCCCACTCCTGGTA |
| 637 | ACCGGGATGGCCGAGTGGTTAAGGCGTTGGACTCTAGATCCAATGGGCTGGT |
| | GCCCGCGTGGGTTCGAACCCCACTCTCGGTA |
| 638 | ACCAGAATGGCCGAGTGGTTAAGGCGTTGGACTCTAGATCCAATGGATTCAT |
| | ATCCGCGTGGGTTCGAACCCCACTTCTGGTA |
| 639 | ACCGGGATGGCTGAGTGGTTAAGGCGTTGGACTCTAGATCCAATGGACAGGT |
| | GTCCGCGTGGGTTCGAGCCCCACTCCCGGTA |
| 640 | ACTCATTTGGCTGAGTGGTTAAGGCATTGGACTCTAGATCCAATGGAGTAGT |
| | GGCTGTGTGGGTTTAAACCCCACTACTGGTA |
| 641 | GAGAAAGTCATCGTAGTTACGAAGTTGGCTCTAACCCAGTTTTGGGAGGTTC |
| | AATTCCTTCCTTTCTCT |
| 642 | ACCAGGATGGCCAAGTAGTTAAAGGCACTGGACTCTAGAGCCAATGGACATA |
| | TGTCTGTGTGGGTTTGAACCCCACTCCTGGTG |
| 643 | GGTAGCGTGGCCGAGCGGTCTAAGGCGCTGGATTCTAGCTCCAGTCTCTTCG |
| | GAGGCGTGGGTTCGAATCCCACCGCTGCCA |
| 644 | GGTAGTGTGGCCGAGCGGTCTAAGGCGCTGGATTCTAGCTCCAGTCTCTTCG |
| | GGGGCGTGGGTTCGAATCCCACCACTGCCA |
| 645 | GGTAGCGTGGCCGAGTGGTCTAAGGCGCTGGATTCTAGCTCCAGTCATTTCG |
| | ATGGCGTGGGTTCGAATCCCACCGCTGCCA |
| 646 | GGTAGTGTGGTTGAATGGTCTAAGGCACTGAATTCTAGCTCCAGTCTCTTTG |
| | GGGACGTGGGTTTAAATCCCACTGCTGCAA |
| 647 | GTTAAGATGGCAGAGCCTGGTAATTGCATCAAACTTAAAATTTTATAATCAG |
| | AGGTTCAACTCCTCTTCTTAACA |
| 648 | GTTAAGATGGCAGAGCCCGGCAATTGCATCAGACTTAAAACTTTATAATCAG |
| | AGGTTCAACTCCTCTCATTAACA |
| 649 | GGTAGCGTGGCCGAGCGGTCTAAGGCGCTGGATTTCAGCTCCAGTCTCTTCG |
| | GGGGCGTGGGTTCAAATCCCACCGCTGCCA |
| 650 | GGTAGCGTGGCCGAGTGGTCTAAGACGCTGGATTTCAGCTCCAGTCTCTTCG |
| | GGGGCGTGGGTTTGAATCCCACCGCTGCCA |
| 651 | GGGCCAGTGGCTCAATGGATAATGCGTCTGACTTCAAATCAGAAGATTCCAG |
| | CCTTGACTCCTGGCTGGCTCA |
| 652 | GGTAGGGTGGCCGAGCGGTCTAAGGCACTGTATTTCAACTCCAGTCTCTTCA |
| | GAGGCATGGGTTTGAATCCCACTGCTGCCA |
| 653 | GCCGAGCGGTCTAAGGCTCCGGATTTCAGCGCCGGTGTCTTCGGAGGCATGG |
| | GTTCGAATTCCAC |
| 654 | GTCAGGATGGCCGAGTGGTCTAAGGCGCCAGACTTCAGCTAAGCTTCCTCCG |
| | CGGTGGGGATTCTGGTCTCCAATGGAGGCGTGGGTTCGAATCCCACTTCTGA |
| | CA |
| 655 | GTCAGGATGGCCGAGTGGTCTAAGGCGCCAGACTTCAGTTCTGGTCTCCAAT |
| | GGAGGCGTGGGTTCGAATCCCACTTCTGACA |
| 656 | GTCAGGATGGCCGAGTGGTCTAAGGCGCCAGACTTCAGCTTGGCTTCCTCGT |
| | GTTGAGGATTCTGGTCTCCAATGGAGGCGTGGGTTCGAATCCCACTTCTGAC |
| | A |
| 657 | GTCAGGATGGCCGAGTGGTCTAAGGCGCCAGACTTCAGTTCTGGTCTCCAAT |
| | GGAGGCGTGGGTTCGAATCCCACTTCTGACA |
| 658 | GTCAGGATGGCCGAGTGGTCTAAGGCGCCAGACTTCAGCTTACTGCTTCCTG |
| | TGTTCGGGTCTTCTGGTCTCCGTATGGAGGCGTGGGTTCGAATCCCACTTCT |
| | GACA |
| 659 | GTCAGGATGGCCGAGTGGTCTAAGGCGCCAGACTTCAGTTCTGGTCTCCGTA |
| | TGGAGGCGTGGGTTCGAATCCCACTTCTGACA |
| 660 | GTCAGGATGGCCGAGTGGTCTAAGGCGCCAGACTTCAGTTGCTACTTCCCAG |
| | GTTTGGGGCTTCTGGTCTCCGCATGGAGGCGTGGGTTCGAATCCCACTTCTG |
| | ACA |
| 661 | GTCAGGATGGCCGAGTGGTCTAAGGCGCCAGACTTCAGTTCTGGTCTCCGCA |
| | TGGAGGCGTGGGTTCGAATCCCACTTCTGACA |
| 662 | GTCAGGATGGCCGAGTGGTCTAAGGCGCCAGACTTCAGGTAAGCACCTTGCC |
| | TGCGGGCTTTCTGGTCTCCGGATGGAGGCGTGGGTTCGAATCCCACTTCTGA |
| | CA |
| 663 | GTCAGGATGGCCGAGTGGTCTAAGGCGCCAGACTTCAGTTTCTGGTCTCCGG |
| | ATGGAGGCGTGGGTTCGAATCCCACTTCTGACA |
| 664 | GCCTCCTTAGTGCAGTAGGTAGCGCATCAGTCTTCAAATCTGAATGGTCCTG |
| | AGTTCAAGCCTCAGAGGGGGCA |
| 665 | GTCAGGATGGCCGAGCAGTCTTAAGGCGCTGCGTTTCAATCGCACCCTCCGC |
| | TGGAGGCGTGGGTTCGAATCCCACTTTTGACA |
| 666 | GGTTCCATGGTGTAATGGTGAGCACTCTGGACTTCAAATCCAGAAGTAGTGC |
| | TGGAACAA |
| 667 | GTCAGGGTGGCTGAGCAGTCTGAGGGGCTGCGTTTCAGTCGCAGTCTGCCCT |
| | GGAGGCGTGGGTTCGAATCCCACTCCTGAAA |
| 668 | ACCAGGATGGCCGAGTGGTTAAGGCGTTGGACTTCAGATCCAATGGACATAT |
| | GTCCGCGTGGGTTCGAACCCCACTCCTGGTA |
| 669 | ACCGGGATGGCCGAGTGGTTAAGGCGTTGGACTTCAGATCCAATGGGCTGGT |
| | GCCCGCGTGGGTTCGAACCCCACTCTCGGTA |
| 670 | ACCAGAATGGCCGAGTGGTTAAGGCGTTGGACTTCAGATCCAATGGATTCAT |
| | ATCCGCGTGGGTTCGAACCCCACTTCTGGTA |
| 671 | ACCGGGATGGCTGAGTGGTTAAGGCGTTGGACTTCAGATCCAATGGACAGGT |
| | GTCCGCGTGGGTTCGAGCCCCACTCCCGGTA |
| 672 | ACTCATTTGGCTGAGTGGTTAAGGCATTGGACTTCAGATCCAATGGAGTAGT |
| | GGCTGTGTGGGTTTAAACCCCACTACTGGTA |
| 673 | GAGAAAGTCATCGTAGTTACGAAGTTGGCTTCAACCCAGTTTTGGGAGGTTC |
| | AATTCCTTCCTTTCTCT |
| 674 | ACCAGGATGGCCAAGTAGTTAAAGGCACTGGACTTCAGAGCCAATGGACATA |
| | TGTCTGTGTGGGTTTGAACCCCACTCCTGGTG |
| 675 | GGTAGCGTGGCCGAGCGGTCTAAGGCGCTGGATTTCAGCTCCAGTCTCTTCG |
| | GAGGCGTGGGTTCGAATCCCACCGCTGCCA |
| 676 | GGTAGTGTGGCCGAGCGGTCTAAGGCGCTGGATTTCAGCTCCAGTCTCTTCG |
| | GGGGCGTGGGTTCGAATCCCACCACTGCCA |
| 677 | GGTAGCGTGGCCGAGTGGTCTAAGGCGCTGGATTTCAGCTCCAGTCATTTCG |
| | ATGGCGTGGGTTCGAATCCCACCGCTGCCA |
| 678 | GGTAGTGTGGTTGAATGGTCTAAGGCACTGAATTTCAGCTCCAGTCTCTTTG |
| | GGGACGTGGGTTTAAATCCCACTGCTGCAA |
| 679 | GAGAAGGTCACAGAGGTTATGGGATTGGCTTTAAACCAGTCTGTGGGGGGTT |
| | CGATTCCCTCCTTTTTCA |
| 680 | GAGAAGGTCATAGAGGTTATGGGATTGGCTTTAAACCAGTCTCTGGGGGGTT |
| | CGATTCCCTCCTTTTTCA |
| 681 | GAAAAAGTCATAGGGGTTATGAGGCTGGCTTTAAACCAGCCTTAGGAGGTTC |
| | AATTCCTTCCTTTTTTG |
| 682 | GGCCGGTTAGCTCAGTTGGTTAGAGCGTGCTGCTTTAAATGCCAGGGTCGAG |
| | GTTTCGATCCCCGTACGGGCCT |
| 683 | GTAGTCGTGGCCGAGTGGTTAAGGCGATGGACTTTAAATCCATTGGGGTTTC |
| | CCCGCGCAGGTTCGAATCCTGCCGACTACG |
| 684 | GTAGTCGTGGCCGAGTGGTTAAGGCGATGGACTTTAAATCCATTGGGGTTTC |
| | CCCACGCAGGTTCGAATCCTGCCGACTACG |
| 685 | GTAGTCGTGGCCGAGTGGTTAAGGTGATGGACTTTAAACCCATTGGGGTCTC |
| | CCCGCGCAGGTTCGAATCCTGCCGACTACG |
| 686 | GGGTGTATGGCTCAGGGGTAGAGAATTTGACTTTAGATCAAGAGGTCCCTGG |
| | TTCAAATCCAGGTGCCCCCT |
| 687 | AGTTGTAGCTGAGTGGTTAAGGCAACGAGCTTTAAATTCGTTGGTTTCTCTC |
| | TGTGCAGGTTTGAATCCTGCTAATTA |
| 688 | CAAGAAATTCATAGAGGTTATGGGATTGGCTTTAAACCAGTTTCAGGAGGTT |
| | CGATTCCTTCCTTTTTGG |
| 689 | GCTGTGATGGCCGAGTGGTTAAGGCGTTGGACTTTAAATCCAATGGGGTCTC |
| | CCCGCGCAGGTTCGAATCCTGCTCACAGCG |
| 690 | GCTGTGATGGCCGAGTGGTTAAGGCGTTGGACTTTAAATCCAATGGGGTCTC |
| | CCCGCGCAGGTTCAAATCCTGCTCACAGCG |
| 691 | GCTGTGATGGCCGAGTGGTTAAGGTGTTGGACTTTAAATCCAATGGGGGTTC |
| | CCCGCGCAGGTTCAAATCCTGCTCACAGCG |
| 692 | GTCACGGTGGCCGAGTGGTTAAGGCGTTGGACTTTAAATCCAATGGGGTTTC |
| | CCCGCACAGGTTCGAATCCTGTTCGTGACG |
| 693 | GACGAGGTGGCCGAGTGGTTAAGGCGATGGACTTTAAATCCATTGTGCTCTG |
| | CACGCGTGGGTTCGAATCCCACCCTCGTCG |
| 694 | GACGAGGTGGCCGAGTGGTTAAGGCGATGGACTTTAAATCCATTGTGCTCTG |
| | CACGCGTGGGTTCGAATCCCACCTTCGTCG |
| 695 | GGCCGGTTAGCTCAGTTGGTTAGAGCGTGCTTTAACTAATGCCAGGGTCGAG |
| | GTTTCGATCCCCGTACGGGCCT |
| 696 | GACGAGGTGGCCGAGTGGTTAAGGCGATGGACTTTAAATCCATTGTGCTCTG |
| | CACACGTGGGTTCGAATCCCATCCTCGTCG |
| 697 | GAGGCCTGGCCGAGTGGTTAAGGCGATGGACTTTAAATCCATTGTGCTCTGC |
| | ACGCGTGGGTTCGAATCCCATCCTCG |
| 698 | GCAGCGATGGCCGAGTGGTTAAGGCGTTGGACTTTAAATCCAATGGGGTCTC |
| | CCCGCGCAGGTTCGAACCCTGCTCGCTGCG |
| 699 | GTAGTCGTGGCCGAGTGGTTAAGGCGATGGACTTTAAATCCATTGGGGTTTC |
| | CCCGCGCAGGTTCGAATCCTGCCGACTACG |
| 700 | GTAGTCGTGGCCGAGTGGTTAAGGCGATGGACTTTAAATCCATTGGGGTCTC |
| | CCCGCGCAGGTTCGAATCCTGCCGACTACG |
| 701 | GTAGTCGTGGCCGAGTGGTTAAGGCGATGGACTTTAAATCCATTGGGGTTTC |
| | CCCGCGCAGGTTCGAATCCTGTCGGCTACG |
| 702 | GAGAAGGTCACAGAGGTTATGGGATTGGCTCTAAACCAGTCTGTGGGGGGTT |
| | CGATTCCCTCCTTTTTCA |
| 703 | GAGAAGGTCATAGAGGTTATGGGATTGGCTCTAAACCAGTCTCTGGGGGGTT |
| | CGATTCCCTCCTTTTTCA |
| 704 | GAAAAAGTCATAGGGGTTATGAGGCTGGCTCTAAACCAGCCTTAGGAGGTTC |
| | AATTCCTTCCTTTTTTG |
| 705 | GGCCGGTTAGCTCAGTTGGTTAGAGCGTGCTGCTCTAAATGCCAGGGTCGAG |
| | GTTTCGATCCCCGTACGGGCCT |
| 706 | GTAGTCGTGGCCGAGTGGTTAAGGCGATGGACTCTAAATCCATTGGGGTTTC |
| | CCCGCGCAGGTTCGAATCCTGCCGACTACG |
| 707 | GTAGTCGTGGCCGAGTGGTTAAGGCGATGGACTCTAAATCCATTGGGGTTTC |
| | CCCACGCAGGTTCGAATCCTGCCGACTACG |
| 708 | GTAGTCGTGGCCGAGTGGTTAAGGTGATGGACTCTAAACCCATTGGGGTCTC |
| | CCCGCGCAGGTTCGAATCCTGCCGACTACG |
| 709 | GGGTGTATGGCTCAGGGGTAGAGAATTTGACTCTAGATCAAGAGGTCCCTGG |
| | TTCAAATCCAGGTGCCCCCT |
| 710 | AGTTGTAGCTGAGTGGTTAAGGCAACGAGCTCTAAATTCGTTGGTTTCTCTC |
| | TGTGCAGGTTTGAATCCTGCTAATTA |
| 711 | CAAGAAATTCATAGAGGTTATGGGATTGGCTCTAAACCAGTTTCAGGAGGTT |
| | CGATTCCTTCCTTTTTGG |
| 712 | GCTGTGATGGCCGAGTGGTTAAGGCGTTGGACTCTAAATCCAATGGGGTCTC |
| | CCCGCGCAGGTTCGAATCCTGCTCACAGCG |
| 713 | GCTGTGATGGCCGAGTGGTTAAGGCGTTGGACTCTAAATCCAATGGGGTCTC |
| | CCCGCGCAGGTTCAAATCCTGCTCACAGCG |
| 714 | GCTGTGATGGCCGAGTGGTTAAGGTGTTGGACTCTAAATCCAATGGGGGTTC |
| | CCCGCGCAGGTTCAAATCCTGCTCACAGCG |
| 715 | GTCACGGTGGCCGAGTGGTTAAGGCGTTGGACTCTAAATCCAATGGGGTTTC |
| | CCCGCACAGGTTCGAATCCTGTTCGTGACG |
| 716 | GACGAGGTGGCCGAGTGGTTAAGGCGATGGACTCTAAATCCATTGTGCTCTG |
| | CACGCGTGGGTTCGAATCCCACCCTCGTCG |
| 717 | GACGAGGTGGCCGAGTGGTTAAGGCGATGGACTCTAAATCCATTGTGCTCTG |
| | CACGCGTGGGTTCGAATCCCACCTTCGTCG |
| 718 | GGCCGGTTAGCTCAGTTGGTTAGAGCGTGCTCTAACTAATGCCAGGGTCGAG |
| | GTTTCGATCCCCGTACGGGCCT |
| 719 | GACGAGGTGGCCGAGTGGTTAAGGCGATGGACTCTAAATCCATTGTGCTCTG |
| | CACACGTGGGTTCGAATCCCATCCTCGTCG |
| 720 | GAGGCCTGGCCGAGTGGTTAAGGCGATGGACTCTAAATCCATTGTGCTCTGC |
| | ACGCGTGGGTTCGAATCCCATCCTCG |
| 721 | GCAGCGATGGCCGAGTGGTTAAGGCGTTGGACTCTAAATCCAATGGGGTCTC |
| | CCCGCGCAGGTTCGAACCCTGCTCGCTGCG |
| 722 | GTAGTCGTGGCCGAGTGGTTAAGGCGATGGACTCTAAATCCATTGGGGTTTC |
| | CCCGCGCAGGTTCGAATCCTGCCGACTACG |
| 723 | GTAGTCGTGGCCGAGTGGTTAAGGCGATGGACTCTAAATCCATTGGGGTCTC |
| | CCCGCGCAGGTTCGAATCCTGCCGACTACG |
| 724 | GTAGTCGTGGCCGAGTGGTTAAGGCGATGGACTCTAAATCCATTGGGGTTTC |
| | CCCGCGCAGGTTCGAATCCTGTCGGCTACG |
| 725 | GAGAAGGTCACAGAGGTTATGGGATTGGCTTCAAACCAGTCTGTGGGGGGTT |
| | CGATTCCCTCCTTTTTCA |
| 726 | GAGAAGGTCATAGAGGTTATGGGATTGGCTTCAAACCAGTCTCTGGGGGGTT |
| | CGATTCCCTCCTTTTTCA |
| 727 | GAAAAAGTCATAGGGGTTATGAGGCTGGCTTCAAACCAGCCTTAGGAGGTTC |
| | AATTCCTTCCTTTTTTG |
| 728 | GGCCGGTTAGCTCAGTTGGTTAGAGCGTGCTGCTTCAAATGCCAGGGTCGAG |
| | GTTTCGATCCCCGTACGGGCCT |
| 729 | GTAGTCGTGGCCGAGTGGTTAAGGCGATGGACTTCAAATCCATTGGGGTTTC |
| | CCCGCGCAGGTTCGAATCCTGCCGACTACG |
| 730 | GTAGTCGTGGCCGAGTGGTTAAGGCGATGGACTTCAAATCCATTGGGGTTTC |
| | CCCACGCAGGTTCGAATCCTGCCGACTACG |
| 731 | GTAGTCGTGGCCGAGTGGTTAAGGTGATGGACTTCAAACCCATTGGGGTCTC |
| | CCCGCGCAGGTTCGAATCCTGCCGACTACG |
| 732 | GGGTGTATGGCTCAGGGGTAGAGAATTTGACTTCAGATCAAGAGGTCCCTGG |
| | TTCAAATCCAGGTGCCCCCT |
| 733 | AGTTGTAGCTGAGTGGTTAAGGCAACGAGCTTCAAATTCGTTGGTTTCTCTC |
| | TGTGCAGGTTTGAATCCTGCTAATTA |
| 734 | CAAGAAATTCATAGAGGTTATGGGATTGGCTTCAAACCAGTTTCAGGAGGTT |
| | CGATTCCTTCCTTTTTGG |
| 735 | GCTGTGATGGCCGAGTGGTTAAGGCGTTGGACTTCAAATCCAATGGGGTCTC |
| | CCCGCGCAGGTTCGAATCCTGCTCACAGCG |
| 736 | GCTGTGATGGCCGAGTGGTTAAGGCGTTGGACTTCAAATCCAATGGGGTCTC |
| | CCCGCGCAGGTTCAAATCCTGCTCACAGCG |
| 737 | GCTGTGATGGCCGAGTGGTTAAGGTGTTGGACTTCAAATCCAATGGGGGTTC |
| | CCCGCGCAGGTTCAAATCCTGCTCACAGCG |
| 738 | GTCACGGTGGCCGAGTGGTTAAGGCGTTGGACTTCAAATCCAATGGGGTTTC |
| | CCCGCACAGGTTCGAATCCTGTTCGTGACG |
| 739 | GACGAGGTGGCCGAGTGGTTAAGGCGATGGACTTCAAATCCATTGTGCTCTG |
| | CACGCGTGGGTTCGAATCCCACCCTCGTCG |
| 740 | GACGAGGTGGCCGAGTGGTTAAGGCGATGGACTTCAAATCCATTGTGCTCTG |
| | CACGCGTGGGTTCGAATCCCACCTTCGTCG |
| 741 | GGCCGGTTAGCTCAGTTGGTTAGAGCGTGCTTCAACTAATGCCAGGGTCGAG |
| | GTTTCGATCCCCGTACGGGCCT |
| 742 | GACGAGGTGGCCGAGTGGTTAAGGCGATGGACTTCAAATCCATTGTGCTCTG |
| | CACACGTGGGTTCGAATCCCATCCTCGTCG |
| 743 | GAGGCCTGGCCGAGTGGTTAAGGCGATGGACTTCAAATCCATTGTGCTCTGC |
| | ACGCGTGGGTTCGAATCCCATCCTCG |
| 744 | GCAGCGATGGCCGAGTGGTTAAGGCGTTGGACTTCAAATCCAATGGGGTCTC |
| | CCCGCGCAGGTTCGAACCCTGCTCGCTGCG |
| 745 | GTAGTCGTGGCCGAGTGGTTAAGGCGATGGACTTCAAATCCATTGGGGTTTC |
| | CCCGCGCAGGTTCGAATCCTGCCGACTACG |
| 746 | GTAGTCGTGGCCGAGTGGTTAAGGCGATGGACTTCAAATCCATTGGGGTCTC |
| | CCCGCGCAGGTTCGAATCCTGCCGACTACG |
| 747 | GTAGTCGTGGCCGAGTGGTTAAGGCGATGGACTTCAAATCCATTGGGGTTTC |
| | CCCGCGCAGGTTCGAATCCTGTCGGCTACG |
| 748 | GCCCAGCTAGCTCAGTCGGTAGAGCATAAGACTTTAAATCTCAGGGTTGTGG |
| | ATTCGTGCCCCATGCTGGGTG |
| 749 | CTGCAGCTAGCTCAGTCGGTAGAGCATGAGACTTTAAATCTCAGGGTCATGG |
| | GTTCGTGCCCCATGTTGGG |
| 750 | CCAGCATGTCTCAGTCGGTATAGTGTGAGACTTTAAATCTCAGGGTCGTGGG |
| | TTCAAGCCCCACATTGGG |
| 751 | GTCTAGCTAGATCAGTTGGTAGAGCATAAGACTTTAAATCTCAGGGTCATGG |
| | GTTTGAGCCCTACGTTGGGCG |
| 752 | GCCCAGCTAGCTCAGCCGGTAGAGCACAAGACTTTAAATCTCAGGGTCGTGG |
| | GTTTGAGCCCTGTGTTGAGCA |
| 753 | CCGAATAGCTTAGTTGATGAAGCGTGAGACTTTAAATCTCAGGGTAGTGGGT |
| | TCAAGCCCCACATTGGA |
| 754 | GCCTGGCTACCTCAGTTGGTAGAGCATGGGACTTTAAATCCCAGAGTCAGTG |
| | GGTTCAAGCCTCACATTGAGTG |
| 755 | GCCCGGCTAGCTCAGTCGGTAGAGCATGAGACCTTAAATCTCAGGGTCGTGG |
| | GTTCGAGCCCCACGTTGGGCG |
| 756 | GCCCGGCTAGCTCAGTCGGTAGAGCATGGGACTTTAAATCTCAGGGTCGTGG |
| | GTTCGAGCCCCACGTTGGGCG |
| 757 | GCCCGGCTAGCTCAGTCGATAGAGCATGAGACTTTAAATCTCAGGGTCGTGG |
| | GTTCGAGCCGCACGTTGGGCG |
| 758 | GCCCAGCTAGCTCAGTCGGTAGAGCATGAGACTTTAAATCTCAGGGTCATGG |
| | GTTTGAGCCCCACGTTTGGTG |
| 759 | GCCTGGCTAGCTCAGTCGGCAAAGCATGAGACTTTAAATCTCAGGGTCGTGG |
| | GCTCGAGCTCCATGTTGGGCG |
| 760 | GCCCGACTACCTCAGTCGGTGGAGCATGGGACTTTACATCCCAGGGTTGTGG |
| | GTTCGAGCCCCACATTGGGCA |
| 761 | CCCCGGCTGGCTCAGTCAGTAGATCATGAGACTTTAAATCTCAGGGTCGTGG |
| | GTTCACGCCCCACACTGGGCG |
| 762 | GCGCTAGTCAGTAGAGCATGAGACTTTAAATCTCAGGGTCGTGGGTTCGAGC |
| | CCCACATCGGGCG |
| 763 | GCCTGGATAGCTCAGTTGGTAGAGCATCAGACTTTAAATCTGAGGGTCCAGG |
| | GTTCAAGTCCCTGTTCAGGCA |
| 764 | GCCAGGATAGTTCAGGTGGTAGAGCATCAGACTTTAAACCTGAGGGTTCAGG |
| | GTTCAAGTCTCTGTTTGGGCG |
| 765 | ACCCAGATAGCTCAGTCAGTAGAGCATCAGACTTTAAATCTGAGGGTCCAAG |
| | GTTCATGTCCCTTTTTGGGTG |
| 766 | ACCTGGGTAGCTTAGTTGGTAGAGCATTGGACTTTAAATTTGAGGGCCCAGG |
| | TTTCAAGTCCCTGTTTGGGTG |
| 767 | GCCTGGGTAGCTCAGTCGGTAGAGCTATCAGACTTTAAGCCTGAGGATTCAG |
| | GGTTCAATCCCTTGCTGGGGCG |
| 768 | GATAGCTCAGTTGATAGAGCATCAGACTTTAAATCTGAGGGTCCAGGGTTCA |
| | TGTCCCTGTT |
| 769 | GTTGGGGTAACTCAGTTGGTAGAGTAGCAGACTTTACATCTGAGGGTCCAGG |
| | GTTTAAGTCCATGTCCAGGCA |
| 770 | GCCTGGATAGCTCAGTTGGTAGAGCATCAGACTTTAAATCTGAGGGTCCAGG |
| | GTTCAAGTCCCTGTTCAGGCG |
| 771 | GCCTGGATAGCTCAGTCGGTAGAGCATCAGACTTTAAATCTGAGGGTCCAGG |
| | GTTCAAGTCCCTGTTCAGGCG |
| 772 | GCCCGGATAGCTCAGTCGGTAGAGCATCAGACTTTAAATCTGAGGGTCCGGG |
| | GTTCAAGTCCCTGTTCGGGCG |
| 773 | GCCTGGGTAGCTCAGTCGGTAGAGCATCAGACTTTAAATCTGAGGGTCCAGG |
| | GTTCAAGTCCCTGTCCAGGCG |
| 774 | GCCTGGATAGCTCAGTTGGTAGAACATCAGACTTTAAATCTGACGGTGCAGG |
| | GTTCAAGTCCCTGTTCAGGCG |
| 775 | GCCCGGAGAGCTCAGTGGGTAGAGCATCAGACTTTAAATCTGAGGGTCCAGG |
| | GTTCAAGTCCTCGTTCGGGCA |
| 776 | ACCTGGGTAGCTCAGTAGGTAGAACATCAGACTTTAAATCTGAGGGTCTAGG |
| | GTTCAAGTCCCTGTCCAGGCG |
| 777 | GCCTGGATAGCTCCTTCGGTAGAGCATCATCAGACTTTAAATGTGAGGGTCC |
| | AGGGTTCAAGTTCCTGTTTGGGCG |
| 778 | GCCCAGCTAGCTCAGTCGGTAGAGCATAAGACTCTAAATCTCAGGGTTGTGG |
| | ATTCGTGCCCCATGCTGGGTG |
| 779 | CTGCAGCTAGCTCAGTCGGTAGAGCATGAGACTCTAAATCTCAGGGTCATGG |
| | GTTCGTGCCCCATGTTGGG |
| 780 | CCAGCATGTCTCAGTCGGTATAGTGTGAGACTCTAAATCTCAGGGTCGTGGG |
| | TTCAAGCCCCACATTGGG |
| 781 | GTCTAGCTAGATCAGTTGGTAGAGCATAAGACTCTAAATCTCAGGGTCATGG |
| | GTTTGAGCCCTACGTTGGGCG |
| 782 | GCCCAGCTAGCTCAGCCGGTAGAGCACAAGACTCTAAATCTCAGGGTCGTGG |
| | GTTTGAGCCCTGTGTTGAGCA |
| 783 | CCGAATAGCTTAGTTGATGAAGCGTGAGACTCTAAATCTCAGGGTAGTGGGT |
| | TCAAGCCCCACATTGGA |
| 784 | GCCTGGCTACCTCAGTTGGTAGAGCATGGGACTCTAAATCCCAGAGTCAGTG |
| | GGTTCAAGCCTCACATTGAGTG |
| 785 | GCCCGGCTAGCTCAGTCGGTAGAGCATGAGACCCTAAATCTCAGGGTCGTGG |
| | GTTCGAGCCCCACGTTGGGCG |
| 786 | GCCCGGCTAGCTCAGTCGGTAGAGCATGGGACTCTAAATCTCAGGGTCGTGG |
| | GTTCGAGCCCCACGTTGGGCG |
| 787 | GCCCGGCTAGCTCAGTCGATAGAGCATGAGACTCTAAATCTCAGGGTCGTGG |
| | GTTCGAGCCGCACGTTGGGCG |
| 788 | GCCCAGCTAGCTCAGTCGGTAGAGCATGAGACTCTAAATCTCAGGGTCATGG |
| | GTTTGAGCCCCACGTTTGGTG |
| 789 | GCCTGGCTAGCTCAGTCGGCAAAGCATGAGACTCTAAATCTCAGGGTCGTGG |
| | GCTCGAGCTCCATGTTGGGCG |
| 790 | GCCCGACTACCTCAGTCGGTGGAGCATGGGACTCTACATCCCAGGGTTGTGG |
| | GTTCGAGCCCCACATTGGGCA |
| 791 | CCCCGGCTGGCTCAGTCAGTAGATCATGAGACTCTAAATCTCAGGGTCGTGG |
| | GTTCACGCCCCACACTGGGCG |
| 792 | GCGCTAGTCAGTAGAGCATGAGACTCTAAATCTCAGGGTCGTGGGTTCGAGC |
| | CCCACATCGGGCG |
| 793 | GCCTGGATAGCTCAGTTGGTAGAGCATCAGACTCTAAATCTGAGGGTCCAGG |
| | GTTCAAGTCCCTGTTCAGGCA |
| 794 | GCCAGGATAGTTCAGGTGGTAGAGCATCAGACTCTAAACCTGAGGGTTCAGG |
| | GTTCAAGTCTCTGTTTGGGCG |
| 795 | ACCCAGATAGCTCAGTCAGTAGAGCATCAGACTCTAAATCTGAGGGTCCAAG |
| | GTTCATGTCCCTTTTTGGGTG |
| 796 | ACCTGGGTAGCTTAGTTGGTAGAGCATTGGACTCTAAATTTGAGGGCCCAGG |
| | TTTCAAGTCCCTGTTTGGGTG |
| 797 | GCCTGGGTAGCTCAGTCGGTAGAGCTATCAGACTCTAAGCCTGAGGATTCAG |
| | GGTTCAATCCCTTGCTGGGGCG |
| 798 | GATAGCTCAGTTGATAGAGCATCAGACTCTAAATCTGAGGGTCCAGGGTTCA |
| | TGTCCCTGTT |
| 799 | GTTGGGGTAACTCAGTTGGTAGAGTAGCAGACTCTACATCTGAGGGTCCAGG |
| | GTTTAAGTCCATGTCCAGGCA |
| 800 | GCCTGGATAGCTCAGTTGGTAGAGCATCAGACTCTAAATCTGAGGGTCCAGG |
| | GTTCAAGTCCCTGTTCAGGCG |
| 801 | GCCTGGATAGCTCAGTCGGTAGAGCATCAGACTCTAAATCTGAGGGTCCAGG |
| | GTTCAAGTCCCTGTTCAGGCG |
| 802 | GCCCGGATAGCTCAGTCGGTAGAGCATCAGACTCTAAATCTGAGGGTCCGGG |
| | GTTCAAGTCCCTGTTCGGGCG |
| 803 | GCCTGGGTAGCTCAGTCGGTAGAGCATCAGACTCTAAATCTGAGGGTCCAGG |
| | GTTCAAGTCCCTGTCCAGGCG |
| 804 | GCCTGGATAGCTCAGTTGGTAGAACATCAGACTCTAAATCTGACGGTGCAGG |
| | GTTCAAGTCCCTGTTCAGGCG |
| 805 | GCCCGGAGAGCTCAGTGGGTAGAGCATCAGACTCTAAATCTGAGGGTCCAGG |
| | GTTCAAGTCCTCGTTCGGGCA |
| 806 | ACCTGGGTAGCTCAGTAGGTAGAACATCAGACTCTAAATCTGAGGGTCTAGG |
| | GTTCAAGTCCCTGTCCAGGCG |
| 807 | GCCTGGATAGCTCCTTCGGTAGAGCATCATCAGACTCTAAATGTGAGGGTCC |
| | AGGGTTCAAGTTCCTGTTTGGGCG |
| 808 | GGCAGAATGGTGCAGCGGTTCAGCACCCAGGCTCTTCAGCCAGCTGTTGCCT |
| | GGGCTCAAATCCCAGCTCTGCCA |
| 809 | GGCTGTATAGCTCAGTGGTAGAGCATTTGACTTCAGAATCCTATACTCAGGG |
| | GAAGGAGAACTGGGGGTTTCTCAGTGGGTCAAAGGACTTGTAGTGGTAAATC |
| | AAAAGCAACTCTATAAGCTATGTAACAAACTTTAAAGTCATATGTAGCTGGG |
| | TTCAAATCCTGTTTCTGCCA |
| 810 | GGCTGTATAGCTCAGTGGTAGAGCATTTGACTTCAGCTTTAAAGTCATATGT |
| | AGCTGGGTTCAAATCCTGTTTCTGCCA |
| 811 | GGGGGCATAGCTCAGTGGTAGAGCATTTGACTTCAGATCAAGAGGTCCCTGG |
| | TTCAAATCCAGGTGCCCCCT |
| 812 | GGGGGTATAGCTCAGGGGTAGAGCATTTGACTTCAGATCAAGAGGTCCCTGG |
| | TTCAAATCCAGGTGCCCCCC |
| 813 | GGGGGTATAGCTTAGCGGTAGAGCATTTGACTTCAGATCAAGAGGTCCCCGG |
| | TTCAAATCCGGGTGCCCCCT |
| 814 | GGGGGTATAGCTTAGGGGTAGAGCATTTGACTTCAGATCAAAAGGTCCCTGG |
| | TTCAAATCCAGGTGCCCCTT |
| 815 | GGGGGTATAGCTCAGGGGTAGAGCATTTGACTTCAGATCAAGAGGTCCCCAG |
| | TTCAAATCTGGGTGCCCCCT |
| 816 | GGGGGTATAGCTCAGGGGTAGAGCATTTGACTTCAGATCAAGAAGTCCCCGG |
| | TTCAAATCCGGGTGCCCCCT |
| 817 | GGGGGTATAGCTCAGGGGTAGAGCATTTGACTTCAGATCAAGAGGTCTCTGG |
| | TTCAAATCCAGGTGCCCCCT |
| 818 | GGGGGTATAGCTCAGGGGTAGAGCACTTGACTTCAGATCAAGAAGTCCTTGG |
| | TTCAAATCCAGGTGCCCCCT |
| 819 | GGGGATATAGCTCAGGGGTAGAGCATTTGACTTCAGATCAAGAGGTCCCCGG |
| | TTCAAATCCGGGTGCCCCCC |
| 820 | GGGGGTATAGTTCAGGGGTAGAGCATTTGACTTCAGATCAAGAGGTCCCTGG |
| | TTCAAATCCAGGTGCCCCCT |
| 821 | GGGGGTATAGCTCAGGGGTAGAGCATTTGACTTCAAATCAAGAGGTCCCTGA |
| | TTCAAATCCAGGTGCCCCCT |
| 822 | GGGCGTATAGCTCAGGGGTAGAGCATTTGACTTCAGATCAAGAGGTCCCCAG |
| | TTCAAATCTGGGTGCCCCCT |
| 823 | GGGGGTATAGCTCACAGGTAGAGCATTTGACTTCAGATCAAGAGGTCCCCGG |
| | TTCAAATCTGGGTGCCCCCT |
| 824 | GGGCGTATAGCTCAGGGGTAGAGCATTTGACTTCAGATCAAGAGGTCCCCAG |
| | TTCAAATCTGGGTGCCCA |
| 825 | GGGGGTATAGCTCACAGGTAGAGCATTTGACTTCAGATCAAGAGGTCCCCGG |
| | TTCAAATCCGGTTACTCCCT |
| 826 | GGGGGTAGGGCTCAGGGATAGAGCATTTGACTTCAGATCAAGAGGTCCCCGG |
| | TTCGAATCTAGGTGCCCCCT |
| 827 | GGTATATCTCAGGGGGCAGAGCATTTGACTTCAGATCAAGAGGTCCCCGGTT |
| | GAAATCCGGGTGCT |
| 828 | GGGGGTATAGCTCAGGGGTAGAGCACTTGACTTCAGATCAAGAGGTCCCTGG |
| | TTCAAATCCAGGTGCCCCCT |
| 829 | GGGGGTATAGCTCAGTGGTAGAGCATTTGACTTCAGATCAAGAGGTCCCTGG |
| | TTCAAATCCGGGTGCCCCCT |
| 830 | GGGGGTATAGCTCAGTGGGTAGAGCATTTGACTTCAGATCAAGAGGTCCCCG |
| | GTTCAAATCCGGGTGCCCCCT |
| 831 | GGGGGTGTAGCTCAGTGGTAGAGCATTTGACTTCAGATCAAGAGGTCCCTGG |
| | TTCAAATCCAGGTGCCCCCT |
| 832 | GGGGGTATAGCTCAGGGGTAGAGCATTTGACTTCAGATCAAGAGGTCCCCGG |
| | TTCAAATCCGGGTGCCCCCT |
| 833 | GGGGGTATAGCTCAGGGGTAGAGCATTTGACTTCAGATCAAGAGGTCCCTGG |
| | TTCAAATCCAGGTGCCCCCT |
| 834 | GACCTCGTGGCGCAATGGTAGCGCGTCTGACTTCAGATCAGAAGGTTGCGTG |
| | TTCAAATCACGTCGGGGTCA |
| 835 | GACCTCGTGGCACAATGGTAGCACGTCTGACTTCAGATCAGAAGGTTGCGTG |
| | TTCAAATCACGTCGGGGTCA |
| 836 | GAAGCGGTGGCTCAATGGTAGAGCTTTCGACTTCAATTAAATCTTGGAAATT |
| | CCACGGAATAAGATTGCAATCGAAGGGTTGCAGGTTCAATTCCTGTCCGTTT |
| | CA |
| 837 | GAAGCGGTGGCTCAATGGTAGAGCTTTCGACTTCAAATCGAAGGGTTGCAGG |
| | TTCAATTCCTGTCCGTTTCA |
| 838 | GGCCTCATGGTGCAACAGTAGTGTGTCTGACTTCAGATCAGAAGGTTGTATG |
| | TTCAAATCACATAGGGGTCA |
| 839 | GACCTCGTGGTGAAATGGTAGCATGTTTGACTTCAAATCAGGAGGTTGTGTG |
| | TTCAAGTCACATCAGGGTCA |
| 840 | GACCTTGTGGCGCAATGGTAGCATGTTTGACTTCAAATCAGGAGGTTGTGTG |
| | TTCAAGTCACATCAGGGTCA |
| 841 | GACCTCGTGGCGCAACGGTAGCGCGTCTGACTTCAGATCAGAAGGCTGCGTG |
| | TTCGAATCACGCCGGGGTCA |
| 842 | GACCTTGTGGCTCAATGGTAGCGCATCTGACTTCAGATCAGGAGGTTGCACG |
| | TTCAAATCATGCCGGGGTCA |
| 843 | GACCTTGTGGCGCAACGGTAGCGCGTCTGACTTCAGATCAGAAGGTTGCGTG |
| | TTCAAATCACGTCGGGGTCA |
| 844 | GACCTCGTGGCGCAACGGTAGCGCGTCTGACTTCAGATCAGAAGGTTGCGTA |
| | TTCAAATCACGTCGGGGTCA |
| 845 | GACCTCGTGGCGCAACGGCAGCGCGTCTGACTTCACATTAGAAGGTTGCGTG |
| | TTCAAATCACGTCGGGGTCA |
| 846 | GACCTCATGGCGCAACGGTAGCGCGTCTGACTTCAGATCAGAAGGTTGCGTG |
| | TTCAAATCACATCGGGGTCA |
| 847 | GACCTCGTGGTGCAACGGTAGCGCGTATGATTTCAGATCAGAAGGTTGCGTG |
| | TTCAAATCACGTCGGGGTCA |
| 848 | GACCTCGTAGCGCAACGGTAGCGCGTCTGACTTCAGATCAGAAGGTTGCGTG |
| | TTCAAATCACGTCGGGGTCA |
| 849 | AGGGGTATAGCTCAATTGGCAGAGCGTCGGTCTTCAAAACCGAAGGTTGTAG |
| | GTTCGATTCCTACTGCCCCTGCCA |
| 850 | GACCTCATGGCGCAACGGTAGCGCGTCTGACTTCAGATCAGAAGGTTGCGTG |
| | TTCAAATCACGTCGGGGTCA |
| 851 | GACCTCGTGGCGCAACGGTAGCGCGTCTAACTTCAGATCAGAAGGTTGCGTG |
| | TTCAAATCACGTCGGGGTCA |
| 852 | ACGGGAGTAGCTCAGTTGGTAGAGCACCGGTCTTCAAAACCGGGTGTCGGGA |
| | GTTCGAGCCTCTCCTCCCGTG |
| 853 | GACCTCGTGGCGCAACGGTAGCGCGTCTGACTTCAGATCAGAAGGTTGCATG |
| | TTCAAATCACGTCGGGGTCA |
| 854 | GACTCCGTGGCGCAACGGTAGCGCGTCCGACTTCAGATCGGAAGGTTGCGTG |
| | TTCAAATCACGTCGGGGTCA |
| 855 | GACTCCGTGGCGCAACGGTAGCGCGTCTGACTTCAGATCAGAAGGTTGCGTG |
| | TTCAAATCACGTCGGGGTCA |
| 856 | GGCCTCGTGGCGCAACGGTAGCGCGTCTGACTCCAGATCAGAAGGTTGCGTG |
| | TTCAAATCACGTCGGGGTCA |
| 857 | GGCCTCGTGGCGCAACGGTAGCACGTCTGACTCCAGATCAGAAGGTTGCGTG |
| | TTCAAATCACGTCGGGGTCA |
| 858 | CGGCCTCGTGGCGCAACGGTAGCACGTCTGACTTCAGATCAGAAGGTTGCGT |
| | GTTCAAATCACGTCGGGGTCA |
| 859 | GGCCTCGTCGCGCAACGGTAGCGCGTCTGACTCCAGATCAGAAGGTTGCGTG |
| | TTCAAATCACGTCGGGGTCA |
| 860 | GGCCTCGTCGCGCAACGGTAGCGCGTCTGACTTCAGATCAGAAGGTTGCGTG |
| | TTCAAATCACGTCGGGGTCA |
| 861 | GGCCTCGTCGCGCAACGGTAGCACGTCTGACTCCAGATCAGAAGGTTGCGTG |
| | TTCAAATCACGTCGGGGTCA |
| 862 | GGCCTCGTCGCGCAACGGTAGCACGTCTGACTTCAGATCAGAAGGTTGCGTG |
| | TTCAAATCACGTCGGGGTCA |

In certain embodiments, the tRNA comprises, consists essentially of, or consists of the nucleotide sequence of SEQ ID NO: 6. In certain embodiments, the tRNA comprises, consists essentially of, or consists of the nucleotide sequence of SEQ ID NO: 7. In certain embodiments, the tRNA comprises, consists essentially of, or consists of the nucleotide sequence of SEQ ID NO: 8. In certain embodiments, the tRNA comprises, consists essentially of, or consists of the nucleotide sequence of SEQ ID NO: 9. In certain embodiments, the tRNA comprises, consists essentially of, or consists of the nucleotide sequence of SEQ ID NO: 11. In certain embodiments, the tRNA comprises, consists essentially of, or consists of the nucleotide sequence of SEQ ID NO: 16. In certain embodiments, the tRNA comprises, consists essentially of, or consists of the nucleotide sequence of SEQ ID NO: 17. In certain embodiments, the tRNA comprises, consists essentially of, or consists of the nucleotide sequence of SEQ ID NO: 18. In certain embodiments, the tRNA comprises, consists essentially of, or consists of the nucleotide sequence of SEQ ID NO: 19. In certain embodiments, the tRNA comprises, consists essentially of, or consists of the nucleotide sequence of SEQ ID NO: 20. In certain embodiments, the tRNA comprises, consists essentially of, or consists of the nucleotide sequence of SEQ ID NO: 21. In certain embodiments, the tRNA comprises, consists essentially of, or consists of the nucleotide sequence of SEQ ID NO: 22. In certain embodiments, the tRNA comprises, consists essentially of, or consists of the nucleotide sequence of SEQ ID NO: 35. In certain embodiments, the tRNA comprises, consists essentially of, or consists of the nucleotide sequence of SEQ ID NO: 36. In certain embodiments, the tRNA comprises, consists essentially of, or consists of the nucleotide sequence of SEQ ID NO: 37. In certain embodiments, the tRNA comprises, consists essentially of, or consists of the nucleotide sequence of SEQ ID NO: 38. In certain embodiments, the tRNA comprises, consists essentially of, or consists of the nucleotide sequence of SEQ ID NO: 39. In certain embodiments, the tRNA comprises, consists essentially of, or consists of the nucleotide sequence of SEQ ID NO: 40. In certain embodiments, the tRNA comprises, consists essentially of, or consists of the nucleotide sequence of SEQ ID NO: 44. In certain embodiments, the tRNA comprises, consists essentially of, or consists of the nucleotide sequence of SEQ ID NO: 45. In certain embodiments, the tRNA comprises, consists essentially of, or consists of the nucleotide sequence of SEQ ID NO: 178. In certain embodiments, the tRNA comprises, consists essentially of, or consists of the nucleotide sequence of SEQ ID NO: 179. In certain embodiments, the tRNA comprises, consists essentially of, or consists of the nucleotide sequence of SEQ ID NO: 180. In certain embodiments, the tRNA comprises, consists essentially of, or consists of the nucleotide sequence of SEQ ID NO: 181. In certain embodiments, the tRNA comprises, consists essentially of, or consists of the nucleotide sequence of SEQ ID NO: 182. In certain embodiments, the tRNA comprises, consists essentially of, or consists of the nucleotide sequence of SEQ ID NO: 186. In certain embodiments, the tRNA comprises, consists essentially of, or consists of the nucleotide sequence of SEQ ID NO: 187.

In certain embodiments, the tRNA may comprise one or more mutations (e.g., nucleotide substitutions, deletions, or insertions) relative to a reference tRNA sequence (e.g., a tRNA disclosed herein). In certain embodiments, the tRNA may comprise, consist, or consist essentially of, a single mutation, or a combination of 2, 3, 4, 5, 6, 7, 8, 9, 10, 11, 12, 13, 14, 15 or more than 15 mutations. It is contemplated that the tRNA may comprise, consist, or consist essentially 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 3-4 mutations.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul (1993) J. MOL. EVOL. 36, 290-300; Altschul et al., (1997) NUCLEIC ACIDS RES. 25:3389-3402) are tailored for sequence similarity searching. For a discussion of basic issues in searching sequence databases see Altschul et al. (1994) NATURE GENETICS 6:119-129. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; -W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and -Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

It is contemplated that a tRNA may comprise one or more modifications. Exemplary modified tRNAs include: acylated tRNA; alkylated tRNA; a tRNA containing one or more bases other than adenine, cytosine, guanine, or uracil; a tRNA covalently modified by the attachment of a specific ligand or antigenic, fluorescent, affinity, reactive, spectral, or other probe moiety; a tRNA containing one or more ribose moieties that are methylated or otherwise modified; aa-tRNAs that are aminoacylated with an amino acid other than the 20 natural amino acids, including non-natural amino acids that function as a carrier for reagents, specific ligands, or as an antigenic, fluorescent, reactive, affinity, spectral, or other probe; or any combination of these compositions. Exemplary modified tRNA molecules are described in Soll et al. (1995) “tRNA: Structure, Biosynthesis, and Function,” ASM Press; El Yacoubi et al. (2012) ANNU. REV. GENET. 46:69-95; Grosjean et al. (1998) “Modification and Editing of RNA.” ASM Press; Hendrickson et al. (2004) ANNU. REV. BIOCHEM. 73:147-176, 2004; Ibba et al. (2000) ANNU. REV. BIOCHEM. 69:617-650; Johnson et al. (1995) COLD SPRING HARBOR SYMP. QUANT. BIOL. 60:71-82; Johnson et al. (1982) J. MOL. BIOL. 156:113-140; Crowley et al. (1994) CELL 78:61-71; Beier et al. (2001) NUCLEIC ACIDS RES. 29:4767-4782; Torres et al. (2014) TRENDS MOL. MED. 20:306-314; Bjork et al. (1987) ANNU. REV. BIOCHEM. 56:263-287; Schaffrath et al. (2017) RNA BIOL. 14 (9): 1209-1222; and Johansson et al. (2008) MOL. CELL. BIOL. 28 (10): 3301-12.

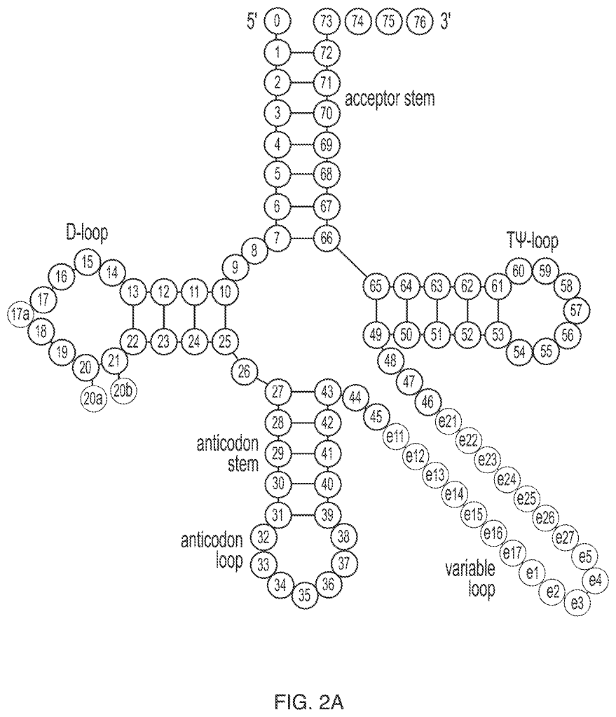

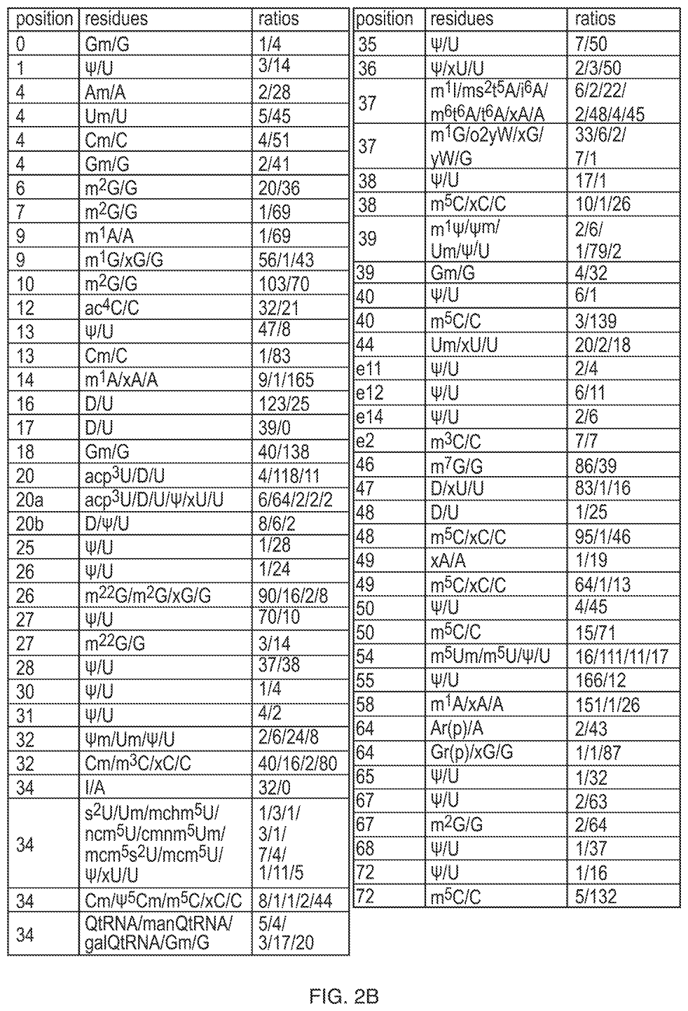

In certain embodiments, a tRNA comprises a naturally occurring nucleotide modification. Naturally occurring tRNAs contain a wide variety of post-transcriptionally modified nucleotides, which are described, for example, in Machnicka et al. (2014) RNA BIOLOGY 11 (12): 1619-1629, and include one or more of the residues as shown in FIG. 2B. In certain embodiments, the tRNA comprises one or more of the residues selected from the group consisting of: 2′-O-methylguanosine or G at position 0; pseudouridine or U at position 1; 2′-O-methyladenosine, A, 2′-O-methyluridine, U, 2′-O-methylcytidine, C, 2′-O-methylguanosine, or G at position 4; N2-methylguanosine or G at position 6; N2-methylguanosine or G at position 7; 1-methyladenosine, A, 1-methylguanosine, G, or a modified G at position 9; N2-methylguanosine or G at position 10; N4-acetylcytidine or C at position 12; pseudouridine, U, 2′-O-methylcytidine, or C at position 13; 1-methyladenosine, A, or a modified A at position 14; dihydrouridine (D) or U at position 16; D or U at position 17; 2′-O-methylguanosine or G at position 18; 3-(3-amino-3-carboxypropyl) uridine, D, or U at position 20; 3-(3-amino-3-carboxypropyl) uridine, D, pseudouridine, U, or a modified U at position 20a; D, pseudouridine, or U at position 20b; pseudouridine or U at position 25; pseudouridine, U, N2,N2-dimethylguanosine, N2-methylguanosine, G, or a modified G at position 26; pseudouridine, U, N2,N2-dimethylguanosine, or G at position 27; pseudouridine or U at position 28; pseudouridine or U at position 30; pseudouridine or U at position 31; 2′-O-methylpseudouridine, 2′-O-methyluridine, pseudouridine, U, 2′-O-methylcytidine, 3-methylcytidine, C, or a modified C at position 32; inosine, A, 2-thiouridine, 2′-O-methyluridine, 5-(carboxyhydroxymethyl) uridine methyl ester, 5-carbamoylmethyluridine, 5-carboxymethylaminomethyl-2′-O-methyluridine, 5-methoxycarbonylmethyl-2-thiouridine, 5-methoxycarbonylmethyluridine, pseudouridine, U, a modified U, 2′-O-methylcytidine, 5-formyl-2′-O-methylcytidine, 5-methylcytidine, C, a modified C, queuosine, mannosyl-queuosine, galactosyl-queuosine, 2′-O-methylguanosine, or G at position 34; pseudouridine or U at position 35; pseudouridine, U, or a modified U at position 36; 1-methylinosine, 2-methylthio-N6-threonylcarbamoyladenosine, N6-isopentenyladenosine, N6-methyl-N6-threonylcarbamoyladenosine, N6-threonylcarbamoyladenosine, A, a modified A, 1-methylguanosine, peroxywybutosine, wybutosine, G, or a modified G at position 37; pseudouridine, U, 5-methylcytidine, C, or a modified C at position 38; 1-methylpseudouridine, 2′-O-methylpseudouridine, 2′-O-methyluridine, pseudouridine, U, 2′-O-methylguanosine, or G at position 39; pseudouridine, U, 5-methylcytidine, or C at position 40; 2′-O-methyluridine, U, or a modified U at position 44; pseudouridine or U at position e11; pseudouridine or U at position e12; pseudouridine or U at position e14; 3-methylcytidine or C at position e2; 7-methylguanosine or G at position 46; D, U, or a modified U at position 47; D, U, 5-methylcytidine, C, or a modified C at position 48; A, a modified A, 5-methylcytidine, C, or a modified C at position 49; pseudouridine, U, 5-methylcytidine, or C at position 50; 5,2′-O-dimethyluridine, 5-methyluridine, pseudouridine, or U at position 54; pseudouridine or U at position 55; 1-methyladenosine, A, or a modified A at position 58; 2′-O-ribosyladenosine (phosphate), A, 2′-O-ribosylguanosine (phosphate), G, or a modified G at position 64; pseudouridine or U at position 65; pseudouridine, U, N2-methylguanosine, or G at position 67; pseudouridine or U at position 68; and, pseudouridine, U, 5-methylcytidine, or C at position 72. A, C, G, and U, refer to unmodified adenine, cytosine, guanine, and uracil, respectively. The numbering of the residues is based on the tRNA numbering system described in Steinberg et al., (1993) NUCLEIC ACIDS RES. 21:3011-15.

In certain embodiments, the tRNA comprises one or more nucleotide modifications selected from 5-methyl uridine, 5-carbamoylmethyluridine, 5-carbamoyl-methyl-2-O-methyluridine, 5-methoxy-carbonylmethyluridine, 5-methoxycarbonylmethyl-2-thiouridine, pseudouridine, dihydrouridine, 1-methyladenosine, and inosine.

### II. Methods of Making tRNAs

It is contemplated the tRNA molecules (e.g., suppressor tRNAs) useful in the practice of the invention can be produced by methods known in the art, including extracellular production by synthetic chemical methods, intracellular production by recombinant DNA methods, or purification from natural sources.

For example, DNA molecules encoding tRNAs can be synthesized chemically or by recombinant DNA methodologies. For example, the sequences of the tRNAs can be synthesized or cloned from libraries by conventional hybridization techniques or polymerase chain reaction (PCR) techniques, using the appropriate synthetic nucleic acid primers. The resulting DNA molecules encoding the tRNAs can be ligated to other appropriate nucleotide sequences, including, for example, expression control sequences to produce conventional gene expression constructs (i.e., expression vectors) encoding the tRNAs. Production of defined gene constructs is within routine skill in the art. Nucleic acids encoding desired tRNAs can be incorporated (ligated) into expression vectors, such as the expression vectors described in the following section, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the tRNAs. Specific expression and purification conditions will vary depending upon the expression system employed.

Alternatively, tRNAs can be chemically synthesized or purified from natural sources by methods known in art. When a tRNA is aminoacylated prior to introduction into the cell or administration to the subject, the tRNA may be aminoacylated with a desired amino acid by any method known in the art, including chemical or enzymatic aminoacylation.

### III. Expression Vectors

The tRNAs of interest may be expressed in a cell of interest by incorporating a gene encoding a tRNA of interest into an appropriate expression vector. As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate the recombinant polynucleotide of interest.

In certain embodiments, the expression vector is a viral vector. The term “virus” is used herein to refer to an obligate intracellular parasite having no protein-synthesizing or energy-generating mechanism. Exemplary viral vectors include retroviral vectors (e.g., lentiviral vectors), adenoviral vectors, adeno-associated viral vectors, herpesviruses vectors, epstein-barr virus (EBV) vectors, polyomavirus vectors (e.g., simian vacuolating virus 40 (SV40) vectors), poxvirus vectors, and pseudotype virus vectors.

The virus may be a RNA virus (having a genome that is composed of RNA) or a DNA virus (having a genome composed of DNA). In certain embodiments, the viral vector is a DNA virus vector. Exemplary DNA viruses include parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillomoviruses (e.g., HPV), polyomaviruses (e.g., simian vacuolating virus 40 (SV40)), and poxviruses (e.g., vaccinia virus, cowpox virus, smallpox virus, fowlpox virus, sheeppox virus, myxoma virus). In certain embodiments, the viral vector is a RNA virus vector. Exemplary RNA viruses include bunyaviruses (e.g., hantavirus), coronaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

In certain embodiments, the expression vector comprises a regulatory sequence or promoter operably linked to the nucleotide sequence encoding the tRNA. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

tRNA genes preferably have strong promoters that are active in a variety of cell types. The promoters for eukaryotic tRNA genes typically are present within the structural sequences encoding the tRNA molecule itself. Although there are elements which regulate transcriptional activity within the 5′ upstream region, the length of an active transcriptional unit may be considerably less than 500 base pairs.

Additional exemplary promoters which may be employed include, but are not limited to, the retroviral LTR, the SV40 promoter, the human cytomegalovirus (CMV) promoter, the U6 promoter, or any other promoter (e.g., cellular promoters such as eukaryotic cellular promoters including, but not limited to, the histone, pol III, and β-actin promoters). Other viral promoters which may be employed include, but are not limited to, adenovirus promoters, TK promoters, and B19 parvovirus promoters. The selection of a suitable promoter will be apparent to those skilled in the art from the teachings contained herein.

In certain embodiments, an expression vector comprises a tRNA coding sequence that encodes a tRNA that comprises, consists essentially of, or consists of a nucleotide sequence shown in TABLE 2 or TABLE 3. In certain embodiments, an expression vector comprises a RNA coding sequence that encodes a tRNA that comprises, consists essentially of, or consists of a nucleotide sequence having 80%, 85%, 86%, 87%, 88%, 89%, 90%, 91%, 92%, 93%, 94%, 95%, 96%, 97%, 98%, or 99% to a nucleotide sequence shown in TABLE 2 or TABLE 3.

In certain embodiments, in addition to a tRNA coding sequence, the expression vector comprises a nucleotide sequence corresponding to a genomic DNA sequence flanking a wild-type tRNA gene (i.e., a DNA sequence from the same genome as a wild-type tRNA gene and which is 5′ or 3′ to the wild-type tRNA gene in the genome, e.g., immediately 5′ or 3′ to the wild-type tRNA gene in the genome). In certain embodiments, in addition to a tRNA coding sequence, the expression vector comprises a nucleotide sequence corresponding to an exogenous promoter.

In certain embodiments, the expression vector comprises a nucleotide sequence shown in TABLE 4. In certain embodiments, the expression vector comprises a nucleotide sequence having 80%, 85%, 86%, 87%, 88%, 89%, 90%, 91%, 92%, 93%, 94%, 95%, 96%, 97%, 98%, or 99% sequence identity to a nucleotide sequence shown in TABLE 4. In certain embodiments, in the expression vector, the nucleotide sequence set forth in TABLE 4 is operably linked to the nucleotide sequence encoding the tRNA. In certain embodiments, in the expression vector, the nucleotide sequence set forth in TABLE 4 is 5′ or 3′ (e.g., immediately 5′ or immediately 3) to the nucleotide sequence encoding the tRNA. In certain embodiments, the expression vector comprises a nucleotide sequence selected from SEQ ID NOs: 869-888, or a nucleotide sequence having 80%, 85%, 86%, 87%, 88%, 89%, 90%, 91%, 92%, 93%, 94%, 95%, 96%, 97%, 98%, or 99% sequence identity to a sequence selected from SEQ ID NOs: 869-888.

TABLE 4

| | Location | |
|---|---|---|
| SEQ | (Relative | |
| ID NO | to tRNA) | Nucleotide Sequence |
| 26 | 5′ | CTACCCAGAGGCAGGCGGGAGACTCCCCCGAGCGTCCAATAAGA |
| | | GCGCCGCCAATGGAGCCGCCCGCCCGCGGGGGTGCAGAGGGACT |
| | | TCCGGGTGAGGTCCTCCGCTACTTCCCTCCCCACGGAAAAGATA |
| | | GACCAGTCTGACGCGAGCCTGAAGGCGGCTACACGCTTTAAGCT |
| | | AAGTAAAGGCACCTTCTCGCTGGC |
| 27 | 3′ | ACTTGTATGTTGTTTTTATCTGTCAGTTTGTTAATCCCAAGATT |
| | | CCCTTTGGAAATAAAGCGAAATTGACCGTAGTGGTTATGACCAA |
| | | CTTCTAGTCTAAACTTAATTCTTGGAACTCAAGGATCTGAGCAA |
| | | ACAACTGTCAGGGTGACACATTGCTTAAACGGTGACAGCGGTCG |
| | | AGAGCCTTGTCCCGGATGGAGAGT |
| 32 | 3′ | ACTTGTATGTTGTTTTTATCTGTCAGTTTGTTAATCCCAAGATT |
| | | CCCTTTGGAAATAAAGCGAAATTGACCGTAGTGGTTATGACCAA |
| | | CTTCTAGTCTAAACTT |
| 33 | 5′ | GAGGGCCTATTTCCCATGATTCCTTCATATTTGCATATACGATA |
| | | CAAGGCTGTTAGAGAGATAATTAGAATTAATTTGACTGTAAACA |
| | | CAAAGATATTAGTACAAAATACGTGACGTAGAAAGTAATAATTT |
| | | CTTGGGTAGTTTGCAGTTTTAAAATTATGTTTTAAAATGGACTA |
| | | TCATATGCTTACCGTAACTTGAAAGTATTTCGATTTCTTGGCTT |
| | | TATATATCTTGTGGAAAGGACGGGCGGAGGAAGGCACCTTCTCG |
| | | CTGGC |
| 34 | 3′ | ACTTGTATGTTGTTTTTATCTGTCAGTTTGTTAATCCCAAGATT |
| | | CC |
| 173 | 5′ | GATCACCGGAAGAGGTGACAGACACCTCGGGGCCCATGAACGTT |
| | | TGGAATTCGTAAGGACATGAGAATCTCGGTGGTTCCGTGTCTGC |
| | | CCGCCATCGCGGCCACCGGCCACGGGCCCAAGCCAAGTGTAGCG |
| | | AAGCTTAGAAAAGGTTGCCCAACGTCATGTGGCTTGAGAAGGCT |
| | | GCCGGGCGCCTTAAGCCGCCAGCA |
| 174 | 3′ | CACTGAACCTTTTTTTGGCCTTAGAATCCCTGTTTTGGGGCCTG |
| | | CAGGAAGTAGCAACCAACCCGAGCCTCCGCAGGGAATGCACTGA |
| | | CCTGTAGAATGGACGTTCAGCTTCCCTCCCTGTGTCTCAACACG |
| | | ATTACATTTCAGGAACAGCCTGGGCTGGGAGGCACTGCGCACGC |
| | | GCGCCGAGTCGGGCGGAAAAATAA |
| 869 | 5′ | CCAAAACATCTTTTACTGTAGTATCTACTTACCATACTACCCAA |
| | | GAATGGCACACTGCTCACATCTTCAAAAGCTTAAACCAAGAGCA |
| | | CTACACAGGTGC |
| 870 | 5′ | TGTGTGTCGGGGCCGGTACCCTGCTTCCGGTTCCCGCACGCATT |
| | | CCCGGATTGCAGTGCGGACCCCTTCTGTAAGCGCGCGATAAAGC |
| | | GCGGTTTTGGAA |
| 871 | 5′ | TCATGTCATATAAGTAGAACCATACAATATATATATAAAATCCA |
| | | GGTTAATAGCCAATCTTACAACATTTCTCATATTTTTTGCAGTT |
| | | GCTAAGCCATGG |
| 872 | 5′ | ACATTACAATACATATCAACATATCACCATAATTAAATTGCAAG |
| | | TCTTCGTCAAAAGCAAGCCTTAAAGGAGTATCCCAAAAACACAT |
| | | TTTCCCCAGAAG |
| 873 | 5′ | AGACCTTTAGAGCGTGGTTAAACCCATATGTTGGGATTTATGCT |
| | | GCTTTTATGGTAGCAATACCCTATATTAAGATTTGAAGTAGACC |
| | | CGGAAAGTTAGT |
| 874 | 5′ | GTTCATGAAAGAATAAATAAATGTTTAAAAAAAAAAAAAACTGA |
| | | GGTAAATTTCTATATTCTTTCATAAAAGCAGTTTAAAGACGAAC |
| | | GTTTTTCGAGGT |
| 875 | 5′ | GCTGGGTCTCGGTGACACTGACGACGGGAGGCGCGGTCGGAAGA |
| | | GCGCGGGGCCGTCGCCTCTGGCTTAACATAGCAGATGCGCTGAG |
| | | ACTCCAACAGGT |
| 876 | 5′ | CAGTGGCGGCGAAAACTCTCTGCGTTCTGGAGGGAGGGTGCGGG |
| | | CAGGAGGAGGTAGAGGATGCCTTGTAAGCGGAGCAAAAACAAGG |
| | | TTCAACGTCTGC |
| 877 | 5′ | CAAATCACTTGCCTCTCGGCGCGAGACCGCGATGCGCGGGGGCG |
| | | GGAGCGTGATGATGGCATCGCGTAAGGAGAGGGTGTGAGAAGCC |
| | | GGATCCTGTGGT |
| 878 | 5′ | CCCTGTGTCCGAAGAGGTCTGCGTTGCGACTTACGTGGTAGTGC |
| | | TTGGAAGGTGCGGAGTAGATGAGAGATAAGTGAATGTGGACAAA |
| | | CCTGTCACGTAG |
| 879 | 5′ | GAGCGGAGCTCAGAGGGTGCGCGCTCCGCCCTTTCGCGGGCCTG |
| | | GCATGAGCGCAGTGGTTGTTACACTAAAGTGTCTCCGCCTGTCG |
| | | AATATTCTCGTG |
| 880 | 5′ | GTGTCACTGGTTTCAAATCAACCTCAATTTTTTTGGAGACGTGA |
| | | GTGCTGAGCATTTTTTCTTCAGTGAAGTGACTTGGCAGCCAAAA |
| | | TCGCCAACGCCC |
| 881 | 5′ | TCCTGGCATGTCCCGCCCAAGTCCCTTAGCCCCGCTCCCCAACC |
| | | CTGCCCCATTCCCACTCTAGTACCCGTAAGCTACAAGACGCCGC |
| | | CGTTCGTCGGGT |
| 882 | 5′ | TGCTCAGTCGTCCTGCCGGGCGGGCCCTGAGGTTGCAAGGGACG |
| | | GAGGAAGTTTCGTGCGTGCGCCCTTCCTATAGCGCCCAGTAGAA |
| | | CTGACAGTACCT |
| 883 | 5′ | TCCTCGGATTACGCATGCTCAGTGCAATCTTCGGTTGCCTGGAC |
| | | TAGCGCTCCGGTTTTTCTGTGCTGAACCTCAGGGGACGCCGACA |
| | | CACGTACACGTC |
| 884 | 5′ | GATAATTTCCTGAAAGAAAAGATCAATTCGATGTTACCAAATCT |
| | | GGGATATCCAGAAAAATTTTCTTCTTCTCCTAGGAGAAAAACTA |
| | | TCAAATGTCAGG |
| 885 | 5′ | TCTCTCACGGCAAACTGTTGCAGACTGTAGAGACGCTATGCCAA |
| | | GAATCTTTTACTTAAAAGCAGGAATAGATTCAATAGGCAACTTC |
| | | ACTGCACATGTA |
| 886 | 5′ | CAACCTCCCCTTCTCAAGGAGCAGGTGGATTGGTCCCGAGCTAG |
| | | CTGGTGGGCGGAGGTGACGTTTTTATAAGTTGCTCAAGAGACGG |
| | | TAACAACCGACG |
| 887 | 5′ | GTGGAACTTCCACTGAATTACTCTTTTCGCATGTAAGATCACTG |
| | | AACCGTGATAATCATTGATCCTATTTGTAGAACTGTATGAAACA |
| | | GTTCCCTAAGGA |
| 888 | 5′ | TCGCTCAACAGGCGGCCAGGGTGCGAGCAGTGAAGCTGCGGCAC |
| | | GCCGGAGCGTTTAATGGCCATCAAATTGGCCTCTCTAGGAGGTA |
| | | GCTGCAGCCGGA |
| 895 | 5′ | AAAGGCACCTTCTCGCTGGC |
| 896 | 3′ | ACTTGTATGTTGTTTTT |
| 897 | 5′ | TCTCGCTGGC |
| 900 | 5′ | AGCGCTCCGGTTTTTCTGTGCTGAACCTCAGGGGACGCCGACAC |
| | | ACGTACACGTC |

### Adeno-Associated Virus (AAV) Vectors

In certain embodiments, an expression vector is an adeno-associated virus (AAV) vector. AAV is a small, nonenveloped icosahedral virus of the genus Dependoparvovirus and family Parvovirus. AAV has a single-stranded linear DNA genome of approximately 4.7 kb. AAV is capable of infecting both dividing and quiescent cells of several tissue types, with different AAV serotypes exhibiting different tissue tropism.

AAV includes numerous serologically distinguishable types including serotypes AAV-1 to AAV-12, as well as more than 100 serotypes from nonhuman primates (See, e.g., Srivastava (2008) J. CELL BIOCHEM., 105 (1): 17-24, and Gao et al. (2004) J. VIROL., 78 (12), 6381-6388). The serotype of the AAV vector used in the present invention can be selected by a skilled person in the art based on the efficiency of delivery, tissue tropism, and immunogenicity. For example, AAV-1, AAV-2, AAV-4, AAV-5, AAV-8, and AAV-9 can be used for delivery to the central nervous system; AAV-1, AAV-8, and AAV-9 can be used for delivery to the heart; AAV-2 can be used for delivery to the kidney; AAV-7, AAV-8, and AAV-9 can be used for delivery to the liver; AAV-4, AAV-5, AAV-6, AAV-9 can be used for delivery to the lung, AAV-8 can be used for delivery to the pancreas, AAV-2, AAV-5, and AAV-8 can be used for delivery to the photoreceptor cells; AAV-1, AAV-2, AAV-4, AAV-5, and AAV-8 can be used for delivery to the retinal pigment epithelium; AAV-1, AAV-6, AAV-7, AAV-8, and AAV-9 can be used for delivery to the skeletal muscle. In certain embodiments, the AAV capsid protein comprises a sequence as disclosed in U.S. Pat. No. 7,198,951, such as, but not limited to, AAV-9 (SEQ ID NOs: 1-3 of U.S. Pat. No. 7,198,951), AAV-2 (SEQ ID NO: 4 of U.S. Pat. No. 7,198,951), AAV-1 (SEQ ID NO: 5 of U.S. Pat. No. 7,198,951), AAV-3 (SEQ ID NO: 6 of U.S. Pat. No. 7,198,951), and AAV-8 (SEQ ID NO: 7 of U.S. Pat. No. 7,198,951). AAV serotypes identified from rhesus monkeys, e.g., rh.8, rh. 10, rh.39, rh.43, and rh. 74, are also contemplated in the instant invention. Besides the natural AAV serotypes, modified AAV capsids have been developed for improving efficiency of delivery, tissue tropism, and immunogenicity. Exemplary natural and modified AAV capsids are disclosed in U.S. Pat. Nos. 7,906,111, 9,493,788, and 7,198,951, and PCT Publication No. WO2017189964A2.

The wild-type AAV genome contains two 145 nucleotide inverted terminal repeats (ITRs), which contain signal sequences directing AAV replication, genome encapsidation and integration. In addition to the ITRs, three AAV promoters, p5, p19, and p40, drive expression of two open reading frames encoding rep and cap genes. Two rep promoters, coupled with differential splicing of the single AAV intron, result in the production of four rep proteins (Rep 78, Rep 68, Rep 52, and Rep 40) from the rep gene. Rep proteins are responsible for genomic replication. The Cap gene is expressed from the p40 promoter, and encodes three capsid proteins (VP1, VP2, and VP3) which are splice variants of the cap gene. These proteins form the capsid of the AAV particle.

Because the cis-acting signals for replication, encapsidation, and integration are contained within the ITRs, some or all of the 4.3 kb internal genome may be replaced with foreign DNA, for example, an expression cassette for an exogenous gene of interest. Accordingly, in certain embodiments, the AAV vector comprises a genome comprising an expression cassette for an exogenous gene flanked by a 5′ ITR and a 3′ ITR. The ITRs may be derived from the same serotype as the capsid or a derivative thereof. Alternatively, the ITRs may be of a different serotype from the capsid, thereby generating a pseudotyped AAV. In certain embodiments, the ITRs are derived from AAV-2. In certain embodiments, the ITRs are derived from AAV-5. At least one of the ITRs may be modified to mutate or delete the terminal resolution site, thereby allowing production of a self-complementary AAV vector.

The rep and cap proteins can be provided in trans, for example, on a plasmid, to produce an AAV vector. A host cell line permissive of AAV replication must express the rep and cap genes, the ITR-flanked expression cassette, and helper functions provided by a helper virus, for example adenoviral genes E1a, E1b55K, E2a, E4orf6, and VA (Weitzman et al., Adeno-associated virus biology. Adeno-Associated Virus: Methods and Protocols, pp. 1-23, 2011). Methods for generating and purifying AAV vectors have been described in detail (See e.g., Mueller et al., (2012) CURRENT PROTOCOLS IN MICROBIOLOGY, 14D.1.1-14D.1.21, Production and Discovery of Novel Recombinant Adeno-Associated Viral Vectors). Numerous cell types are suitable for producing AAV vectors, including HEK293 cells, COS cells, HeLa cells, BHK cells, Vero cells, as well as insect cells (See e.g. U.S. Pat. Nos. 6,156,303, 5,387,484, 5,741,683, 5,691, 176, 5,688,676, and 8,163,543, U.S. Patent Publication No. 20020081721, and PCT Publication Nos. WO00/47757, WO00/24916, and WO96/17947). AAV vectors are typically produced in these cell types by one plasmid containing the ITR-flanked expression cassette, and one or more additional plasmids providing the additional AAV and helper virus genes.

AAV of any serotype may be used in the present invention. Similarly, it is contemplated that any adenoviral type may be used, and a person of skill in the art will be able to identify AAV and adenoviral types suitable for the production of their desired recombinant AAV vector (rAAV). AAV particles may be purified, for example by affinity chromatography, iodixonal gradient, or CsCl gradient.

AAV vectors may have single-stranded genomes that are 4.7 kb in size, or are larger or smaller than 4.7 kb, including oversized genomes that are as large as 5.2 kb, or as small as 3.0 kb. Thus, where the exogenous gene of interest to be expressed from the AAV vector is small, the AAV genome may comprise a stuffer sequence. Further, vector genomes may be substantially self-complementary thereby allowing for rapid expression in the cell. In certain embodiments, the genome of a self-complementary AAV vector comprises from 5′ to 3′: a 5′ ITR; a first nucleic acid sequence comprising a promoter and/or enhancer operably linked to a coding sequence of a gene of interest; a modified ITR that does not have a functional terminal resolution site; a second nucleic acid sequence complementary or substantially complementary to the first nucleic acid sequence; and a 3′ ITR. AAV vectors containing genomes of all types are suitable for use in the method of the present invention.

Non-limiting examples of AAV vectors include pAAV-MCS (Agilent Technologies), pAAVK-EF1α-MCS (System Bio Catalog #AAV502A-1), pAAVK-EF1α-MCS1-CMV-MCS2 (System Bio Catalog #AAV503A-1), pAAV-ZsGreen1 (Clontech Catalog #6231), pAAV-MCS2 (Addgene Plasmid #46954), AAV-Stuffer (Addgene Plasmid #106248), pAAVscCBPIGpluc (Addgene Plasmid #35645), AAVSI_Puro_PGK1_3×FLAG_Twin_Strep (Addgene Plasmid #68375), pAAV-RAM-d2TTA::TRE-MCS-WPRE-pA (Addgene Plasmid #63931), pAAV-UbC (Addgene Plasmid #62806), pAAVS1-P-MCS (Addgene Plasmid #80488), pAAV-Gateway (Addgene Plasmid #32671), pAAV-Puro_siKD (Addgene Plasmid #86695), pAAVS1-Nst-MCS (Addgene Plasmid #80487), pAAVS1-Nst-CAG-DEST (Addgene Plasmid #80489), pAAVS1-P-CAG-DEST (Addgene Plasmid #80490), pAAVf-EnhCB-lacZnls (Addgene Plasmid #35642), and pAAVS1-shRNA (Addgene Plasmid #82697). These vectors can be modified to be suitable for therapeutic use. For example, an exogenous gene of interest can be inserted in a multiple cloning site, and a selection marker (e.g., puro or a gene encoding a fluorescent protein) can be deleted or replaced with another (same or different) exogenous gene of interest. Further examples of AAV vectors are disclosed in U.S. Pat. Nos. 5,871,982, 6,270,996, 7,238,526, 6,943,019, 6,953,690, 9,150,882, and 8,298,818, U.S. Patent Publication No. 2009/0087413, and PCT Publication Nos. WO2017075335A1, WO2017075338A2, and WO2017201258A1.

In certain embodiments, the expression vector is an AAV vector capable of targeting the nervous system, e.g., the central nervous system, in a subject, e.g., a human subject. Exemplary AAV vectors that can target the nervous system include the AAV9 variants AAV-PHP.B (See, e.g., Deverman et al. (2016) NAT. BIOTECHNOL. 34 (2): 204-209), AAV-AS (See, e.g., Choudhury et al. (2016) MOL. THER. 24:726-35), and AAV-PHP.eB (See, e.g., Chan et al. (2017) NAT. NEUROSCI. 20:1172-79). Additional exemplary AAV-based strategies for targeting the nervous system are described in Bedrook et al. (2018) ANNU REV NEUROSCI. 41:323-348. In certain embodiments, the AAV vector is an AAV-PHP.eB vector.

### Lentivirus Vectors

In certain embodiments, the viral vector can be a retroviral vector. Examples of retroviral vectors include moloney murine leukemia virus vectors, spleen necrosis virus vectors, and vectors derived from retroviruses such as rous sarcoma virus, harvey sarcoma virus, avian leukosis virus, human immunodeficiency virus, myeloproliferative sarcoma virus, and mammary tumor virus. Retroviral vectors are useful as agents to mediate retroviral-mediated gene transfer into eukaryotic cells.

In certain embodiments, the retroviral vector is a lentiviral vector. Exemplary lentiviral vectors include vectors derived from human immunodeficiency virus-1 (HIV-1), human immunodeficiency virus-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Retroviral vectors typically are constructed such that the majority of sequences coding for the structural genes of the virus are deleted and replaced by the gene(s) of interest. Often, the structural genes (i.e., gag, pol, and env), are removed from the retroviral backbone using genetic engineering techniques known in the art. Accordingly, a minimum retroviral vector comprises from 5′ to 3′: a 5′ long terminal repeat (LTR), a packaging signal, an optional exogenous promoter and/or enhancer, an exogenous gene of interest, and a 3′ LTR. If no exogenous promoter is provided, gene expression is driven by the 5′ LTR, which is a weak promoter and requires the presence of Tat to activate expression. The structural genes can be provided in separate vectors for manufacture of the lentivirus, rendering the produced virions replication-defective. Specifically, with respect to lentivirus, the packaging system may comprise a single packaging vector encoding the Gag, Pol, Rev, and Tat genes, and a third, separate vector encoding the envelope protein Env (usually VSV-G due to its wide infectivity). To improve the safety of the packaging system, the packaging vector can be split, expressing Rev from one vector, Gag and Pol from another vector. Tat can also be eliminated from the packaging system by using a retroviral vector comprising a chimeric 5′ LTR, wherein the U3 region of the 5′ LTR is replaced with a heterologous regulatory element.

The genes can be incorporated into the proviral backbone in several general ways. The most straightforward constructions are ones in which the structural genes of the retrovirus are replaced by a single gene that is transcribed under the control of the viral regulatory sequences within the LTR. Retroviral vectors have also been constructed which can introduce more than one gene into target cells. Usually, in such vectors one gene is under the regulatory control of the viral LTR, while the second gene is expressed either off a spliced message or is under the regulation of its own, internal promoter.

Accordingly, the new gene(s) are flanked by 5′ and 3′ LTRs, which serve to promote transcription and polyadenylation of the virion RNAs, respectively. The term “long terminal repeat” or “LTR” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals, and sequences needed for replication and integration of the viral genome. The U3 region contains the enhancer and promoter elements. The U5 region is the sequence between the primer binding site and the R region and contains the polyadenylation sequence. The R (repeat) region is flanked by the U3 and U5 regions. In certain embodiments, the R region comprises a trans-activation response (TAR) genetic element, which interacts with the trans-activator (tat) genetic element to enhance viral replication. This element is not required in embodiments wherein the U3 region of the 5′ LTR is replaced by a heterologous promoter.

In certain embodiments, the retroviral vector comprises a modified 5′ LTR and/or 3′ LTR. Modifications of the 3′ LTR are often made to improve the safety of lentiviral or retroviral systems by rendering viruses replication-defective. In specific embodiments, the retroviral vector is a self-inactivating (SIN) vector. As used herein, a SIN retroviral vector refers to a replication-defective retroviral vector in which the 3′ LTR U3 region has been modified (e.g., by deletion or substitution) to prevent viral transcription beyond the first round of viral replication. This is because the 3′ LTR U3 region is used as a template for the 5′ LTR U3 region during viral replication and, thus, the viral transcript cannot be made without the U3 enhancer-promoter. In a further embodiment, the 3′ LTR is modified such that the U5 region is replaced, for example, with an ideal polyadenylation sequence. It should be noted that modifications to the LTRs such as modifications to the 3′ LTR, the 5′ LTR, or both 3′ and 5′ LTRs, are also contemplated to be useful in the practice of the invention.

In certain embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus, because there is no complete U3 sequence in the virus production system.

Adjacent the 5′ LTR are sequences necessary for reverse transcription of the genome (the tRNA primer binding site) and for efficient packaging of viral RNA into particles (the Psi site). As used herein, the term “packaging signal” or “packaging sequence” refers to sequences located within the retroviral genome which are required for encapsidation of retroviral RNA strands during viral particle formation (see e.g., Clever et al., 1995 J. VIROLOGY, 69 (4): 2101-09). The packaging signal may be a minimal packaging signal (also referred to as the psi [Ψ] sequence) needed for encapsidation of the viral genome.

In certain embodiments, the retroviral vector (e.g., lentiviral vector) further comprises a FLAP. As used herein, the term “FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou et al. (2000) CELL, 101:173. During reverse transcription, central initiation of the plus-strand DNA at the cPPT and central termination at the CTS lead to the formation of a three-stranded DNA structure: a central DNA flap. While not wishing to be bound by any theory, the DNA flap may act as a cis-active determinant of lentiviral genome nuclear import and/or may increase the titer of the virus. In particular embodiments, the retroviral vector backbones comprise one or more FLAP elements upstream or downstream of the heterologous genes of interest in the vectors. For example, in particular embodiments, a transfer plasmid includes a FLAP element. In one embodiment, a vector of the invention comprises a FLAP element isolated from HIV-1.

In certain embodiments, the retroviral vector (e.g., lentiviral vector) further comprises an export element. In one embodiment, retroviral vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) RRE (see e.g., Cullen et al., (1991) J. VIROL. 65:1053; and Cullen et al., (1991) CELL 58:423) and the hepatitis B virus post-transcriptional regulatory element (HPRE). Generally, the RNA export element is placed within the 3′ UTR of a gene, and can be inserted as one or multiple copies.

In certain embodiments, the retroviral vector (e.g., lentiviral vector) further comprises a posttranscriptional regulatory element. A variety of posttranscriptional regulatory elements can increase expression of a heterologous nucleic acid, e.g., woodchuck hepatitis virus posttranscriptional regulatory element (WPRE; see Zufferey et al., (1999) J. VIROL., 73:2886); the posttranscriptional regulatory element present in hepatitis B virus (HPRE) (Huang et al., MOL. CELL. BIOL., 5:3864); and the like (Liu et al., (1995), GENES DEV., 9:1766). The posttranscriptional regulatory element is generally positioned at the 3′ end the heterologous nucleic acid sequence. This configuration results in synthesis of an mRNA transcript whose 5′ portion comprises the heterologous nucleic acid coding sequences and whose 3′ portion comprises the posttranscriptional regulatory element sequence. In certain embodiments, vectors of the invention lack or do not comprise a posttranscriptional regulatory element such as a WPRE or HPRE, because in some instances these elements increase the risk of cellular transformation and/or do not substantially or significantly increase the amount of mRNA transcript or increase mRNA stability. Therefore, in certain embodiments, vectors of the invention lack or do not comprise a WPRE or HPRE as an added safety measure.

Elements directing the efficient termination and polyadenylation of the heterologous nucleic acid transcripts increase heterologous gene expression. Transcription termination signals are generally found downstream of the polyadenylation signal. Accordingly, in certain embodiments, the retroviral vector (e.g., lentiviral vector) further comprises a polyadenylation signal. The term “polyadenylation signal” or “polyadenylation sequence” as used herein denotes a DNA sequence which directs both the termination and polyadenylation of the nascent RNA transcript by RNA polymerase H. Efficient polyadenylation of the recombinant transcript is desirable as transcripts lacking a polyadenylation signal are unstable and are rapidly degraded. Illustrative examples of polyadenylation signals that can be used in a vector of the invention, includes an ideal polyadenylation sequence (e.g., AATAAA, ATTAAA AGTAAA), a bovine growth hormone polyadenylation sequence (BGHpA), a rabbit β-globin polyadenylation sequence (rβgpA), or another suitable heterologous or endogenous polyadenylation sequence known in the art.

In certain embodiments, a retroviral vector further comprises an insulator element. Insulator elements may contribute to protecting retrovirus-expressed sequences, e.g., therapeutic genes, from integration site effects, which may be mediated by cis-acting elements present in genomic DNA and lead to deregulated expression of transferred sequences (i.e., position effect; see, e.g., Burgess-Beusse et al., (2002) PROC. NATL. ACAD. SCI., USA, 99:16433; and Zhan et al., 2001, HUM. GENET., 109:471). In certain embodiments, the retroviral vector comprises an insulator element in one or both LTRs or elsewhere in the region of the vector that integrates into the cellular genome. Suitable insulators for use in the invention include, but are not limited to, the chicken β-globin insulator (see Chung et al., (1993). CELL 74:505; Chung et al., (1997) PROC. NATL. ACAD. SCI., USA 94:575; and Bell et al., 1999. CELL 98:387). Examples of insulator elements include, but are not limited to, an insulator from a β-globin locus, such as chicken HS4.

Non-limiting examples of lentiviral vectors include pL VX-EF1alpha-AcGFP1-C1 (Clontech Catalog #631984), pL VX-EF1alpha-IRES-mCherry (Clontech Catalog #631987), pLVX-Puro (Clontech Catalog #632159), pL VX-IRES-Puro (Clontech Catalog #632186), pLenti6/V5-DEST™ (Thermo Fisher), pLenti6.2/V5-DEST™ (Thermo Fisher), pLKO.1 (Plasmid #10878 at Addgene), pLKO.3G (Plasmid #14748 at Addgene), pSico (Plasmid #11578 at Addgene), pLJM1-EGFP (Plasmid #19319 at Addgene), FUGW (Plasmid #14883 at Addgene), pLVTHM (Plasmid #12247 at Addgene), pL VUT-tTR-KRAB (Plasmid #11651 at Addgene), pLL3.7 (Plasmid #11795 at Addgene), pLB (Plasmid #11619 at Addgene), pWPXL (Plasmid #12257 at Addgene), pWPI (Plasmid #12254 at Addgene), EF.CMV.RFP (Plasmid #17619 at Addgene), pLenti CMV Puro DEST (Plasmid #17452 at Addgene), pLenti-puro (Plasmid #39481 at Addgene), pULTRA (Plasmid #24129 at Addgene), pLX301 (Plasmid #25895 at Addgene), pHIV-EGFP (Plasmid #21373 at Addgene), pLV-mCherry (Plasmid #36084 at Addgene), pLionII (Plasmid #1730 at Addgene), pInducer10-mir-RUP-PheS (Plasmid #44011 at Addgene). These vectors can be modified to be suitable for therapeutic use. For example, a selection marker (e.g., puro, EGFP, or mCherry) can be deleted or replaced with a second exogenous gene of interest. Further examples of lentiviral vectors are disclosed in U.S. Pat. Nos. 7,629,153, 7,198,950, 8,329,462, 6,863,884, 6,682,907, 7,745,179, 7,250,299, 5,994,136, 6,287,814, 6,013,516, 6,797,512, 6,544,771, 5,834,256, 6,958,226, 6,207,455, 6,531,123, and 6,352,694, and PCT Publication No. WO2017/091786.

### Adenoviral Vectors

In certain embodiments, the viral vector can be an adenoviral vector. Adenoviruses are medium-sized (90-100 nm), non-enveloped (naked), icosahedral viruses composed of a nucleocapsid and a double-stranded linear DNA genome. The term “adenovirus” refers to any virus in the genus Adenoviridiae including, but not limited to, human, bovine, ovine, equine, canine, porcine, murine, and simian adenovirus subgenera. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

A human adenovirus can be used as the source of the adenoviral genome for the adenoviral vector. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serogroup or serotype. Adenoviral serotypes 1 through 51 are available from the American Type Culture Collection (ATCC, Manassas, Virginia). Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and PCT Publication Nos. WO1997/012986 and WO1998/053087.

Non-human adenovirus (e.g., ape, simian, avian, canine, ovine, or bovine adenoviruses) can be used to generate the adenoviral vector (i.e., as a source of the adenoviral genome for the adenoviral vector). For example, the adenoviral vector can be based on a simian adenovirus, including both new world and old world monkeys (see, e.g., Virus Taxonomy: VHIth Report of the International Committee on Taxonomy of Viruses (2005)). A phylogeny analysis of adenoviruses that infect primates is disclosed in, e.g., Roy et al. (2009) PLOS PATHOG. 5 (7): e1000503. A gorilla adenovirus can be used as the source of the adenoviral genome for the adenoviral vector. Gorilla adenoviruses and adenoviral vectors are described in, e.g., PCT Publication Nos. WO2013/052799, WO2013/052811, and WO2013/052832. The adenoviral vector can also comprise a combination of subtypes and thereby be a “chimeric” adenoviral vector.

The adenoviral vector can be replication-competent, conditionally replication-competent, or replication-deficient. A replication-competent adenoviral vector can replicate in typical host cells, i.e., cells typically capable of being infected by an adenovirus. A conditionally-replicating adenoviral vector is an adenoviral vector that has been engineered to replicate under pre-determined conditions. For example, replication-essential gene functions, e.g., gene functions encoded by the adenoviral early regions, can be operably linked to an inducible, repressible, or tissue-specific transcription control sequence, e.g., a promoter. Conditionally-replicating adenoviral vectors are further described in U.S. Pat. No. 5,998,205. A replication-deficient adenoviral vector is an adenoviral vector that requires complementation of one or more gene functions or regions of the adenoviral genome that are required for replication, as a result of, for example, a deficiency in one or more replication-essential gene function or regions, such that the adenoviral vector does not replicate in typical host cells, especially those in a human to be infected by the adenoviral vector.

Preferably, the adenoviral vector is replication-deficient, such that the replication-deficient adenoviral vector requires complementation of at least one replication-essential gene function of one or more regions of the adenoviral genome for propagation (e.g., to form adenoviral vector particles). The adenoviral vector can be deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenovector (HC-Ad)). See, e.g., Morsy et al. (1998) PROC. NATL. ACAD. SCI. USA 95:965-976, Chen et al. (1997) PROC. NATL. ACAD. SCI. USA 94:1645-1650, and Kochanek et al. (1999) HUM. GENE THER. 10 (15): 2451-9. Examples of replication-deficient adenoviral vectors are disclosed in U.S. Pat. Nos. 5,837,511, 5,851,806, 5,994, 106, 6,127,175, 6,482,616, and 7,195,896, and PCT Publication Nos. WO1994/028152, WO1995/002697, WO1995/016772, WO1995/034671, WO1996/022378, WO1997/012986, WO1997/021826, and WO2003/022311.

The replication-deficient adenoviral vector of the invention can be produced in complementing cell lines that provide gene functions not present in the replication-deficient adenoviral vector, but required for viral propagation, at appropriate levels in order to generate high titers of viral vector stock. Such complementing cell lines are known and include, but are not limited to, 293 cells (described in, e.g., Graham et al. (1977) J. GEN. VIROL. 36:59-72), PER.C6 cells (described in, e.g., PCT Publication No. WO1997/000326, and U.S. Pat. Nos. 5,994,128 and 6,033,908), and 293-ORF6 cells (described in, e.g., PCT Publication No. WO1995/034671 and Brough et al. (1997) J. VIROL. 71:9206-9213). Other suitable complementing cell lines to produce the replication-deficient adenoviral vector of the invention include complementing cells that have been generated to propagate adenoviral vectors encoding transgenes whose expression inhibits viral growth in host cells (see, e.g., U.S. Patent Publication No. 2008/0233650). Additional suitable complementing cells are described in, for example, U.S. Pat. Nos. 6,677,156 and 6,682,929, and PCT Publication No. WO2003/020879. Formulations for adenoviral vector-containing compositions are further described in, for example, U.S. Pat. Nos. 6,225,289, and 6,514,943, and PCT Publication No. WO2000/034444.

Additional exemplary adenoviral vectors, and/or methods for making or propagating adenoviral vectors are described in U.S. Pat. Nos. 5,559,099, 5,837,511, 5,846,782, 5,851,806, 5,994,106, 5,994,128, 5,965,541, 5,981,225, 6,040,174, 6,020,191, 6,083,716, 6,113,913, 6,303,362, 7,067,310, and 9,073,980.

Commercially available adenoviral vector systems include the ViraPower™ Adenoviral Expression System available from Thermo Fisher Scientific, the AdEasy™ adenoviral vector system available from Agilent Technologies, and the Adeno-X™ Expression System 3 available from Takara Bio USA, Inc.

### Viral Vector Production

Methods for producing viral vectors are known in the art. Typically, a virus of interest is produced in a suitable host cell line using conventional techniques including culturing a transfected or infected host cell under suitable conditions so as to allow the production of infectious viral particles. Nucleic acids encoding viral genes and/or tRNAs can be incorporated into plasmids and introduced into host cells through conventional transfection or transformation techniques. Exemplary suitable host cells for production of disclosed viruses include human cell lines such as HeLa, Hela-S3, HEK293, 911, A549, HER96, or PER-C6 cells. Specific production and purification conditions will vary depending upon the virus and the production system employed.

In certain embodiments, producer cells may be directly administered to a subject, however, in other embodiments, following production, infectious viral particles are recovered from the culture and optionally purified. Typical purification steps may include plaque purification, centrifugation, e.g., cesium chloride gradient centrifugation, clarification, enzymatic treatment, e.g., benzonase or protease treatment, chromatographic steps, e.g., ion exchange chromatography or filtration steps.

### IV. Pharmaceutical Compositions

For therapeutic use, a tRNA and/or expression vector preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (See Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1:10-29). In certain embodiments, the composition does not comprise (or is substantially free of, for example, the composition comprises less than 5%, 4%, 3%, 2%, 1%, 0.5% or 0.1% of) a nanoparticle or an aminolipid delivery compound, e.g., as described in U.S. Patent Publication No. 2017/0354672. In certain embodiments, the tRNA or expression vector introduced into the cell or administered to the subject is not conjugated to or associated with another moiety, e.g., a carrier particle, e.g., an aminolipid particle. As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. Typically the moieties are attached either by one or more covalent bonds or by a mechanism that involves specific binding. Alternately, a sufficient number of weaker interactions can provide sufficient stability for moieties to remain physically associated.

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

Pharmaceutical compositions containing a tRNA and/or expression vector disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, a tRNA and/or expression vector is administered intrathecally. In certain embodiments, a tRNA and/or expression vector is administered by injection. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

In general, any method of delivering a nucleic acid molecule can be adapted for use with a tRNA (see e.g., Akhtar et al. (1992) TRENDS CELL. BIOL. 2 (5): 139-144 and PCT Publication No. WO94/02595). The tRNA can be modified or alternatively delivered using a drug delivery system to prevent the rapid degradation of the tRNA by endo- and exo-nucleases in vivo. tRNA molecules can be modified by chemical conjugation to lipophilic groups such as cholesterol to enhance cellular uptake and prevent degradation. tRNA molecules can also be conjugated to or otherwise associated with an aptamer. A tRNA can also be delivered using drug delivery systems such as a nanoparticle, a dendrimer, a polymer, liposomes, or a cationic delivery system. Positively charged cationic delivery systems facilitate binding of a tRNA molecule (negatively charged) and also enhance interactions at the negatively charged cell membrane to permit efficient uptake of a tRNA by the cell. Cationic lipids, dendrimers, or polymers can either be bound to the RNA, e.g., tRNA, or induced to form a vesicle or micelle (see e.g., Kim et al. (2008) JOURNAL OF CONTROLLED RELEASE 129 (2): 107-116) that encases the RNA. Methods for making and administering cationic-RNA complexes are well within the abilities of one skilled in the art (see, e.g., Sorensen et al. (2003) J. MOL. BIOL 327:761-766; Verma et al. (2003) CLIN. CANCER RES. 9:1291-1300; Arnold et al. (2007) J. HYPERTENS. 25:197-205). Some non-limiting examples of drug delivery systems useful for systemic delivery of RNAs, e.g., tRNAs include DOTAP (Sorensen et al. (2003) supra; Verma et al. (2003), supra), Oligofectamine, solid nucleic acid lipid particles (Zimmermann et al. (2006) NATURE 441:111-114), cardiolipin (Chien et al. (2005) CANCER GENE THER. 12:321-328; Pal et al. (2005) INT J. ONCOL. 26:1087-1091), polyethyleneimine (Bonnet et al. (2008) PHARM. RES. 25(12):2972-82; Aigner (2006) J. BIOMED. BIOTECHNOL. 71659), Arg-Gly-Asp (RGD) peptides (Liu (2006) MOL. PHARM. 3:472-487), and polyamidoamines (Tomalia et al. (2007) BIOCHEM. SOC. TRANS. 35:61-67; Yoo et al. (1999) PHARM. RES. 16:1799-1804). In certain embodiments, a tRNA forms a complex with cyclodextrin for systemic administration. Methods for administration and pharmaceutical compositions of RNAs and cyclodextrins can be found in U.S. Pat. No. 7,427,605.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

The compositions described herein may be administered locally or systemically. Administration will generally be parenteral administration. In a preferred embodiment, the pharmaceutical composition is administered subcutaneously and in an even more preferred embodiment intravenously. Preparations for parenteral administration include sterile aqueous or non-aqueous solutions, suspensions, and emulsions.

Generally, a therapeutically effective amount of active component, for example, a tRNA and/or expression vector, is in the range of 0.1 mg/kg to 100 mg/kg, e.g., 1 mg/kg to 100 mg/kg, 1 mg/kg to 10 mg/kg. In certain embodiments, a therapeutically effective amount of a viral expression vector is in the range of 10² to 10¹⁵ plaque forming units (pfus), e.g., 10² to 10¹⁰, 10² to 10⁵, 10⁵ to 10¹⁵, 10⁵ to 10¹⁰, or 10¹⁰ to 10¹⁵ plaque forming units. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life, and the disease being treated. Exemplary dosing frequencies are once per day, once per week and once every two weeks. A preferred route of administration is parenteral, e.g., intravenous infusion. In certain embodiments, a polypeptide and/or multimeric protein is lyophilized, and then reconstituted in buffered saline, at the time of administration.

In certain embodiments, the tRNA or expression vector is not conjugated to or associated with another moiety, e.g., a carrier particle, e.g., an aminolipid particle. In certain embodiments, the tRNA or expression vector is introduced into the cell or administered to subject in a dosage form lacking a nanoparticle. In certain embodiments, the tRNA or expression vector is introduced into the cell or administered to subject in a dosage form lacking an aminolipid delivery compound, e.g., as described in U.S. Patent Publication No. 2017/0354672.

### V. Therapeutic Uses

The compositions and methods disclosed herein can be used to treat a premature termination codon (PTC)-mediated disorder in a subject. As used herein, the term “PTC-mediated disorder” refers to a disorder that is mediated, enhanced, exacerbated, or otherwise facilitated by or associated with a PTC in a gene.

The invention provides a method of treating a PTC-mediated disorder in a subject in need thereof. The method comprises administering to the subject an effective amount of a tRNA and/or expression vector, e.g., a tRNA and/or expression vector disclosed herein, either alone or in a combination with another therapeutic agent to treat the PTC-mediated disorder in the subject.

In certain embodiments, the premature termination codon-mediated disorder is a disorder listed in TABLE 5 below, and/or the gene with a premature termination codon is a gene listed in the corresponding row of TABLE 5 below.

TABLE 5

| Gene | Disorder |
|---|---|
| SCN1A | Dravet Syndrome; Genetic Epilepsy with Febrile Seizures |
| | (GEFS) |
| KCNQ2 | Benign Familial Infantile Epilepsy (BFIE); Early Infantile |
| | Epileptic Encephalopathy (EIEE) |
| SCN2A | Benign Familial Infantile Epilepsy (BFIE); Early Infantile |
| | Epileptic Encephalopathy (EIEE) |
| CDKL5 | Early Infantile Epileptic Encephalopathy (EIEE); Lennox- |
| | Gastaut Syndrome; CDKL5 deficiency disorder |
| MECP2 | Rett Syndrome; PPM-X Syndrome |
| STXBP1 | Early Infantile Epileptic Encephalopathy (EIEE); Ohtahara |
| | Syndrome; Dravet Syndrome |
| SCN8A | Benign Familial Infantile Epilepsy (BFIE); Early Infantile |
| | Epileptic Encephalopathy (EIEE) |
| CACNA1A | Episodic Ataxia; Hemiplegic Migraine |
| SLC2A1 | Iditiopathic Generalized Epilepsy |
| FOXG1 | FOXG1 Syndrome |
| PCDH19 | Early Infantile Epileptic Encephalopathy (EIEE) |
| GRIN2B | Early Infantile Epileptic Encephalopathy (EIEE) |
| DEPDC5 | Familial Focal Epilepsy with Variable Foci (FFEVF) |
| GRIN2A | Early Infantile Epileptic Encephalopathy (EIEE); Lennox- |
| | Gastaut Syndrome |
| CHD2 | Childhood-onset epileptic encephalopathy |
| SCN9A | Congenital insensitivity to pain, etc |
| SYNGAP1 | SYNGAP1-related intellectual disability |
| ALDH7A1 | Pyridoxine-dependent epilepsy |
| GRIN1 | Early Infantile Epileptic Encephalopathy (EIEE); Lennox- |
| | Gastaut Syndrome |
| TBC1D24 | Early Infantile Epileptic Encephalopathy (EIEE); Familial |
| | Infantile Myoclonic Epilepsy (FIME) |
| SLC6A1 | Myoclonic Astatic Epilepsy |
| DNM1 | Early Infantile Epileptic Encephalopathy (EIEE) |
| ARX | Early Infantile Epileptic Encephalopathy (EIEE); X-linked |
| | Intellectual Disability |
| KCNB1 | Early Infantile Epileptic Encephalopathy (EIEE) |
| KCNA1 | Partial Epilepsy and Episodic Ataxia |
| GABRG2 | Genetic Epilepsy with Febrile Seizures (GEFS); Early |
| | Infantile Epileptic Encephalopathy (EIEE); Febrile seizures |
| WWOX | Early Infantile Epileptic Encephalopathy (EIEE) |
| GABRB3 | Early Infantile Epileptic Encephalopathy (EIEE); Lennox- |
| | Gastaut Syndrome |
| SZT2 | Early Infantile Epileptic Encephalopathy (EIEE) |
| LGI1 | Autosomal Dominant Partial Epilepsy with Auditory |
| | Features (ADPEAF) |
| PNPO | PNPO-Deficiency |
| SCN1B | Genetic Epilepsy with Febrile Seizures (GEFS); Early |
| | Infantile Epileptic Encephalopathy (EIEE) |
| UBA5 | Early Infantile Epileptic Encephalopathy (EIEE) |
| KCTD7 | Progressive Myoclonus Epilepsy |
| SCARB2 | Action Myoclonus - Renal Failure (AMRF); Progressive |
| | Myoclonic Epilepsy |
| SLC13A5 | Early Infantile Epileptic Encephalopathy (EIEE) |
| CSTB | Progressive Myoclonic Epilepsy |
| EPM2A | Progressive Myoclonic Epilepsy |
| PRRT2 | Benign Familial Infantile Seizures (BFIS) |
| NHLRC1 | Progressive Myoclonic Epilepsy |
| SLC25A22 | Early Infantile Epileptic Encephalopathy (EIEE) |
| PRRT2 | Benign Familial Infantile Seizures (BFIS) |
| ALG13 | Early Infantile Epileptic Encephalopathy (EIEE) |

In certain embodiments, the premature termination codon-mediated disorder is a disorder listed in TABLE 6 below, and/or the gene with a premature termination codon is a gene listed in the corresponding row of TABLE 6 below.

TABLE 6

| Gene | Disorder |
|---|---|
| β-globin | β-thalassemia |
| CHM | Choroideremia |
| CFTR | Cystic Fibrosis |
| dystrophin | Duchenne Muscular Dystrophy |
| α-L-iduronidase | Hurler Syndrome |
| KIF1A | KIF1A |
| FBN1 | Marfan Syndrome |
| ARSB | Maroteaux-Lamy Syndrome |
| SMPD1 | Niemann Pick Disease |
| NAGLU | Sanfilippo Syndrome |
| DHCR7 | Smith-Lemli-Opitz Syndrome |
| SCN5A | Brugada Syndrome |
| KCNH2 (hERG) | Long QT Syndrome type 2 |
| KCNQ1 | Long QT Syndrome type 1 |
| TTN | Dilated Cardiomyopathy |
| MYBPC3 | Familial Hypertrophic |
| | Cardiomyopathy |
| LMNA | Dilated Cardiomyopathy |
| | (sometimes Emery-Dreifuss |
| | Muscular Dystrophy) |
| PKP2 | Familial Arrythmogenic Right |
| | Ventricular Dysplasia |
| PLN | Familial Isolated Dilated |
| | Cardiomyopathy |
| TSC1/2 | Tuberous Sclerosis |
| LDLR | Familial Hypercholesterolemia |
| SMN1 | Spinal Muscular Atrophy |

In certain embodiments, the PTC-mediated disorder is an epilepsy (e.g., Dravet syndrome), wherein the method reduces seizure frequency, seizure severity, and/or cognitive impairment in the subject. For example, in certain embodiments, the method reduces seizure frequency in the subject by at least 10%, 20%, 30%, 40%, 50%, 60%, 70%, 80%, 90% or 100% over the period of, e.g., a day, a week, or a month. In certain embodiments, the method reduces seizure frequency by 50% over the period of, e.g., a day, a week, or a month.

In certain embodiments, the PTC-mediated disorder is Dravet and/or the gene with a premature termination codon is SCN1A. In certain embodiments, a premature termination codon in the SCN1A gene is caused by a mutation, or a combination of mutations, selected from c.5745C>G, c.5713G>T, c.5701C>T, c.5677C>T, c.5641C>T, c.5629C>T, c.5623C>T, c.5503A>T, c.5473G>T, c.5437G>T, c.5428C>T, c.5403G>A, c.5402G>A, c.5383G>T, c.5371G>T, c.5049T>G, c.4921G>T, c.4900C>T, c.4873C>T, c.4779del, c.4778G>A, c.4774G>T, c.4761T>G, c.4648G>T, c.4540C>T, c.4516A>T, c.4514C>A, c.4508T>G, c.4488C>G, c.4471G>T, c.4300A>T, c.4269G>A, c.4268G>A, c.4233T>A, c.4222G>T, c.4191G>A, c.4190G>A, c.4186C>T, c.4159A>T, c.4155C>A, c.3964del, c.3952C>T, c.3825G>A, c.3824G>A, c.3819G>A, c.3818G>A, c.3795T>A, c.3789T>G, c.3779G>A, c.3750C>G, c.3724G>T, c.3700C>T, c.3697C>T, c.3657dup, c.3624G>A, c.3604C>T, c.3582G>A, c.3578G>A, c.3574C>T, c.3463C>T, c.3454del, c.3424G>T, c.3422C>A, c.3406G>T, c.3328G>T, c.3273C>A, c.3262G>T, c.3073C>T, c.3060T>A, c.2844T>A, c.2749C>T, c.2695C>T, c.2645T>A, c.2560C>T, c.2551C>T, c.2546C>A, c.2462G>A, c.2298del, c.2228G>A, c.2181G>A, c.2180G>A, c.2101C>T, c.2038A>T, c.1958T>A, c.1837C>T, c.1834C>T, c.1804G>T, c.1795G>T, c.1738C>T, c.1702C>T, c.1660C>T, c.1624C>T, c.1516C>T, c.1378C>T, c.1363C>T, c.1354A>T, c.1348C>T, c.1345G>T, c.1344dup, c.1306G>T, c.1278C>A, c.1278C>G, c.1151G>A, c.1129C>T, c.1118T>A, c.942del, c.751del, c.644T>A, c.327C>G, c.249C>A, c.121A>T, c.4846_4850dup, c.4787_4788del, c.4578_4612dup, c.4211_4212del, c.4125_4130delinsATAATCATACTGATTGCCTAAAACTAAT, c.3690_3693del, c.3338_3339del, c.1247_1248insGTAGA, c.825_826insGTATA, and c.278_279dup. In certain embodiments, a premature termination codon in the SCN1A gene is caused by a mutation, or a combination of mutations, selected from c.58G>T, c.575G>A, c.664C>T, c.962C>G, c.1095dupT, c.1129C>T, c.1315C>T, c.1348C>T, c.1366G>T, c.1492A>T, c.1537G>T, c.1624C>T, c.1738C>T, c.1804G>T, c.1837C>T, c.2134C>T, c.2370T>A, c.2495G>A, c.2593C>T, c.2635delC, c.2904C>A, c.3295G>T, c.3311C>A, c.3452C>G, c.3637C>T, c.3656G>A, c.3733C>T, c.3783C>A, c.3829C>T, c.3985C>T, c.4359T>G, c.4547C>A, c.4573C>T, c.4721C>G, c.4954G>T, c.5641G>T, c.5656C>T, and c.5734C>T. In certain embodiments, a premature termination codon in the SCN1A gene is caused by a mutation selected from c.664C>T, c.1129C>T, c.1492A>T, c.1624C>T, c.1738C>T, c.1837C>T, c.2134C>T, c.2593C>T, c.3637C>T, c.3733C>T, c.3985C>T, c.4573C>T, c.5656C>T, and c.5734C>T. In certain embodiments, a premature termination codon in the SCN1A gene is caused by a mutation selected from c.1738C>T and c.3985C>T.

In certain embodiments, a premature termination codon in the SCN1A gene is caused by a mutation set forth in TABLE 7, or a combination of mutations set forth in TABLE 7.

TABLE 7

| Mutation | Mutation | Suppressor |
|---|---|---|
| (coding DNA) | (Protein) | Class |
| c.664C > T | Arg222Ter | Arg > TGA |
| c.3637C > T | Arg1213Ter | Arg > TGA |
| c.3733C > T | Arg1245Ter | Arg > TGA |
| c.2134C > T | Arg712Ter | Arg > TGA |
| c.1837C > T | Arg613Ter | Arg > TGA |
| c.4188C > A | Cys1396Ter | Cys > TGA |
| c.2877T > A | Cys959Ter | Cys > TGA |
| c.3183T > A | Cys1061Ter | Cys > TGA |
| c.3607C > T | Gln1203Ter | Gln > TAA |
| c.2782C > T | Gln928Ter | Gln > TAA |
| c.3829C > T | Gln1277Ter | Gln > TAA |
| c.2893C > T | Gln965Ter | Gln > TAA |
| c.3106C > T | Gln1036Ter | Gln > TAG |
| c.3496C > T | Gln1166Ter | Gln > TAG |
| c.5662C > T | Gln1888Ter | Gln > TAG |
| c.5461C > T | Gln1821Ter | Gln > TAG |
| c.3730C > T | Gln1244Ter | Gln > TAG |
| c.5506G > T | Glu1836Ter | Glu > TAA |
| c.5470G > T | Glu1824Ter | Glu > TAA |
| c.3757G > T | Glu1253Ter | Glu > TAA |
| c.3439G > T | Glu1147Ter | Glu > TAA |
| c.1345G > T | Glu449Ter | Glu > TAA |
| c.5404G > T | Glu1802Ter | Glu > TAG |
| c.1804G > T | Glu602Ter | Glu > TAG |
| c.5416G > T | Glu1806Ter | Glu > TAG |
| c.1795G > T | Glu599Ter | Glu > TAG |
| c.1549G > T | Glu517Ter | Glu > TAG |
| c.4255G > T | Gly1419Ter | Gly > TGA |
| c.4954G > T | Gly1652Ter | Gly > TGA |
| c.4807G > T | Gly1603Ter | Gly > TGA |
| c.487G > T | Gly163Ter | Gly > TGA |
| c.1843G > T | Gly615Ter | Gly > TGA |
| c.539T > A | Leu180Ter | Leu > TAA |
| c.2678T > A | Leu893Ter | Leu > TAA |
| c.644T > A | Leu215Ter | Leu > TAG |
| c.1958T > A | Leu653Ter | Leu > TAG |
| c.1118T > A | Leu373Ter | Leu > TAG |
| c.4541T > G | Leu1514Ter | Leu > TGA |
| c.2627T > G | Leu876Ter | Leu > TGA |
| c.4549A > T | Lys1517Ter | Lys > TAA |
| c.5536A > T | Lys1846Ter | Lys > TAA |
| c.121A > T | Lys41Ter | Lys > TAA |
| c.4192A > T | Lys1398Ter | Lys > TAA |
| c.1354A > T | Lys452Ter | Lys > TAA |
| c.2071A > T | Lys691Ter | Lys > TAG |
| c.3455C > A | Ser1152Ter | Ser > TAA |
| c.2579C > A | Ser860Ter | Ser > TAA |
| c.1883C > A | Ser628Ter | Ser > TAA |
| c.4547C > A | Ser1516Ter | Ser > TAG |
| c.2213G > A | Trp738Ter | Trp > TAG |
| c.3611G > A | Trp1204Ter | Trp > TAG |
| c.4811G > A | Trp1604Ter | Trp > TAG |
| c.4223G > A | Trp1408Ter | Trp > TAG |
| c.5435G > A | Trp1812Ter | Trp > TAG |
| c.3615G > A | Trp1205Ter | Trp > TGA |
| c.4224G > A | Trp1408Ter | Trp > TGA |
| c.4302G > A | Trp1434Ter | Trp > TGA |
| c.3858G > A | Trp1286Ter | Trp > TGA |
| c.5436G > A | Trp1812Ter | Trp > TGA |
| c.3762T > A | Tyr1254Ter | Tyr > TAA |
| c.3828T > A | Tyr1276Ter | Tyr > TAA |
| c.4266T > A | Tyr1422Ter | Tyr > TAA |
| c.3306C > A | Tyr1102Ter | Tyr > TAA |
| c.249C > A | Tyr83Ter | Tyr > TAA |
| c.5082T > G | Tyr1694Ter | Tyr > TAG |
| c.4794T > G | Tyr1598Ter | Tyr > TAG |
| c.4521C > G | Tyr1507Ter | Tyr > TAG |
| c.3822T > G | Tyr1274Ter | Tyr > TAG |
| c.5778C > G | Tyr1926Ter | Tyr > TAG |

Additional exemplary mutations, including exemplary mutations causing a premature termination codon in a gene, e.g., the SCN1A gene, can be found in ClinVar (available on the world wide web at ncbi.nlm.nih.gov/clinvar/), “A catalog of SCN1A variants” Lossin et al. (2009) BRAIN DEV. 2009 31 (2): 114-30, the SCN1A Registry (available on the world wide web at scnla.net/scn1a-registry/), the SCN1A Mutation Database (available on the world wide web at gzneurosci.com/scn1adatabase), and the Leiden Open Variation Database (LOVD v.3.0; available on the world wide web at databases.lovd.nl/shared/genes/SCN1A). Unless indicated otherwise, any SCN1A mutations described herein are relative to SCNla isoform 1 (NCBI reference sequence NM_001165963, SEQ ID NO: 863).

In another aspect, the invention provides a method of treating Dravet syndrome in a subject in need thereof wherein the subject has a SCN1A gene with a mutation set forth in a row TABLE 7, the method comprising administering to the subject an effective amount of a suppressor tRNA of the suppressor class indicated in the same row of TABLE 7 as the mutation, or an expression vector comprising a nucleotide sequence encoding the tRNA. “Suppressor Class” as used in TABLE 7 (e.g., Arg>TGA) refers to the endogenous tRNA type from which the suppressor tRNA is derived (e.g., an arginine tRNA) and the termination codon recognized by the suppressor tRNA (e.g., TGA). Exemplary Arg>TGA suppressor tRNAs include tRNAs comprising a nucleotide sequence selected from SEQ ID NOs: 6-9, 11, 16-18, 19-22, and 35. Exemplary Gln>TAA suppressor tRNAs include tRNAs comprising a nucleotide sequence selected from SEQ ID NOs: 36-40, 44, and 45. Exemplary Gln>TAG suppressor tRNAs include tRNAs comprising a nucleotide sequence selected from SEQ ID NOs: 178-182, 186, and 187.

For example, in certain embodiments, the subject has a SCN1A gene with a premature termination codon selected from c.664C>T, c.3637C>T, c.3733C>T, c.2134C>T, and c.1837C>T, and the method comprises administering to the subject an effective amount of a suppressor tRNA comprising a nucleotide sequence selected from SEQ ID NOs: 6-9, 11, 16-18, 19-22, and 35. In certain embodiments, the subject has a SCN1A gene with a premature termination codon selected from c.3607C>T, c.2782C>T, c.3829C>T, and c.2893C>T, and the method comprises administering to the subject an effective amount of a suppressor tRNA comprising a nucleotide sequence selected from SEQ ID NOs: 36-40, 44, and 45. In certain embodiments, the subject has a SCN1A gene with a premature termination codon selected from c.3106C>T, c.3496C>T, c.5662C>T, c.5461C>T, and c.3730C>T, and the method comprises administering to the subject an effective amount of a suppressor tRNA comprising a nucleotide sequence selected from SEQ ID NOs: 178-182, 186, and 187.

In certain embodiments, wherein the gene is a SCN1A gene, the SCN1A gene product produced with the tRNA is a functional SCN1A gene product. In certain embodiments, the functional SCN1A gene product has greater activity than the truncated SCN1A gene product, e.g., greater voltage-gated sodium channel activity. In certain embodiments, the method increases voltage-gated sodium channel activity in a cell, tissue, or subject by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 250%, about 300%, about 350%, about 400%, about 450%, about 500%, about 600%, about 700%, about 800%, about 900%, or about 1000% relative to a cell, tissue, or subject without the tRNA. In certain embodiments, the method increases voltage-gated sodium channel activity in a cell, tissue, or subject by from about 20% to about 200%, about 20% to about 180%, about 20% to about 160%, about 20% to about 140%, about 20% to about 120%, about 20% to about 100%, about 20% to about 80%, about 20% to about 60%, about 20% to about 40%, about 40% to about 200%, about 40% to about 180%, about 40% to about 160%, about 40% to about 140%, about 40% to about 120%, about 40% to about 100%, about 40% to about 80%, about 40% to about 60%, about 60% to about 200%, about 60% to about 180%, about 60% to about 160%, about 60% to about 140%, about 60% to about 120%, about 60% to about 100%, about 60% to about 80%, about 80% to about 200%, about 80% to about 180%, about 80% to about 160%, about 80% to about 140%, about 80% to about 120%, about 80% to about 100%, about 100% to about 200%, about 100% to about 180%, about 100% to about 160%, about 100% to about 140%, about 100% to about 120%, about 120% to about 200%, about 120% to about 180%, about 120% to about 160%, about 120% to about 140%, about 140% to about 200%, about 140% to about 180%, about 140% to about 160%, about 160% to about 200%, about 160% to about 180%, or about 180% to about 200% relative to a cell, tissue, or subject without the tRNA. Voltage-gated sodium channel activity may be measured by any method known in the art, for example, as described in Kalume et al. (2007) J. NEUROSCI. 27 (41): 11065-74, Yu et al. (2007) NAT. NEUROSCI. 9 (9): 1142-9, and Han et al. (2012) NATURE 489 (7416): 385-390.

In certain embodiments, the functional SCN1A gene product is the Nav1.1 protein. In certain embodiments, the functional SCN1A gene product comprises, consists essentially of, or consists of the amino acid sequence of any one of the following amino acid sequences, or an amino acid sequence having 80%, 85%, 86%, 87%, 88%, 89%, 90%, 91%, 92%, 93%, 94%, 95%, 96%, 97%, 98%, or 99% sequence identity to any one of the following amino acid sequences (each corresponding to different isoforms of SCN1A):

(SEQ ID NO: 863)
MEQTVLVPPGPDSFNFFTRESLAAIERRIAEEKAKNPKPDKKDDDENGPKPNSDLEAGKNLPFI
YGDIPPEMVSEPLEDLDPYYINKKTFIVLNKGKAIFRFSATSALYILTPFNPLRKIAIKILVHS
LFSMLIMCTILTNCVFMTMSNPPDWTKNVEYTFTGIYTFESLIKIIARGFCLEDFTFLRDPWNW
LDFTVITFAYVTEFVDLGNVSALRTFRVLRALKTISVIPGLKTIVGALIQSVKKLSDVMILTVF
CLSVFALIGLQLEMGNLRNKCIQWPPTNASLEEHSIEKNITVNYNGTLINETVFEEDWKSYIQD
SRYHYFLEGELDALLCGNSSDAGQCPEGYMCVKAGRNPNYGYTSFDTFSWAFLSLFRLMTQDFW
ENLYQLTLRAAGKTYMIFFVLVIFLGSFYLINLILAVVAMAYEEQNQATLEEAEQKEAEFQQMI
EQLKKQQEAAQQAATATASEHSREPSAAGRLSDSSSEASKLSSKSAKERRNRRKKRKQKEQSGG
EEKDEDEFQKSESEDSIRRKGFRESIEGNRLTYEKRYSSPHQSLLSIRGSLFSPRRNSRTSLES
FRGRAKDVGSENDFADDEHSTFEDNESRRDSLFVPRRHGERRNSNLSQTSRSSRMLAVEPANGK
MHSTVDCNGVVSLVGGPSVPTSPVGQLLPEVIIDKPATDDNGTTTETEMRKRRSSSFHVSMDEL
EDPSQRQRAMSIASILINTVEELEESRQKCPPCWYKFSNIFLIWDCSPYWLKVKHVVNLVVMDP
FVDLAITICIVLNTLFMAMEHYPMTDHFNNVLTVGNLVFTGIFTAEMELKIIAMDPYYYFQEGW
NIFDGFIVTLSLVELGLANVEGLSVLRSFRLLRVFKLAKSWPTLNMLIKIIGNSVGALGNLTLV
LAIIVFIFAVVGMQLFGKSYKDCVCKIASDCQLPRWHMNDFFHSFLIVERVLCGEWIETMWDCM
EVAGQAMCLTVEMMVMVIGNLVVLNLFLALLLSSFSADNLAATDDDNEMNNLQIAVDRMHKGVA
YVKRKIYEFIQQSFIRKQKILDEIKPLDDLNNKKDSCMSNHTAEIGKDLDYLKDVNGTTSGIGT
GSSVEKYIIDESDYMSFINNPSLTVTVPIAVGESDFENLNTEDESSESDLEESKEKLNESSSSS
EGSTVDIGAPVEEQPVVEPEETLEPEACFTEGCVQRFKCCQINVEEGRGKQWWNLRRTCFRIVE
HNWFETFIVEMILLSSGALAFEDIYIDQRKTIKTMLEYADKVETYIFILEMLLKWVAYGYQTYF
TNAWCWLDFLIVDVSLVSLTANALGYSELGAIKSLRTLRALRPLRALSRFEGMRVVVNALLGAI
PSIMNVLLVCLIFWLIFSIMGVNLFAGKFYHCINTTTGDRFDIEDVNNHTDCLKLIERNETARW
KNVKVNFDNVGFGYLSLLQVATFKGWMDIMYAAVDSRNVELQPKYEESLYMYLYFVIFIIFGSF
FTLNLFIGVIIDNFNQQKKKFGGQDIFMTEEQKKYYNAMKKLGSKKPQKPIPRPGNKFQGMVFD
FVTRQVFDISIMILICLNMVTMMVETDDQSEYVTTILSRINLVFIVLFTGECVLKLISLRHYYF
TIGWNIFDFVVVILSIVGMFLAELIEKYFVSPTLERVIRLARIGRILRLIKGAKGIRTLLFALM
MSLPALFNIGLLLFLVMFIYAIFGMSNFAYVKREVGIDDMFNFETFGNSMICLFQITTSAGWDG
LLAPILNSKPPDCDPNKVNPGSSVKGDCGNPSVGIFFFVSYIIISELVVVNMYIAVILENFSVA
TEESAEPLSEDDFEMFYEVWEKFDPDATQFMEFEKLSQFAAALEPPLNLPQPNKLQLIAMDLPM
VSGDRIHCLDILFAFTKRVLGESGEMDALRIQMEERFMASNPSKVSYQPITTTLKRKQEEVSAV
IIQRAYRRHLLKRTVKQASFTYNKNKIKGGANLLIKEDMIIDRINENSITEKTDLTMSTAACPP
SYDRVTKPIVEKHEQEGKDEKAKGK;
(SEQ ID NO: 864)
MEQTVLVPPGPDSFNFFTRESLAAIERRIAEEKAKNPKPDKKDDDENGPKPNSDLEAGKNLPFI
YGDIPPEMVSEPLEDLDPYYINKKTFIVLNKGKAIFRFSATSALYILTPENPLRKIAIKILVHS
LFSMLIMCTILTNCVFMTMSNPPDWTKNVEYTFTGIYTFESLIKIIARGFCLEDFTFLRDPWNW
LDFTVITFAYVTEFVDLGNVSALRTFRVLRALKTISVIPGLKTIVGALIQSVKKLSDVMILTVE
CLSVFALIGLQLEMGNLRNKCIQWPPTNASLEEHSIEKNITVNYNGTLINETVFEFDWKSYIQD
SRYHYFLEGFLDALLCGNSSDAGQCPEGYMCVKAGRNPNYGYTSFDTFSWAFLSLFRLMTQDFW
ENLYQLTLRAAGKTYMIFFVLVIFLGSFYLINLILAVVAMAYEEQNQATLEEAEQKEAEFQQMI
EQLKKQQEAAQQAATATASEHSREPSAAGRLSDSSSEASKLSSKSAKERRNRRKKRKQKEQSGG
EEKDEDEFQKSESEDSIRRKGFRFSIEGNRLTYEKRYSSPHQSLLSIRGSLFSPRRNSRTSLES
FRGRAKDVGSENDFADDEHSTFEDNESRRDSLFVPRRHGERRNSNLSQTSRSSRMLAVEPANGK
MHSTVDCNGVVSLVGGPSVPTSPVGQLLPEGTTTETEMRKRRSSSFHVSMDFLEDPSQRQRAMS
IASILTNTVEELEESRQKCPPCWYKFSNIFLIWDCSPYWLKVKHVVNLVVMDPFVDLAITICIV
LNTLFMAMEHYPMTDHFNNVLTVGNLVFTGIFTAEMFLKIIAMDPYYYFQEGWNIFDGFIVTLS
LVELGLANVEGLSVLRSFRLLRVFKLAKSWPTLNMLIKIIGNSVGALGNLTLVLAIIVFIFAVV
GMQLFGKSYKDCVCKIASDCQLPRWHMNDFFHSFLIVFRVLCGEWIETMWDCMEVAGQAMCLTV
FMMVMVIGNLVVLNLFLALLLSSFSADNLAATDDDNEMNNLQIAVDRMHKGVAYVKRKIYEFIQ
QSFIRKQKILDEIKPLDDLNNKKDSCMSNHTAEIGKDLDYLKDVNGTTSGIGTGSSVEKYIIDE
SDYMSFINNPSLTVTVPIAVGESDFENLNTEDESSESDLEESKEKLNESSSSSEGSTVDIGAPV
EEQPVVEPEETLEPEACFTEGCVQRFKCCQINVEEGRGKQWWNLRRTCFRIVEHNWFETFIVEM
ILLSSGALAFEDIYIDQRKTIKTMLEYADKVFTYIFILEMLLKWVAYGYQTYFTNAWCWLDFLI
VDVSLVSLTANALGYSELGAIKSLRTLRALRPLRALSRFEGMRVVVNALLGAIPSIMNVLLVCL
IFWLIFSIMGVNLFAGKFYHCINTTTGDRFDIEDVNNHTDCLKLIERNETARWKNVKVNEDNVG
FGYLSLLQVATFKGWMDIMYAAVDSRNVELQPKYEESLYMYLYFVIFIIFGSFFTLNLFIGVII
DNFNQQKKKFGGQDIFMTEEQKKYYNAMKKLGSKKPQKPIPRPGNKFQGMVFDFVTRQVFDISI
MILICLNMVTMMVETDDQSEYVTTILSRINLVFIVLFTGECVLKLISLRHYYFTIGWNIFDFVV
VILSIVGMFLAELIEKYFVSPTLFRVIRLARIGRILRLIKGAKGIRTLLFALMMSLPALFNIGL
LLFLVMFIYAIFGMSNFAYVKREVGIDDMENFETEGNSMICLFQITTSAGWDGLLAPILNSKPP
DCDPNKVNPGSSVKGDCGNPSVGIFFFVSYIIISFLVVVNMYIAVILENFSVATEESAEPLSED
DFEMFYEVWEKFDPDATQFMEFEKLSQFAAALEPPLNLPQPNKLQLIAMDLPMVSGDRIHCLDI
LFAFTKRVLGESGEMDALRIQMEERFMASNPSKVSYQPITTTLKRKQEEVSAVIIQRAYRRHLL
KRTVKQASFTYNKNKIKGGANLLIKEDMIIDRINENSITEKTDLTMSTAACPPSYDRVTKPIVE
KHEQEGKDEKAKGK;
(SEQ ID NO: 865)
MEQTVLVPPGPDSFNFFTRESLAAIERRIAEEKAKNPKPDKKDDDENGPKPNSDLEAGKNLPFI
YGDIPPEMVSEPLEDLDPYYINKKTFIVLNKGKAIFRFSATSALYILTPFNPLRKIAIKILVHS
LFSMLIMCTILTNCVFMTMSNPPDWTKNVEYTFTGIYTFESLIKIIARGFCLEDFTFLRDPWNW
LDFTVITFAYVTEFVDLGNVSALRTFRVLRALKTISVIPGLKTIVGALIQSVKKLSDVMILTVE
CLSVFALIGLQLEMGNLRNKCIQWPPTNASLEEHSIEKNITVNYNGTLINETVFEFDWKSYIQD
SRYHYFLEGFLDALLCGNSSDAGQCPEGYMCVKAGRNPNYGYTSFDTFSWAFLSLFRLMTQDFW
ENLYQLTLRAAGKTYMIFFVLVIFLGSFYLINLILAVVAMAYEEQNQATLEEAEQKEAEFQQMI
EQLKKQQEAAQQAATATASEHSREPSAAGRLSDSSSEASKLSSKSAKERRNRRKKRKQKEQSGG
EEKDEDEFQKSESEDSIRRKGFRFSIEGNRLTYEKRYSSPHQSLLSIRGSLESPRRNSRTSLES
FRGRAKDVGSENDFADDEHSTFEDNESRRDSLFVPRRHGERRNSNLSQTSRSSRMLAVFPANGK
MHSTVDCNGVVSLGTTTETEMRKRRSSSFHVSMDFLEDPSQRQRAMSIASILINTVEELEESRQ
KCPPCWYKFSNIFLIWDCSPYWLKVKHVVNLVVMDPFVDLAITICIVLNTLFMAMEHYPMTDHE
NNVLTVGNLVFTGIFTAEMFLKIIAMDPYYYFQEGWNIFDGFIVTLSLVELGLANVEGLSVLRS
FRLLRVFKLAKSWPTLNMLIKIIGNSVGALGNLTLVLAIIVFIFAVVGMQLFGKSYKDCVCKIA
SDCQLPRWHMNDFFHSFLIVFRVLCGEWIETMWDCMEVAGQAMCLTVEMMVMVIGNLVVLNLEL
ALLLSSFSADNLAATDDDNEMNNLQIAVDRMHKGVAYVKRKIYEFIQQSFIRKQKILDEIKPLD
DLNNKKDSCMSNHTAEIGKDLDYLKDVNGTTSGIGTGSSVEKYIIDESDYMSFINNPSLTVTVP
IAVGESDFENLNTEDESSESDLEESKEKLNESSSSSEGSTVDIGAPVEEQPVVEPEETLEPEAC
FTEGCVQRFKCCQINVEEGRGKQWWNLRRTCFRIVEHNWFETFIVEMILLSSGALAFEDIYIDQ
RKTIKTMLEYADKVFTYIFILEMLLKWVAYGYQTYFTNAWCWLDFLIVDVSLVSLTANALGYSE
LGAIKSLRTLRALRPLRALSRFEGMRVVVNALLGAIPSIMNVLLVCLIFWLIFSIMGVNLFAGK
FYHCINTTTGDRFDIEDVNNHTDCLKLIERNETARWKNVKVNFDNVGFGYLSLLQVATFKGWMD
IMYAAVDSRNVELQPKYEESLYMYLYFVIFIIFGSFFTLNLFIGVIIDNFNQQKKKFGGQDIFM
TEEQKKYYNAMKKLGSKKPQKPIPRPGNKFQGMVFDFVTRQVFDISIMILICLNMVTMMVETDD
QSEYVTTILSRINLVFIVLFTGECVLKLISLRHYYFTIGWNIFDFVVVILSIVGMFLAELIEKY
FVSPTLFRVIRLARIGRILRLIKGAKGIRTLLFALMMSLPALFNIGLLLFLVMFIYAIFGMSNF
AYVKREVGIDDMFNFETFGNSMICLFQITTSAGWDGLLAPILNSKPPDCDPNKVNPGSSVKGDC
GNPSVGIFFFVSYIIISFLVVVNMYIAVILENFSVATEESAEPLSEDDFEMFYEVWEKFDPDAT
QFMEFEKLSQFAAALEPPLNLPQPNKLQLIAMDLPMVSGDRIHCLDILFAFTKRVLGESGEMDA
LRIQMEERFMASNPSKVSYQPITTTLKRKQEEVSAVIIQRAYRRHLLKRTVKQASFTYNKNKIK
GGANLLIKEDMIIDRINENSITEKTDLTMSTAACPPSYDRVTKPIVEKHEQEGKDEKAKGK;
(SEQ ID NO: 866)
MEQTVLVPPGPDSFNFFTRESLAAIERRIAEEKAKNPKPDKKDDDENGPKPNSDLEAGKNLPFI
YGDIPPEMVSEPLEDLDPYYINKKTFIVLNKGKAIFRFSATSALYILTPENPLRKIAIKILVHS
LFSMLIMCTILTNCVEMTMSNPPDWTKNVEYTFTGIYTFESLIKIIARGFCLEDFTFLRDPWNW
LDFTVITFAYVTEFVDLGNVSALRTFRVLRALKTISVIPGLKTIVGALIQSVKKLSDVMILTVE
CLSVFALIGLQLFMGNLRNKCIQWPPTNASLEEHSIEKNITVNYNGTLINETVFEFDWKSYIQD
SRYHYFLEGELDALLCGNSSDAGQCPEGYMCVKAGRNPNYGYTSFDTFSWAFLSLFRLMTQDFW
ENLYQLTLRAAGKTYMIFFVLVIFLGSFYLINLILAVVAMAYEEQNQATLEEAEQKEAEFQQMI
EQLKKQQEAAQAATATASEHSREPSAAGRLSDSSSEASKLSSKSAKERRNRRKKRKQKEQSGGE
EKDEDEFQKSESEDSIRRKGFRFSIEGNRLTYEKRYSSPHQSLLSIRGSLFSPRRNSRTSLFSF
RGRAKDVGSENDFADDEHSTFEDNESRRDSLFVPRRHGERRNSNLSQTSRSSRMLAVFPANGKM
HSTVDCNGVVSLVGGPSVPTSPVGQLLPEGTTTETEMRKRRSSSFHVSMDFLEDPSQRQRAMSI
ASILTNTVEELEESRQKCPPCWYKFSNIFLIWDCSPYWLKVKHVVNLVVMDPFVDLAITICIVL
NTLFMAMEHYPMTDHFNNVLTVGNLVFTGIFTAEMFLKIIAMDPYYYFQEGWNIFDGFIVTLSL
VELGLANVEGLSVLRSFRLLRVFKLAKSWPTLNMLIKIIGNSVGALGNLTLVLAIIVFIFAVVG
MQLFGKSYKDCVCKIASDCQLPRWHMNDFFHSFLIVFRVLCGEWIETMWDCMEVAGQAMCLIVE
MMVMVIGNLVVLNLFLALLLSSFSADNLAATDDDNEMNNLQIAVDRMHKGVAYVKRKIYEFIQQ
SFIRKQKILDEIKPLDDLNNKKDSCMSNHTAEIGKDLDYLKDVNGTTSGIGTGSSVEKYIIDES
DYMSFINNPSLTVTVPIAVGESDFENLNTEDESSESDLEESKEKLNESSSSSEGSTVDIGAPVE
EQPVVEPEETLEPEACFTEGCVQRFKCCQINVEEGRGKQWWNLRRTCFRIVEHNWFETFIVEMI
LLSSGALAFEDIYIDQRKTIKTMLEYADKVFTYIFILEMLLKWVAYGYQTYFTNAWCWLDFLIV
DVSLVSLTANALGYSELGAIKSLRTLRALRPLRALSRFEGMRVVVNALLGAIPSIMNVLLVCLI
FWLIFSIMGVNLFAGKFYHCINTTTGDRFDIEDVNNHTDCLKLIERNETARWKNVKVNFDNVGF
GYLSLLQVATFKGWMDIMYAAVDSRNVELQPKYEESLYMYLYFVIFIIFGSFFTLNLFIGVIID
NFNQQKKKFGGQDIFMTEEQKKYYNAMKKLGSKKPQKPIPRPGNKFQGMVEDEVTRQVFDISIM
ILICLNMVTMMVETDDQSEYVTTILSRINLVFIVLFTGECVLKLISLRHYYFTIGWNIFDFVVV
ILSIVGMFLAELIEKYFVSPTLFRVIRLARIGRILRLIKGAKGIRTLLFALMMSLPALFNIGLL
LFLVMFIYAIFGMSNFAYVKREVGIDDMFNFETFGNSMICLFQITTSAGWDGLLAPILNSKPPD
CDPNKVNPGSSVKGDCGNPSVGIFFFVSYIIISELVVVNMYIAVILENFSVATEESAEPLSEDD
FEMFYEVWEKFDPDATQFMEFEKLSQFAAALEPPLNLPQPNKLQLIAMDLPMVSGDRIHCLDIL
FAFTKRVLGESGEMDALRIQMEERFMASNPSKVSYQPITTTLKRKQEEVSAVIIQRAYRRHLLK
RTVKQASFTYNKNKIKGGANLLIKEDMIIDRINENSITEKTDLTMSTAACPPSYDRVTKPIVEK
HEQEGKDEKAKGK;
(SEQ ID NO: 867)
MEQTVLVPPGPDSFNFFTRESLAAIERRIAEEKAKNPKPDKKDDDENGPKPNSDLEAGKNLPFI
YGDIPPEMVSEPLEDLDPYYINKKTFIVLNKGKAIFRFSATSALYILTPFNPLRKIAIKILVHS
LFSMLIMCTILTNCVFMTMSNPPDWTKNVEYTFTGIYTFESLIKIIARGFCLEDFTFLRDPWNW
LDFTVITFAYVTEFVDLGNVSALRTFRVLRALKTISVIPGLKTIVGALIQSVKKLSDVMILTVF
CLSVFALIGLQLFMGNLRNKCIQWPPTNASLEEHSIEKNITVNYNGTLINETVFEFDWKSYIQD
SRYHYFLEGFLDALLCGNSSDAGQCPEGYMCVKAGRNPNYGYTSFDTFSWAFLSLFRLMTQDFW
ENLYQLTLRAAGKTYMIFFVLVIFLGSFYLINLILAVVAMAYEEQNQATLEEAEQKEAEFQQMI
EQLKKQQEAAQAATATASEHSREPSAAGRLSDSSSEASKLSSKSAKERRNRRKKRKQKEQSGGE
EKDEDEFQKSESEDSIRRKGFRFSIEGNRLTYEKRYSSPHQSLLSIRGSLFSPRRNSRTSLFSF
RGRAKDVGSENDFADDEHSTFEDNESRRDSLFVPRRHGERRNSNLSQTSRSSRMLAVFPANGKM
HSTVDCNGVVSLGTTTETEMRKRRSSSFHVSMDFLEDPSQRQRAMSIASILINTVEELEESRQK
CPPCWYKFSNIFLIWDCSPYWLKVKHVVNLVVMDPFVDLAITICIVLNTLFMAMEHYPMTDHFN
NVLTVGNLVFTGIFTAEMELKIIAMDPYYYFQEGWNIFDGFIVTLSLVELGLANVEGLSVLRSF
RLLRVFKLAKSWPTLNMLIKIIGNSVGALGNLTLVLAIIVFIFAVVGMQLFGKSYKDCVCKIAS
DCQLPRWHMNDFFHSFLIVFRVLCGEWIETMWDCMEVAGQAMCLTVFMMVMVIGNLVVLNLFLA
LLLSSFSADNLAATDDDNEMNNLQIAVDRMHKGVAYVKRKIYEFIQQSFIRKQKILDEIKPLDD
LNNKKDSCMSNHTAEIGKDLDYLKDVNGTTSGIGTGSSVEKYIIDESDYMSFINNPSLTVTVPI
AVGESDFENLNTEDFSSESDLEESKEKLNESSSSSEGSTVDIGAPVEEQPVVEPEETLEPEACF
TEGCVQRFKCCQINVEEGRGKQWWNLRRTCFRIVEHNWFETFIVFMILLSSGALAFEDIYIDQR
KTIKTMLEYADKVFTYIFILEMLLKWVAYGYQTYFTNAWCWLDFLIVDVSLVSLTANALGYSEL
GAIKSLRTLRALRPLRALSRFEGMRVVVNALLGAIPSIMNVLLVCLIFWLIFSIMGVNLFAGKF
YHCINTTTGDRFDIEDVNNHTDCLKLIERNETARWKNVKVNFDNVGFGYLSLLQVATFKGWMDI
MYAAVDSRNVELQPKYEESLYMYLYFVIFIIFGSFFTLNLFIGVIIDNFNQQKKKFGGQDIFMT
EEQKKYYNAMKKLGSKKPQKPIPRPGNKFQGMVFDFVTRQVEDISIMILICLNMVTMMVETDDQ
SEYVTTILSRINLVFIVLFTGECVLKLISLRHYYFTIGWNIFDFVVVILSIVGMFLAELIEKYF
VSPTLFRVIRLARIGRILRLIKGAKGIRTLLFALMMSLPALFNIGLLLFLVMFIYAIFGMSNFA
YVKREVGIDDMFNFETFGNSMICLFQITTSAGWDGLLAPILNSKPPDCDPNKVNPGSSVKGDCG
NPSVGIFFFVSYIIISFLVVVNMYIAVILENFSVATEESAEPLSEDDFEMFYEVWEKFDPDATQ
FMEFEKLSQFAAALEPPLNLPQPNKLQLIAMDLPMVSGDRIHCLDILFAFTKRVLGESGEMDAL
RIQMEERFMASNPSKVSYQPITTTLKRKQEEVSAVIIQRAYRRHLLKRTVKQASFTYNKNKIKG
GANLLIKEDMIIDRINENSITEKTDLTMSTAACPPSYDRVTKPIVEKHEQEGKDEKAKGK;
or
(SEQ ID NO: 868)
MFLKIIAMDPYYYFQEGWNIFDGFIVTLSLVELGLANVEGLSVLRSFRLLRVFKLAKSWPTLNM
LIKIIGNSVGALGNLTLVLAIIVFIFAVVGMQLFGKSYKDCVCKIASDCQLPRWHMNDFFHSFL
IVFRVLCGEWIETMWDCMEVAGQAMCLTVFMMVMVIGNLVVLNLFLALLLSSFSADNLAATDDD
NEMNNLQIAVDRMHKGVAYVKRKIYEFIQQSFIRKQKILDEIKPLDDLNNKKDSCMSNHTAEIG
KDLDYLKDVNGTTSGIGTGSSVEKYIIDESDYMSFINNPSLTVTVPIAVGESDFENLNTEDFSS
ESDLEESKEKLNESSSSSEGSTVDIGAPVEEQPVVEPEETLEPEACFTEGCVQRFKCCQINVEE
GRGKQWWNLRRTCFRIVEHNWFETFIVEMILLSSGALAFEDIYIDQRKTIKTMLEYADKVFTYI
FILEMLLKWVAYGYQTYFTNAWCWLDFLIVDVSLVSLTANALGYSELGAIKSLRTLRALRPLRA
LSRFEGMRVVVNALLGAIPSIMNVLLVCLIFWLIFSIMGVNLFAGKFYHCINTTTGDRFDIEDV
NNHTDCLKLIERNETARWKNVKVNFDNVGFGYLSLLQVATFKGWMDIMYAAVDSRNVELQPKYE
ESLYMYLYFVIFIIFGSFFTLNLFIGVIIDNFNQQKKKFGGQDIFMTEEQKKYYNAMKKLGSKK
PQKPIPRPGNKFQGMVFDFVTRQVFDISIMILICLNMVTMMVETDDQSEYVTTILSRINLVFIV
LFTGECVLKLISLRHYYFTIGWNIFDFVVVILSIVGMFLAELIEKYFVSPTLERVIRLARIGRI
LRLIKGAKGIRTLLFALMMSLPALFNIGLLLFLVMFIYAIFGMSNFAYVKREVGIDDMENFETF
GNSMICLFQITTSAGWDGLLAPILNSKPPDCDPNKVNPGSSVKGDCGNPSVGIFFFVSYIIISF
LVVVNMYIAVILENFSVATEESAEPLSEDDFEMFYEVWEKFDPDATQFMEFEKLSQFAAALEPP
LNLPQPNKLQLIAMDLPMVSGDRIHCLDILFAFTKRVLGESGEMDALRIQMEEREMASNPSKVS
YQPITTTLKRKQEEVSAVIIQRAYRRHLLKRTVKQASFTYNKNKIKGGANLLIKEDMIIDRINE
NSITEKTDLTMSTAACPPSYDRVTKPIVEKHEQEGKDEKAKGK.

The term “effective amount” as used herein refers to the amount of an active agent (e.g., tRNA or expression vector according to the present invention or a secondary active agent in a combination therapy) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein is administered in combination with one or more additional therapeutic agents, e.g., DIACOMIT® (stiripentol), EPIODOLEX® (cannabidiol), a ketogenic diet, ONFI® (clobazam), TOPAMAX® (topiramate), fenfluramine, or valproic acid. For example, during the treatment of Dravet Syndrome, a method or composition described herein is administered in combination with one or more additional therapeutic agents, e.g., DIACOMIT® (stiripentol), EPIODOLEX® (cannabidiol), a ketogenic diet, ONFI® (clobazam), TOPAMAX® (topiramate), fenfluramine, or valproic acid.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This Example describes an analysis of nonsense mutation frequency in patient populations.

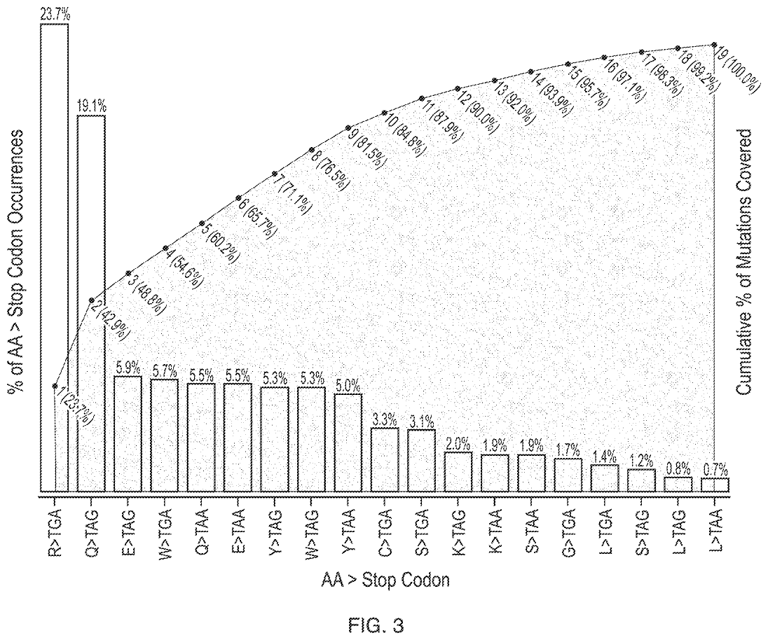

FIG. 3 is a plot depicting the relative share of each nonsense mutation based on global submissions to ClinVar that have been annotated as “pathogenic,” “likely pathogenic,” and “pathogenic/likely pathogenic” (dark columns). A cumulative density plot (light gray region) illustrates the fraction of the total patient population with disorders caused by nonsense mutations who could potentially be treated using combinations of suppressor tRNAs that target each nonsense mutation, starting with the most prevalent and progressing to the least prevalent.

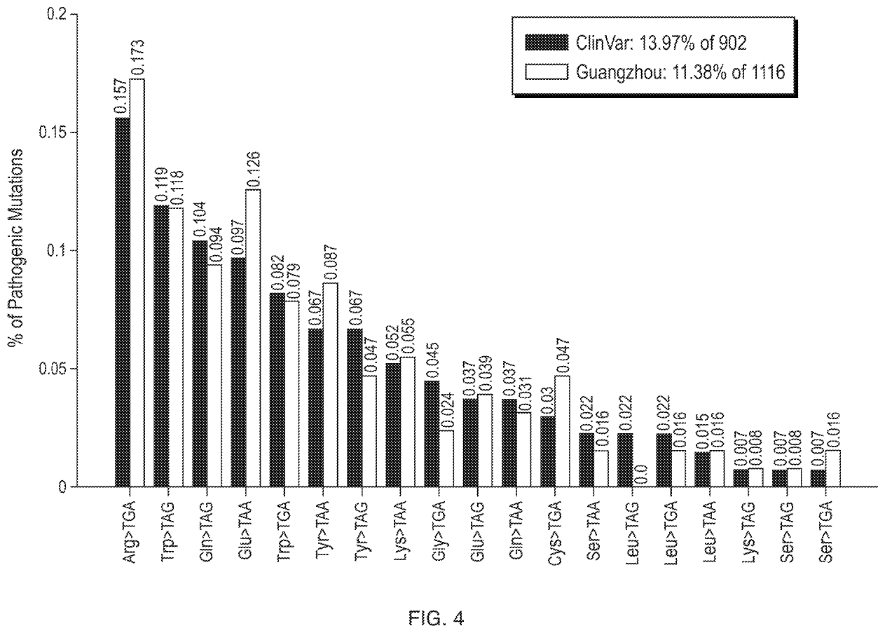

FIG. 4 is plot depicting the relative share of each potential nonsense mutation from SCN1A patient data found on ClinVar and the Guangzhou SCN1A mutation database. All Clin Var nonsense mutations annotated as “pathogenic,” “likely pathogenic,” or “pathogenic/likely pathogenic” are included. All Guangzhou database nonsense mutations tagged as “severe myoclonic epilepsy in infancy” are included.

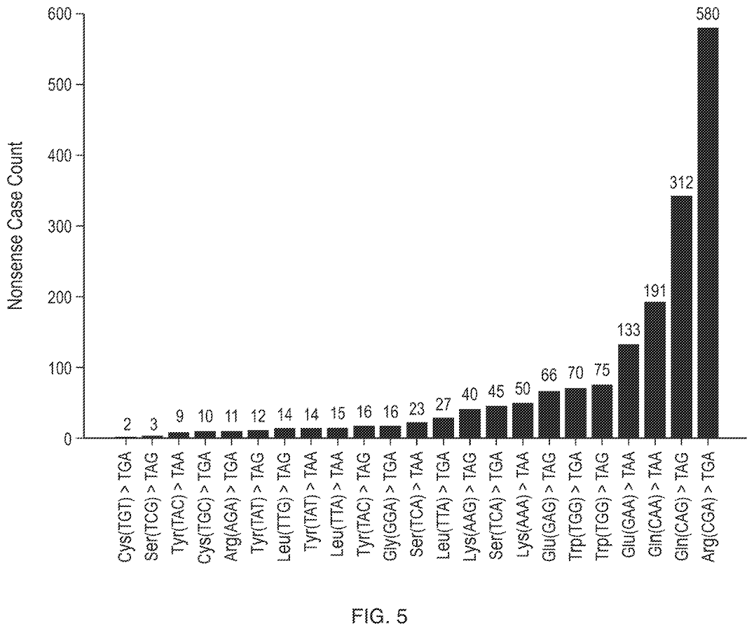

FIG. 5 is a plot depicting the breakdown of nonsense mutations tagged in human Duchenne muscular dystrophy (DMD) cases from the Leiden LOVD mutation database.

Together, the data provide a rationale for selecting combinations of two or three suppressor tRNAs that can be encoded on a single expression vector in order to maximize coverage of the total patient population with disorders caused by nonsense mutations.

### Example 2

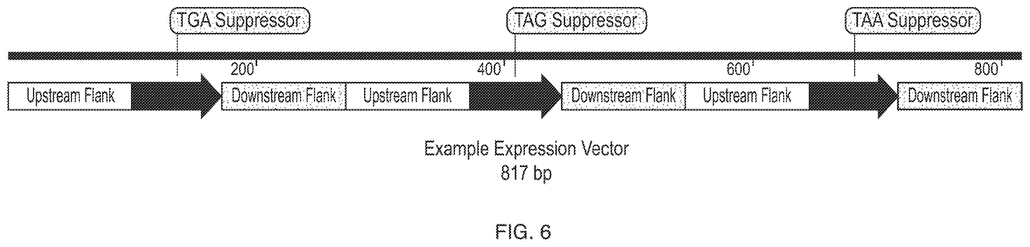

This Example describes the generation of an expression vector encoding three suppressor tRNAs that facilitate read-through of three different premature termination codons (PTC). A schematic representation of such an expression vector is shown in FIG. 6.

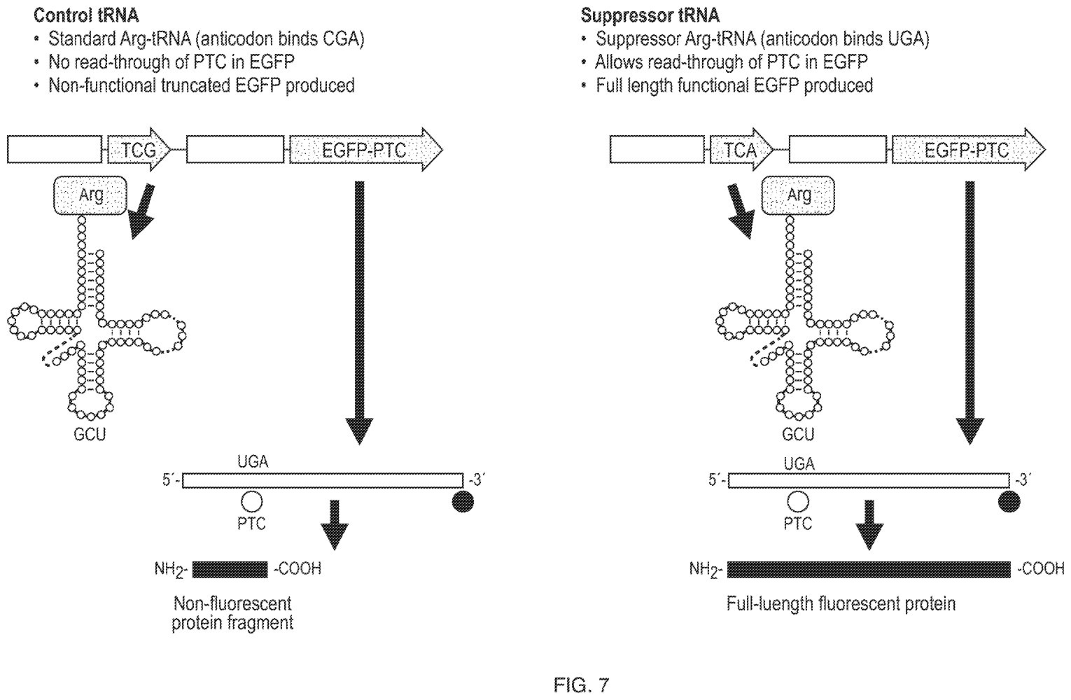

Readthrough activity of suppressor tRNAs was measured using constructs containing EGFP reporters with PTCs (TAG, TAA, or TAG) in place of various amino acid codons that are required for fluorescence. FIG. 7 depicts an exemplary EGFP reporter with a PTC (TGA) in place of an Arginine codon (CGA) and an accompanying suppressor tRNA. This approach can be generalized to create EGFP reporter constructs for other classes of suppressor tRNAs by converting an appropriate amino acid codon within the EGFP open reading frame to a termination codon.

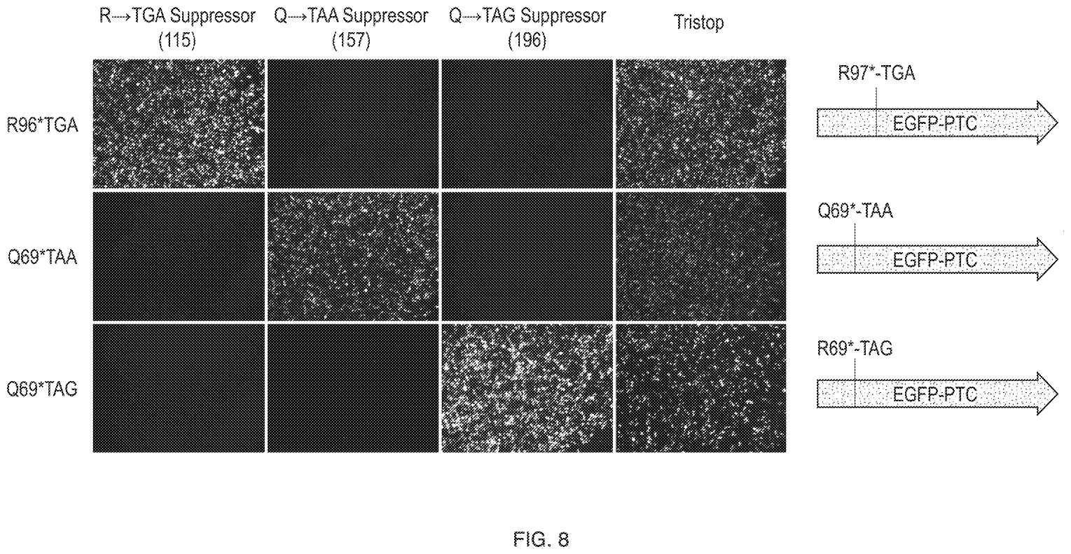

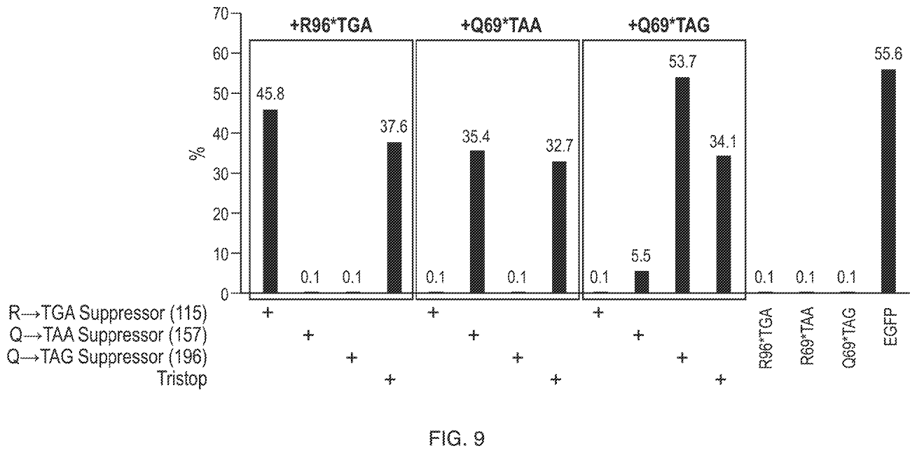

The activity of a single expression vector (designated the “Tristop” suppressor) encoding an Arginine to TGA (R>TGA) suppressor (including SEQ ID NO: 18), a Glutamine to TAA (Q>TAA) suppressor (including SEQ ID NO: 39), and a Glutamine to TAG (Q>TAG) suppressor (including SEQ ID NO: 178) was assessed in HEK293 cells transiently co-transfected with (i) a plasmid encoding the Tristop suppressor and (ii) a plasmid encoding either an EGFP reporter with a PTC (TGA) in place of an Arginine codon (CGA), an EGFP reporter with a PTC (TAA) in place of an Glutamine codon (CAG), or an EGFP reporter with a PTC (TAG) in place of an Glutamine codon (CAG). The readthrough activity of the Tristop suppressor was compared to the activity of separate expression vectors encoding the three individual suppressors included in the Tristop suppressor: an Arginine to TGA (R>TGA) suppressor only vector, a Glutamine to TAA (Q>TAA) suppressor only vector, and a Glutamine to TAG (Q>TAG) suppressor only vector. Transfections were done using the Lipofectamine 3000 Transfection Reagent according to the manufacturer's protocol. Co-transfections were done using equal amounts of the suppressor tRNA plasmid and the EGFP reporter plasmid. Results are shown in FIG. 8 (fluorescent images of EGFP reporter expression) and FIG. 9 (in which EGFP expression was analyzed by flow cytometry and readthrough activity is presented as the percentage of viable cells that express EGFP above background). As depicted, in each instance, the Tristop expression construct facilitated readthrough of the PTC.

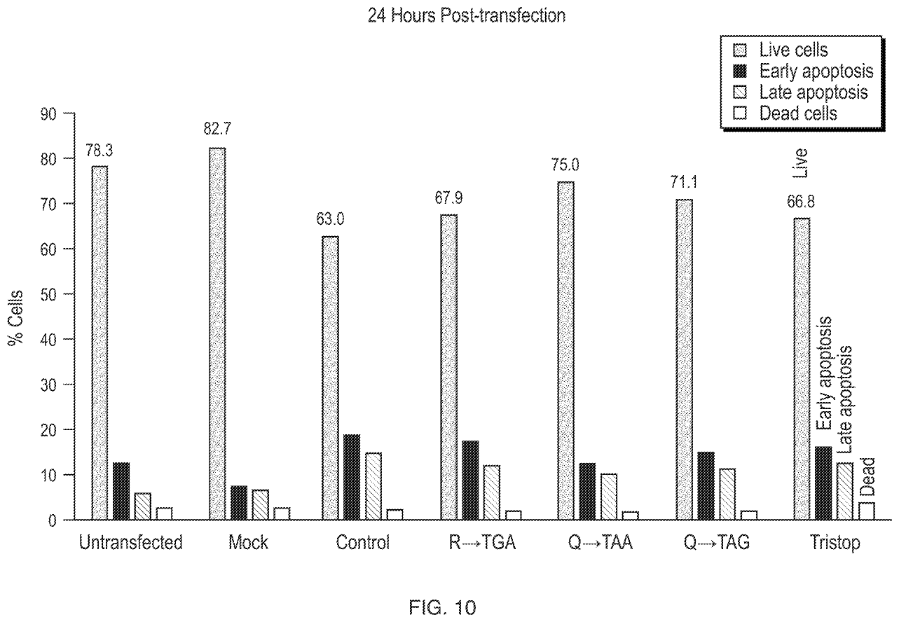

The effect of the Tristop suppressor on cell viability was compared to the effect of separate expression vectors comprising only an Arginine to TGA suppressor (“R→TGA”), only a Glutamine to TAA suppressor (“Q→TAA”), and only a Glutamine to TAG suppressor (“Q→TAG”). HEK293 cells were transiently transfected using the Lipofectamine 3000 Transfection Reagent according to the manufacturer's protocol and cell viability was assessed at 24 hours post-transfection using a Pacific Blue Annexin V/SYTOX AADvanced Apoptosis Kit (Thermofisher). This kit detects the externalization of phosphatidylserine in apoptotic cells using annexin V conjugated to violet-fluorescent Pacific Blue dye. Dead cells are detected using SYTOX AADvanced stain. After staining, apoptotic cells show violet fluorescence, dead cells show red fluorescence, and live cells show little or no fluorescence. Staining was performed according to the manufacturer's protocol and cells were assessed by flow cytometry. Results are shown in FIG. 10.

Together, the results demonstrate that Tristop suppressor tRNAs produce readthrough of nonsense mutations that is equivalent to expression vectors that comprise only single suppressor tRNAs. Additionally, the results show that treatment with Tristop suppressor tRNAs is not accompanied by a decrease in cell viability relative to individual suppressor tRNAs or control vectors.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1-63. (canceled)

64. An expression vector comprising:
(a) a first nucleotide sequence encoding a first suppressor tRNA that comprises an anticodon that hybridizes to a TGA premature stop codon, and is capable of being aminoacylated with a first amino acid;
(b) a second nucleotide sequence encoding a second suppressor tRNA that comprises an anticodon that hybridizes to a TAG premature stop codon, and is capable of being aminoacylated with a second amino acid; and
(c) a third nucleotide sequence encoding a third suppressor tRNA that comprises an anticodon that hybridizes to a TAA premature stop codon, and is capable of being aminoacylated with a third amino acid.

65. The expression vector of claim 64, wherein the expression vector comprises in a 5′ to 3′ orientation, the third nucleotide sequence, the first nucleotide sequence, and the second nucleotide sequence.

66. The expression vector of claim 64, wherein:
(a) the first amino acid:
(i) is selected from arginine, tryptophan, cysteine, serine, glycine, and leucine; and/or
(ii) is arginine;
(b) the second amino acid:
(i) is selected from glutamine, glutamic acid, tyrosine, tryptophan, lysine, serine, and leucine; and/or
(ii) is glutamine;
(c) the third amino acid:
(i) is selected from glutamine, glutamic acid, tyrosine, lysine, serine, and leucine; and/or
(ii) is glutamine;
(d) the second and third amino acid are the same; and/or
(e) the first amino acid is arginine, the second amino acid is glutamine, and the third amino acid is glutamine.

67. The expression vector of claim 64, wherein:
(a) the first, second, and/or third suppressor tRNA comprises a nucleotide sequence set forth in any one of SEQ ID NOs: 1-25, 35-81, 83-86, 88-89, 91-92, 94-95, 97-98, 100, 103, 105, 107-109, 112-113, 115-116, 118-119, 121-122, 124, 126-128, 130-132, 134-159, 161, 164, 167, 169-172, 178-190, 196-216, 218-221, 223-225, 228, 231, 234, 237, 240, 243, 245-248, 250-253, 255-261, 265, 270, 280-281, 285-862;
(b) the first amino acid is arginine and the first suppressor tRNA comprises a nucleotide sequence selected from any one of SEQ ID NOs: 18, 22, 6, 8, 9, 35, 17, 21, 20, 16, 19, 7, 11, 1, 2, 387, and 388;
(c) the second amino acid is glutamine and the second suppressor tRNA comprises a nucleotide sequence selected from any one of SEQ ID NOs: 181, 178, 187, 180, 179, 186, and 182; and/or
(d) the third amino acid is glutamine and the third suppressor tRNA comprises a nucleotide sequence selected from any one of SEQ ID NOs: 451, 457, 447, 39, 36, 45, 38, 37, 44, and 40.

68. The expression vector of claim 64, wherein:
(a) the first nucleotide sequence encoding the first suppressor tRNA that comprises an anticodon that hybridizes to a TGA premature stop codon is capable of being aminoacylated with arginine;
(b) the second nucleotide sequence encoding the second suppressor tRNA that comprises an anticodon that hybridizes to a TAG premature stop codon is capable of being aminoacylated with a glutamine; and
(c) the third nucleotide sequence encoding the third suppressor tRNA that comprises an anticodon that hybridizes to a TAA premature stop codon is capable of being aminoacylated with glutamine,
wherein the expression vector comprises in a 5′ to 3′ orientation, the third nucleotide sequence, the first nucleotide sequence, and the second nucleotide sequence.

69. The expression vector of claim 64, wherein:
(a) the first suppressor tRNA comprises a nucleotide sequence selected from any one of SEQ ID NOs: 18, 22, 6, 8, 9, 35, 17, 21, 20, 16, 19, 7, 11, 1, 2, 387, and 388;
(b) the second suppressor tRNA comprises a nucleotide sequence selected from any one of SEQ ID NOs: 181, 178, 187, 180, 179, 186, and 182; and/or
(c) third suppressor tRNA comprises a nucleotide sequence selected from any one of SEQ ID NOs: 451, 457, 447, 39, 36, 45, 38, 37, 44, and 40.

70. The expression vector of claim 64, wherein the expression vector comprises 1, 2, 3, 4, or more than 4 copy numbers of the nucleotide sequence encoding the first, second, and/or third suppressor tRNA.

71. The expression vector of claim 64, wherein:
(a) the expression vector further comprises a nucleotide sequence set forth in any one of SEQ ID NOs: 26-27, 32-34, 173-174, 869-888, 895-897, and 900;
(b) the expression vector comprises a nucleotide sequence selected from any one of SEQ ID NOs: 869-888;
(c) the first nucleotide sequence, the second nucleotide sequence, and the third nucleotide sequence are each operably linked to a nucleotide sequence set forth in any one of SEQ ID NOs: 26-27, 32-34, 173-174, 869-888, 895-897, and 900; and/or
(d) the first nucleotide sequence, the second nucleotide sequence, and the third nucleotide sequence are each operably linked to a nucleotide sequence selected from any one of SEQ ID NOs: 869-888.

72. The expression vector of claim 64, wherein the expression vector comprises a viral vector, a DNA virus vector, and an adeno-associated virus (AAV) vector.

73. A pharmaceutical composition comprising the expression vector of claim 64 and a pharmaceutically acceptable excipient.

74. The pharmaceutical composition of claim 73, wherein the first, second, and/or third suppressor tRNA comprises a naturally occurring nucleotide modification and/or the first, second, and/or third suppressor tRNA comprises one or more nucleotide modifications selected from 5-methyl uridine, 5-carbamoylmethyluridine, 5-carbamoyl-methyl-2-O-methyluridine, 5-methoxy-carbonylmethyluridine, 5-methoxycarbonylmethyl-2-thiouridine, pseudouridine, dihydrouridine, 1-methyladenosine, and inosine.

75. The pharmaceutical composition of claim 73, wherein:
(a) the expression vector or tRNA is not conjugated to or associated with another moiety or a carrier particle; and/or
(b) the composition does not comprise an aminolipid particle, an aminolipid delivery compound, or a nanoparticle.

76. A method of expressing in a mammalian cell a functional gene product encoded by a gene containing a premature termination codon, the method comprising contacting the cell with an effective amount of the pharmaceutical composition of claim 73, thereby permitting an amino acid to be incorporated into the gene product at a position that would otherwise result in a truncated gene product caused by the premature termination codon.

77. The method of claim 76, wherein the gene:
(a) is a gene selected from the group consisting of SCN1A, KCNQ2, SCN2A, CDKL5, MECP2, STXBP1, SCN8A, CACNAIA, SLC2A1, FOXG1, PCDH19, GRIN2B, DEPDC5, GRIN2A, CHD2, SCN9A, SYNGAP1, ALDH7A1, GRIN1, TBC1D24, SLC6A1, DNM1, ARX, KCNB1, KCNA1, GABRG2, WWOX, GABRB3, SZT2, LGI1, PNPO, SCNIB, UBA5, KCTD7, SCARB2, SLC13A5, CSTB, EPM2A, PRRT2, NHLRC1, SLC25A22, PPRT2, ALG13, β-globin, CHM, CFTR, dystrophin, α-L-iduronidase, KIF1A, FBN1, ARSB, SMPD1, NAGLU, DHCR7, SCN5A, KCNH2 (hERG), KCNQ1, TTN, MYBPC3, LMNA, PKP2, PLN, TSC1/2, LDLR, and SMN1;
(b) is a gene selected from the group consisting of SCN1A, KCNQ2, SCN2A, CDKL5, MECP2, STXBP1, SCN8A, CACNAIA, SLC2A1, FOXG1, PCDH19, GRIN2B, DEPDC5, GRIN2A, CHD2, SCN9A, SYNGAP1, ALDH7A1, GRIN1, TBC1D24, SLC6A1, DNM1, ARX, KCNB1, KCNA1, GABRG2, WWOX, GABRB3, SZT2, LGI1, PNPO, SCNIB, UBA5, KCTD7, SCARB2, SLC13A5, CSTB, EPM2A, PRRT2, NHLRC1, SLC25A22, PPRT2, or ALG13; and/or
(c) is SCN1A or dystrophin.

78. The method of claim 76, wherein:
(a) the cell is a human cell; and/or
(b) the tRNA becomes aminoacylated in the cell.

79. A method of treating a premature termination codon-mediated disorder in a subject in need thereof wherein the subject has a gene with a first, second, and/or third premature termination codon, the method comprising administering to the subject an effective amount of the pharmaceutical composition of claim 73, thereby to treat the disorder in the subject.

80. A method of treating a premature termination codon-mediated disorder in a subject in need thereof wherein the subject has a gene with a first, second, and/or third premature termination codon, the method comprising administering to the subject an effective amount of an expression vector comprising:
(a) a first nucleotide sequence encoding a first suppressor tRNA that comprises an anticodon that hybridizes to a TGA premature stop codon, and is capable of being aminoacylated with a first amino acid;
(b) a second nucleotide sequence encoding a second suppressor tRNA that comprises an anticodon that hybridizes to a TAG premature stop codon, and is capable of being aminoacylated with a second amino acid; and
(c) a third nucleotide sequence encoding a third suppressor tRNA that comprises an anticodon that hybridizes to a TAA premature stop codon, and is capable of being aminoacylated with a third amino acid,
wherein the expression vector comprises in a 5′ to 3′ orientation, the third nucleotide sequence, the first nucleotide sequence, and the second nucleotide sequence.

81. The method of claim 79, wherein:
(a) the first, second, and/or third suppressor tRNA comprises a nucleotide sequence set forth in any one of SEQ ID NOs: 1-25, 35-81, 83-86, 88-89, 91-92, 94-95, 97-98, 100, 103, 105, 107-109, 112-113, 115-116, 118-119, 121-122, 124, 126-128, 130-132, 134-159, 161, 164, 167, 169-172, 178-190, 196-216, 218-221, 223-225, 228, 231, 234, 237, 240, 243, 245-248, 250-253, 255-261, 265, 270, 280-281, 285-862;
(b) the first amino acid is arginine and the first suppressor tRNA comprises a nucleotide sequence selected from SEQ ID NOs: 18, 22, 6, 8, 9, 35, 17, 21, 20, 16, 19, 7, 11, 1, 2, 387, and 388;
(c) the second amino acid is glutamine and the second suppressor tRNA comprises a nucleotide sequence selected from SEQ ID NOs: 181, 178, 187, 180, 179, 186, and 182; and/or
(d) the third amino acid is glutamine and the third suppressor tRNA comprises a nucleotide sequence selected from SEQ ID NOs: 451, 457, 447, 39, 36, 45, 38, 37, 44, and 40.

82. The method of claim 80, wherein the disorder:
(a) is a disorder selected from the group consisting of Dravet Syndrome, Genetic Epilepsy with Febrile Seizures (GEFS), Benign Familial Infantile Epilepsy (BFIE); Early Infantile Epileptic Encephalopathy (EIEE), Lennox-Gastaut Syndrome, CDKL5 deficiency disorder, Rett Syndrome, PPM-X Syndrome, Ohtahara Syndrome, Episodic Ataxia, Hemiplegic Migraine, Iditiopathic Generalized Epilepsy, FOXG1 Syndrome, Familial Focal Epilepsy with Variable Foci (FFEVF), Childhood-onset epileptic encephalopathy, Congenital insensitivity to pain, SYNGAP1-related intellectual disability, Pyridoxine-dependent epilepsy, Familial Infantile Myoclonic Epilepsy (FUME), Myoclonic Astatic Epilepsy, X-linked Intellectual Disability, Partial Epilepsy and Episodic Ataxia, Febrile seizures, Autosomal Dominant Partial Epilepsy with Auditory Features (ADPEAF), PNPO-Deficiency, Action Myoclonus-Renal Failure (AMRF), Progressive Myoclonic Epilepsy, Benign Familial Infantile Seizures (BFIS), β-thalassemia, Choroideremia, Cystic Fibrosis, Duchenne Muscular Dystrophy, Hurler Syndrome, KIFIA, Marfan Syndrome, Maroteaux-Lamy Syndrome, Niemann Pick Disease, Sanfilippo Syndrome, Smith-Lemli-Opitz Syndrome, Brugada Syndrome, Long QT Syndrome type 1, Long QT Syndrome type 2, Dilated Cardiomyopathy, Familial Hypertrophic Cardiomyopathy, Emery-Dreifuss Muscular Dystrophy, Familial Arrythmogenic Right Ventricular Dysplasia, Familial Isolated Dilated Cardiomyopathy, Tuberous Sclerosis, Familial Hypercholesterolemia, or Spinal Muscular Atrophy;
(b) is a disorder is selected from the group consisting of Dravet Syndrome, Genetic Epilepsy with Febrile Seizures (GEFS), Benign Familial Infantile Epilepsy (BFIE); Early Infantile Epileptic Encephalopathy (EIEE), Lennox-Gastaut Syndrome, CDKL5 deficiency disorder, Rett Syndrome, PPM-X Syndrome, Ohtahara Syndrome, Episodic Ataxia, Hemiplegic Migraine, Iditiopathic Generalized Epilepsy, FOXG1 Syndrome, Familial Focal Epilepsy with Variable Foci (FFEVF), Childhood-onset epileptic encephalopathy, Congenital insensitivity to pain, SYNGAP1-related intellectual disability, Pyridoxine-dependent epilepsy, Familial Infantile Myoclonic Epilepsy (FUME), Myoclonic Astatic Epilepsy, X-linked Intellectual Disability, Partial Epilepsy and Episodic Ataxia, Febrile seizures, Autosomal Dominant Partial Epilepsy with Auditory Features (ADPEAF), PNPO-Deficiency, Action Myoclonus-Renal Failure (AMRF), Progressive Myoclonic Epilepsy, Benign Familial Infantile Seizures (BFIS); and/or
(c) is Dravet Syndrome or Duchenne Muscular Dystrophy.

83. The method of claim 80, wherein the subject is a human.
